(19) United States
(12) Patent Application Publication
(10) Pub. No.: US 2013/0184270 A1
(43) Pub. Date: Jul. 18, 2013

(54) SUBSTITUTED 2,3-DIHYDROIMIDAZO[1,2-C]QUINAZOLINE-CONTAINING COMBINATIONS

(71) Applicant: Ningshu Liu, Berlin (DE)

(72) Inventor: Ningshu Liu, Berlin (DE)

(73) Assignee: BAYER INTELLECTUAL PROPERTY GMBH, MONHEIM (DE)

(21) Appl. No.: 13/640,994

(22) PCT Filed: Apr. 14, 2011
(86) PCT No.: PCT/EP2011/055917

§ 371 Date: Mar. 13, 2013

(30) Foreign Application Priority Data
Apr. 16, 2010 (EP) 10160109.4

(51) Int. Cl. A61K 31/519; A61K 31/18; A61K 31/5377

(52) U.S. Cl. CPC A61K 31/519; A61K 31/5377; A61K 31/18
USPC 514/232.5; 514/267; 514/233.2; 514/252.16; 514/255.05

(57) ABSTRACT

The present invention relates to: combinations of: component A: one or more 2,3-dihydroimidazo[1,2-c]quinazoline compounds of general formula (A1) or (A2), or a physiologically acceptable salt, solvate, hydrate or stereoisomer thereof; component B: one or more N-(2-arylamino)aryl sulfonamide compounds of general formula (B), or Lapatinib, or Paclitaxel, or a physiologically acceptable salt, solvate, hydrate or stereoisomer thereof; and, optionally, component C: one or more further pharmaceutical agents; in which optionally some or all of the components are in the form of a pharmaceutical formulation which is ready for use to be administered simultaneously, concurrently, separately or sequentially. dependently of one another by the oral, intravenous, topical, local installations, intraperitoneal or nasal route; use of such combinations for the preparation of a medicament for the treatment or prophylaxis of a cancer; and a kit comprising such a combination.

The present invention relates:
to combinations of:
   component A: one or more 2,3-dihydroimidazo[1,2-c]quinazoline compounds of general formula (A1) or (A2), or a physiologically acceptable salt, solvate, hydrate or stereoisomer thereof;
   component B: one or more N-(2-arylamino)aryl sulfonamide compounds of general formula (B), or a physiologically acceptable salt, solvate, hydrate or stereoisomer thereof; and, optionally,
   component C: one or more further pharmaceutical agents;
to combinations of:
   component A: one or more 2,3-dihydroimidazo[1,2-c]quinazoline compounds of general formula (A1) or (A2), or a physiologically acceptable salt, solvate, hydrate or stereoisomer thereof;
   component B: N-[3-chloro-4-[(3-fluorophenyl)methoxy]phenyl]-6-[5-[(2-methyl-sulfonylethylamino)methyl]-2-furyl]quinazolin-4-amine (hereinafter referred to as Lapatinib); and, optionally,
   component C: one or more further pharmaceutical agents;
and to combinations of:
   component A: one or more 2,3-dihydroimidazo[1,2-c]quinazoline compounds of general formula (A1) or (A2), or a physiologically acceptable salt, solvate, hydrate or stereoisomer thereof;
   component B: 5β,20-Epoxy-1,2α,4,7β,10β,13α-hexahydroxytax-11-en-9-one 4,10-diacetate 2-benzoate 13-ester with (2R,3S)—N-benzoyl-3-phenylisoserine (hereinafter referred to as paclitaxel); and, optionally,
   component C: one or more further pharmaceutical agents;
   in which optionally either or both of components A and B in any of the above-mentioned combinations are in the form of a pharmaceutical formulation which is ready for use to be administered simultaneously, concurrently, separately or sequentially. The components may be administered independently of one another by the oral, intravenous, topical, local installations, intraperitoneal or nasal route.

Another aspect of the present invention relates to the use of such combinations as described supra for the preparation of a medicament for the treatment or prophylaxis of a cancer, particularly lung cancer, in particular non-small cell lung carcinoma (abbreviated to and hereinafter referred to as “NSCLC”), colorectal cancer (abbreviated to and hereinafter referred to as “CRC”), melanoma, pancreatic cancer, hepatocyte carcinoma, pancreatic cancer, hepatocyte carcinoma or breast cancer.

Further, the present invention relates to:

a kit comprising:
a combination of:
   component A: one or more 2,3-dihydroimidazo[1,2-c]quinazoline compounds of general formula (A1) or (A2), or a physiologically acceptable salt, solvate, hydrate or stereoisomer thereof;
   component B: one or more N-(2-arylamino)aryl sulfonamide compounds of general formula (B), or a physiologically acceptable salt, solvate, hydrate or stereoisomer thereof; and, optionally,
   component C: one or more further pharmaceutical agents;
or a combination of:
   component A: one or more 2,3-dihydroimidazo[1,2-c]quinazoline compounds of general formula (A1) or (A2), or a physiologically acceptable salt, solvate, hydrate or stereoisomer thereof;
   component B: Lapatinib; and, optionally,
   component C: one or more further pharmaceutical agents;
or a combination of:
   component A: one or more 2,3-dihydroimidazo[1,2-c]quinazoline compounds of general formula (A1) or (A2), or a physiologically acceptable salt, solvate, hydrate or stereoisomer thereof;
   component B: Paclitaxel; and, optionally,
   component C: one or more further pharmaceutical agents;
   in which optionally either or both of said components A) and B) in any of the above-mentioned combinations are in the form of a pharmaceutical formulation which is ready for use to be administered simultaneously, concurrently, separately or sequentially. The components may be administered independently of one another by the oral, intravenous, topical, local installations, intraperitoneal or nasal route.

### BACKGROUND OF THE INVENTION

### Combinations of PI3K Inhibitors and MEK Inhibitors:

Deregulated activation of protein kinases, such as the epidermal growth factor receptor (EGFR), and downstream signalling kinases (PI3K and MAPK pathways) are associated with human cancers. Although inhibitors of such activated kinases have proved to be of therapeutic benefit in individuals, some patients manifest intrinsic or acquired resistance to these drugs. Developing new agents or novel combination therapies, clearly represents therefore a long-felt need to overcome this intrinsic and acquired drug resistance.

Recent insights into the molecular pathogenesis of CRC and NSCLC have given rise to specific target-directed therapies, including kinase inhibitors and monoclonal antibodies (mAb) against epidermal growth factor receptor (EGFR) and vascular endothelial growth factor (VEGF). Activating mutations of KRAS and BRAF genes are genetic events in tumorigenesis and these mutations are implicated as negative predictive factors in determining response to anti-EGFR drugs. Additional data suggest that other EGFR downstream molecules such as PI3K/PTEN/AKT are also important when considering mechanisms of EGFR antibody resistance.

Unexpectedly, and this represents a basis of the present invention, when combinations of:
component A: a 2,3-dihydroimidazo[1,2-c]quinazoline compound of general formula (A1) or (A2), or a physiologically acceptable salt, solvate, hydrate or stereoisomer thereof, as described and defined herein; with
component B: which is:
   either an N-(2-arylamino)aryl sulfonamide compound of general formula (B), or a physiologically acceptable salt, solvate, hydrate or stereoisomer thereof, as described and defined herein;
   or N-[3-chloro-4-[(3-fluorophenyl)methoxy]phenyl]-6-[5-[(2-methyl-sulfonylethylamino)methyl]-2-furyl]quinazolin-4-amine:

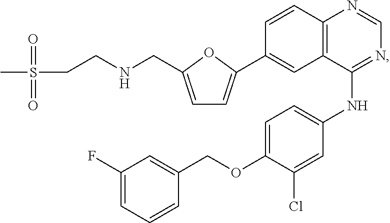 [SMILES: CS(=O)(=O)CCNCc1ccc(-c2ccc3ncnc(Nc4ccc(OCc5cccc(F)c5)c(Cl)c4)c3c2)o1]

(which is herein referred to as Lapatinib);
or 5β,20-Epoxy-1,2α,4,7β,10β,13α-hexahydroxytax-1′-en-9-one 4,10-diacetate 2-benzoate 13-ester with (2R,3S)—N-benzoyl-3-phenylisoserine (which is hereinafter referred to as Paclitaxel);
   were evaluated for the treatment of CRC, NSCLC, pancreatic cancer, hepatocyte carcinoma and breast cancer, synergistically increased anti-tumor activities were demonstrated with these combinations compared to each monotherapy, providing a fundamental rationale for the clinical combination therapy using PI3K inhibitors-MEK inhibitors, PI3K inhibitors-Lapatinib or PI3K inhibitors-Paclitaxel.

The same mechanism and rationale can also be applied in other cancer indications with genetic alteration in RTKs, RAS/RAF/MEK and PI3K/PTEN/AKT pathway molecules, or with activation of in RTKs, RAS/RAF/MEK and PI3K/PTEN/AKT pathway molecules through other mechanisms.

To the Applicant's knowledge, no generic or specific disclosure or suggestion in the prior art is known that either combinations of:

component A: one or more 2,3-dihydroimidazo[1,2-c]quinazoline compounds of general formula (A1) or (A2), or a physiologically acceptable salt, solvate, hydrate or stereoisomer thereof;
component B: one or more N-(2-arylamino)aryl sulfonamide compounds of general formula (B), or a physiologically acceptable salt, solvate, hydrate or stereoisomer thereof; and, optionally,
component C: one or more further pharmaceutical agents;
or combinations of:
component A: one or more 2,3-dihydroimidazo[1,2-c]quinazoline compounds of general formula (A1) or (A2), or a physiologically acceptable salt, solvate, hydrate or stereoisomer thereof;
component B: Lapatinib; and, optionally,
component C: one or more further pharmaceutical agents;
or combinations of:
component A: one or more 2,3-dihydroimidazo[1,2-c]quinazoline compounds of general formula (A1) or (A2), or a physiologically acceptable salt, solvate, hydrate or stereoisomer thereof;
component B: Paclitaxel; and, optionally,
component C: one or more further pharmaceutical agents;
in which optionally either or both of said components A and B of any of the above-mentioned combinations are in the form of a pharmaceutical formulation which is ready for use to be administered simultaneously, concurrently, separately or sequentially, would be effective in the treatment or prophylaxis of cancer, particularly NSCLC, CRC, melanoma, pancreatic cancer, hepatocyte carcinoma or breast cancer.

Based on the action of the testing compounds described in this invention, the combinations of the present invention as described and defined herein, show a beneficial effect in the treatment of cancer, particularly NSCLC, CRC, melanoma, pancreatic cancer, hepatocyte carcinoma or breast cancer.

Accordingly, in accordance with a first aspect, the present invention relates: to combinations of:

component A: one or more 2,3-dihydroimidazo[1,2-c]quinazoline compounds of general formula (A1) or (A2), or a physiologically acceptable salt, solvate, hydrate or stereoisomer thereof;
component B: one or more N-(2-arylamino)aryl sulfonamide compounds of general formula (B), or a physiologically acceptable salt, solvate, hydrate or stereoisomer thereof; and, optionally,
component C: one or more further pharmaceutical agents; to combinations of:
component A: one or more 2,3-dihydroimidazo[1,2-c]quinazoline compounds of general formula (A1) or (A2), or a physiologically acceptable salt, solvate, hydrate or stereoisomer thereof;
component B: Lapatinib; and, optionally,
component C: one or more further pharmaceutical agents; and to combinations of:
component A: one or more 2,3-dihydroimidazo[1,2-c]quinazoline compounds of general formula (A1) or (A2), or a physiologically acceptable salt, solvate, hydrate or stereoisomer thereof;
component B: Paclitaxel; and, optionally,
component C: one or more further pharmaceutical agents;
in which optionally either or both of said components A and B) of any of the above-mentioned combinations are in the form of a pharmaceutical formulation which is ready for use to be administered simultaneously, concurrently, separately or sequentially. The components may be administered independently of one another by the oral, intravenous, topical, local installations, intraperitoneal or nasal route.

In accordance with a second aspect, of the present invention relates to the use of any of such combinations as described supra for the preparation of a medicament for the treatment or prophylaxis of a cancer, particularly NSCLC, CRC, melanoma, pancreatic cancer, hepatocyte carcinoma or breast cancer.

Further, in accordance with a third aspect, the present invention relates to a kit comprising:

a combination of:
component A: one or more 2,3-dihydroimidazo[1,2-c]quinazoline compounds of general formula (A1) or (A2), or a physiologically acceptable salt, solvate, hydrate or stereoisomer thereof;
component B: one or more N-(2-arylamino)aryl sulfonamide compounds of general formula (B), or a physiologically acceptable salt, solvate, hydrate or stereoisomer thereof; and, optionally,
component C: one or more further pharmaceutical agents;
or a combination of:
component A: one or more 2,3-dihydroimidazo[1,2-c]quinazoline compounds of general formula (A1) or (A2), or a physiologically acceptable salt, solvate, hydrate or stereoisomer thereof;
component B: Lapatinib; and, optionally,
component C: one or more further pharmaceutical agents;
or a combination of:
component A: one or more 2,3-dihydroimidazo[1,2-c]quinazoline compounds of general formula (A1) or (A2), or a physiologically acceptable salt, solvate, hydrate or stereoisomer thereof;
component B: Paclitaxel; and, optionally,
component C: one or more further pharmaceutical agents;
in which optionally either or both of components A and B in any of the above-mentioned combinations are in the form of a pharmaceutical formulation which is ready for use to be administered simultaneously, concurrently, separately or sequentially. The components may be administered independently of one another by the oral, intravenous, topical, local installations, intraperitoneal or nasal route.

### DETAILED DESCRIPTION OF THE INVENTION

In accordance with an embodiment of the above-mentioned aspects of the present invention, said combinations are of:

component A: which is one or more 2,3-dihydroimidazo[1,2-c]quinazoline compounds of general formula (A1):

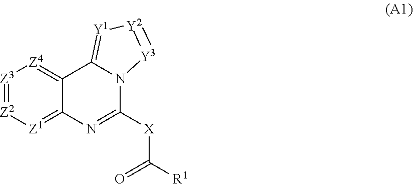

wherein
X represents CR⁵R⁶ or NH;
Y¹ represents CR³ or N;
Chemical bond between [symbol] represents a single bond or double bond,
with the proviso that when the [symbol] represents a double bond,
Y² and Y³ independently represent CR⁴ or N, and
when [symbol] represents a single bond, Y² and Y³ independently represent CR³R⁴ or NR⁴;
Z¹, Z², Z³ and Z⁴ independently represent CH, CR² or N;
R¹ represents aryl optionally having 1 to 3 substituents selected from R¹¹, C₃₋₈ cycloalkyl optionally having 1 to 3 substituents selected from R¹¹, C₁₋₆ alkyl optionally substituted by
   aryl, heteroaryl, C₁₋₆ alkoxyaryl, aryloxy, heteroaryloxy or one or more halogen,
   C₁₋₆ alkoxy optionally substituted by
   carboxy, aryl, heteroaryl, C₁₋₆ alkoxyaryl, aryloxy, heteroaryloxy or one or more halogen,
   or
   a 3 to 15 membered mono- or bi-cyclic heterocyclic ring that is saturated or unsaturated, and contains 1 to 3 heteroatoms selected from the group consisting of N, O and S, and optionally having 1 to 3 substituents selected from R¹¹
   wherein
   R¹¹ represents
   halogen, nitro, hydroxy, cyano, carboxy, amino, N—(C₁₋₆alkyl)amino, N-(hydroxyC₁₋₆alkyl)amino, N,N-di(C₁₋₆alkyl)amino, N—(C₁₋₆acyl)amino, N-(formyl)-N—(C₁₋₆alkyl)amino, N—(C₁₋₆alkanesulfonyl)amino, N-(carboxyC₁₋₆₋alkyl)-N—(C₁₋₆alkyl)amino, N—(C₁₋₆alkoxycarbonyl)amino, N—[N,N-di(C₁₋₆alkyl)amino methylene]amino, N—[N,N-di(C₁₋₆alkyl)amino (C₁₋₆ alkyl)methylene]amino, N—[N,N-di(C₁₋₆alkyl)amino C₂₋₆alkenyl]amino, aminocarbonyl, N—(C₁₋₆alkyl)aminocarbonyl, N,N-di(C₁₋₆alkyl)aminocarbonyl, C₃₋₈cycloalkyl, C₁₋₆ alkylthio, C₁₋₆alkanesulfonyl, sulfamoyl, C₁₋₆alkoxycarbonyl,
   N-arylamino wherein said aryl moiety is optionally having 1 to 3 substituents selected from R¹⁰¹, N-(aryl C₁₋₆alkyl)amino wherein said aryl moiety is optionally having 1 to 3 substituents selected from R¹⁰¹, aryl C₁₋₆alkoxycarbonyl wherein said aryl moiety is optionally having 1 to 3 substituents selected from R¹⁰¹,
   C₁₋₆alkyl optionally substituted by
   mono-, di- or tri-halogen, amino, N—(C₁₋₆alkyl)amino or N,N-di(C₁₋₆alkyl)amino,
   C₁₋₆alkoxy optionally substituted by
   mono-, di- or tri-halogen, N—(C₁₋₆alkyl)sulfonamide, or N-(aryl)sulfonamide,
   or
   a 5 to 7 membered saturated or unsaturated ring having 1 to 3 heteroatoms selected from the group consisting of O, S and N, and optionally having 1 to 3 substituents selected from R¹⁰¹
   wherein
   R¹⁰¹ represents
   halogen, carboxy, amino, N—(C₁₋₆ alkyl)amino, N,N-di(C₁₋₆alkyl)amino, aminocarbonyl, N—(C₁₋₆alkyl)aminocarbonyl, N,N-di(C₁₋₆alkyl)amino-carbonyl, pyridyl,
   C₁₋₆ alkyl optionally substituted by cyano or mono- di- or tri-halogen,
   or
   C₁₋₆alkoxy optionally substituted by cyano, carboxy, amino, N—(C₁₋₆ alkyl)amino, N,N-di(C₁₋₆alkyl)amino, aminocarbonyl, N—(C₁₋₆alkyl)amino-carbonyl, N,N-di(C₁₋₆alkyl)aminocarbonyl or mono-, di- or tri-halogen;
R² represents hydroxy, halogen, nitro, cyano, amino, N—(C₁₋₆alkyl)amino, N,N-di(C₁₋₆alkyl)amino, N-(hydroxyC₁₋₆alkyl)amino, N-(hydroxyC₁₋₆alkyl)-N—(C₁₋₆alkyl)amino, C₁₋₆ acyloxy, aminoC₁₋₆ acyloxy, C₂₋₆alkenyl, aryl,
   a 5-7 membered saturated or unsaturated heterocyclic ring having 1 to 3 heteroatoms selected from the group consisting O, S and N, and optionally substituted by
   hydroxy, C₁₋₆ alkyl, C₁₋₆ alkoxy, oxo, amino, amino C₁₋₆alkyl, N—(C₁₋₆alkyl)amino, N,N-di(C₁₋₆alkyl)amino, N—(C₁₋₆ acyl)amino, N—(C₁₋₆alkyl)carbonylamino, phenyl, phenyl C₁₋₆ alkyl, carboxy, C₁₋₆alkoxycarbonyl, aminocarbonyl, N—(C₁₋₆alkyl)aminocarbonyl, or N,N-di(C₁₋₆alkyl)amino,
   —C(O)—R²⁰
   wherein
   R²⁰ represents C₁₋₆ alkyl, C₁₋₆ alkoxy, amino, N—(C₁₋₆alkyl)amino, N,N-di(C₁₋₆alkyl)amino, N—(C₁₋₆ acyl)amino, or a 5-7 membered saturated or unsaturated heterocyclic ring having 1 to 3 heteroatoms selected from the group consisting O, S and N, and optionally substituted by
      C₁₋₆ alkyl, C₁₋₆ alkoxy, oxo, amino, N—(C₁₋₆alkyl)amino, N,N-di(C₁₋₆alkyl)amino, N—(C₁₋₆ acyl)amino, phenyl, or benzyl,
   C₁₋₆ alkyl optionally substituted by R²¹
   or
   C₁₋₆ alkoxy optionally substituted by R²¹
   wherein
   R²¹ represents cyano, mono-, di or tri-halogen, hydroxy, amino, N—(C₁₋₆alkyl)amino, N,N-di(C₁₋₆alkyl)amino, N-(hydroxyC₁₋₆ alkyl)amino, N-(halophenylC₁₋₆ alkyl)amino, amino C₂₋₆ alkylenyl, C₁₋₆ alkoxy, hydroxyC₁₋₆ alkoxy, —C(O)—R²⁰¹, —NHC(O)—R²⁰¹, C₃₋₈cycloalkyl, isoindolino, phthalimidyl, 2-oxo-1,3-oxazolidinyl, aryl or a 5 or 6 membered saturated or unsaturated heterocyclic ring having 1 to 4 heteroatoms selected from the group consisting O, S and N optionally substituted by
      hydroxy, C₁₋₆ alkyl, C₁₋₆ alkoxy, C₁₋₆ alkoxycarbonyl, hydroxyC₁₋₆ alkoxy, oxo, amino, aminoC₁₋₆alkyl, N—(C₁₋₆alkyl)amino, N,N-di(C₁₋₆alkyl)amino, N—(C₁₋₆ acyl)amino, or benzyl,
      wherein
      R²⁰¹ represents hydroxy, amino, N—(C₁₋₆alkyl)amino, N,N-di(C₁₋₆alkyl)amino, N-(halophenylC₁₋₆ alkyl)amino, C₁₋₆alkyl, aminoC₁₋₆ alkyl, aminoC₂₋₆ alkylenyl, C₁₋₆ alkoxy, a 5 or 6 membered saturated or unsaturated heterocyclic ring having 1 to 4 heteroatoms selected from the group consisting O, S and N optionally substituted by
      hydroxy, C₁₋₆ alkyl, C₁₋₆ alkoxy, C₁₋₆ alkoxycarbonyl, hydroxyC₁₋₆ alkoxy, oxo, amino, N—(C₁₋₆alkyl)amino, N,N-di(C₁₋₆alkyl)amino, N—(C₁₋₆ acyl)amino or benzyl;
   R³ represents hydrogen, halogen, aminocarbonyl, or C₁₋₆ alkyl optionally substituted by aryl C₁₋₆ alkoxy or mono-, di- or tri-halogen;
   R⁴ represents hydrogen or C₁₋₆ alkyl;
   R⁵ represents hydrogen or C₁₋₆ alkyl; and
R⁶ represents halogen, hydrogen or C₁₋₆ alkyl; or a physiologically acceptable salt, solvate, hydrate or stereoisomer thereof;
   said compounds are published as compounds of general formulae I, I-a, and I-b in International patent application PCT/EP2003/010377, published as WO 04/029055 A1 on Apr. 8, 2004, which is incorporated herein by reference in its entirety. In WO 04/029055, said compounds of general formula I, I-a and I-b are described on pp. 6 et seq., they may be synthesized according to the methods given therein on pp. 26 et seq., and are exemplified as specific compound Examples 1-1 to 1-210 on pp. 47 to 106, specific compound Examples 2-1 to 2-368 on pp. 107 to 204, specific compound Examples 3-1 to 3-2 on pp. 205 to 207, and as specific compound Examples 4-1 to 4-2 on pp. 208 to 210, therein.

Said component A may be in the form of a pharmaceutical formulation which is ready for use to be administered simultaneously, concurrently, separately or sequentially. The components may be administered independently of one another by the oral, intravenous, topical, local installations, intraperitoneal or nasal route.

In accordance with another embodiment of the above-mentioned aspects of the present invention, said combinations are of:

component A: which is one or more 2,3-dihydroimidazo[1,2-c]quinazoline compounds of general formula (A1), supra, which is selected from the list consisting of specific compound Examples 1-1 to 1-210 on pp. 47 to 106, specific compound Examples 2-1 to 2-368 on pp. 107 to 204, specific compound Examples 3-1 to 3-2 on pp. 205 to 207, and specific compound Examples 4-1 to 4-2 on pp. 208 to 210, of in International patent application PCT/EP2003/010377, published as WO 04/029055 A1 on Apr. 8, 2004, which is incorporated herein by reference in its entirety,
or a physiologically acceptable salt, solvate, hydrate or stereoisomer thereof.

Said component A may be in the form of a pharmaceutical formulation which is ready for use to be administered simultaneously, concurrently, separately or sequentially. The components may be administered independently of one another by the oral, intravenous, topical, local installations, intraperitoneal or nasal route.

As mentioned supra, said specific compound Examples may be synthesized according to the methods given in WO 04/029055 A1 on pp. 26 et seq.

In accordance with another embodiment of the above-mentioned aspects of the present invention, said combinations are of:

component A: which is one or more 2,3-dihydroimidazo[1,2-c]quinazoline compounds of general formula (A2):

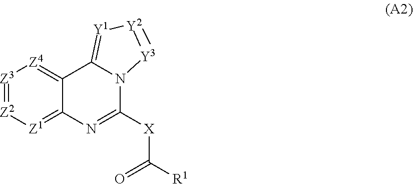

in which:
X represents CR⁵R⁶ or NH;
Y¹ represents CR³ or N;
the chemical bond between [symbol] represents a single bond or double bond, with the proviso that when the [symbol] represents a double bond, Y² and Y³ independently represent CR⁴ or N, and
when [symbol] represents a single bond, Y² and Y³ independently represent CR³R⁴ or NR⁴;
Z¹, Z², Z³ and Z⁴ independently represent CH, CR² or N;
R¹ represents aryl optionally having 1 to 3 substituents selected from R¹¹, C₃₋₈ cycloalkyl optionally having 1 to 3 substituents selected from R¹¹,
   C₁₋₆ alkyl optionally substituted by aryl, heteroaryl, C₁₋₆ alkoxyaryl, aryloxy, heteroaryloxy or one or more halogen,
   C₁₋₆ alkoxy optionally substituted by carboxy, aryl, heteroaryl, C₁₋₆ alkoxyaryl, aryloxy, heteroaryloxy or one or more halogen,
   or
   a 3 to 15 membered mono- or bi-cyclic heterocyclic ring that is saturated or unsaturated, optionally having 1 to 3 substituents selected from R¹¹, and contains 1 to 3 heteroatoms selected from the group consisting of N, O and S,
   wherein
   R¹¹ represents halogen, nitro, hydroxy, cyano, carboxy, amino, N—(C₁₋₆alkyl)amino, N-(hydroxyC₁₋₆alkyl)amino, N,N-di(C₁₋₆alkyl)amino, N—(C₁₋₆acyl)amino, N-(formyl)-N—(C₁₋₆alkyl)amino, N—(C₁₋₆alkanesulfonyl)amino, N-(carboxyC₁₋₆alkyl)-N—(C₁₋₆alkyl)amino, N—(C₁₋₆alkoxycarbonyl)amino, N—[N,N-di(C₁₋₆alkyl)amino methylene]amino, N—[N,N-di(C₁₋₆alkyl)amino (C₁₋₆alkyl)methylene]amino, N—[N,N-di(C₁₋₆alkyl)amino C₂₋₆alkenyl]amino, aminocarbonyl, N—(C₁₋₆alkyl)aminocarbonyl, N,N-di(C₁₋₆alkyl)aminocarbonyl, C₃₋₈cycloalkyl, C₁₋₆ alkylthio, C₁₋₆alkanesulfonyl, sulfamoyl, C₁₋₆alkoxycarbonyl,
   N-arylamino wherein said aryl moiety is optionally having 1 to 3 substituents selected from R¹⁰¹, N-(aryl C₁₋₆alkyl)amino wherein said aryl moiety is optionally having 1 to 3 substituents selected from R¹⁰¹, aryl C₁₋₆alkoxycarbonyl wherein said aryl moiety is optionally having 1 to 3 substituents selected from R¹⁰¹,
   C₁₋₆alkyl optionally substituted by mono-, di- or tri-halogen, amino, N—(C₁₋₆alkyl)amino or N,N-di(C₁₋₆alkyl)amino,
   C₁₋₆alkoxy optionally substituted by mono-, di- or tri-halogen, N—(C₁₋₆alkyl)sulfonamide, or N-(aryl)sulfonamide,
   or
   a 5 to 7 membered saturated or unsaturated ring having 1 to 3 heteroatoms selected from the group consisting of O, S and N, and optionally having 1 to 3 substituents selected from R¹⁰¹ wherein
   R¹⁰¹ represents halogen, carboxy, amino, N—(C₁₋₆ alkyl)amino, N,N-di(C₁₋₆alkyl)amino, aminocarbonyl, N—(C₁₋₆alkyl)amino-carbonyl, N,N-di(C₁₋₆alkyl)aminocarbonyl, pyridyl,
      C₁₋₆ alkyl optionally substituted by cyano or mono- di- or tri-halogen,
      and
      C₁₋₆alkoxy optionally substituted by cyano, carboxy, amino, N—(C₁₋₆ alkyl)amino, N,N-di(C₁₋₆alkyl)amino, aminocarbonyl, N—(C₁₋₆alkyl)aminocarbonyl, N,N-di(C₁₋₆alkyl)aminocarbonyl or mono-, di- or tri-halogen;
R² represents hydroxy, halogen, nitro, cyano, amino, N—(C₁₋₆alkyl)amino, N,N-di(C₁₋₆alkyl)amino, N-(hydroxyC₁₋₆alkyl)amino, N-(hydroxyC₁₋₆alkyl)-N—(C₁₋₆alkyl)amino, C₁₋₆ acyloxy, aminoC₁₋₆ acyloxy, C₂₋₆alkenyl, aryl,
   a 5-7 membered saturated or unsaturated heterocyclic ring having 1 to 3 heteroatoms selected from the group consisting O, S and N, and optionally substituted by
   hydroxy, C₁₋₆ alkyl, C₁₋₆ alkoxy, oxo, amino, amino C₁₋₆alkyl, N—(C₁₋₆alkyl)amino, N,N-di(C₁₋₆alkyl)amino, N—(C₁₋₆ acyl)amino, N—(C₁₋₆alkyl)carbonylamino, phenyl, phenyl C₁₋₆ alkyl, carboxy, C₁₋₆alkoxycarbonyl, aminocarbonyl, N—(C₁₋₆alkyl)aminocarbonyl, or N,N-di(C₁₋₆alkyl)amino, —C(O)—R²⁰
   wherein
   R²⁰ represents C₁₋₆ alkyl, C₁₋₆ alkoxy, amino, N—(C₁₋₆alkyl)amino, N,N-di(C₁₋₆alkyl)amino, N—(C₁₋₆ acyl)amino, or a 5-7 membered saturated or unsaturated heterocyclic ring having 1 to 3 heteroatoms selected from the group consisting O, S and N, and optionally substituted by C₁₋₆ alkyl, C₁₋₆ alkoxy, oxo, amino, N—(C₁₋₆alkyl)amino, N,N-di(C₁₋₆alkyl)amino, N—(C₁₋₆ acyl)amino, phenyl, or benzyl,
      C₁₋₆ alkyl optionally substituted by R²¹,
      or
      C₁₋₆ alkoxy optionally substituted by R²¹,
      wherein
      R²¹ represents cyano, mono-, di or tri-halogen, hydroxy, amino, N—(C₁₋₆alkyl)amino, N,N-di(C₁₋₆alkyl)amino, N-(hydroxyC₁₋₆ alkyl)amino, N-(halophenylC₁₋₆ alkyl)amino, amino C₂₋₆ alkylenyl, C₁₋₆ alkoxy, hydroxyC₁₋₆ alkoxy, —C(O)—R²⁰¹, —NHC(O)—R²⁰¹, C₃₋₈cycloalkyl, isoindolino, phthalimidyl, 2-oxo-1,3-oxazolidinyl, aryl or a 5 or 6 membered saturated or unsaturated heterocyclic ring having 1 to 4 heteroatoms selected from the group consisting O, S and N, and optionally substituted by hydroxy, C₁₋₆ alkyl, C₁₋₆ alkoxy, C₁₋₆ alkoxycarbonyl, hydroxyC₁₋₆ alkoxy, oxo, amino, aminoC₁₋₆alkyl, N—(C₁₋₆alkyl)amino, N,N-di(C₁₋₆alkyl)amino, N—(C₁₋₆ acyl)amino, or benzyl,
         wherein
R²⁰¹ represents hydroxy, amino, N—(C₁₋₆alkyl)amino, N,N-di(C₁₋₆alkyl)amino, N-(halophenylC₁₋₆ alkyl)amino, C₁₋₆alkyl, aminoC₁₋₆ alkyl, aminoC₂₋₆ alkylenyl, C₁₋₆ alkoxy, a 5 or 6 membered saturated or unsaturated heterocyclic ring having 1 to 4 heteroatoms selected from the group consisting O, S and N, and optionally substituted by hydroxy, C₁₋₆ alkyl, C₁₋₆ alkoxy, C₁₋₆ alkoxycarbonyl, hydroxyC₁₋₆ alkoxy, oxo, amino, N—(C₁₋₆alkyl)amino, N,N-di(C₁₋₆alkyl)amino, N—(C₁₋₆ acyl)amino or benzyl;
R³ represents hydrogen, halogen, aminocarbonyl, or C₁₋₆ alkyl optionally substituted by aryl C₁₋₆ alkoxy or mono-, di- or tri-halogen;
R⁴ represents hydrogen or C₁₋₆ alkyl;
R⁵ represents hydrogen or C₁₋₆ alkyl; and
R⁶ represents halogen, hydrogen or C₁₋₆ alkyl;
   or a physiologically acceptable salt, solvate, hydrate or stereoisomer thereof; said compounds are published as compounds of general formulae I, Ia, Ib, Ic, Id and Ie in International patent application PCT/US2007/024985, published as WO 2008/070150 A1 on Jun. 12, 2008, which is incorporated herein by reference in its entirety. In WO 2008/070150, said compounds of general formula I, Ia, Ib, Ic, Id and le are described on pp. 9 et seq., they may be synthesized according to the methods given therein on pp. 42, et seq., and are exemplified as specific compound Examples 1 to 103 therein on pp. 65 to 101. Biological test data for certain of said compounds are given therein on pp. 101 to 107.

Said component A may be in the form of a pharmaceutical formulation which is ready for use to be administered simultaneously, concurrently, separately or sequentially. The components may be administered independently of one another by the oral, intravenous, topical, local installations, intraperitoneal or nasal route.

In accordance with another embodiment of the above-mentioned aspects of the present invention, said combinations are of:

component A: which is one or more 2,3-dihydroimidazo[1,2-c]quinazoline compounds of general formula (A2), supra, which is selected from the list is consisting of:
Example 1: N-[7-methoxy-8-(3-morpholin-4-ylpropoxy)-2,3-dihydroimidazo[1,2-c]quinazolin-5-yl]pyrimidine-5-carboxamide
Example 2: N-(8-{3-[(2R,6S)-2,6-dimethylmorpholin-4-yl]propoxy}-7-methoxy-2,3-dihydroimidazo[1,2-c]quinazolin-5-yl)nicotinamide
Example 3: N-(8-{3-[(2R,6S)-2,6-dimethylmorpholin-4-yl]propoxy}-7-methoxy-2,3-dihydroimidazo[1,2-c]quinazolin-5-yl)-2,4-dimethyl-1,3-thiazole-5-carboxamide
Example 4: 2-amino-N-[7-methoxy-8-(3-morpholin-4-ylpropoxy)-2,3-dihydroimidazo[1,2-c]quinazolin-5-yl]-1,3-thiazole-5-carboxamide.
Example 5: 2-amino-N-[7-methoxy-8-(3-morpholin-4-ylpropoxy)-2,3-dihydroimidazo[1,2-c]quinazolin-5-yl]isonicotinamide
Example 6: 2-amino-N-[7-methoxy-8-(3-morpholin-4-ylpropoxy)-2,3-dihydroimidazo[1,2-c]quinazolin-5-yl]-4-methyl-1,3-thiazole-5-carboxamide
Example 7: 2-amino-N-[7-methoxy-8-(3-morpholin-4-ylpropoxy)-2,3-dihydroimidazo[1,2-c]quinazolin-5-yl]-4-propylpyrimidine-5-carboxamide
Example 8: N-{8-[2-(4-ethylmorpholin-2-yl)ethoxy]-7-methoxy-2,3-dihydroimidazo[1,2-c]quinazolin-5-yl}nicotinamide
Example 9: N-{8-[2-(dimethylamino)ethoxy]-7-methoxy-2,3-dihydroimidazo[1,2-c]quinazolin-5-yl}pyrimidine-5-carboxamide
Example 10: N-(8-{3-[2-(hydroxymethyl)morpholin-4-yl]propoxy}-7-methoxy-2,3-dihydroimidazo[1,2-c]quinazolin-5-yl)nicotinamide
Example 11: N-(8-{3-[2-(hydroxymethyl)morpholin-4-yl]propoxy}-7-methoxy-2,3-dihydroimidazo[1,2-c]quinazolin-5-yl)nicotinamide
Example 12: N-{8-[3-(dimethylamino)propoxy]-7-methoxy-2,3-dihydroimidazo[1,2-c]quinazolin-5-yl}nicotinamide 1-oxide
Example 13: 2-amino-N-[7-methoxy-8-(3-morpholin-4-ylpropoxy)-2,3-dihydroimidazo[1,2-c]quinazolin-5-yl]pyrimidine-5-carboxamide.
Example 14: N-[7-methoxy-8-(3-morpholin-4-ylpropoxy)-2,3-dihydroimidazo[1,2-c]quinazolin-5-yl]-6-(2-pyrrolidin-1-ylethyl)nicotinamide.
Example 15: 6-(cyclopentylamino)-N-[7-methoxy-8-(3-morpholin-4-ylpropoxy)-2,3-dihydroimidazo[1,2-c]quinazolin-5-yl]nicotinamide

| Example | Structure |
|---|---|
| 16 | 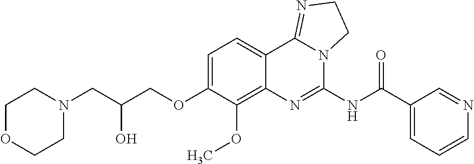 [SMILES: COc1c(OCC(O)CN2CCOCC2)ccc2c1N=C(NC(=O)c1cccnc1)N1CCN=C21] |
| 17 | 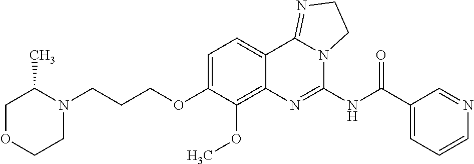 [SMILES: COc1c(OCCCN2CCOC[C@@H]2C)ccc2c1N=C(NC(=O)c1cccnc1)N1CCN=C21] |
| 18 | 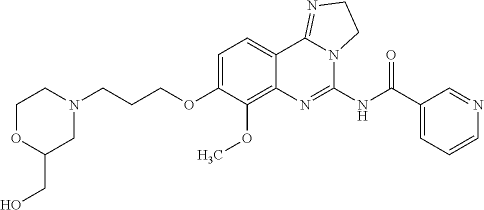 [SMILES: COc1c(OCCCN2CCOC(CO)C2)ccc2c1N=C(NC(=O)c1cccnc1)N1CCN=C21] |
| 19 | 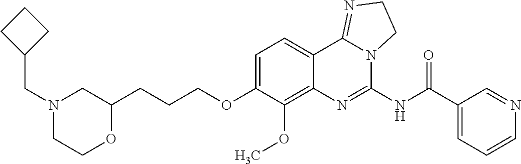 [SMILES: COc1c(OCCCC2CN(CC3CCC3)CCO2)ccc2c1N=C(NC(=O)c1cccnc1)N1CCN=C21] |
| 20 | 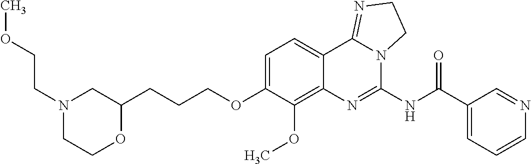 [SMILES: COCCN1CCOC(CCCOc2ccc3c(c2OC)N=C(NC(=O)c2cccnc2)N2CCN=C32)C1] |
| 21 | 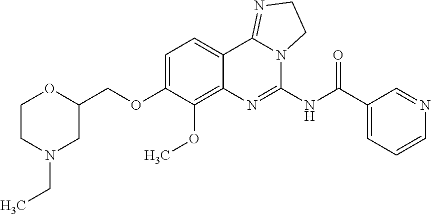 [SMILES: CCN1CCOC(COc2ccc3c(c2OC)N=C(NC(=O)c2cccnc2)N2CCN=C32)C1] |
| 22 | 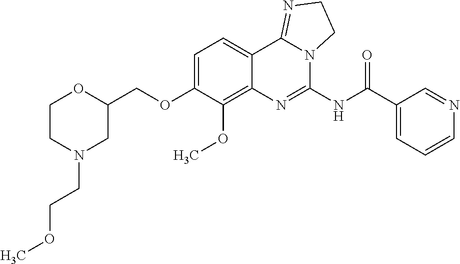 [SMILES: COCCN1CCOC(COc2ccc3c(c2OC)N=C(NC(=O)c2cccnc2)N2CCN=C32)C1] |
| 23 | 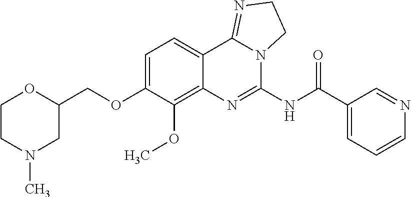 [SMILES: COc1c(OCC2CN(C)CCO2)ccc2c1N=C(NC(=O)c1cccnc1)N1CCN=C21] |
| 24 | 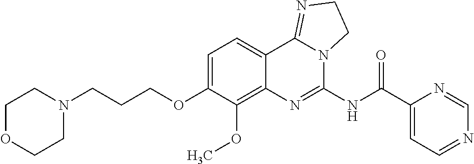 [SMILES: COc1c(OCCCN2CCOCC2)ccc2c1N=C(NC(=O)c1ccncn1)N1CCN=C21] |
| 25 | 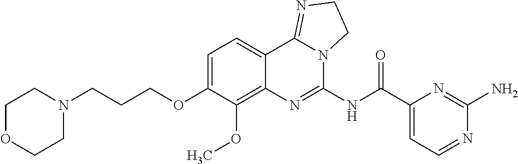 [SMILES: COc1c(OCCCN2CCOCC2)ccc2c1N=C(NC(=O)c1ccnc(N)n1)N1CCN=C21] |
| 26 | 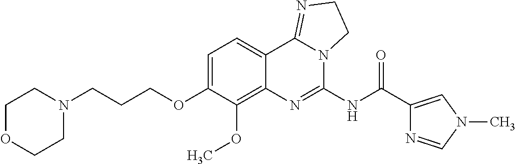 [SMILES: COc1c(OCCCN2CCOCC2)ccc2c1N=C(NC(=O)c1cn(C)cn1)N1CCN=C21] |
| 27 | 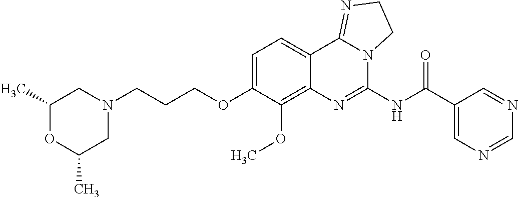 [SMILES: COc1c(OCCCN2C[C@@H](C)O[C@@H](C)C2)ccc2c1N=C(NC(=O)c1cncnc1)N1CCN=C21] |
| 28 | 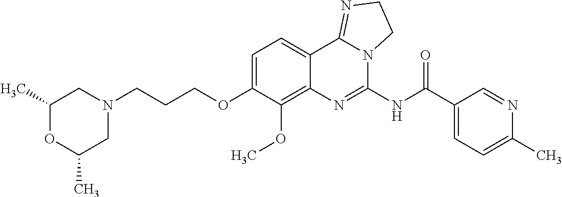 |
| 29 | 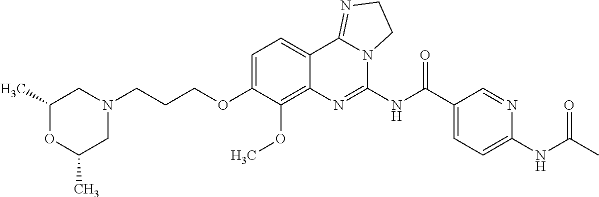 [SMILES: COc1c(OCCCN2C[C@@H](C)O[C@@H](C)C2)ccc2c1N=C(NC(=O)c1ccc(NC(C)=O)nc1)N1CCN=C21] |
| 30 | 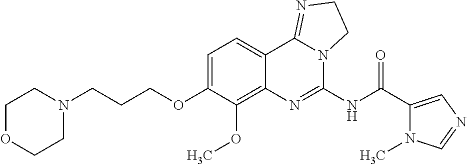 [SMILES: COc1c(OCCCN2CCOCC2)ccc2c1N=C(NC(=O)c1cncn1C)N1CCN=C21] |
| 31 | 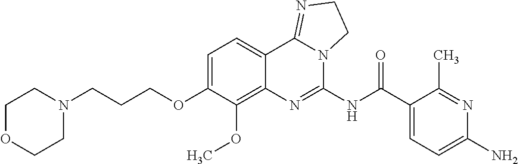 [SMILES: COc1c(OCCCN2CCOCC2)ccc2c1N=C(NC(=O)c1ccc(N)nc1C)N1CCN=C21] |
| 32 | 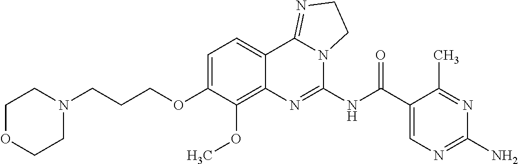 [SMILES: COc1c(OCCCN2CCOCC2)ccc2c1N=C(NC(=O)c1cnc(N)nc1C)N1CCN=C21] |
| 33 | 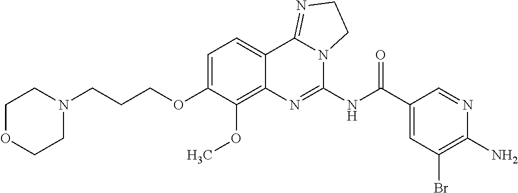 [SMILES: COc1c(OCCCN2CCOCC2)ccc2c1N=C(NC(=O)c1cnc(N)c(Br)c1)N1CCN=C21] |
| 34 | 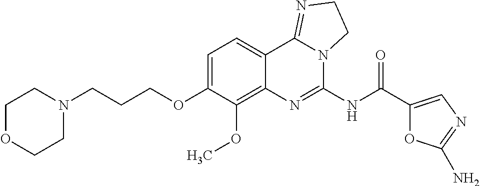 [SMILES: COc1c(OCCCN2CCOCC2)ccc2c1N=C(NC(=O)c1cnc(N)o1)N1CCN=C21] |
| 35 | 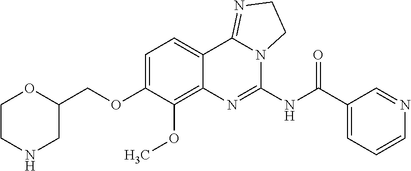 [SMILES: COc1c(OCC2CNCCO2)ccc2c1N=C(NC(=O)c1cccnc1)N1CCN=C21] |
| 36 | 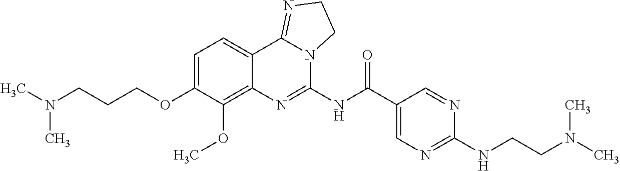 |
| 37 | 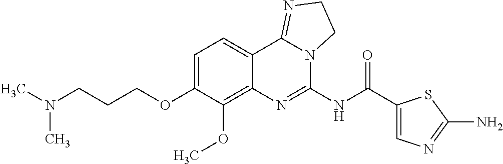 [SMILES: COc1c(OCCCN(C)C)ccc2c1N=C(NC(=O)c1cnc(N)s1)N1CCN=C21] |
| 38 | 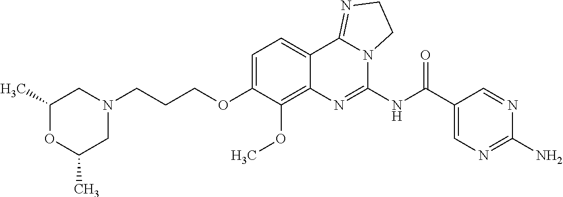 [SMILES: COc1c(OCCCN2C[C@@H](C)O[C@@H](C)C2)ccc2c1N=C(NC(=O)c1cnc(N)nc1)N1CCN=C21] |
| 39 | 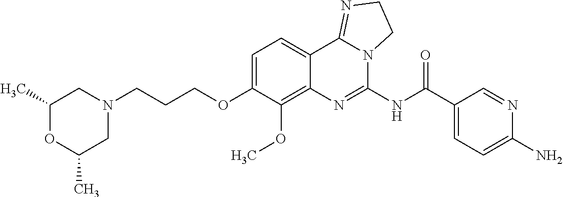 [SMILES: COc1c(OCCCN2C[C@@H](C)O[C@@H](C)C2)ccc2c1N=C(NC(=O)c1ccc(N)nc1)N1CCN=C21] |
| 40 | 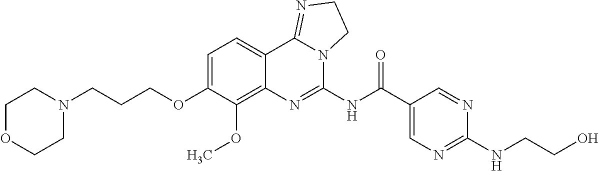 [SMILES: COc1c(OCCCN2CCOCC2)ccc2c1N=C(NC(=O)c1cnc(NCCO)nc1)N1CCN=C21] |
| 41 | 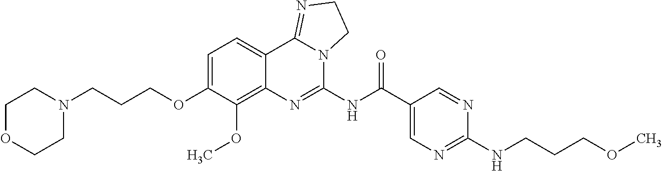 [SMILES: COCCCNc1ncc(C(=O)NC2=Nc3c(ccc(OCCCN4CCOCC4)c3OC)C3=NCCN23)cn1] |
| 42 | 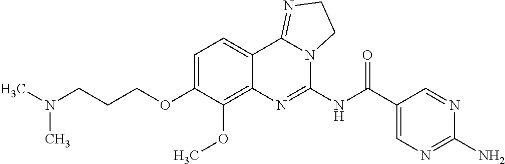 [SMILES: COc1c(OCCCN(C)C)ccc2c1N=C(NC(=O)c1cnc(N)nc1)N1CCN=C21] |
| 43 | 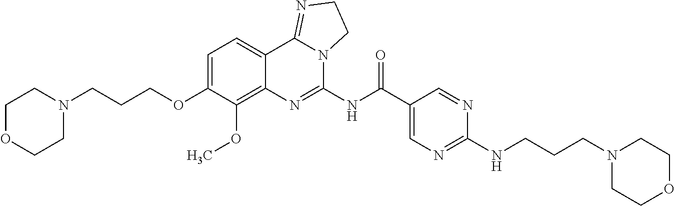 [SMILES: COc1c(OCCCN2CCOCC2)ccc2c1N=C(NC(=O)c1cnc(NCCCN3CCOCC3)nc1)N1CCN=C21] |
| 44 | 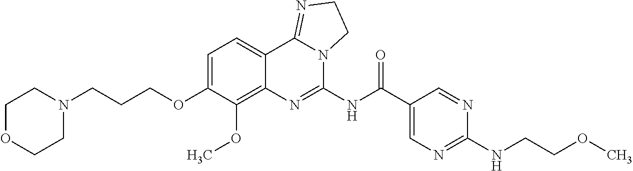 [SMILES: COCCNc1ncc(C(=O)NC2=Nc3c(ccc(OCCCN4CCOCC4)c3OC)C3=NCCN23)cn1] |
| 45 | 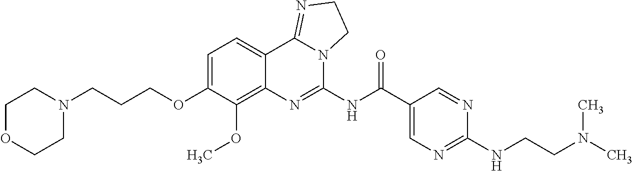 [SMILES: COc1c(OCCCN2CCOCC2)ccc2c1N=C(NC(=O)c1cnc(NCCN(C)C)nc1)N1CCN=C21] |
| 46 | 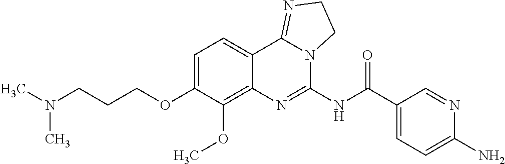 [SMILES: COc1c(OCCCN(C)C)ccc2c1N=C(NC(=O)c1ccc(N)nc1)N1CCN=C21] |
| 47 | 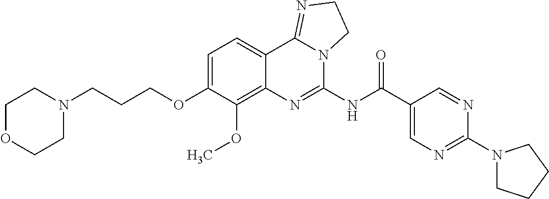 [SMILES: COc1c(OCCCN2CCOCC2)ccc2c1N=C(NC(=O)c1cnc(N3CCCC3)nc1)N1CCN=C21] |
| 48 | 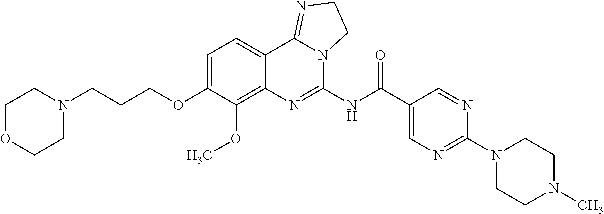 [SMILES: COc1c(OCCCN2CCOCC2)ccc2c1N=C(NC(=O)c1cnc(N3CCN(C)CC3)nc1)N1CCN=C21] |
| 49 | 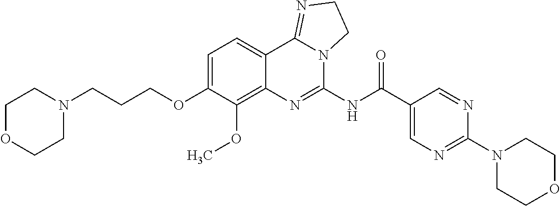 [SMILES: COc1c(OCCCN2CCOCC2)ccc2c1N=C(NC(=O)c1cnc(N3CCOCC3)nc1)N1CCN=C21] |
| 50 | 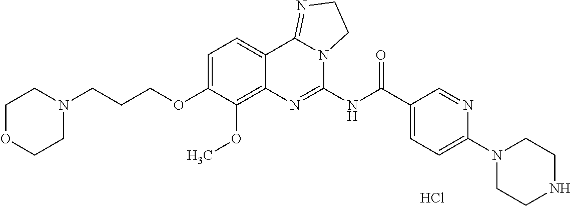 [SMILES: COc1c(OCCCN2CCOCC2)ccc2c1N=C(NC(=O)c1ccc(N3CCNCC3)nc1)N1CCN=C21 ; Cl] |
| 51 | 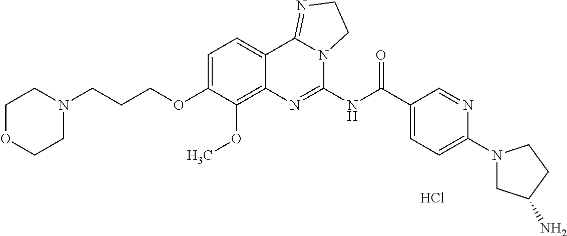 [SMILES: COc1c(OCCCN2CCOCC2)ccc2c1N=C(NC(=O)c1ccc(N3CC[C@H](N)C3)nc1)N1CCN=C21 ; Cl] |
| 52 | 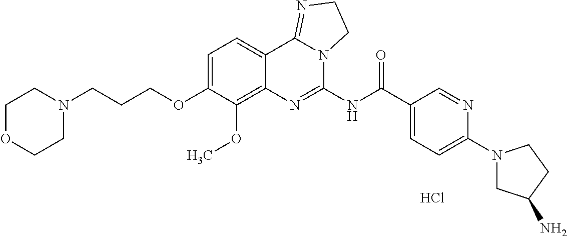 [SMILES: COc1c(OCCCN2CCOCC2)ccc2c1N=C(NC(=O)c1ccc(N3CC[C@@H](N)C3)nc1)N1CCN=C21 ; Cl] |
| 53 | 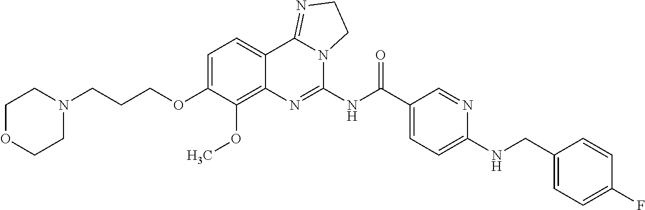 [SMILES: COc1c(OCCCN2CCOCC2)ccc2c1N=C(NC(=O)c1ccc(NCc3ccc(F)cc3)nc1)N1CCN=C21] |
| 54 | 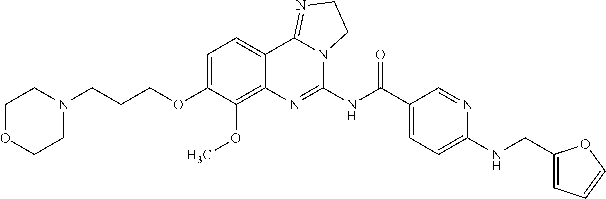 [SMILES: COc1c(OCCCN2CCOCC2)ccc2c1N=C(NC(=O)c1ccc(NCc3ccco3)nc1)N1CCN=C21] |
| 55 | 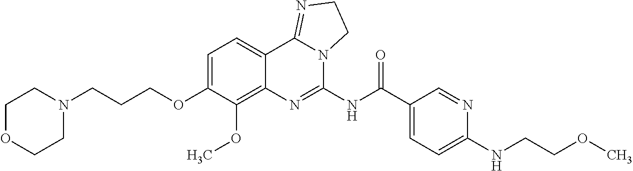 [SMILES: COCCNc1ccc(C(=O)NC2=Nc3c(ccc(OCCCN4CCOCC4)c3OC)C3=NCCN23)cn1] |
| 56 | 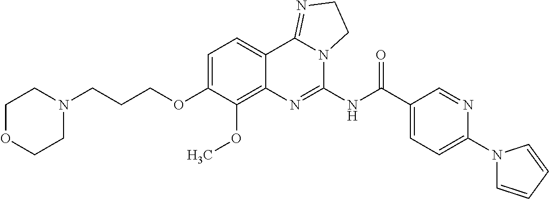 [SMILES: COc1c(OCCCN2CCOCC2)ccc2c1N=C(NC(=O)c1ccc(-n3cccc3)nc1)N1CCN=C21] |
| 57 | 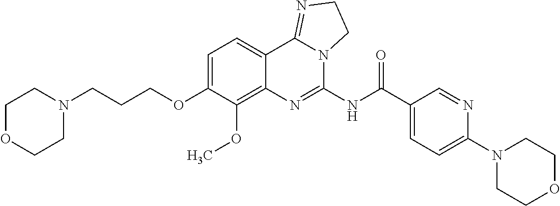 [SMILES: COc1c(OCCCN2CCOCC2)ccc2c1N=C(NC(=O)c1ccc(N3CCOCC3)nc1)N1CCN=C21] |
| 58 | 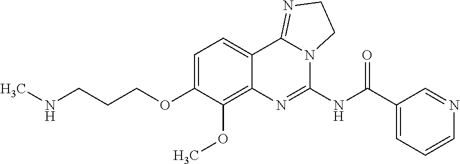 [SMILES: CNCCCOc1ccc2c(c1OC)N=C(NC(=O)c1cccnc1)N1CCN=C21] |
| 59 | 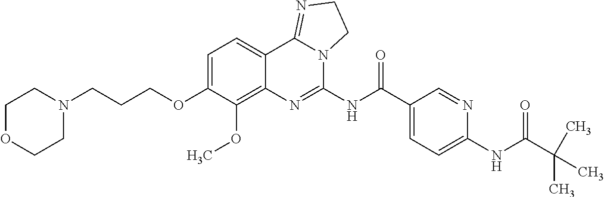 [SMILES: COc1c(OCCCN2CCOCC2)ccc2c1N=C(NC(=O)c1ccc(NC(=O)C(C)(C)C)nc1)N1CCN=C21] |
| 60 | 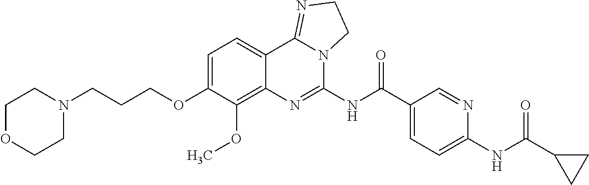 [SMILES: COc1c(OCCCN2CCOCC2)ccc2c1N=C(NC(=O)c1ccc(NC(=O)C3CC3)nc1)N1CCN=C21] |
| 61 | 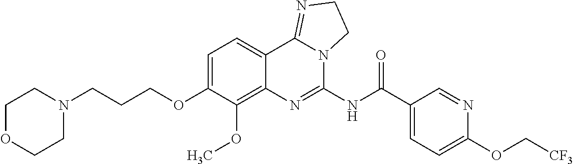 [SMILES: COc1c(OCCCN2CCOCC2)ccc2c1N=C(NC(=O)c1ccc(OCC(F)(F)F)nc1)N1CCN=C21] |
| 62 | 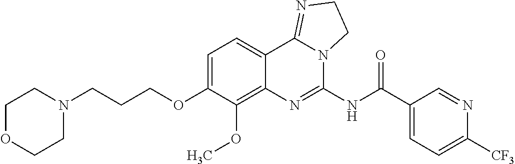 [SMILES: COc1c(OCCCN2CCOCC2)ccc2c1N=C(NC(=O)c1ccc(C(F)(F)F)nc1)N1CCN=C21] |
| 63 | 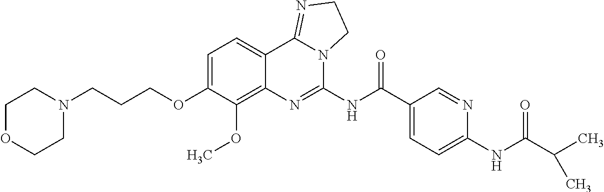 [SMILES: COc1c(OCCCN2CCOCC2)ccc2c1N=C(NC(=O)c1ccc(NC(=O)C(C)C)nc1)N1CCN=C21] |
| 64 | 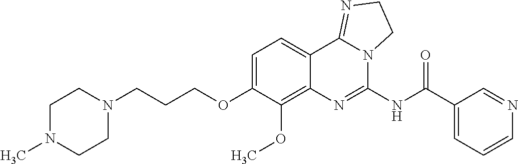 [SMILES: COc1c(OCCCN2CCN(C)CC2)ccc2c1N=C(NC(=O)c1cccnc1)N1CCN=C21] |
| 65 | 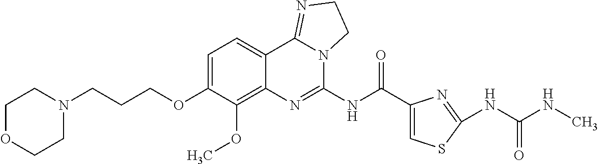 [SMILES: CNC(=O)Nc1nc(C(=O)NC2=Nc3c(ccc(OCCCN4CCOCC4)c3OC)C3=NCCN23)cs1] |
| 66 | 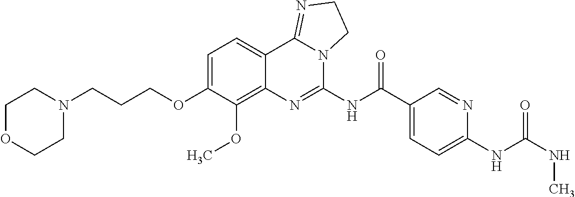 [SMILES: CNC(=O)Nc1ccc(C(=O)NC2=Nc3c(ccc(OCCCN4CCOCC4)c3OC)C3=NCCN23)cn1] |
| 67 | 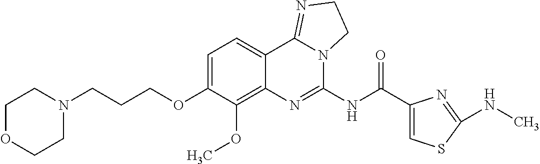 [SMILES: CNc1nc(C(=O)NC2=Nc3c(ccc(OCCCN4CCOCC4)c3OC)C3=NCCN23)cs1] |
| 68 | 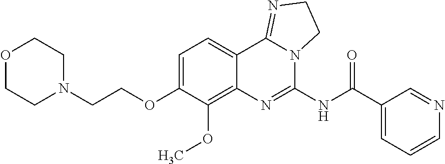 [SMILES: COc1c(OCCN2CCOCC2)ccc2c1N=C(NC(=O)c1cccnc1)N1CCN=C21] |
| 69 | 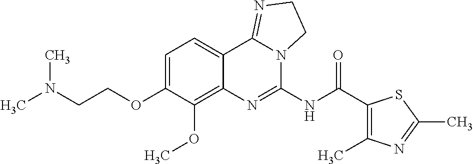 |
| 70 | 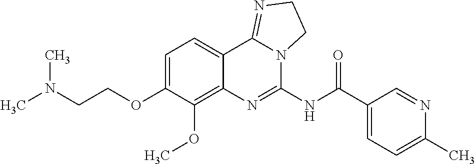 |
| 71 | 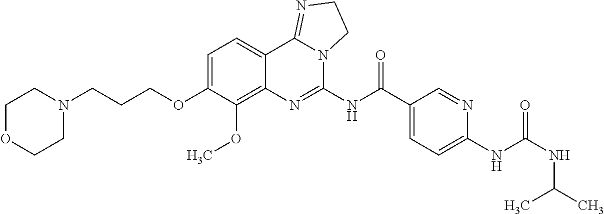 [SMILES: COc1c(OCCCN2CCOCC2)ccc2c1N=C(NC(=O)c1ccc(NC(=O)NC(C)C)nc1)N1CCN=C21] |
| 72 | 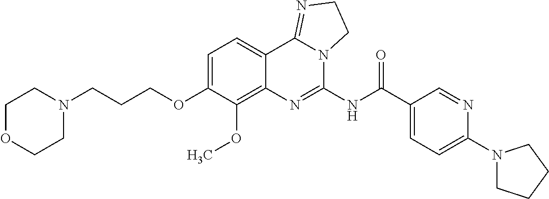 [SMILES: COc1c(OCCCN2CCOCC2)ccc2c1N=C(NC(=O)c1ccc(N3CCCC3)nc1)N1CCN=C21] |
| 73 | 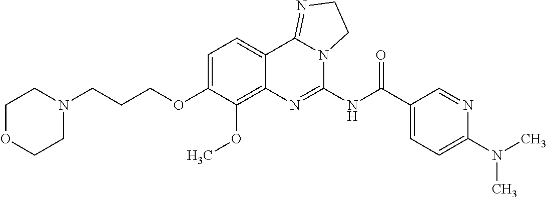 [SMILES: COc1c(OCCCN2CCOCC2)ccc2c1N=C(NC(=O)c1ccc(N(C)C)nc1)N1CCN=C21] |
| 74 | 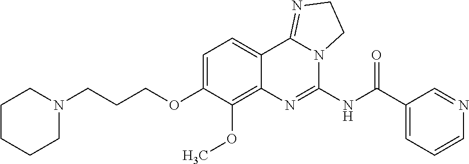 [SMILES: COc1c(OCCCN2CCCCC2)ccc2c1N=C(NC(=O)c1cccnc1)N1CCN=C21] |
| 75 | 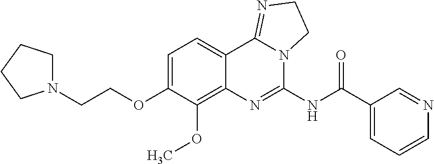 [SMILES: COc1c(OCCN2CCCC2)ccc2c1N=C(NC(=O)c1cccnc1)N1CCN=C21] |
| 76 | 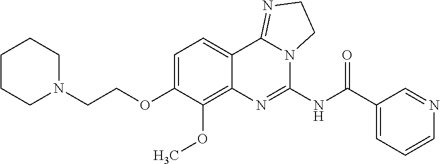 [SMILES: COc1c(OCCN2CCCCC2)ccc2c1N=C(NC(=O)c1cccnc1)N1CCN=C21] |
| 77 | 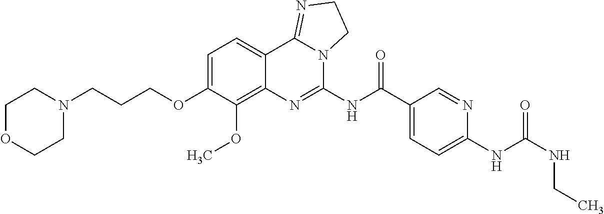 [SMILES: CCNC(=O)Nc1ccc(C(=O)NC2=Nc3c(ccc(OCCCN4CCOCC4)c3OC)C3=NCCN23)cn1] |
| 78 | 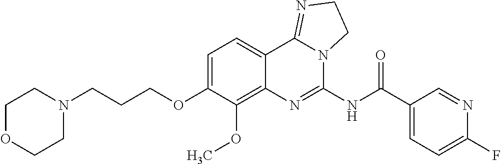 [SMILES: COc1c(OCCCN2CCOCC2)ccc2c1N=C(NC(=O)c1ccc(F)nc1)N1CCN=C21] |
| 79 | 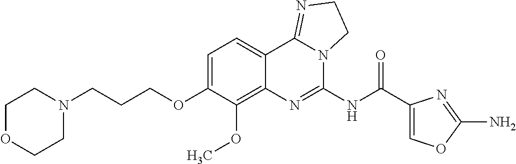 [SMILES: COc1c(OCCCN2CCOCC2)ccc2c1N=C(NC(=O)c1coc(N)n1)N1CCN=C21] |
| 80 | 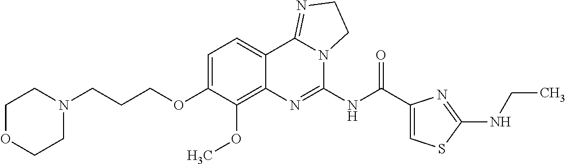 [SMILES: CCNc1nc(C(=O)NC2=Nc3c(ccc(OCCCN4CCOCC4)c3OC)C3=NCCN23)cs1] |
| 81 | 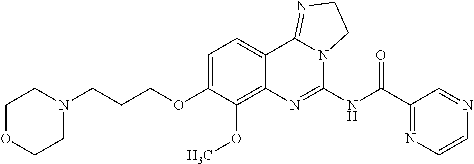 [SMILES: COc1c(OCCCN2CCOCC2)ccc2c1N=C(NC(=O)c1cnccn1)N1CCN=C21] |
| 82 | 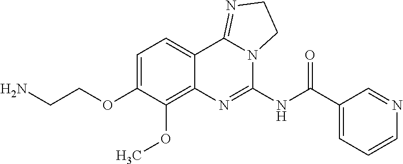 [SMILES: COc1c(OCCN)ccc2c1N=C(NC(=O)c1cccnc1)N1CCN=C21] |
| 83 | 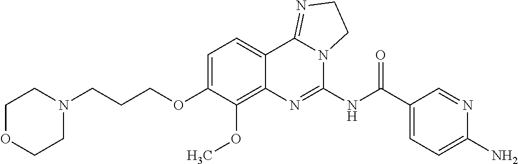 [SMILES: COc1c(OCCCN2CCOCC2)ccc2c1N=C(NC(=O)c1ccc(N)nc1)N1CCN=C21] |
| 84 | 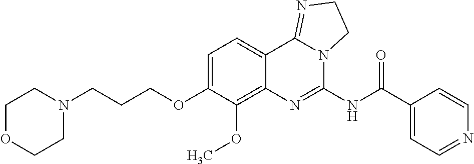 [SMILES: COc1c(OCCCN2CCOCC2)ccc2c1N=C(NC(=O)c1ccncc1)N1CCN=C21] |
| 85 | 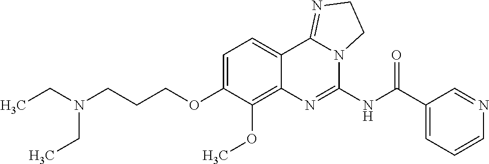 [SMILES: CCN(CC)CCCOc1ccc2c(c1OC)N=C(NC(=O)c1cccnc1)N1CCN=C21] |
| 86 | 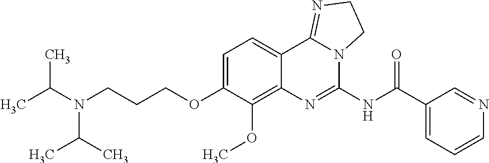 [SMILES: COc1c(OCCCN(C(C)C)C(C)C)ccc2c1N=C(NC(=O)c1cccnc1)N1CCN=C21] |
| 87 | 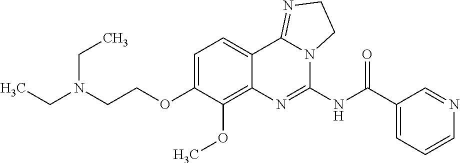 [SMILES: CCN(CC)CCOc1ccc2c(c1OC)N=C(NC(=O)c1cccnc1)N1CCN=C21] |
| 88 | 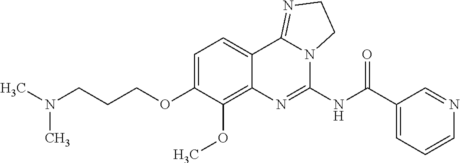 [SMILES: COc1c(OCCCN(C)C)ccc2c1N=C(NC(=O)c1cccnc1)N1CCN=C21] |
| 89 | 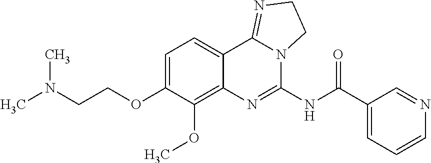 [SMILES: COc1c(OCCN(C)C)ccc2c1N=C(NC(=O)c1cccnc1)N1CCN=C21] |
| 90 | 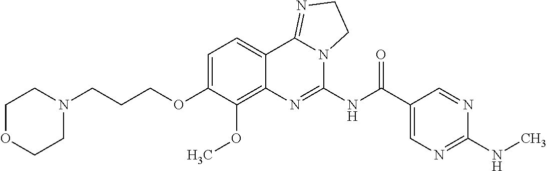 [SMILES: CNc1ncc(C(=O)NC2=Nc3c(ccc(OCCCN4CCOCC4)c3OC)C3=NCCN23)cn1] |
| 91 | 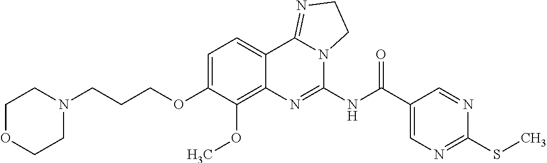 [SMILES: COc1c(OCCCN2CCOCC2)ccc2c1N=C(NC(=O)c1cnc(SC)nc1)N1CCN=C21] |
| 92 | 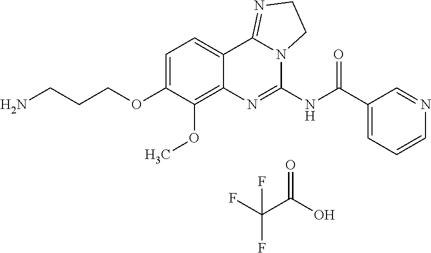 [SMILES: COc1c(OCCCN)ccc2c1N=C(NC(=O)c1cccnc1)N1CCN=C21 ; O=C(O)C(F)(F)F] |
| 93 | 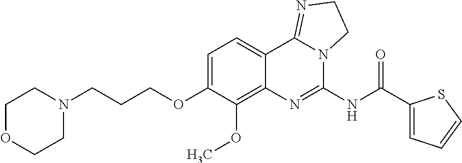 [SMILES: COc1c(OCCCN2CCOCC2)ccc2c1N=C(NC(=O)c1cccs1)N1CCN=C21] |
| 94 | 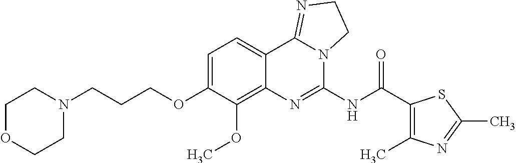 [SMILES: COc1c(OCCCN2CCOCC2)ccc2c1N=C(NC(=O)c1sc(C)nc1C)N1CCN=C21] |
| 95 | 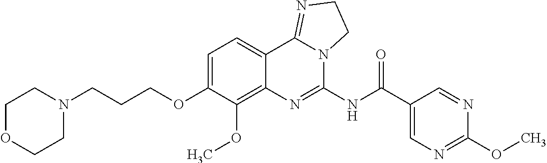 [SMILES: COc1ncc(C(=O)NC2=Nc3c(ccc(OCCCN4CCOCC4)c3OC)C3=NCCN23)cn1] |
| 96 | 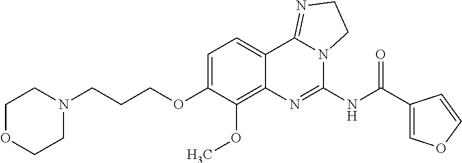 [SMILES: COc1c(OCCCN2CCOCC2)ccc2c1N=C(NC(=O)c1ccoc1)N1CCN=C21] |
| 97 | 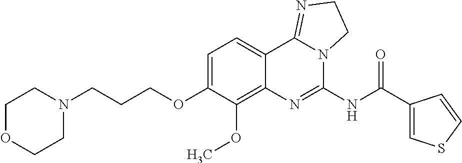 [SMILES: COc1c(OCCCN2CCOCC2)ccc2c1N=C(NC(=O)c1ccsc1)N1CCN=C21] |
| 98 | 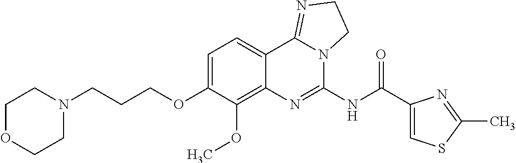 [SMILES: COc1c(OCCCN2CCOCC2)ccc2c1N=C(NC(=O)c1csc(C)n1)N1CCN=C21] |
| 99 | 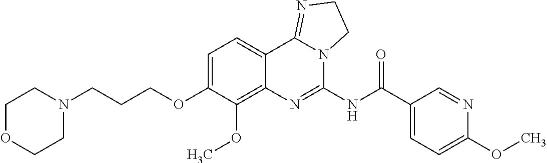 [SMILES: COc1ccc(C(=O)NC2=Nc3c(ccc(OCCCN4CCOCC4)c3OC)C3=NCCN23)cn1] |
| 100 | 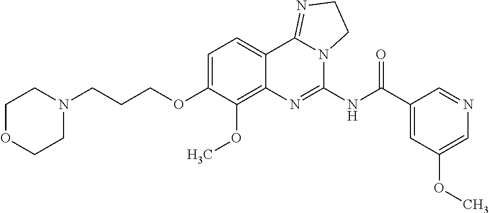 [SMILES: COc1cncc(C(=O)NC2=Nc3c(ccc(OCCCN4CCOCC4)c3OC)C3=NCCN23)c1] |
| 101 | 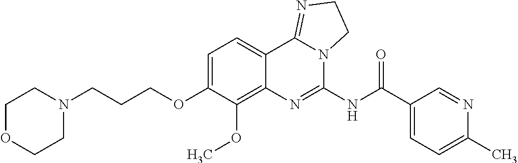 [SMILES: COc1c(OCCCN2CCOCC2)ccc2c1N=C(NC(=O)c1ccc(C)nc1)N1CCN=C21] |
| 102 | 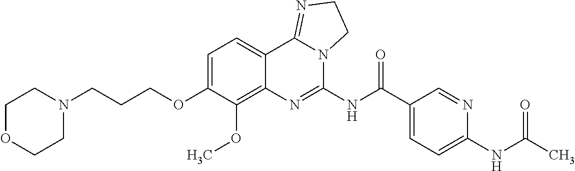 [SMILES: COc1c(OCCCN2CCOCC2)ccc2c1N=C(NC(=O)c1ccc(NC(C)=O)nc1)N1CCN=C21] |
| 103 | 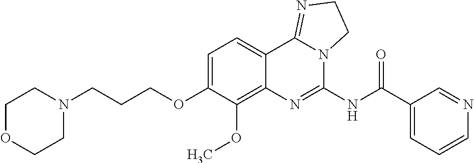 [SMILES: COc1c(OCCCN2CCOCC2)ccc2c1N=C(NC(=O)c1cccnc1)N1CCN=C21] |

or a physiologically acceptable salt, solvate, hydrate or stereoisomer thereof, said compounds are published as specific compound Examples 1 to 103 in International patent application PCT/US2007/024985, published as WO 2008/070150 A1 on Jun. 12, 2008, which is incorporated herein by reference in its entirety. In WO 2008/070150, said specific compound Examples may be synthesized according to the Examples. Biological test data for certain of said compounds are given therein on pp. 101 to 107.

Said component A may be in the form of a pharmaceutical formulation which is ready for use to be administered simultaneously, concurrently, separately or sequentially. The components may be administered independently of one another by the oral, intravenous, topical, local installations, intraperitoneal or nasal route.

In accordance with an embodiment of the above-mentioned aspects of the present invention, said combinations are of:

component B: which is one or more N-(2-arylamino)aryl sulfonamide compounds of general formula (B):

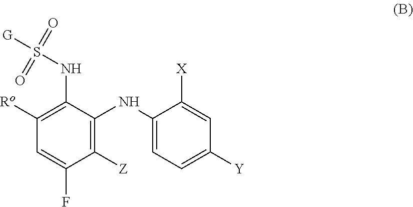

where G is R₁ₐ, R_{1b}, R_{1c}, R_{1d}, R₁ₑ, Ar₁, Ar₂ or Ar₃; R^{o} is H, halogen, C₁-C₆ alkyl, C₁-C₄ alkoxy, C₃-C₆ cycloalkyl, C₂-C₆ alkenyl, C₂-C₆ alkynyl, said alkyl, cycloalkyl, alkenyl, and alkynyl groups optionally substituted with 1-3 substituents selected independently from halogen, OH, CN, cyanomethyl, nitro, phenyl, and trifluoromethyl, and said C₁-C₆ alkyl and C₁-C₄ alkoxy groups also optionally substituted with OCH₃ or OCH₂CH₃; X is F, Cl or methyl; Y is I, Br, Cl, CF₃, C₁-C₃ alkyl, C₂-C₃ alkenyl, C₂-C₃ alkynyl, cyclopropyl, phenyl, pyridyl, pyrazolyl, OMe, OEt, or SMe, where all said methyl, ethyl, C₁-C₃ alkyl, and cyclopropyl groups of X and Y are optionally substituted with OH, all said phenyl, pyridyl, pyrazolyl groups of Y are optionally substituted with halogen, acetyl, methyl, and trifluoromethyl, and all said methyl groups of X and Y are optionally substituted with one, two, or three F atoms; and Z is H or F,
where R₁ₐ is methyl, optionally substituted with 1-3 fluorine atoms or 1-3 chlorine atoms, or with OH, cyclopropoxy, or C₁-C₄ alkoxy, where the C₁-C₄ alkyl moieties of said C₁-C₄ alkoxy groups are optionally substituted with one hydroxy or methoxy group, and where all C₂-C₄ alkyl groups within said C₁-C₄ alkoxy are optionally further substituted with a second OH group;
R_{1b} is CH(CH₃)—C₁₋₃ alkyl or C₃-C₆ cycloalkyl, said methyl, alkyl, and cycloalkyl is groups optionally substituted with 1-3 substituents selected independently from F, Cl, Br, I, OH, C₁-C₄ alkoxy, and CN;
R_{1c} is (CH₂)ₙOₘR′, where m is 0 or 1; where, when m is 1, n is 2 or 3, and when m is 0, n is 1 or 2; and where R′ is C₁-C₆ alkyl, optionally substituted with 1-3 substituents selected independently from F, Cl, OH, OCH₃, OCH₂CH₃, and C₃-C₆ cycloalkyl;
R_{1d} is C(A)(A′)(B)— where B, A, and A′ are, independently, H or C₁₋₄ alkyl, optionally substituted with one or two OH groups or halogen atoms, or A and A′, together with the carbon atom to which they are attached, form a 3- to 6-member saturated ring, said ring optionally containing one or two heteroatoms selected, independently, from O, N, and S and optionally substituted with one or two groups selected independently from methyl, ethyl, and halo;
R₁ₑ is benzyl or 2-phenyl ethyl, in which the phenyl group is optionally substituted

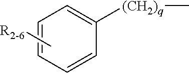

where q is 1 or 2, R₂, R₃ and R₄ are, independently, H, F, Cl, Br, CH₃, CH₂F, CHF₂, CF₃, OCH₃, OCH₂F, OCHF₂, OCF₃, ethyl, n-propyl, isopropyl, cyclopropyl, isobutyl, sec-butyl, tert-butyl, and methylsulfonyl, and R₄ may also be nitro, acetamido, amidinyl, cyano, carbamoyl, methylcarbamoyl, dimethylcarbamoyl, 1,3,4-oxadiazol-2-yl, 5-methyl-1,3,4-oxadiazolyl, 1,3,4-thiadiazolyl, 5-methyl-1,3,4-thiadiazol-1H-tetrazolyl, N-morpholinyl carbonylamino, N-morpholinylsulfonyl, and N-pyrrolidinylcarbonylamino; R₅ and R₆ are, independently, H, F, Cl, or methyl;

### Ar₁ is

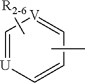

where U and V are, independently, N, CR₂ or CR₃; R₂, R₃ and R₄ are, independently, H, F, Cl, Br, CH₃, CH₂F, CHF₂, CF₃, OCH₃, OCH₂F, OCHF₂, OCF₃, ethyl, n-propyl, isopropyl, cyclopropyl, isobutyl, sec-butyl, tert-butyl, and methylsulfonyl, and R₄ may also be nitro, acetamido, amidinyl, cyano, carbamoyl, methylcarbamoyl, dimethylcarbamoyl, 1,3,4-oxadiazol-2-yl, 5-methyl-1,3,4-oxadiazol, 1,3,4-thiadiazol, 5-methyl-1,3,4-thiadiazol 1H-tetrazolyl, N-morpholinylcarbonylamino, N-morpholinylsulfonyl and N-pyrrolidinylcarbonylamino; R₅ and R₆ are, independently, H, F, Cl or methyl;

### Ar₂ is

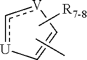

### Ar₂

where the dashed line represents a double bond which may be located formally either between V and the carbon between U and V, or between U and the carbon between U and V; where U is —S—, —O— or —N═ and where, when U is —O— or —S—, V is —CH═, —CCl═ or —N═; and when U is —N═, V CH═, or —NCH₃—; R₇ and R₈ are, independently, H, methoxycarbonyl, methylcarbamoyl, acetamido, acetyl, methyl, ethyl, trifluoromethyl, or halogen.

### Ar₃ is

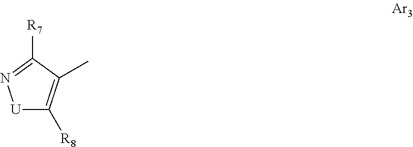

where U is —NH—, —NCH₃— or —O—; and R₇ and R₈ are, independently, H, F, Cl, or methyl;
or a physiologically acceptable salt, solvate, hydrate or stereoisomer thereof; said compounds are published as compounds of general formulae I, IA-1, IA-2, IB-1, IB-2, IC-1, IC-2, ID-1, ID-2, IE-1, IE-2, IIA-1, IIA-2, IIA-3, II-B, III-A, and III-B in International patent application PCT/US2006/028326, published as WO 2007/014011A2 on Jul. 21, 2006, which is incorporated herein by reference in its entirety. In WO 2007/014011A2, said compounds of general formulae I, IA-1, IA-2, IB-1, IB-2, IC-1, IC-2, ID-1, ID-2, IE-1, IE-2, IIA-1, IIA-2, IIA-3, II-B, III-A, and III-B are described on pp. 4 et seq., and pp. 19 et seq., they may be synthesized according to the methods given therein on pp. 39, et seq., and are exemplified as specific compound Examples 1 to 71 therein on pp. 41 to 103. Biological test data for certain of said compounds are given therein on pp. 104 to 111.

Said component B may be in the form of a pharmaceutical formulation which is ready for use to be administered simultaneously, concurrently, separately or sequentially. The components may be administered independently of one another by the oral, intravenous, topical, local installations, intraperitoneal or nasal route.

In accordance with another embodiment of the above-mentioned aspects of the present invention, said combinations are of:

component B: which is one or more 2,3-dihydroimidazo[1,2-c]quinazoline compounds of general formula (B), supra, which is selected from the list consisting of:
Example 1: N-(3,4-difluoro-2-(2-fluoro-4-iodophenylamino)phenyl)-methanesulfonamide:
Example 2: N-(3,4-difluoro-2-(2-fluoro-4-iodophenylamino)phenyl)cyclopropanesulfonamide:
Example 3: N-(3,4-difluoro-2-(2-fluoro-4-iodophenylamino)phenyl)propane-2-sulfonamide:
Example 4: N-(3,4-difluoro-2-(2-fluoro-4-iodophenylamino)phenyl)butane-1-sulfonamide:
Example 5: N-(3,4-difluoro-2-(2-fluoro-4-iodophenylamino)phenyl)-2,2,2-trifluoro ethane sulfonamide:
Example 6: N-(3,4-difluoro-2-(2-fluoro-4-iodophenylamino)phenyl)butane-2-sulfonamide:
Example 7: N-(3,4-difluoro-2-(2-fluoro-4-iodophenylamino)phenyl)-N-methyl cyclopropane sulfonamide:
Example 8: 1-Chloro-N-(3,4-difluoro-2-(2-fluoro-4-iodophenylamino)phenyl) methane sulfonamide:
Example 9: N-(3,4-difluoro-2-(2-fluoro-4-iodophenylamino)phenyl)-2-methylpropane-2-sulfonamide:
Example 10: N-(3,4-difluoro-2-(2-fluoro-4-iodophenylamino)phenyl)cyclopentanesulfonamide:
Example 11: N-(3,4-difluoro-2-(2-fluoro-4-iodophenylamino)phenyl)cyclohexanesulfonamide:
Example 12: N-(3,4-difluoro-2-(2-fluoro-4-iodophenylamino)phenyl)-1-methylcyclopropane-1-sulfonamide:
Example 13: N-(3,4-difluoro-2-(2-fluoro-4-iodophenylamino)phenyl)-1-(2,3-dihydroxypropyl)cyclopropane-1-sulfonamide:
Example 14: (S)—N-(3,4-difluoro-2-(2-fluoro-4-iodophenylamino)phenyl)-1-(2,3-dihydroxypropyl)cyclopropane-1-sulfonamide:
Example 15: (R)—N-(3,4-difluoro-2-(2-fluoro-4-iodophenylamino)phenyl)-1-(2,3-dihydroxypropyl)cyclopropane-1-sulfonamide:
Example 16: N-(3,4-difluoro-2-(2-fluoro-4-iodophenylamino)phenyl)-1-(2-hydroxyethyl)cyclopropane-1-sulfonamide:
Example 17: N-(3,4-difluoro-2-(2-fluoro-4-iodophenylamino)phenyl)-3-hydroxypropane-1-sulfonamide:
Example 18: N-(3,4-difluoro-2-(2-fluoro-4-iodophenylamino)phenyl)-2-methyl-5-(trifluoromethyl)furan-3-sulfonamide:
Example 19: N-(5-(N-(3,4-difluoro-2-(2-fluoro-4-iodophenylamino)phenyl)sulfamoyl)-methylthiazol-2-yl)acetamide:
Example 20: 5-(5-Chloro-1,2,4-thiadiazol-3-yl)-N-(3,4-difluoro-2-(2-fluoro-4-iodophenylamino)phenyl)thiophene-2-sulfonamide:
Example 21: N-(3,4-difluoro-2-(2-fluoro-4-iodophenylamino)phenyl)-3,5-dimethylisoxazole-4-sulfonamide:
Example 22: 5-Chloro-N-(3,4-difluoro-2-(2-fluoro-4-iodophenylamino)phenyl)-1,3-dimethyl-1H-pyrazole-4-sulfonamide:
Example 23: N-(3,4-difluoro-2-(2-fluoro-4-iodophenylamino)phenyl)-2,5-dimethylfuran-3-sulfonamide:
Example 24: N-(3,4-difluoro-2-(2-fluoro-4-iodophenylamino)phenyl)-1-methyl-3-(trifluoromethyl)-1H-pyrazole-4-sulfonamide:
Example 25: N-(3,4-difluoro-2-(2-fluoro-4-iodophenylamino)phenyl)-2,4-dimethylthiazole-5-sulfonamide:
Example 26: N-(3,4-difluoro-2-(2-fluoro-4-iodophenylamino)phenyl)-1,2-dimethyl-1H-imidazole-4-sulfonamide:
Example 27: N-(3,4-difluoro-2-(2-fluoro-4-iodophenylamino)phenyl)thiophene-3-sulfonamide:
Example 28: N-(3,4-difluoro-2-(2-fluoro-4-iodophenylamino)phenyl)furan-2-sulfonamide:
Example 29: N-(3,4-difluoro-2-(2-fluoro-4-iodophenylamino)phenyl)-5-methylthiophene-2-sulfonamide:
Example 30: 5-Chloro-N-(3,4-difluoro-2-(2-fluoro-4-iodophenylamino)phenyl)thiophene-2-sulfonamide:
Example 31: 5-Bromo-N-(3,4-difluoro-2-(2-fluoro-4-iodophenylamino)phenyl)thiophene-2-sulfonamide:
Example 32: 4-Bromo-N-(3,4-difluoro-2-(2-fluoro-4-iodophenylamino)phenyl)thiophene-3-sulfonamide:
Example 33: 4-Bromo-5-chloro-N-(3,4-difluoro-2-(2-fluoro-4-iodophenylamino)phenyl)thiophene-2-sulfonamide:
Example 34: 3-Bromo-5-chloro-N-(3,4-difluoro-2-(2-fluoro-4-iodophenylamino) phenyl)thiophene-2-sulfonamide:
Example 35: N-(3,4-difluoro-2-(2-fluoro-4-iodophenylamino)phenyl)-2,5-dimethylthiophene-3-sulfonamide:
Example 36: 2,5-Dichloro-N-(3,4-difluoro-2-(2-fluoro-4-iodophenylamino)phenyl)thiophene-3-sulfonamide:
Example 37: Methyl 3-(N-(3,4-difluoro-2-(2-fluoro-4-iodophenylamino)phenyl) sulfamoyl)thiophene-2-carboxylate:
Example 38: Methyl 5-(N-(3,4-difluoro-2-(2-fluoro-4-iodophenylamino)phenyl)sulfamoyl)-1-methyl-1H-pyrrole-2-carboxylate:
Example 39: N-(3,4-difluoro-2-(2-fluoro-4-iodophenylamino)phenyl)-5-methylisoxazole-4-sulfonamide:
Example 40: 3-Chloro-N-(3,4-difluoro-2-(2-fluoro-4-iodophenylamino)phenyl)propane-1-sulfonamide:
Example 41: N-(2-(4-chloro-2-fluorophenylamino)-3,4-difluorophenyl)cyclopropanesulfonamide:
Example 42: N-(3,4-difluoro-2-(4-iodo-2-methylphenylamino)phenyl)cyclopropanesulfonamide:
Example 43: N-(2-(4-tert-butyl-2-chlorophenylamino)-3,4-difluorophenyl)cyclopropanesulfonamide:
Example 44: N-(2-(2,4-dichlorophenylamino)-3,4-difluorophenyl)cyclopropanesulfonamide:
Example 45: 3-Chloro-N-(3,4-difluoro-2-(2-fluoro-4-trifluoromethyl) phenylamino)phenyl)propane-1-sulfonamide:
Example 46. N-(3,4-difluoro-2-(2-chloro-4-trifluoromethyl)phenylamino)methanesulfonamide:
Example 47: 3-Chloro-N-(3,4-difluoro-2-(2-chloro-4-trifluoromethyl) phenylamino)phenyl)propane-1-sulfonamide:
Example 48: 3-Chloro-N-(3,4-difluoro-2-(2-bromo-4-trifluoromethyl) phenylamino)phenyl)propane-1-sulfonamide:
Example 49: Cyclopropanesulfonic acid (3,4,6-trifluoro-2-(2-fluoro-4-iodo-phenylamino)-phenyl)-amide:
Example 50: N-(3,4-difluoro-2-(4-fluoro-2-iodophenylamino)-6-ethoxyphenyl)cyclopropane sulfonamide:
Example 51: Methylsulfonic acid (3,4-difluoro-2-(2-fluoro-4-iodo-phenylamino)-6-methoxy-phenyl)-amide:
Example 52: 1-(2,3-Dihydroxy-propyl)-cyclopropanesulfonic acid [3,4,6-trifluoro-2-(4-fluoro-2-iodo-phenylamino)-phenyl]-amide:
Example 53: (S)-1-(2,3-dihydroxypropyl)-N-(3,4,6-trifluoro-2-(2-fluoro-4-iodophenylamino)phenyl)cyclopropane-1-sulfonamide:
Example 54: (R)-1-(2,3-dihydroxypropyl)-N-(3,4,6-trifluoro-2-(2-fluoro-4-iodophenylamino)phenyl)cyclopropane-1-sulfonamide:
Example 55: N-(3,4-difluoro-2-(2-fluoro-4-iodophenylamino)phenyl)-1-(2,3-dihydroxypropyl)cyclopropane-1-sulfonamide:
Example 56: (S)—N-(3,4-difluoro-2-(2-fluoro-4-iodophenylamino)-6-methoxyphenyl)-1-(2,3-dihydroxypropyl)cyclopropane-1-sulfonamide:
Example 57: (R)—N-(3,4-difluoro-2-(2-fluoro-4-iodophenylamino)-6-methoxyphenyl)-1-(2,3-dihydroxypropyl)cyclopropane-1-sulfonamide:
Example 58: 1-(2-hydroxyethyl)-N-(3,4,6-trifluoro-2-(2-fluoro-4-iodophenylamino)phenyl)cyclopropane-1-sulfonamide:
Example 59. N-(3,4-difluoro-2-(2-fluoro-4-iodophenylamino)-6-methoxyphenyl)-1-(2-hydroxyethyl)cyclopropane-1-sulfonamide:
Example 60. N-(3,4-difluoro-2-(2-fluoro-4-iodophenylamino)-6-methoxyphenyl)-1-(3-hydroxy-2-(hydroxymethyl)propyl)cyclopropane-1-sulfonamide:
Example 61: N-(3,4-difluoro-2-(2-fluoro-4-iodophenylamino)-6-methoxyphenyl)cyclobutane sulfonamide:
Example 62: N-(3,4-difluoro-2-(2-fluoro-4-iodophenylamino)-6-methylphenyl)-1-(2,3-dihydroxypropyl)cyclopropane-1-sulfonamide:
Example 63: 1-(2,3-Dihydroxypropyl)-N-(6-ethyl-3,4-difluoro-2-(2-fluoro-4-iodophenylamino)phenyl)cyclopropane-1-sulfonamide:
Example 64: N-(3,4-difluoro-2-(2-fluoro-4-iodophenylamino)-6-(2-methoxyethoxy)phenyl)-1-(2,3-dihydroxypropyl)cyclopropane-1-sulfonamide:
Example 65: 2,4-dichloro-N-(3,4-difluoro-2-(2-fluoro-4-iodophenylamino)phenyl)benzene sulfonamide:
Example 66: 2-chloro-N-(3,4-difluoro-2-(2-fluoro-4-iodophenylamino)phenyl)-4-(trifluoromethyl)benzenesulfonamide:
Example 67: N-(3,4-difluoro-2-(2-fluoro-4-iodophenylamino)phenyl)-2-(trifluoromethoxy)benzene sulfonamide:
Example 68: 4-(N-(3,4-difluoro-2-(2-fluoro-4-iodophenylamino)phenyl)sulfamoyl)benzoic acid:
Example 69: N-(3,4-difluoro-2-(2-fluoro-4-iodophenylamino)phenyl)benzenesulfonamide:
Example 70: N-(3,4-difluoro-2-(2-fluoro-4-iodophenylamino)phenyl)-2-fluorobenzene sulfonamide:
Example 71: N-(3,4-difluoro-2-(2-fluoro-4-methylphenylamino)phenyl)cyclopropanesulfonamide; or a physiologically acceptable salt, solvate, hydrate or stereoisomer thereof.

In accordance with an embodiment of the above-mentioned aspects of the present invention, said combinations are of:

component B: which is Lapatinib;

In accordance with an embodiment of the above-mentioned aspects of the present invention, said combinations are of:

component B: which is Paclitaxel;

In accordance with an embodiment of the above-mentioned aspects of the present invention, said combinations are of:

component A: 2-amino-N-[7-methoxy-8-(3-morpholin-4-ylpropoxy)-2,3-dihydroimidazo[1,2-c]quinazolin-5-yl]pyrimidine-5-carboxamide; and
component B: (S)—N-(3,4-difluoro-2-(2-fluoro-4-iodophenylamino)-6-methoxyphenyl)-1-(2,3-dihydroxypropyl)cyclopropane-1-sulfonamide.

In accordance with an embodiment of the above-mentioned aspects of the present invention, said combinations are of:

component A: 2-amino-N-[7-methoxy-8-(3-morpholin-4-ylpropoxy)-2,3-dihydroimidazo[1,2-c]quinazolin-5-yl]pyrimidine-5-carboxamide; and
component B: lapatinib.

In accordance with an embodiment of the above-mentioned aspects of the present invention, said combinations are of:

component A: 2-amino-N-[7-methoxy-8-(3-morpholin-4-ylpropoxy)-2,3-dihydroimidazo[1,2-c]quinazolin-5-yl]pyrimidine-5-carboxamide; and
component B: paclitaxel.

Said component B may be in the form of a pharmaceutical formulation which is ready for use to be administered simultaneously, concurrently, separately or sequentially. The components may be administered independently of one another by the oral, intravenous, topical, local installations, intraperitoneal or nasal route.

In accordance with an embodiment, the present invention relates to a combination of any component A mentioned herein with any component B mentioned herein, optionally with any component C mentioned herein.

In a particular embodiment, the present invention relates to a combination of a component A with a component B, optionally with a component C, as mentioned in the Examples section herein.

### Useful Forms of Components A and B of the Combinations of the Present Invention

As mentioned supra, either or both of components A and B of any of the combinations of the present invention may be in a useful form, such as pharmaceutically acceptable salts, co-precipitates, metabolites, hydrates, solvates and prodrugs of all the compounds of examples. The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and chorine salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

Representative salts of the compounds of this invention include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, chloride, bromide, iodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, sulfate, tartrate, thiocyanate, tosylate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, or butyl chlorides, bromides and iodides; dialkyl sulfates like dimethyl, diethyl, dibutyl sulfate, or diamyl sulfates, long chain halides such as decyl, lauryl, myristyl and strearyl chlorides, bromides and iodides, aralkyl halides like benzyl and phenethyl bromides and others.

A solvate for the purpose of this invention is a complex of a solvent and a compound of the invention in the solid state. Exemplary solvates would include, but are not limited to, complexes of a compound of the invention with ethanol or methanol. Hydrates are a specific form of solvate wherein the solvent is water.

### Pharmaceutical Formulations of Components A and B of the Combinations of the Present Invention

As mentioned supra, the components A or B may, independently from one another, be in the form of a pharmaceutical formulation which is ready for use to be administered simultaneously, concurrently, separately or sequentially. The components may be administered independently of one another by the oral, intravenous, topical, local installations, intraperitoneal or nasal route.

Said compositions can be utilized to achieve the desired pharmacological effect by administration to a patient in need thereof. A patient, for the purpose of this invention, is a mammal, including a human, in need of treatment for the particular condition or disease. Therefore, the present invention includes combinations in which components A and B, independently of one another, are pharmaceutical formulations compositions that are comprised of a pharmaceutically acceptable carrier and a pharmaceutically effective amount of a said component. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of component, and/or combination. A pharmaceutically effective amount of a combination is preferably that amount which produces a result or exerts an influence on the particular condition being treated. The combinations of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

For oral administration, the combinations can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatin type containing, for example, surfactants, lubricants, and inert fillers such as lactose, sucrose, calcium phosphate, and corn starch.

In another embodiment, the combinations of this invention may be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavoring and coloring agents.

The combinations of this invention may also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quaternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)—Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);
alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoal);
aerosol propellants (examples include but are not limited to carbon dioxide, CCl₂F₂, F₂ClC—CClF₂ and CClF₃)
air displacement agents (examples include but are not limited to nitrogen and argon);
antifungal preservatives (examples include but are not limited to benzoic acid, butylparaben, ethylparaben, methylparaben, propylparaben, sodium benzoate);
antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);
antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);
binding materials (examples include but are not limited to block polymers, natural and synthetic rubber, polyacrylates, polyurethanes, silicones, polysiloxanes and styrene-butadiene copolymers);
buffering agents (examples include but are not limited to potassium metaphosphate, dipotassium phosphate, sodium acetate, sodium citrate anhydrous and sodium citrate dihydrate)
carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)
chelating agents (examples include but are not limited to edetate disodium and edetic acid)
colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);
clarifying agents (examples include but are not limited to bentonite);
emulsifying agents (examples include but are not limited to acacia, cetomacrogol, cetyl alcohol, glyceryl monostearate, lecithin, sorbitan monooleate, polyoxyethylene 50 monostearate);
encapsulating agents (examples include but are not limited to gelatin and cellulose acetate phthalate)
flavorants (examples include but are not limited to anise oil, cinnamon oil, cocoa, menthol, orange oil, peppermint oil and vanillin);
humectants (examples include but are not limited to glycerol, propylene glycol and sorbitol);
levigating agents (examples include but are not limited to mineral oil and glycerin);
oils (examples include but are not limited to arachis oil, mineral oil, olive oil, peanut oil, sesame oil and vegetable oil);
ointment bases (examples include but are not limited to lanolin, hydrophilic ointment, polyethylene glycol ointment, petrolatum, hydrophilic petrolatum, white ointment, yellow ointment, and rose water ointment);
penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)
plasticizers (examples include but are not limited to diethyl phthalate and glycerol);
solvents (examples include but are not limited to ethanol, corn oil, cottonseed oil, glycerol, isopropanol, mineral oil, oleic acid, peanut oil, purified water, water for injection, sterile water for injection and sterile water for irrigation);
stiffening agents (examples include but are not limited to cetyl alcohol, cetyl esters wax, microcrystalline wax, paraffin, stearyl alcohol, white wax and yellow wax);
suppository bases (examples include but are not limited to cocoa butter and polyethylene glycols (mixtures));
surfactants (examples include but are not limited to benzalkonium chloride, nonoxynol 10, oxtoxynol 9, polysorbate 80, sodium lauryl sulfate and sorbitan mono-palmitate);
suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);
sweetening agents (examples include but are not limited to aspartame, dextrose, glycerol, mannitol, propylene glycol, saccharin sodium, sorbitol and sucrose);
tablet anti-adherents (examples include but are not limited to magnesium stearate and talc);
tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);
tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);
tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);
tablet direct compression excipients (examples include but are not limited to dibasic calcium phosphate);
tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);
tablet glidants (examples include but are not limited to colloidal silica, corn starch and talc);
tablet lubricants (examples include but are not limited to calcium stearate, magnesium stearate, mineral oil, stearic acid and zinc stearate);
tablet/capsule opaquants (examples include but are not limited to titanium dioxide);
tablet polishing agents (examples include but are not limited to carnuba wax and white wax);
thickening agents (examples include but are not limited to beeswax, cetyl alcohol and paraffin);
tonicity agents (examples include but are not limited to dextrose and sodium chloride);
viscosity increasing agents (examples include but are not limited to alginic acid, bentonite, carbomers, carboxymethylcellulose sodium, methylcellulose, polyvinyl pyrrolidone, sodium alginate and tragacanth); and
wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be illustrated as follows:

Sterile IV Solution: A 5 mg/mL solution of the desired compound of this invention can be made using sterile, injectable water, and the pH is adjusted if necessary. The solution is diluted for administration to 1-2 mg/mL with sterile 5% dextrose and is administered as an IV infusion over about 60 minutes.
Lyophilized powder for IV administration: A sterile preparation can be prepared with (i) 100-1000 mg of the desired compound of this invention as a lypholized powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 minutes.
Intramuscular suspension: The following solution or suspension can be prepared, for intramuscular injection:
50 mg/mL of the desired, water-insoluble compound of this invention
5 mg/mL sodium carboxymethylcellulose
4 mg/mL TWEEN 80
9 mg/mL sodium chloride
9 mg/mL benzyl alcohol
Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.
Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.
Tablets: A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.
Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### Method of Treating Cancer

Within the context of the present invention, the term “cancer” includes, but is not limited to, cancers of the breast, lung, brain, reproductive organs, digestive tract, urinary tract, liver, eye, skin, head and neck, thyroid, parathyroid and their distant metastases. Those disorders also include multiple myeloma, lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypothalamic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell.

Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

The present invention relates to a method for using the combinations of the present invention, to treat cancer, as described infra, particularly mammalian NSCLC, CRC, melanoma, pancreatic cancer, hepatocyte or breast cancer. Combinations can be utilized to inhibit, block, reduce, decrease, etc., cell proliferation and/or cell division, and/or produce apoptosis, in the treatment or prophylaxis of cancer, in particular NSCLC, CRC, melanoma, pancreatic cancer, hepatocyte carcinoma or breast cancer. This method comprises administering to a mammal in need thereof, including a human, an amount of a combination of this invention, or a pharmaceutically acceptable salt, isomer, polymorph, metabolite, hydrate, solvate or ester thereof; etc. which is effective for the treatment or prophylaxis of cancer, in particular NSCLC, CRC, melanoma, pancreatic cancer, hepatocyte carcinoma or breast cancer.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

### Dose and Administration

Based upon standard laboratory techniques known to evaluate compounds useful for the treatment or prophylaxis of cancer, in particular NSCLC, CRC, melanoma, pancreatic cancer, hepatocyte carcinoma or breast cancer, by standard toxicity tests and by standard pharmacological assays for the determination of treatment of the conditions identified above in mammals, and by comparison of these results with the results of known medicaments that are used to treat these conditions, the effective dosage of the combinations of this invention can readily be determined for treatment of the indication. The amount of the active ingredient to be administered in the treatment of the condition can vary widely according to such considerations as the particular combination and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1,500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific combination employed, the age and general condition of the patient, time of administration, route of administration, rate of excretion of the drug, drug combinations, and the like. The desired mode of treatment and number of doses of a combination of the present invention or a pharmaceutically acceptable salt or ester or composition thereof can be ascertained by those skilled in the art using conventional treatment tests.

### Therapies Using Combinations of Component A as Described Supra, Component B as Described Supra, and Component C: One or More Further Pharmaceutical Agents.

The combinations of component A and component B of this invention can be administered as the sole pharmaceutical agent or in combination with one or more further pharmaceutical agents where the resulting combination of components A, B and C causes no unacceptable adverse effects. For example, the combinations of components A and B of this invention can be combined with component C, i.e. one or more further pharmaceutical agents, such as known anti-angiogenesis, anti-hyper-proliferative, antiinflammatory, analgesic, immunoregulatory, diuretic, antiarrhythmic, anti-hypercholesterolemia, anti-dyslipidemia, anti-diabetic or antiviral agents, and the like, as well as with admixtures and combinations thereof.

Component C, can be one or more pharmaceutical agents such as aldesleukin, alendronic acid, alfaferone, alitretinoin, allopurinol, aloprim, aloxi, altretamine, aminoglutethimide, amifostine, amrubicin, amsacrine, anastrozole, anzmet, aranesp, arglabin, arsenic trioxide, aromasin, 5-azacytidine, azathioprine, BCG or tice BCG, bestatin, betamethasone acetate, betamethasone sodium phosphate, bexarotene, bleomycin sulfate, broxuridine, bortezomib, busulfan, calcitonin, campath, capecitabine, carboplatin, casodex, cefesone, celmoleukin, cerubidine, chlorambucil, cisplatin, cladribine, cladribine, clodronic acid, cyclophosphamide, cytarabine, dacarbazine, dactinomycin, DaunoXome, decadron, decadron phosphate, delestrogen, denileukin diftitox, depo-medrol, deslorelin, dexomethasone, dexrazoxane, diethylstilbestrol, diflucan, docetaxel, doxifluridine, doxorubicin, dronabinol, DW-166HC, eligard, elitek, ellence, emend, epirubicin, epoetin alfa, epogen, eptaplatin, erlotinib (when component B is not itself erlotinib), ergamisol, estrace, estradiol, estramustine phosphate sodium, ethinyl estradiol, ethyol, etidronic acid, etopophos, etoposide, fadrozole, farston, filgrastim, finasteride, fligrastim, floxuridine, fluconazole, fludarabine, 5-fluorodeoxyuridine monophosphate, 5-fluorouracil (5-FU), fluoxymesterone, flutamide, formestane, fosteabine, fotemustine, fulvestrant, gammagard, gemcitabine, gemtuzumab, gleevec, gliadel, goserelin, granisetron HCl, histrelin, hycamtin, hydrocortone, erythro-hydroxynonyladenine, hydroxyurea, ibritumomab tiuxetan, idarubicin, ifosfamide, interferon alpha, interferon-alpha 2, interferon alfa-2A, interferon alfa-2B, interferon alfa-n1, interferon alfa-n3, interferon beta, interferon gamma-1a, interleukin-2, intron A, iressa, irinotecan, kytril, lapatinib (when component B is not itself lapatinib), lentinan sulphate, letrozole, leucovorin, leuprolide, leuprolide acetate, lenalidomide, levamisole, levofolinic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, Mesna, methotrexate, metvix, miltefosine, minocycline, mitomycin C, mitotane, mitoxantrone, Modrenal, Myocet, nedaplatin, neulasta, neumega, neupogen, nilutamide, nolvadex, NSC-631570, OCT-43, octreotide, ondansetron HCl, orapred, oxaliplatin, paclitaxel (when component B is not itself paclitaxel), pediapred, pegaspargase, Pegasys, pentostatin, picibanil, pilocarpine HCl, pirarubicin, plicamycin, porfimer sodium, prednimustine, prednisolone, prednisone, premarin, procarbazine, procrit, raltitrexed, rebif, rhenium-186 etidronate, rituximab, roferon-A, romurtide, salagen, sandostatin, sargramostim, semustine, sizofiran, sobuzoxane, solu-medrol, sparfosic acid, stem-cell therapy, streptozocin, strontium-89 chloride, sunitinib, synthroid, tamoxifen, tamsulosin, tasonermin, tastolactone, taxotere, teceleukin, temozolomide, teniposide, testosterone propionate, testred, thioguanine, thiotepa, thyrotropin, tiludronic acid, topotecan, toremifene, tositumomab, trastuzumab, treosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin stimalamer, zofran, ABI-007, acolbifene, actimmune, affinitak, aminopterin, arzoxifene, asoprisnil, atamestane, atrasentan, BAY 43-9006 (sorafenib), avastin, CCl-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon gamma, intron-PEG, ixabepilone, keyhole limpet hemocyanin, L-651582, lanreotide, lasofoxifene, libra, lonafarnib, miproxifene, minodronate, MS-209, liposomal MTP-PE, MX-6, nafarelin, nemorubicin, neovastat, nolatrexed, oblimersen, onco-TCS, osidem, paclitaxel polyglutamate, pamidronate disodium, PN-401, QS-21, quazepam, R-1549, raloxifene, ranpirnase, 13-cis-retinoic acid, satraplatin, seocalcitol, T-138067, tarceva, taxoprexin, thalidomide, thymosin alpha 1, tiazofurine, tipifarnib, tirapazamine, TLK-286, toremifene, TransMID-107R, valspodar, vapreotide, vatalanib, verteporfin, vinflunine, Z-100, zoledronic acid or combinations thereof.

Alternatively, said component C can be one or more further pharmaceutical agents selected from gemcitabine, paclitaxel (when component B is not itself paclitaxel), cisplatin, carboplatin, sodium butyrate, 5-FU, doxirubicin, tamoxifen, etoposide, trastumazab, gefitinib, intron A, rapamycin, 17-MG, U0126, insulin, an insulin derivative, a PPAR ligand, a sulfonylurea drug, an α-glucosidase inhibitor, a biguanide, a PTP-1B inhibitor, a DPP-IV inhibitor, a 11-beta-HSD inhibitor, GLP-1, a GLP-1 derivative, GIP, a GIP derivative, PACAP, a PACAP derivative, secretin or a secretin derivative.

Optional anti-hyper-proliferative agents which can be added as component C to the combination of components A and B of the present invention include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use as component C with the combination of components A and B of the present invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyl adenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel (when component B is not itself paclitaxel), pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use as component C with the combination of components A and B of the present invention include but are not limited to other anti-cancer agents such as epothilone and its derivatives, irinotecan, raloxifen and topotecan.

Generally, the use of cytotoxic and/or cytostatic agents as component C in combination with a combination of components A and B of the present invention will serve to:

(1) yield better efficacy in reducing the growth of a tumor or even eliminate the tumor as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumor progression, and/or
(8) yield efficacy and tolerability results at least as good as those of the agents used alone, compared to known instances where other cancer agent combinations produce antagonistic effects.

### EXAMPLES

The following abbreviations are used in the Examples:

“cA” means compound Example 13 of WO 2008/070150 A1 as shown herein (which is an Example of component A as described and defined herein).
“cB” means compound Example 56 of WO 2007/014011 A2, i.e. (S)—N-(3,4-difluoro-2-(2-fluoro-4-iodophenylamino)-6-methoxyphenyl)-1-(2,3-dihydroxypropyl)cyclopropane-1-sulfonamide, of structure:

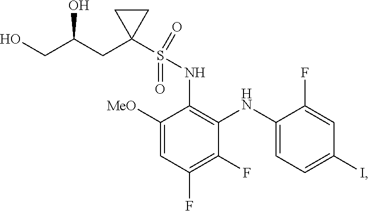 [SMILES: COc1cc(F)c(F)c(Nc2ccc(I)cc2F)c1NS(=O)(=O)C1(C[C@H](O)CO)CC1]

(which is an Example of component B as described and defined herein).
“BB” means cB, Lapatininb or paclitaxel (as examples of component B).

The effects of combinations of the present invention were evaluated using combination index isobologram analysis for in vitro assessment. The efficacy parameters were the effects in a 72-hour cell proliferation assay or in a 48-hour caspase 3/7 activation assay. Briefly, cells were plated in 384-well plate with 25 μL medium. After 24 hours, 5 μL of experimental media containing either cA alone, or BB (such as cB, or Lapatinib, paclitaxel (example of component B) alone, etc.), or the combination of cA (as component A) plus either cB, or Lapatinib, or paclitaxel, (as component B), at different ratios (0.8×cA+0.2×BB, 0.6×cA+0.4×BB, 0.4×cA+0.6×BB, 0.2×cA+0.8×BB, 0.1×cA+0.9×BB) were used to make serial three-fold dilutions to generate response curves at 7 concentrations. Experiments were conducted in triplicates. The mapping EC50/IC50 and EC90/IC90 values were calculated using Analyze5 computer program. The corresponding component concentrations of cA and BB (either cB, or Lapatinib, or paclitaxel (as component B)) at the E(I)C50/E(I)C90 were calculated and used for plotting isobolograms. Effects were analyzed as described by Chou (Pharmacology Reviews 2006) and the combination index was calculated using the formula:

Combination Index=[cAx]/cA′+[BBx]/BB′

[cAx] and [BBx] refer to cA and BB (either cB, or Lapatinib, or paclitaxel (as component B)), concentration at EC50/IC50 or EC90/IC90, respectively, in combination. cA′ and BB′ refer to the EC50/IC50 or EC90/IC90 values of cA and BB, respectively, as a single agent. Combination indices of 0-0.3, 0.3-0.6, and 0.6-0.9 were defined to indicate very strong synergy, strong synergy and synergy, respectively.

The in vivo combination effects were evaluated in tumor xenograft models in nude mice with either established human tumor cell lines or patient-derived primary tumor models at the MTD and sub-MTD dosages.

The invention is demonstrated in the following examples which are not meant to limit the invention in any way:

### Example 1

To investigate if combining the PI3K inhibitor with MEK inhibitor and/or established therapies could result in synergistic or additive effects, and/or overcome the resistance to the chemotherapies in cancer (include but not limited to NSCLC, melanoma, pancreatic cancer, hepatocyte carcinoma breast, or CRC) treatment, we conducted combination studies to assess the anti-tumor activities of single agent versus combination therapy in vitro and in vivo.

The drugs having potential combinability and synergy with the 2,3-dihydroimidazo[1,2-c]quinazoline compounds are described above, particularly, but not limited to Dexamethasone, Thalidomide, Bortezomib, Melphalan, Rapalogs (temsirolimus, everolimus, and AP23573), drugs inhibiting MAPK pathway, Stat1-5 pathways, IKK-NFkappaB pathways, AKT-mTOR pathway, integrin pathways, antiangiognic drugs, etc.

The combination with 2,3-dihydroimidazo[1,2-c]quinazoline compounds can also include more than one compound: it could be two, or more compounds.

Table 1 shows the combination index of cA (as component A) with cB, Erlotinib, Lapatinib, and Paclitaxel (as component B), in CRC, lung and breast tumor cell lines, respectively. Very strong (combination index<0.3) to strong synergy (combination index 0.3<CI<0.6) were demonstrated in all the tumor cell lines and combination drugs listed in Tablet, except the combination of cA (as component A) with erlotinib (as component B) in NCI-H1975, a cell line has double EGFR mutations and resistant to erlotinib, showed moderate synergy (CI=0.60-0.65). Importantly, in most of the cases, stronger synergy was observed with IC90, indicating these combinations greatly enhanced maximum tumor growth inhibition compared to monotherapy.

TABLE 1
Summary of combination effects of cA (as component A)* with either
cB, Erlotinib, Lapatinib or paclitaxel (as component B) in proliferation assays

| | | Combination Index | | | | | | | |
|---|---|---|---|---|---|---|---|---|---|
| | | | | cA/ | | cA/ | | cA/ | |
| Cancer | | cA/cB | | Erlotinib | | Lapatinib | | Paclitaxel | |
| Indication | Cell Line | IC50 | IC90 | IC50 | IC90 | IC50 | IC90 | IC50 | IC90 |
| Lung | A549 | 0.28 | 0.18 | | | | | 0.81 | 0.46 |
| | NCI-H460 | 0.16 | 0.11 | | | | | | |
| | NCI-H1975 | 0.38 | 0.08 | 0.62 | 0.65 | | | | |
| | NCI-H1650 | 0.59 | 0.34 | 0.17 | 0.48 | | | | |
| | NCI-H23 | 0.12 | 0.12 | | | | | | |
| CRC | Colo205 | 0.38 | 0.31 | | | | | | |
| | SW620 | 0.21 | 0.03 | | | | | | |
| | LoVo | 0.31 | 0.18 | | | | | | |
| | HCT116 | 0.11 | 0.05 | | | | | 0.37 | 0.03 |
| | DLD1 | 0.06 | NA | | | | | | |
| Breast | MDA-MB-231 | 0.23 | 0.09 | | | | | | |
| | MDA-MB-468 | | | 0.38 | 0.59 | | | | |
| | T47D | | | | | 0.64 | 0.44 | | |
| | BT474 | | | | | 0.49 | 0.43 | | |

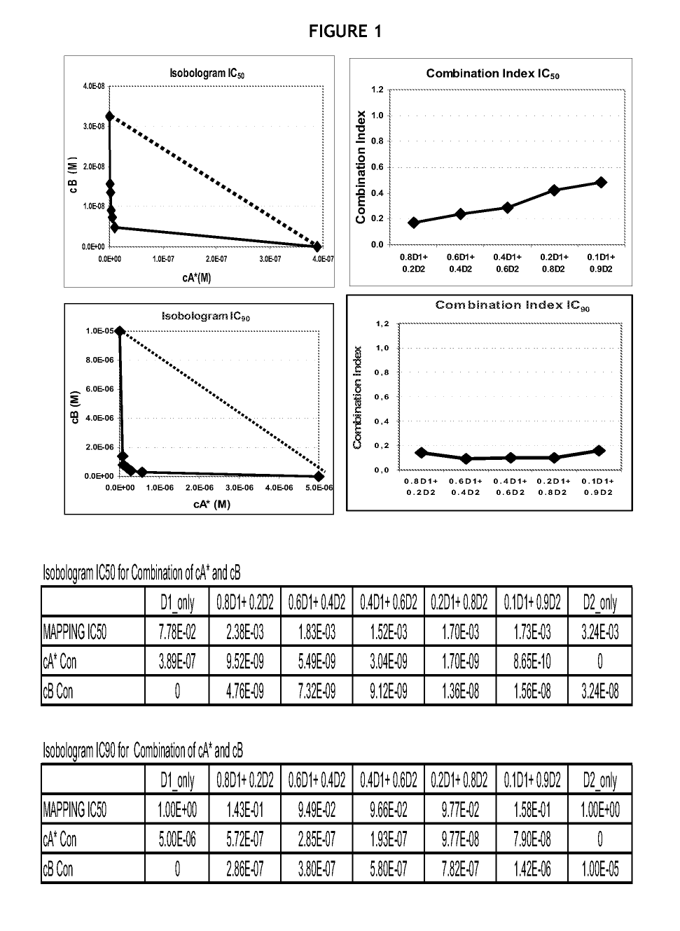

For example, combining cA (as component A) with cB (as component B) showed not only strong synergy with respect to IC50s (combination index 0.21-0.90), but also dramatically lowered IC90s, with combination indices of 0.02-0.18 across the entire tested range of concentrations. As a result, while neither cA nor cB as a single agent could inhibit proliferation by 90% at 5 μM, the IC90 was reached when combining e.g. 285 nM of cA (as component A) and 380 nM of cB (as component B) (see FIG. 1).

FIG. 1: Isobologram/Combination Index Analysis on the Combination of cA* and cB Against Proliferation in CRC SW620 Tumor Cell Line.
*A 72-hour proliferation assay was conducted using Cell Titer Glo (Promega). The top concentrations of cA* and cB were 5 μM and 10 μM, respectively. MAPPING IC₅₀ and IC₉₀ refers to the IC₅₀ and IC₉₀ obtained from the dose-response curve of either cA* or cB alone, or cA* plus cB with the ratio indicated in the table, where the top relative concentration is defined as 1.

A similar result was obtained when testing for activation of caspase 3/7 as a marker of apoptosis induction in NCI-H1975 (NSCLC), NCI-H1650 (NSCLC), HCT116 (CRC), Colo205 (CRC), and MDA-MB-468 (breast cancer) cell lines Table 2. For example, Neither 10 μM of cB nor 5 μM of cA as single agents could induce apoptosis, while the combination of the two drugs led to the activation of caspase 3/7 with combination indexes of 0.09-0.18 across all concentrations.

TABLE 2
Summary of combination effects of
cA* and cB in caspase 3/7 assays

Combination Index

| Cancer | | cA/cB | cA/Erlotinib |
|---|---|---|---|
| Indication | Cell Line | IC50 | IC50 |
| NSCLC | NCI-H1975 | 0.04 | |
| | NCI-H1650 | 0.85 | |
| CRC | Colo205 | 0.06 | |
| | HCT116 | 0.09 | |
| Breast | MDA-MB-468 | 0.50 | |
| | BT474 | | 0.17 |

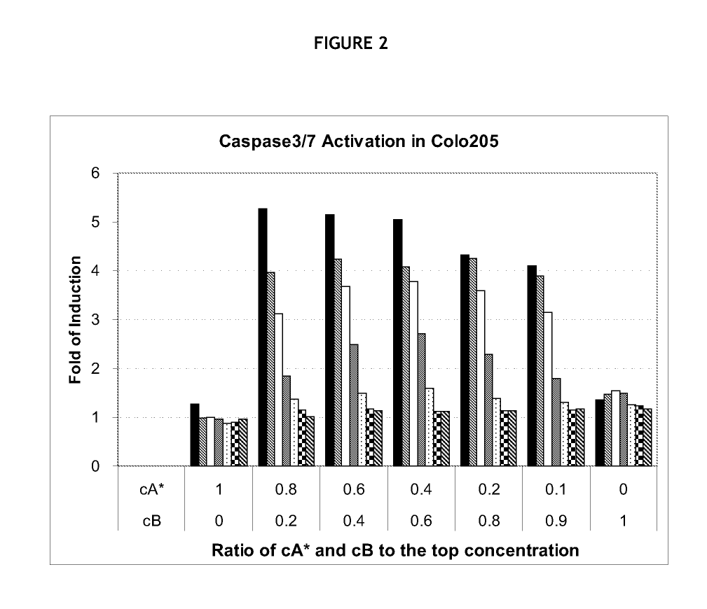

FIG. 2: Activation of caspase3/7 by combined treatment of cA* and cB.

Caspase 3/7 assay was conducted at 48 h after compound exposure to HCT116 (A) and at 24 h after compound exposure to Colo205 (B). Method for compound combination and dilution were described in 3.3.1.2.1. The top concentrations of cA* and cB were 5 μM and 10 μM, respectively. The dose-response curve of either cA* or cB alone, or cA* (as component A) plus cB (as component B) with the ratio indicated in the figure, where the top relative concentrations are defined as 1.

### Example 2

### Synergistic Combination of cA* (as Component A) with cB and Paclitaxel In Vivo

To confirm the synergy demonstrated in the in vitro studies, the combination of cA with cB and paclitaxel was tested in patient-derived primary NSCLC and CRC xenografts in nude mice.

The first model was Co5841 (resistant to Cetuximab). cB was dosed daily at 12.5 (half-MTD) and 25 mg/kg (MTD) from day 6 to day 23. cA (MTD) was dosed weekly (day 6, 13, and 20) at 10 mg/kg BID (MTD) and at 14 mg/kg with Q2D schedule (from day 6 to day 22). Tumor size was monitored twice weekly. cB as a single agent seemed more effective (T/C=0.35 for 12.5 mg/kg group; T/C=0.20 for 25 mg/kg group) than cA* (T/C=0.49 and 0.39 for weekly and Q2D dosing schedules, respectively,

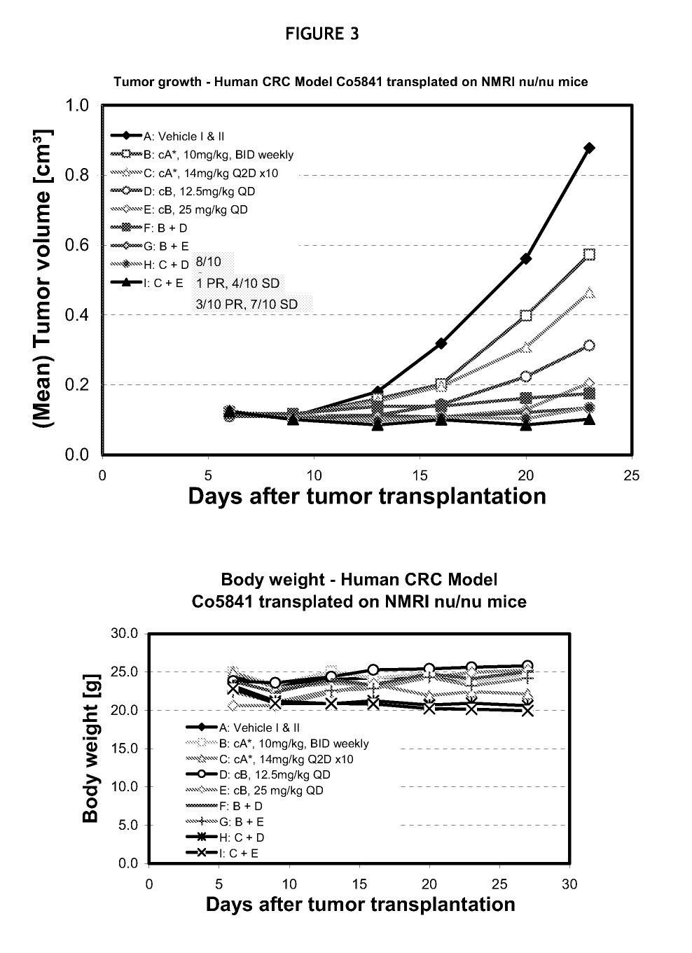

FIG. 3). Clear synergistic effects were observed in the combination. The combination groups with cB at 25 mg/kg showed the best efficacy (T/C=0.13 and 0.10 for weekly and Q2D dosing cA, respectively). More importantly, these two combination groups significantly improved the disease control rates in comparison to each monotherapy. Thus, only 0% and 20% animals showed disease progression (DP) in the groups with 25 mg/kg of cB plus 14 mg/kg (Q2D) or 10 mg/kg (BID weekly) of cA, respectively. In the corresponding monotherapy groups, 70% animals treated with 25 mg/kg of cB, and 100% and 90% of animals treated with 14 mg/kg (Q2D) or 10 mg/kg (BID weekly) of cA* exhibited DP.

FIG. 3: Dose-dependent tumor growth inhibition in Co5841 primary human xenograft CRC model. Co5841 primary human tumor was derived from a patient with CRC and was xenografted in nude mice. The tumor was propagated in vivo and tumor tissue from one in vivo passage was used for s.c. implantation in the inguinal region of male nude mice. Treatment was started when the tumors were approximately 0.1 cm³ in size. Treatment was continued until progression of the tumors. Tumor diameters and body weight were monitored weekly. cA* was dosed at 14 mg/kg, Q2D×7 from day 6 to day 22 (group C, H and I), or 10 mg/kg, BID×1 weekly on day 6, 13, and 20. cB was dosed at either 12.5 mg/kg (group D, F and H), QD, or 25 mg/kg (group E, G and I), QD from day 6 to day 22.

The synergistic combination of cA* and cB was also confirmed in a patient-derived NSCLC xenograft model—Lu7187. This NSCLC model is resistant to erlotinib, paclitaxel, and etoposid, while Cetuximab and carboplatin (T/C=0.21-0.35) are moderately efficacious. cB was dosed daily at 12.5 (half-MTD) and 25 mg/kg (MTD) from day 7 to day 35. cA (MTD) was dosed weekly (day 7, 14, 21 and 28) at 10 mg/kg BID (MTD).

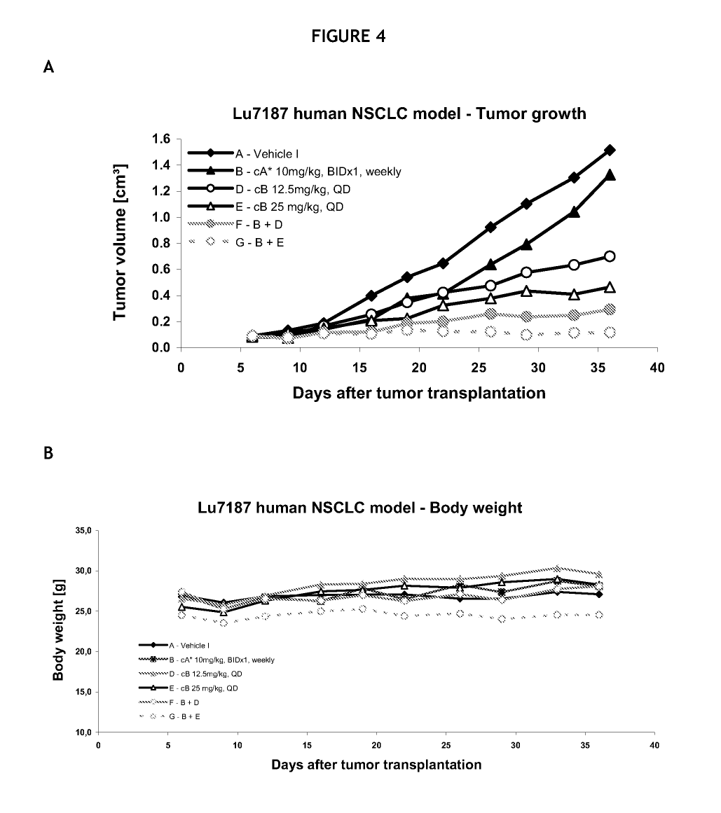

Similar to the CRC model described above, cB as a single agent was more effective (12.5 mg/kg: T/C=0.46; 25 mg/kg: T/C=0.31) than cA* (T/C=0.88; see FIG. 4). Clear synergistic effects were observed in the combination (T/C=0.08), which resulted in effective suppression of tumor growth in this model. cA in combination with 25 mg/kg of cB resulted in 3 PR and 3 SDs while 100% of the animals in the respective monotherapy groups exhibited disease progression. Weekly dosing of cA* showed similar efficacy to the Q2D dosing schedule, but exhibited less body weight loss.

Clear synergistic effects were observed in the combination (T/C=0.08), which resulted in effective suppression of tumor growth in this model. cA in combination with 25 mg/kg of cB resulted in 3 PR and 3 SDs while 100% of the animals in the respective monotherapy groups exhibited disease progression. Weekly dosing of cA* showed similar efficacy to the Q2D dosing schedule, but exhibited less body weight loss.

FIG. 4: Dose-dependent tumor growth inhibition in Lu7187 primary human xenograft NSCLC model. Lu7187 primary human tumor was derived from a patient with NSCLC and was xenografted in nude mice. The tumor was propagated in vivo and tumor tissue from one in vivo passage was used for s.c. implantation in the inguinal region of male nude mice. Treatment was started when the tumors were approximately 0.1 cm³ in size. Treatment was continued until progression of the tumors. Tumor diameters and body weight were monitored t weekly. cA* was dosed at 14 mg/kg, Q2D×10 from day 7 to day 25 (group C, H and I), or 10 mg/kg, BID×1 weekly on day 7, 14, 21 and 28. cB was dosed at either 12.5 mg/kg (group D, F and H), QD, or 25 mg/kg (group E, G and I), QD from day 7 to day 35.

The synergistic combination of cA* and palitaxel was also confirmed in a patient-derived NSCLC xenograft model—Lu7187. This NSCLC model is resistant to etoposid, Cetuximab and erlotinib (T/C>0.5). Paclitaxel as a single agent was very efficacious at 25 mg/kg (MTD). However, 60% of the mice exhibited disease progression after stopping the treatment. In contrast, the corresponding combination group (25 mg/kg paclitaxel and 10 mg/kg cA*) demonstrated complete 100% disease control rate (30% complete tumor regression and 70% partial regression), indicating clear synergistic effects using cA in combination with paclitaxel.

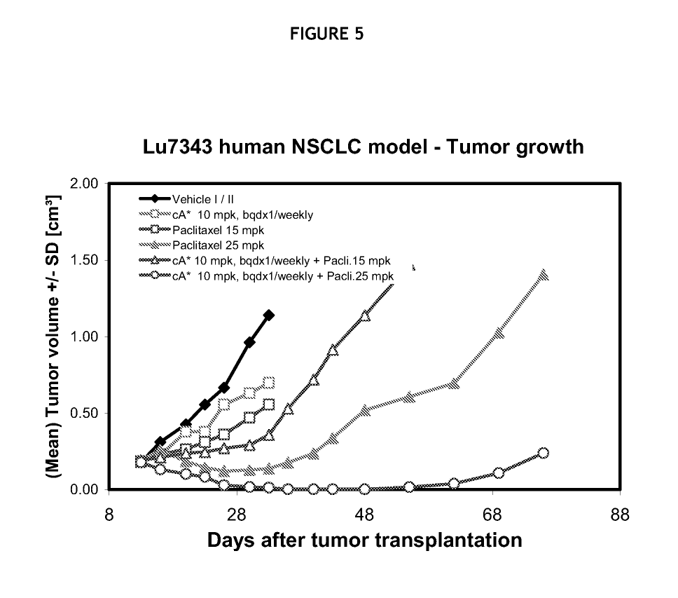

FIG. 5: Dose-dependent tumor growth inhibition in Lu7343 primary human xenograft NSCLC model. Lu7343 primary human tumor was derived from a patient with NSCLC and was xenografted in nude mice. The tumor was propagated in vivo and tumor tissue from one in vivo passage was used for s.c. implantation in the inguinal region of male nude mice. Treatment was started when the tumors were approximately 0.1 cm³ in size. Treatment was continued until progression of the tumors. Tumor diameters and body weight were monitored t weekly. cA* was dosed at 10 mg/kg, BIDx1 weekly on day 15, 22, and 29. paclitaxel was dosed at either 15 mg/kg, or 25 mg/kg, once a week on day 14, 21 and day 28.

1. A combination of:
component A: one or more 2,3-dihydroimidazo[1,2-c]quinazoline compounds of general formula (A1): 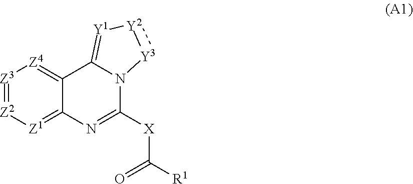
wherein
X represents CR⁵R⁶ or NH;
Y¹ represents CR³ or N;
Chemical bond between [symbol] represents a single bond or double bond,
with the proviso that when the [symbol] represents a double bond,
Y² and Y³ independently represent CR⁴ or N, and
when [symbol] represents a single bond, Y² and Y³ independently represent CR³R⁴ or NR⁴;
Z¹, Z², Z³ and Z⁴ independently represent CH, CR² or N;
R¹ represents aryl optionally having 1 to 3 substituents selected from R¹¹, C₃₋₈ cycloalkyl optionally having 1 to 3 substituents selected from R¹¹,
C₁₋₆ alkyl optionally substituted by
aryl, heteroaryl, C₁₋₆ alkoxyaryl, aryloxy, heteroaryloxy or one or more halogen,
C₁₋₆ alkoxy optionally substituted by
carboxy, aryl, heteroaryl, C₁₋₆ alkoxyaryl, aryloxy, heteroaryloxy or one or more halogen,
or
a 3 to 15 membered mono- or bi-cyclic heterocyclic ring that is saturated or unsaturated, and contains 1 to 3 heteroatoms selected from the group consisting of N, O and S, and optionally having 1 to 3 substituents selected from R¹¹
wherein
R¹¹ represents
halogen, nitro, hydroxy, cyano, carboxy, amino, N—(C₁₋₆alkyl)amino, N-(hydroxyC₁₋₆alkyl)amino, N,N-di(C₁₋₆alkyl)amino, N—(C₁₋₆acyl)amino, N-(formyl)-N—(C₁₋₆alkyl)amino, N—(C₁₋₆alkanesulfonyl)amino, N-(carboxyC₁₋₆alkyl)-N—(C₁₋₆alkyl)amino, N—(C₁₋₆alkoxycarbonyl)amino, N—[N,N-di(C₁₋₆alkyl)amino methylene]amino, N—[N,N-di(C₁₋₆alkyl)amino (C₁₋₆ alkyl)methylene]amino, N—[N,N-di(C₁₋₆alkyl)amino C₂₋₆alkenyl]amino, aminocarbonyl, N—(C₁₋₆alkyl)aminocarbonyl, N,N-di(C₁₋₆alkyl)aminocarbonyl, C₃₋₈cycloalkyl, C₁₋₆ alkylthio, C₁₋₆alkanesulfonyl, sulfamoyl, C₁₋₆alkoxycarbonyl,
N-arylamino wherein said aryl moiety is optionally having 1 to 3 substituents selected from R¹⁰¹, N-(aryl C₁₋₆alkyl)amino wherein said aryl moiety is optionally having 1 to 3 substituents selected from R¹⁰¹, aryl C₁₋₆alkoxy-carbonyl wherein said aryl moiety is optionally having 1 to 3 substituents selected from R¹⁰¹,
C₁₋₆alkyl optionally substituted by
mono-, di- or tri-halogen, amino, N—(C₁₋₆alkyl)amino or N,N-di(C₁₋₆alkyl)amino, C₁₋₆alkoxy optionally substituted by
mono-, di- or tri-halogen, N—(C₁₋₆alkyl)sulfonamide, or N-(aryl)sulfonamide,
or
a 5 to 7 membered saturated or unsaturated ring having 1 to 3 heteroatoms selected from the group consisting of O, S and N, and optionally having 1 to 3 substituents selected from R¹⁰¹
wherein
R¹⁰¹ represents
halogen, carboxy, amino, N—(C₁₋₆ alkyl)amino, N,N-di(C₁₋₆alkyl)amino, aminocarbonyl, N—(C₁₋₆alkyl)aminocarbonyl, N,N-di(C₁₋₆alkyl)aminocarbonyl, pyridyl,
C₁₋₆ alkyl optionally substituted by cyano or mono- di- or tri-halogen,
or
C₁₋₆alkoxy optionally substituted by cyano, carboxy, amino, N—(C₁₋₆ alkyl)amino, N,N-di(C₁₋₆alkyl)amino, aminocarbonyl, N—(C₁₋₆alkyl)aminocarbonyl, N,N-di(C₁₋₆alkyl)aminocarbonyl or mono-, di- or tri-halogen;
R² represents hydroxy, halogen, nitro, cyano, amino, N—(C₁₋₆alkyl)amino, N,N-di(C₁₋₆alkyl)amino, N-(hydroxyC₁₋₆alkyl)amino, N-(hydroxyC₁₋₆alkyl)amino, C₁₋₆ acyloxy, aminoC₁₋₆ acyloxy, C₂₋₆alkenyl, aryl,
a 5-7 membered saturated or unsaturated heterocyclic ring having 1 to 3 heteroatoms selected from the group consisting O, S and N, and optionally substituted by
hydroxy, C₁₋₆ alkyl, C₁₋₆ alkoxy, oxo, amino, amino C₁₋₆alkyl, N—(C₁₋₆alkyl)amino, N,N-di(C₁₋₆alkyl)amino, N—(C₁₋₆ acyl)amino, N—(C₁₋₆alkyl)carbonylamino, phenyl, phenyl C₁₋₆ alkyl, carboxy, C₁₋₆alkoxycarbonyl, aminocarbonyl, N—(C₁₋₆alkyl)aminocarbonyl, or N,N-di(C₁₋₆alkyl)amino,
—C(O)—R²⁰
wherein
R²⁰ represents C₁₋₆ alkyl, C₁₋₆ alkoxy, amino, N—(C₁₋₆alkyl)amino, 6alkyl)amino, N—(C₁₋₆ acyl)amino, or a 5-7 membered saturated or unsaturated heterocyclic ring having 1 to 3 heteroatoms selected from the group consisting O, S and N, and optionally substituted by
C₁₋₆ alkyl, C₁₋₆ alkoxy, oxo, amino, N—(C₁₋₆alkyl)amino, N,N-di(C₁₋₆alkyl)amino, N—(C₁₋₆ acyl)amino, phenyl, or benzyl,
C₁₋₆ alkyl optionally substituted by R²¹
or
C₁₋₆ alkoxy optionally substituted by R²¹
wherein
R²¹ represents cyano, mono-, di or tri-halogen, hydroxy, amino, N—(C₁₋₆alkyl)amino, N,N-di(C₁₋₆alkyl)amino, N-(hydroxyC₁₋₆ alkyl)amino, N-(halophenylC₁₋₆ alkyl)amino, amino C₂₋₆ alkylenyl, C₁₋₆ alkoxy, hydroxyC₁₋₆ alkoxy, —C(O)—R²⁰¹, —NHC(O)—R²⁰¹, C₃₋₈cycloalkyl, iso-indolino, phthalimidyl, 2-oxo-1,3-oxazolidinyl, aryl or a 5 or 6 membered saturated or unsaturated heterocyclic ring having 1 to 4 heteroatoms selected from the group consisting O, S and N optionally substituted by
hydroxy, C₁₋₆ alkyl, C₁₋₆ alkoxy, C₁₋₆ alkoxycarbonyl, hydroxyC₁₋₆ alkoxy, oxo, amino, aminoC₁₋₆alkyl, N—(C₁₋₆alkyl)amino, N,N-di(C₁₋₆alkyl)amino, N—(C₁₋₆ acyl)amino, or benzyl,
wherein
R²⁰¹ represents hydroxy, amino, N—(C₁₋₆alkyl)amino, N,N-di(C₁₋₆alkyl)amino, N-(halophenylC₁₋₆ alkyl)amino, C₁₋₆alkyl, aminoC₁₋₆ alkyl, aminoC₂₋₆ alkylenyl, C₁₋₆ alkoxy, a 5 or 6 membered saturated or unsaturated heterocyclic ring having 1 to 4 heteroatoms selected from the group consisting O, S and N optionally substituted by
hydroxy, C₁₋₆ alkyl, C₁₋₆ alkoxy, C₁₋₆ alkoxycarbonyl, hydroxyC₁₋₆ alkoxy, oxo, amino, N—(C₁₋₆alkyl)amino, N,N-di(C₁₋₆alkyl)amino, N—(C₁₋₆ acyl)amino or benzyl;
R³ represents hydrogen, halogen, aminocarbonyl, or C₁₋₆ alkyl optionally substituted by aryl C₁₋₆ alkoxy or mono-, di- or tri-halogen;
R⁴ represents hydrogen or C₁₋₆ alkyl;
R⁵ represents hydrogen or C₁₋₆ alkyl; and
R⁶ represents halogen, hydrogen or C₁₋₆ alkyl;
or a physiologically acceptable salt, solvate, hydrate or stereoisomer thereof;
and
component B: one or more N-(2-arylamino)aryl sulfonamide compounds of general formula (B): 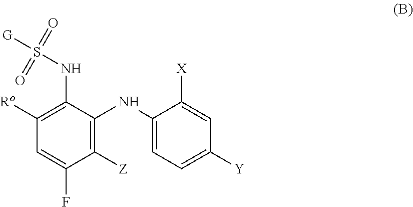
where G is R₁ₐ, R_{1b}, R_{1c}, R_{1d}, R₁ₑ, Ar₁, Ar₂ or Ar₃; R^{o} is H, halogen, C₁-C₆ alkyl, C₁-C₄ alkoxy, C₃-C₆ cycloalkyl, C₂-C₆ alkenyl, C₂-C₆ alkynyl, said alkyl, cycloalkyl, alkenyl, and alkynyl groups optionally substituted with 1-3 substituents selected independently from halogen, OH, CN, cyanomethyl, nitro, phenyl, and trifluoromethyl, and said C₁-C₆ alkyl and C₁-C₄ alkoxy groups also optionally substituted with OCH₃ or OCH₂CH₃; X is F, Cl or methyl; Y is I, Br, Cl, CF₃, C₁-C₃ alkyl, C₂-C₃ alkenyl, C₂-C₃ alkynyl, cyclopropyl, phenyl, pyridyl, pyrazolyl, OMe, OEt, or SMe, where all said methyl, ethyl, C₁-C₃ alkyl, and cyclopropyl groups of X and Y are optionally substituted with OH, all said phenyl, pyridyl, pyrazolyl groups of Y are optionally substituted with halogen, acetyl, methyl, and trifluoromethyl, and all said methyl groups of X and Y are optionally substituted with one, two, or three F atoms; and Z is H or F,
where R₁ₐ is methyl, optionally substituted with 1-3 fluorine atoms or 1-3 chlorine atoms, or with OH, cyclopropoxy, or C₁-C₄ alkoxy, where the C₁-C₄ alkyl moieties of said C₁-C₄ alkoxy groups are optionally substituted with one hydroxy or methoxy group, and where all C₂-C₄ alkyl groups within said C₁-C₄ alkoxy are optionally further substituted with a second OH group;
R_{1b} is CH(CH₃)—C₁₋₃ alkyl or C₃-C₆ cycloalkyl, said methyl, alkyl, and cycloalkyl groups optionally substituted with 1-3 substituents selected independently from F, Cl, Br, I, OH, C₁-C₄ alkoxy, and CN;
R_{1c} is (CH₂)ₙOₘR′, where m is 0 or 1; where, when m is 1, n is 2 or 3, and when m is 0, n is 1 or 2; and where R′ is C₁-C₆ alkyl, optionally substituted with 1-3 substituents selected independently from F, Cl, OH, OCH₃, OCH₂CH₃, and C₃-C₆ cycloalkyl;
R_{1d} is C(A)(A′)(B)— where B, A, and A′ are, independently, H or C₁₋₄ alkyl, optionally substituted with one or two OH groups or halogen atoms, or A and A′, together with the carbon atom to which they are attached, form a 3- to 6-member saturated ring, said ring optionally containing one or two heteroatoms selected, independently, from O, N, and S and optionally substituted with one or two groups selected independently from methyl, ethyl, and halo;
R₁ₑ is benzyl or 2-phenyl ethyl, in which the phenyl group is optionally substituted 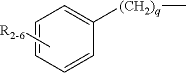
where q is 1 or 2, R₂, R₃ and R₄ are, independently, H, F, Cl, Br, CH₃, CH₂F, CHF₂, CF₃, OCH₃, OCH₂F, OCHF₂, OCF₃, ethyl, n-propyl, isopropyl, cyclopropyl, isobutyl, sec-butyl, tert-butyl, and methylsulfonyl, and R₄ may also be nitro, acetamido, amidinyl, cyano, carbamoyl, methylcarbamoyl, dimethylcarbamoyl, 1,3,4-oxadiazol-2-yl, 5-methyl-1,3,4-oxadiazolyl, 1,3,4-thiadiazolyl, 5-methyl-1,3,4-thiadiazol-1H-tetrazolyl, N-morpholinyl carbonylamino, N-morpholinylsulfonyl, and N-pyrrolidinylcarbonylamino; R₅ and R₆ are, independently, H, F, Cl, or methyl;
Ar₁ is 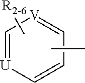
where U and V are, independently, N, CR₂ or CR₃; R₂, R₃ and R₄ are, independently, H, F, Cl, Br, CH₃, CH₂F, CHF₂, CF₃, OCH₃, OCH₂F, OCHF₂, OCF₃, ethyl, n-propyl, isopropyl, cyclopropyl, isobutyl, sec-butyl, tert-butyl, and methylsulfonyl, and R₄ may also be nitro, acetamido, amidinyl, cyano, carbamoyl, methylcarbamoyl, dimethylcarbamoyl, 1,3,4-oxadiazol-2-yl, 5-methyl-1,3,4-oxadiazol, 1,3,4-thiadiazol, 5-methyl-1,3,4-thiadiazol 1H-tetrazolyl, N-morpholinylcarbonylamino, N-morpholinylsulfonyl and N-pyrrolidinylcarbonylamino; R₅ and R₆ are, independently, H, F, Cl or methyl;
Ar₂ is 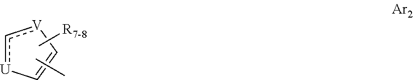
where the dashed line represents a double bond which may be located formally either between V and the carbon between U and V, or between U and the carbon between U and V; where U is —S—, —O— or —N═ and where, when U is —O— or —S—, V is —CH═, —CCl═ or —N═; and when U is —N═, V CH═, or —NCH₃—; R₇ and R₈ are, independently, H, methoxycarbonyl, methylcarbamoyl, acetamido, acetyl, methyl, ethyl, trifluoromethyl, or halogen.
Ar₃ is 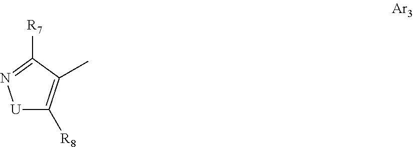
where U is —NH—, —NCH₃— or —O—; and R⁷ and R⁸ are, independently, H, F, Cl, or methyl;
or a physiologically acceptable salt, solvate, hydrate or stereoisomer thereof;
and, optionally,
component C: one or more further pharmaceutical agents,
wherein components A and B are optionally formulated for simultaneous, concurrent, separate, or sequential administration.

2. The combination according to claim 1, wherein:
said component A is one or more 2,3-dihydroimidazo[1,2-c]quinazoline compounds of general formula (A2): 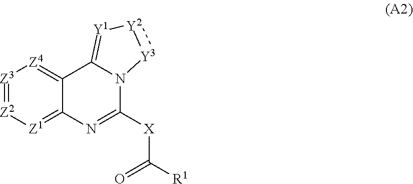
in which:
X represents CR⁵R⁶ or NH;
Y¹ represents CR³ or N;
the chemical bond between [symbol] represents a single bond or double bond,
with the proviso that when the [symbol] represents a double bond, Y² and Y³ independently represent CR⁴ or N, and
when [symbol] represents a single bond, Y² and Y³ independently represent CR³R⁴ or NR⁴;
Z¹, Z², Z³ and Z⁴ independently represent CH, CR² or N;
R¹ represents aryl optionally having 1 to 3 substituents selected from R¹¹, C₃₋₈ cycloalkyl optionally having 1 to 3 substituents selected from R¹¹, C₁₋₆ alkyl optionally substituted by aryl, heteroaryl, C₁₋₆ alkoxyaryl, aryloxy, heteroaryloxy or one or more halogen,
C₁₋₆ alkoxy optionally substituted by carboxy, aryl, heteroaryl, C₁₋₆ alkoxyaryl, aryloxy, heteroaryloxy or one or more halogen,
or
a 3 to 15 membered mono- or bi-cyclic heterocyclic ring that is saturated or unsaturated, optionally having 1 to 3 substituents selected from R¹¹, and contains 1 to 3 heteroatoms selected from the group consisting of N, O and S,
wherein
R¹¹ represents halogen, nitro, hydroxy, cyano, carboxy, amino, N—(C₁₋₆alkyl)amino, N-(hydroxyC₁₋₆alkyl)amino, N,N-di(C₁₋₆alkyl)amino, N—(C₁₋₆acyl)amino, N-(formyl)-N—(C₁₋₆alkyl)amino, N—(C₁₋₆alkanesulfonyl)amino, N-(carboxyC₁₋₆alkyl)-N—(C₁₋₆alkyl)amino, N—(C₁₋₆alkoxycarbonyl)amino, N—[N,N-di(C₁₋₆alkyl)amino methylene]amino, N—[N,N-di(C₁₋₆alkyl)amino (C₁₋₆alkyl)methylene]amino, N—[N,N-di(C₁₋₆alkyl)amino C₂₋₆alkenyl]amino, aminocarbonyl, N—(C₁₋₆alkyl)aminocarbonyl, N,N-di(C₁₋₆alkyl)aminocarbonyl, C₃₋₈cycloalkyl, C₁₋₆ alkylthio, C₁₋₆alkanesulfonyl, sulfamoyl, C₁₋₆alkoxycarbonyl, N-arylamino wherein said aryl moiety is optionally having 1 to 3 substituents selected from R¹⁰¹, N-(aryl C₁₋₆alkyl)amino wherein said aryl moiety is optionally having 1 to 3 substituents selected from R¹⁰¹, aryl C₁₋₆alkoxycarbonyl wherein said aryl moiety is optionally having 1 to 3 substituents selected from R¹⁰¹,
C₁₋₆alkyl optionally substituted by mono-, di- or tri-halogen, amino, N—(C₁₋₆alkyl)amino or N,N-di(C₁₋₆alkyl)amino,
C₁₋₆alkoxy optionally substituted by mono-, di- or tri-halogen, N—(C₁₋₆alkyl)sulfonamide, or N-(aryl)sulfonamide,
or
a 5 to 7 membered saturated or unsaturated ring having 1 to 3 heteroatoms selected from the group consisting of O, S and N, and optionally having 1 to 3 substituents selected from R¹⁰¹
wherein
R¹⁰¹ represents halogen, carboxy, amino, N—(C₁₋₆ alkyl)amino, 6alkyl)amino, aminocarbonyl, N—(C₁₋₆alkyl)aminocarbonyl, N,N-di(C₁₋₆alkyl)aminocarbonyl, pyridyl,
C₁₋₆ alkyl optionally substituted by cyano or mono-di- or tri-halogen,
and
C₁₋₆alkoxy optionally substituted by cyano, carboxy, amino, N—(C₁₋₆ alkyl)amino, N,N-di(C₁₋₆alkyl)amino, aminocarbonyl, 6alkyl)aminocarbonyl, N,N-di(C₁₋₆alkyl)aminocarbonyl or mono-, di- or tri-halogen;
R² represents hydroxy, halogen, nitro, cyano, amino, N—(C₁₋₆alkyl)amino, N,N-di(C₁₋₆alkyl)amino, N-(hydroxyC₁₋₆alkyl)amino, N-(hydroxyC₁₋₆alkyl)-N—(C₁₋₆alkyl)amino, C₁₋₆ acyloxy, aminoC₁₋₆ acyloxy, C₂₋₆alkenyl, aryl,
a 5-7 membered saturated or unsaturated heterocyclic ring having 1 to 3 heteroatoms selected from the group consisting O, S and N, and optionally substituted by
hydroxy, C₁₋₆ alkyl, C₁₋₆ alkoxy, oxo, amino, amino C₁₋₆alkyl, N—(C₁₋₆alkyl)amino, N,N-di(C₁₋₆alkyl)amino, N—(C₁₋₆ acyl)amino, N—(C₁₋₆alkyl)carbonylamino, phenyl, phenyl C₁₋₆ alkyl, carboxy, C₁₋₆alkoxycarbonyl, aminocarbonyl, N—(C₁₋₆alkyl)aminocarbonyl, or N,N-di(C₁₋₆alkyl)amino, —C(O)—R²⁰
wherein
R²⁰ represents C₁₋₆ alkyl, C₁₋₆ alkoxy, amino, N—(C₁₋₆alkyl)amino, N,N-di(C₁₋₆alkyl)amino, N—(C₁₋₆ acyl)amino, or a 5-7 membered saturated or unsaturated heterocyclic ring having 1 to 3 heteroatoms selected from the group consisting O, S and N, and optionally substituted by C₁₋₆ alkyl, C₁₋₆ alkoxy, oxo, amino, N—(C₁₋₆alkyl)amino, N,N-di(C₁₋₆alkyl)amino, N—(C₁₋₆ acyl)amino, phenyl, or benzyl,
C₁₋₆ alkyl optionally substituted by R²¹,
or
C₁₋₆ alkoxy optionally substituted by R²¹,
wherein
R²¹ represents cyano, mono-, di or tri-halogen, hydroxy, amino, N—(C₁₋₆alkyl)amino, N,N-di(C₁₋₆alkyl)amino, N-(hydroxyC₁₋₆ alkyl)amino, N-(halophenylC₁₋₆ alkyl)amino, amino C₂₋₆ alkylenyl, C₁₋₆ alkoxy, hydroxyC₁₋₆ alkoxy, —C(O)—R²⁰¹, —NHC(O)—R²⁰¹, C₃₋₈cycloalkyl, isoindolino, phthalimidyl, 2-oxo-1,3-oxazolidinyl, aryl or a 5 or 6 membered saturated or unsaturated heterocyclic ring having 1 to 4 heteroatoms selected from the group consisting O, S and N, and optionally substituted by hydroxy, C₁₋₆ alkyl, C₁₋₆ alkoxy, C₁₋₆ alkoxycarbonyl, hydroxyC₁₋₆ alkoxy, oxo, amino, aminoC₁₋₆alkyl, N—(C₁₋₆alkyl)amino, N,N-di(C₁₋₆alkyl)amino, N—(C₁₋₆ acyl)amino, or benzyl,
wherein
R²⁰¹ represents hydroxy, amino, N—(C₁₋₆alkyl)amino, N,N-di(C₁₋₆alkyl)amino, N-(halophenylC₁₋₆ alkyl)amino, aminoC₁₋₆ alkyl, aminoC₂₋₆ alkylenyl, C₁₋₆ alkoxy, a 5 or 6 membered saturated or unsaturated heterocyclic ring having 1 to 4 heteroatoms selected from the group consisting O, S and N, and optionally substituted by hydroxy, C₁₋₆ alkyl, C₁₋₆ alkoxy, C₁₋₆ alkoxycarbonyl, hydroxyC₁₋₆ alkoxy, oxo, amino, N—(C₁₋₆alkyl)amino, N,N-di(C₁₋₆alkyl)amino, N—(C₁₋₆ acyl)amino or benzyl;
R³ represents hydrogen, halogen, aminocarbonyl, or C₁₋₆ alkyl optionally substituted by aryl C₁₋₆ alkoxy or mono-, di- or tri-halogen;
R⁴ represents hydrogen or C₁₋₆ alkyl;
R⁵ represents hydrogen or C₁₋₆ alkyl; and
R⁶ represents halogen, hydrogen or C₁₋₆ alkyl;
or a physiologically acceptable salt, solvate, hydrate or stereoisomer thereof optionally in the form of a pharmaceutical formulation which is ready for use to be administered simultaneously, concurrently, separately or sequentially.

3. The combination according to claim 1, wherein:
said component A is one or more 2,3-dihydroimidazo[1,2-c]quinazoline compounds selected from

| Ex. No. | Structure |
|---|---|
| 1-1 | 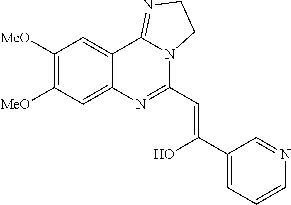 [SMILES: COc1cc2c(cc1OC)C1=NCCN1C(/C=C(\O)c1cccnc1)=N2] |
| 1-2 | 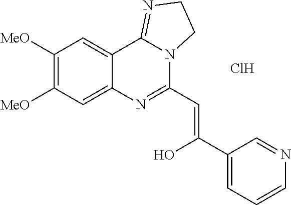 [SMILES: COc1cc2c(cc1OC)C1=NCCN1C(/C=C(\O)c1cccnc1)=N2 ; Cl] |
| 1-3 | 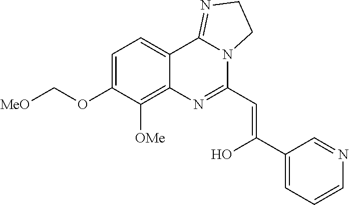 [SMILES: COCOc1ccc2c(c1OC)N=C(/C=C(\O)c1cccnc1)N1CCN=C21] |
| 1-4 | 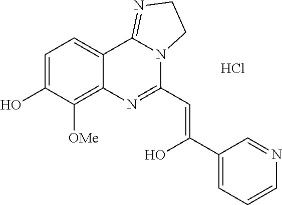 [SMILES: COc1c(O)ccc2c1N=C(/C=C(\O)c1cccnc1)N1CCN=C21 ; Cl] |
| 1-5 | 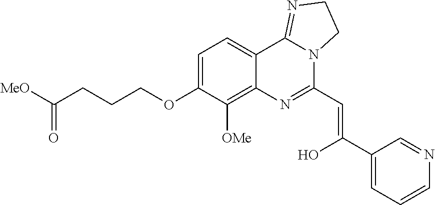 [SMILES: COC(=O)CCCOc1ccc2c(c1OC)N=C(/C=C(\O)c1cccnc1)N1CCN=C21] |
| 1-6 | 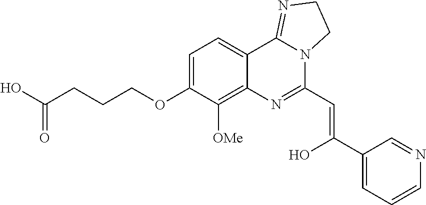 [SMILES: COc1c(OCCCC(=O)O)ccc2c1N=C(/C=C(\O)c1cccnc1)N1CCN=C21] |
| 1-7 | 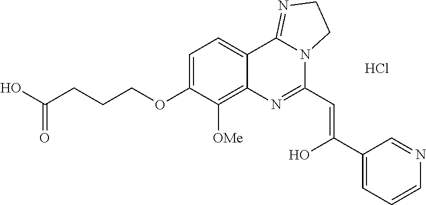 [SMILES: COc1c(OCCCC(=O)O)ccc2c1N=C(/C=C(\O)c1cccnc1)N1CCN=C21 ; Cl] |
| 1-8 | 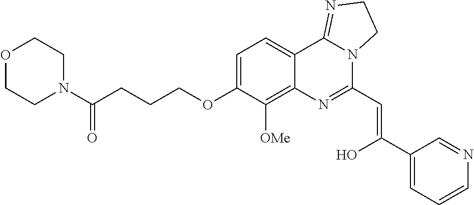 [SMILES: COc1c(OCCCC(=O)N2CCOCC2)ccc2c1N=C(/C=C(\O)c1cccnc1)N1CCN=C21] |
| 1-9 | 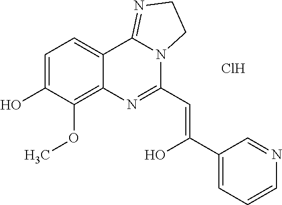 [SMILES: COc1c(O)ccc2c1N=C(/C=C(\O)c1cccnc1)N1CCN=C21 ; Cl] |
| 1-10 | 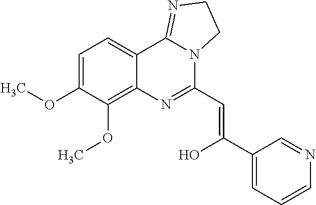 [SMILES: COc1ccc2c(c1OC)N=C(/C=C(\O)c1cccnc1)N1CCN=C21] |
| 1-11 | 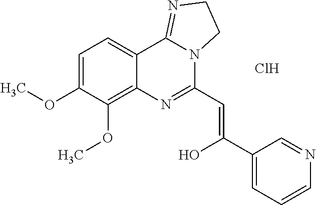 [SMILES: COc1ccc2c(c1OC)N=C(/C=C(\O)c1cccnc1)N1CCN=C21 ; Cl] |
| 1-12 | 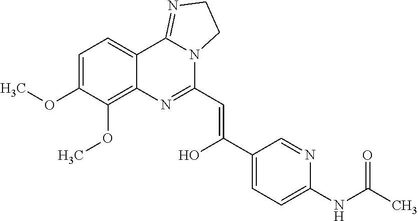 [SMILES: COc1ccc2c(c1OC)N=C(/C=C(\O)c1ccc(NC(C)=O)nc1)N1CCN=C21] |
| 1-13 | 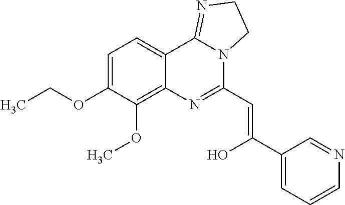 [SMILES: CCOc1ccc2c(c1OC)N=C(/C=C(\O)c1cccnc1)N1CCN=C21] |
| 1-14 | 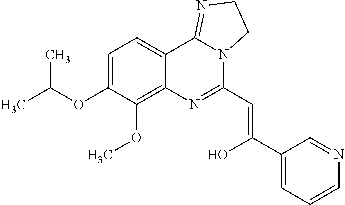 [SMILES: COc1c(OC(C)C)ccc2c1N=C(/C=C(\O)c1cccnc1)N1CCN=C21] |
| 1-15 | 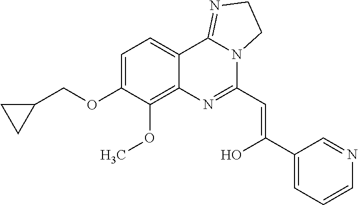 [SMILES: COc1c(OCC2CC2)ccc2c1N=C(/C=C(\O)c1cccnc1)N1CCN=C21] |
| 1-16 | 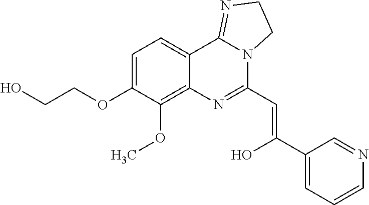 [SMILES: COc1c(OCCO)ccc2c1N=C(/C=C(\O)c1cccnc1)N1CCN=C21] |
| 1-17 | 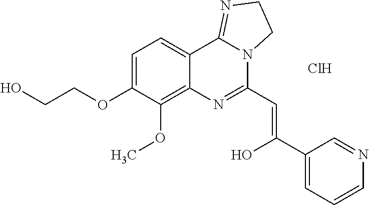 [SMILES: COc1c(OCCO)ccc2c1N=C(/C=C(\O)c1cccnc1)N1CCN=C21 ; Cl] |
| 1-18 | 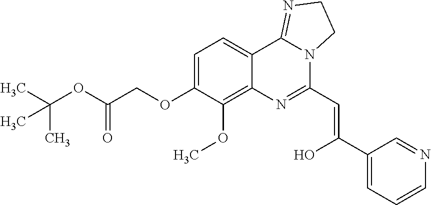 [SMILES: COc1c(OCC(=O)OC(C)(C)C)ccc2c1N=C(/C=C(\O)c1cccnc1)N1CCN=C21] |
| 1-19 | 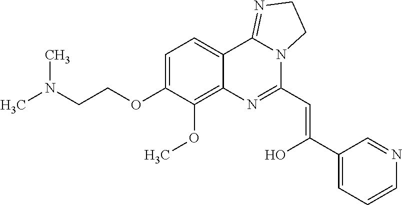 [SMILES: COc1c(OCCN(C)C)ccc2c1N=C(/C=C(\O)c1cccnc1)N1CCN=C21] |
| 1-20 | 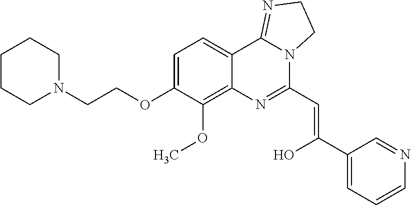 [SMILES: COc1c(OCCN2CCCCC2)ccc2c1N=C(/C=C(\O)c1cccnc1)N1CCN=C21] |
| 1-21 | 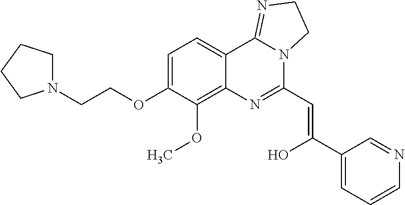 [SMILES: COc1c(OCCN2CCCC2)ccc2c1N=C(/C=C(\O)c1cccnc1)N1CCN=C21] |
| 1-22 | 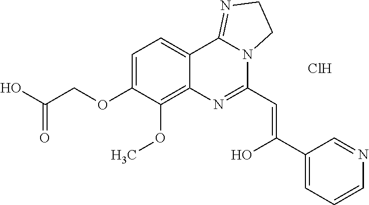 [SMILES: COc1c(OCC(=O)O)ccc2c1N=C(/C=C(\O)c1cccnc1)N1CCN=C21 ; Cl] |
| 1-23 | 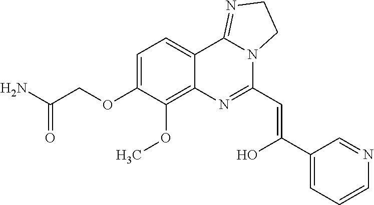 [SMILES: COc1c(OCC(N)=O)ccc2c1N=C(/C=C(\O)c1cccnc1)N1CCN=C21] |
| 1-24 | 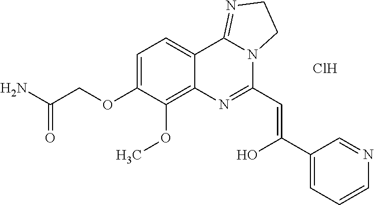 [SMILES: COc1c(OCC(N)=O)ccc2c1N=C(/C=C(\O)c1cccnc1)N1CCN=C21 ; Cl] |
| 1-25 | 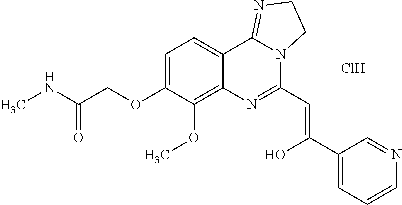 [SMILES: CNC(=O)COc1ccc2c(c1OC)N=C(/C=C(\O)c1cccnc1)N1CCN=C21 ; Cl] |
| 1-26 | 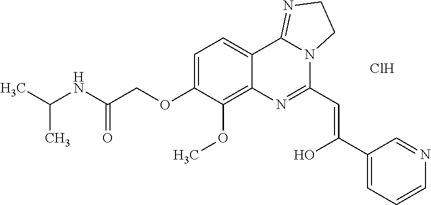 [SMILES: COc1c(OCC(=O)NC(C)C)ccc2c1N=C(/C=C(\O)c1cccnc1)N1CCN=C21 ; Cl] |
| 1-27 | 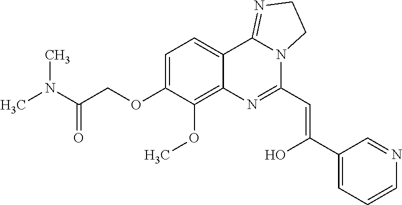 [SMILES: COc1c(OCC(=O)N(C)C)ccc2c1N=C(/C=C(\O)c1cccnc1)N1CCN=C21] |
| 1-28 | 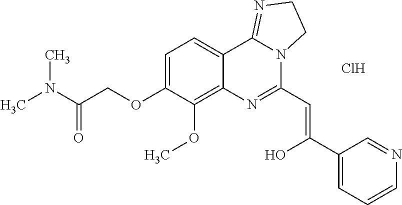 [SMILES: COc1c(OCC(=O)N(C)C)ccc2c1N=C(/C=C(\O)c1cccnc1)N1CCN=C21 ; Cl] |
| 1-29 | 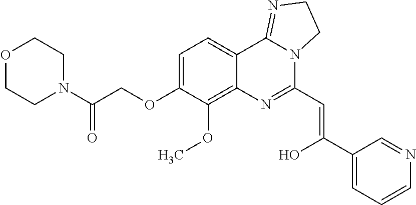 [SMILES: COc1c(OCC(=O)N2CCOCC2)ccc2c1N=C(/C=C(\O)c1cccnc1)N1CCN=C21] |
| 1-30 | 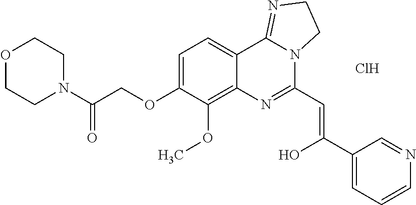 [SMILES: COc1c(OCC(=O)N2CCOCC2)ccc2c1N=C(/C=C(\O)c1cccnc1)N1CCN=C21 ; Cl] |
| 1-31 | 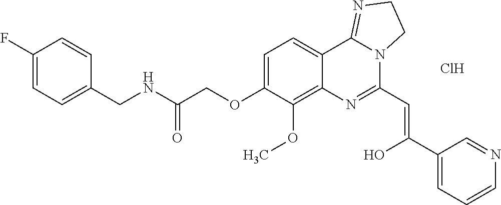 [SMILES: COc1c(OCC(=O)NCc2ccc(F)cc2)ccc2c1N=C(/C=C(\O)c1cccnc1)N1CCN=C21 ; Cl] |
| 1-32 | 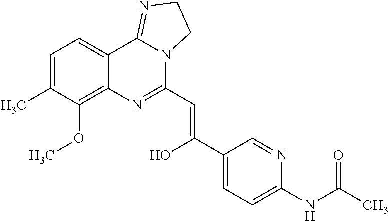 [SMILES: COc1c(C)ccc2c1N=C(/C=C(\O)c1ccc(NC(C)=O)nc1)N1CCN=C21] |
| 1-33 | 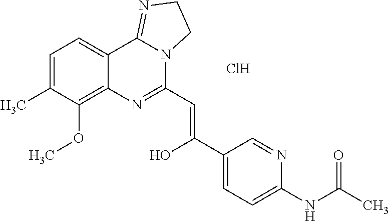 [SMILES: COc1c(C)ccc2c1N=C(/C=C(\O)c1ccc(NC(C)=O)nc1)N1CCN=C21 ; Cl] |
| 1-34 | 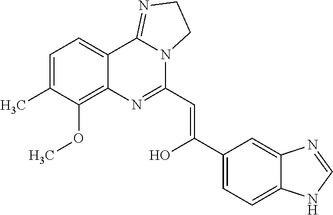 [SMILES: COc1c(C)ccc2c1N=C(/C=C(\O)c1ccc3[nH]cnc3c1)N1CCN=C21] |
| 1-35 | 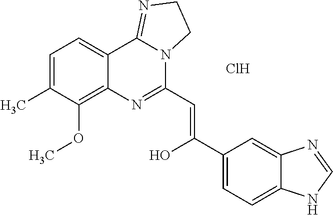 [SMILES: COc1c(C)ccc2c1N=C(/C=C(\O)c1ccc3[nH]cnc3c1)N1CCN=C21 ; Cl] |
| 1-36 | 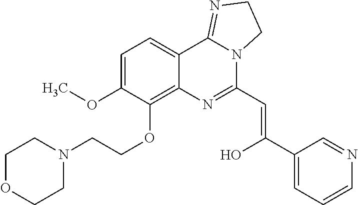 [SMILES: COc1ccc2c(c1OCCN1CCOCC1)N=C(/C=C(\O)c1cccnc1)N1CCN=C21] |
| 1-37 | 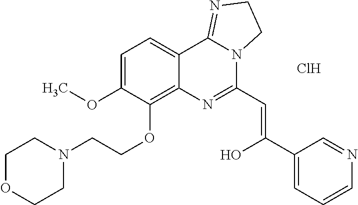 [SMILES: COc1ccc2c(c1OCCN1CCOCC1)N=C(/C=C(\O)c1cccnc1)N1CCN=C21 ; Cl] |
| 1-38 | 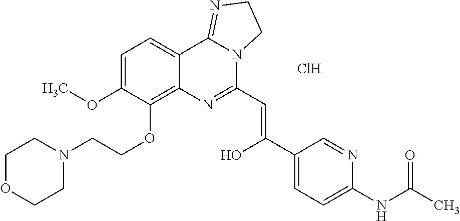 [SMILES: COc1ccc2c(c1OCCN1CCOCC1)N=C(/C=C(\O)c1ccc(NC(C)=O)nc1)N1CCN=C21 ; Cl] |
| 1-39 | 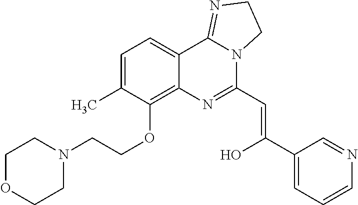 [SMILES: Cc1ccc2c(c1OCCN1CCOCC1)N=C(/C=C(\O)c1cccnc1)N1CCN=C21] |
| 1-40 | 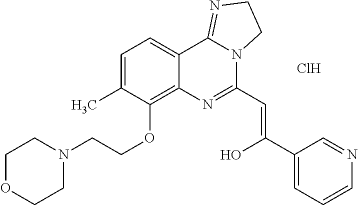 [SMILES: Cc1ccc2c(c1OCCN1CCOCC1)N=C(/C=C(\O)c1cccnc1)N1CCN=C21 ; Cl] |
| 1-41 | 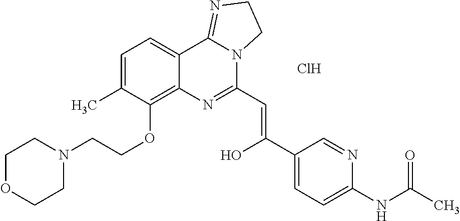 [SMILES: CC(=O)Nc1ccc(/C(O)=C/C2=Nc3c(ccc(C)c3OCCN3CCOCC3)C3=NCCN23)cn1 ; Cl] |
| 1-42 | 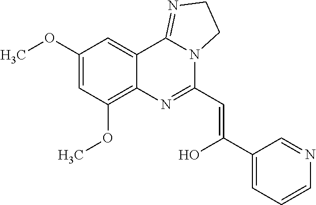 [SMILES: COc1cc(OC)c2c(c1)C1=NCCN1C(/C=C(\O)c1cccnc1)=N2] |
| 1-43 | 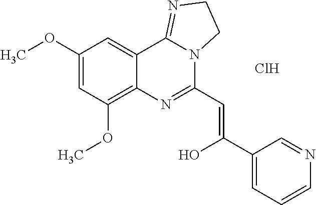 [SMILES: COc1cc(OC)c2c(c1)C1=NCCN1C(/C=C(\O)c1cccnc1)=N2 ; Cl] |
| 1-44 | 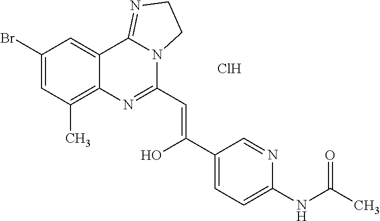 [SMILES: CC(=O)Nc1ccc(/C(O)=C/C2=Nc3c(C)cc(Br)cc3C3=NCCN23)cn1 ; Cl] |
| 1-45 | 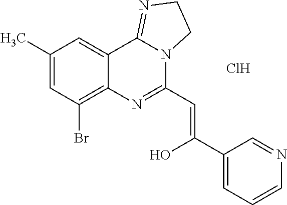 [SMILES: Cc1cc(Br)c2c(c1)C1=NCCN1C(/C=C(\O)c1cccnc1)=N2 ; Cl] |
| 1-46 | 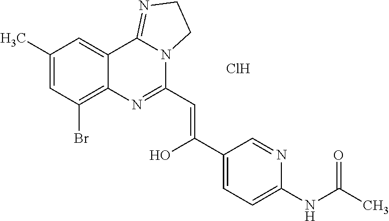 [SMILES: CC(=O)Nc1ccc(/C(O)=C/C2=Nc3c(Br)cc(C)cc3C3=NCCN23)cn1 ; Cl] |
| 1-47 | 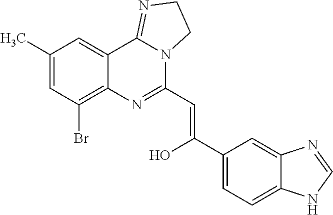 [SMILES: Cc1cc(Br)c2c(c1)C1=NCCN1C(/C=C(\O)c1ccc3[nH]cnc3c1)=N2] |
| 1-48 | 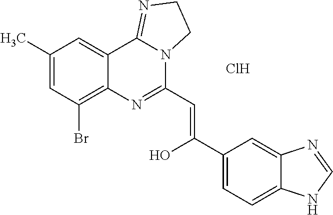 [SMILES: Cc1cc(Br)c2c(c1)C1=NCCN1C(/C=C(\O)c1ccc3[nH]cnc3c1)=N2 ; Cl] |
| 1-49 | 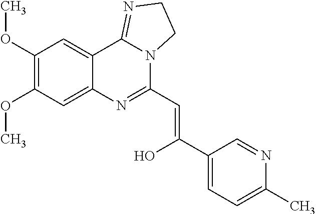 [SMILES: COc1cc2c(cc1OC)C1=NCCN1C(/C=C(\O)c1ccc(C)nc1)=N2] |
| 1-50 | 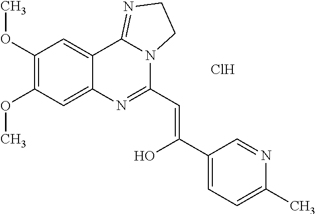 [SMILES: COc1cc2c(cc1OC)C1=NCCN1C(/C=C(\O)c1ccc(C)nc1)=N2 ; Cl] |
| 1-51 | 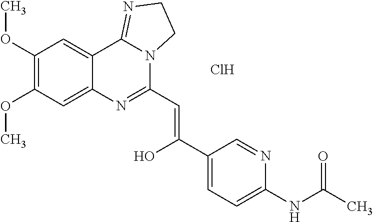 [SMILES: COc1cc2c(cc1OC)C1=NCCN1C(/C=C(\O)c1ccc(NC(C)=O)nc1)=N2 ; Cl] |
| 1-52 | 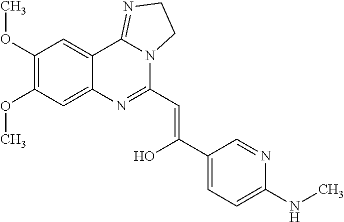 [SMILES: CNc1ccc(/C(O)=C/C2=Nc3cc(OC)c(OC)cc3C3=NCCN23)cn1] |
| 1-53 | 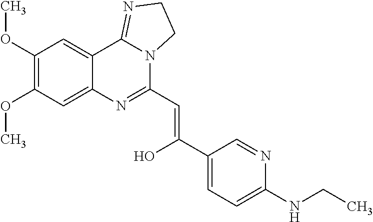 [SMILES: CCNc1ccc(/C(O)=C/C2=Nc3cc(OC)c(OC)cc3C3=NCCN23)cn1] |
| 1-54 | 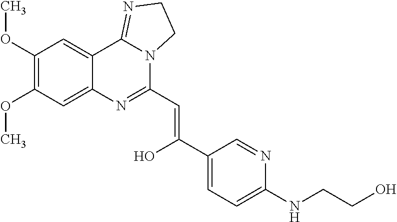 [SMILES: COc1cc2c(cc1OC)C1=NCCN1C(/C=C(\O)c1ccc(NCCO)nc1)=N2] |
| 1-55 | 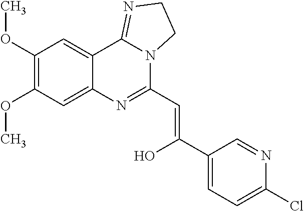 [SMILES: COc1cc2c(cc1OC)C1=NCCN1C(/C=C(\O)c1ccc(Cl)nc1)=N2] |
| 1-56 | 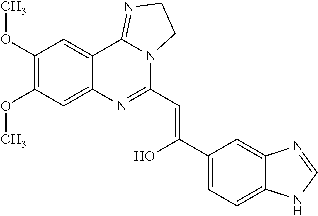 [SMILES: COc1cc2c(cc1OC)C1=NCCN1C(/C=C(\O)c1ccc3[nH]cnc3c1)=N2] |
| 1-57 | 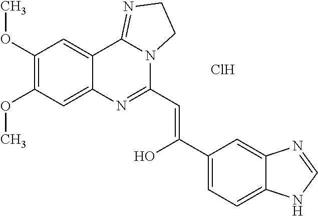 [SMILES: COc1cc2c(cc1OC)C1=NCCN1C(/C=C(\O)c1ccc3[nH]cnc3c1)=N2 ; Cl] |
| 1-58 | 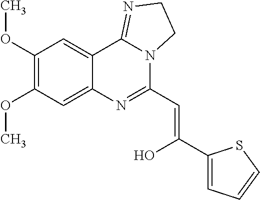 [SMILES: COc1cc2c(cc1OC)C1=NCCN1C(/C=C(\O)c1cccs1)=N2] |
| 1-59 | 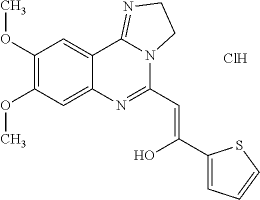 [SMILES: COc1cc2c(cc1OC)C1=NCCN1C(/C=C(\O)c1cccs1)=N2 ; Cl] |
| 1-60 | 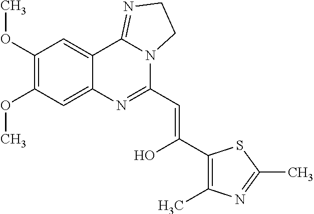 [SMILES: COc1cc2c(cc1OC)C1=NCCN1C(/C=C(\O)c1sc(C)nc1C)=N2] |
| 1-61 | 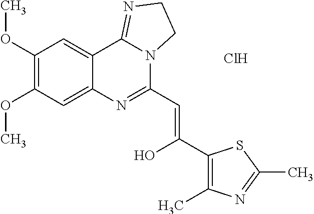 [SMILES: COc1cc2c(cc1OC)C1=NCCN1C(/C=C(\O)c1sc(C)nc1C)=N2 ; Cl] |
| 1-62 | 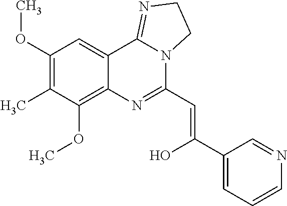 [SMILES: COc1cc2c(c(OC)c1C)N=C(/C=C(\O)c1cccnc1)N1CCN=C21] |
| 1-63 | 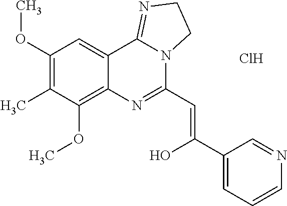 [SMILES: COc1cc2c(c(OC)c1C)N=C(/C=C(\O)c1cccnc1)N1CCN=C21 ; Cl] |
| 1-64 | 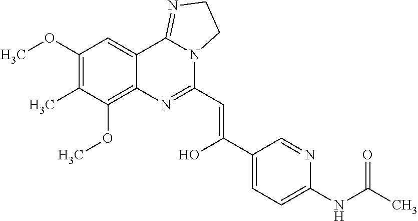 [SMILES: COc1cc2c(c(OC)c1C)N=C(/C=C(\O)c1ccc(NC(C)=O)nc1)N1CCN=C21] |
| 1-65 | 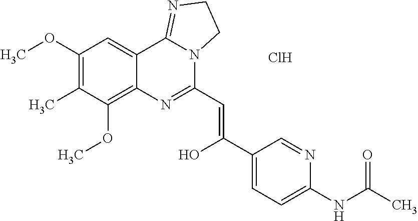 [SMILES: COc1cc2c(c(OC)c1C)N=C(/C=C(\O)c1ccc(NC(C)=O)nc1)N1CCN=C21 ; Cl] |
| 1-66 | 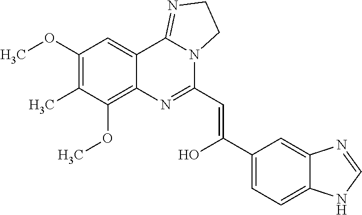 [SMILES: COc1cc2c(c(OC)c1C)N=C(/C=C(\O)c1ccc3[nH]cnc3c1)N1CCN=C21] |
| 1-67 | 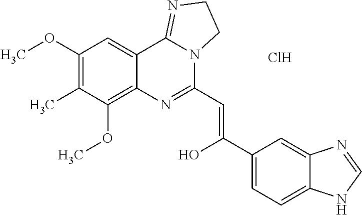 [SMILES: COc1cc2c(c(OC)c1C)N=C(/C=C(\O)c1ccc3[nH]cnc3c1)N1CCN=C21 ; Cl] |
| 1-68 | 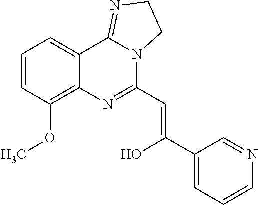 [SMILES: COc1cccc2c1N=C(/C=C(\O)c1cccnc1)N1CCN=C21] |
| 1-69 | 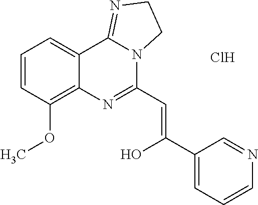 [SMILES: COc1cccc2c1N=C(/C=C(\O)c1cccnc1)N1CCN=C21 ; Cl] |
| 1-70 | 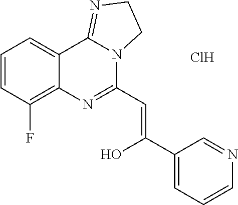 [SMILES: O/C(=C\C1=Nc2c(F)cccc2C2=NCCN12)c1cccnc1 ; Cl] |
| 1-71 | 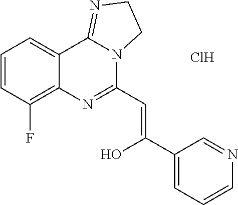 [SMILES: O/C(=C\C1=Nc2c(F)cccc2C2=NCCN12)c1cccnc1 ; Cl] |
| 1-72 | 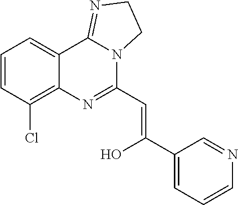 [SMILES: O/C(=C\C1=Nc2c(Cl)cccc2C2=NCCN12)c1cccnc1] |
| 1-73 | 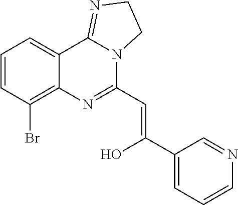 [SMILES: O/C(=C\C1=Nc2c(Br)cccc2C2=NCCN12)c1cccnc1] |
| 1-74 | 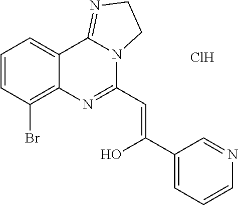 [SMILES: O/C(=C\C1=Nc2c(Br)cccc2C2=NCCN12)c1cccnc1 ; Cl] |
| 1-75 | 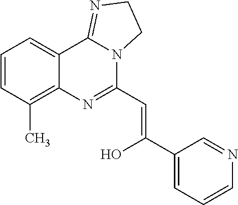 [SMILES: Cc1cccc2c1N=C(/C=C(\O)c1cccnc1)N1CCN=C21] |
| 1-76 | 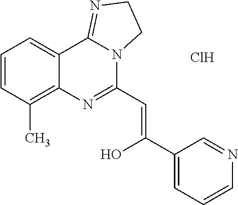 [SMILES: Cc1cccc2c1N=C(/C=C(\O)c1cccnc1)N1CCN=C21 ; Cl] |
| 1-77 | 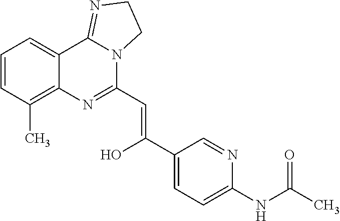 [SMILES: CC(=O)Nc1ccc(/C(O)=C/C2=Nc3c(C)cccc3C3=NCCN23)cn1] |
| 1-78 | 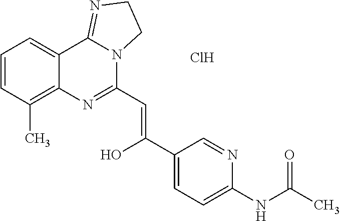 [SMILES: CC(=O)Nc1ccc(/C(O)=C/C2=Nc3c(C)cccc3C3=NCCN23)cn1 ; Cl] |
| 1-79 | 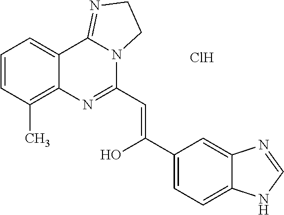 [SMILES: Cc1cccc2c1N=C(/C=C(\O)c1ccc3[nH]cnc3c1)N1CCN=C21 ; Cl] |
| 1-80 | 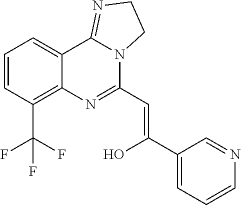 [SMILES: O/C(=C\C1=Nc2c(cccc2C(F)(F)F)C2=NCCN12)c1cccnc1] |
| 1-81 | 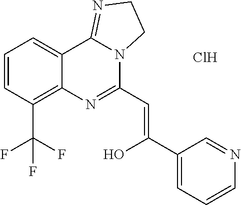 [SMILES: O/C(=C\C1=Nc2c(cccc2C(F)(F)F)C2=NCCN12)c1cccnc1 ; Cl] |
| 1-82 | 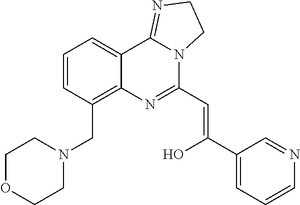 [SMILES: O/C(=C\C1=Nc2c(CN3CCOCC3)cccc2C2=NCCN12)c1cccnc1] |
| 1-83 | 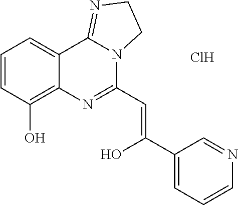 [SMILES: O/C(=C\C1=Nc2c(O)cccc2C2=NCCN12)c1cccnc1 ; Cl] |
| 1-84 | 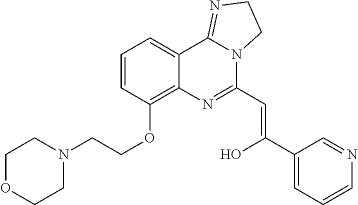 [SMILES: O/C(=C\C1=Nc2c(OCCN3CCOCC3)cccc2C2=NCCN12)c1cccnc1] |
| 1-85 | 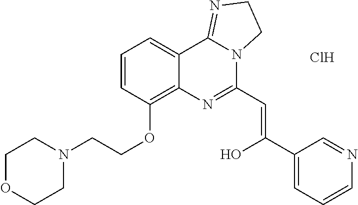 [SMILES: O/C(=C\C1=Nc2c(OCCN3CCOCC3)cccc2C2=NCCN12)c1cccnc1 ; Cl] |
| 1-86 | 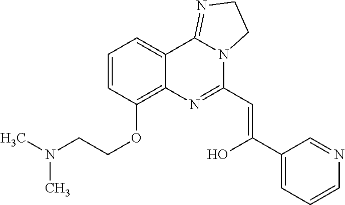 [SMILES: CN(C)CCOc1cccc2c1N=C(/C=C(\O)c1cccnc1)N1CCN=C21] |
| 1-87 | 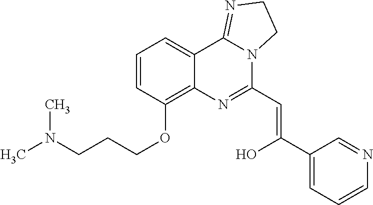 [SMILES: CN(C)CCCOc1cccc2c1N=C(/C=C(\O)c1cccnc1)N1CCN=C21] |
| 1-88 | 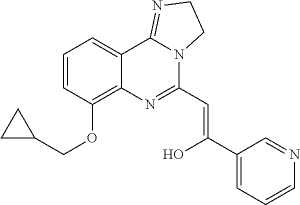 [SMILES: O/C(=C\C1=Nc2c(OCC3CC3)cccc2C2=NCCN12)c1cccnc1] |
| 1-89 | 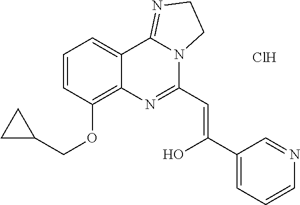 [SMILES: O/C(=C\C1=Nc2c(OCC3CC3)cccc2C2=NCCN12)c1cccnc1 ; Cl] |
| 1-90 | 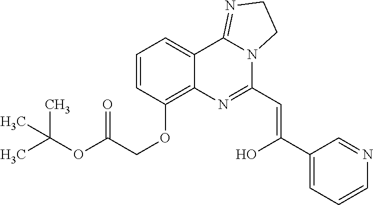 [SMILES: CC(C)(C)OC(=O)COc1cccc2c1N=C(/C=C(\O)c1cccnc1)N1CCN=C21] |
| 1-91 | 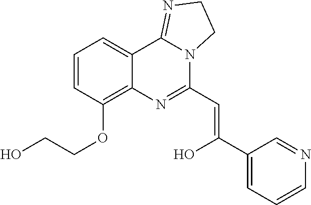 [SMILES: OCCOc1cccc2c1N=C(/C=C(\O)c1cccnc1)N1CCN=C21] |
| 1-92 | 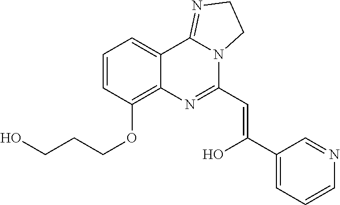 [SMILES: OCCCOc1cccc2c1N=C(/C=C(\O)c1cccnc1)N1CCN=C21] |
| 1-93 | 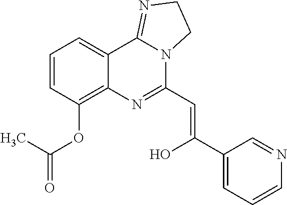 [SMILES: CC(=O)Oc1cccc2c1N=C(/C=C(\O)c1cccnc1)N1CCN=C21] |
| 1-94 | 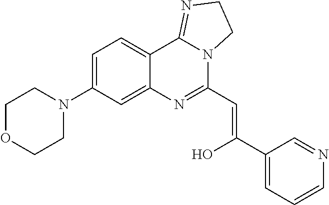 [SMILES: O/C(=C\C1=Nc2cc(N3CCOCC3)ccc2C2=NCCN12)c1cccnc1] |
| 1-95 | 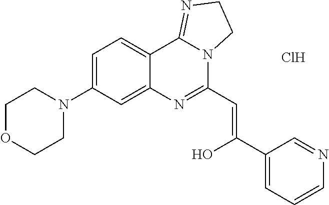 [SMILES: O/C(=C\C1=Nc2cc(N3CCOCC3)ccc2C2=NCCN12)c1cccnc1 ; Cl] |
| 1-96 | 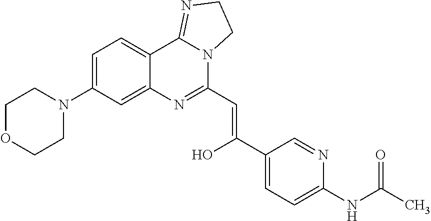 [SMILES: CC(=O)Nc1ccc(/C(O)=C/C2=Nc3cc(N4CCOCC4)ccc3C3=NCCN23)cn1] |
| 1-97 | 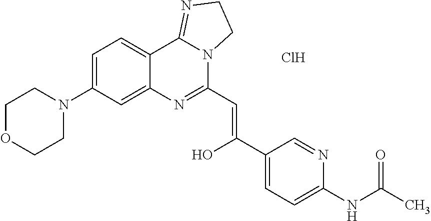 [SMILES: CC(=O)Nc1ccc(/C(O)=C/C2=Nc3cc(N4CCOCC4)ccc3C3=NCCN23)cn1 ; Cl] |
| 1-98 | 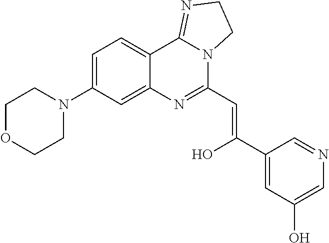 [SMILES: O/C(=C\C1=Nc2cc(N3CCOCC3)ccc2C2=NCCN12)c1cncc(O)c1] |
| 1-99 | 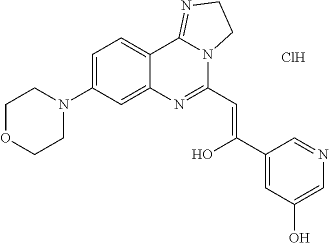 [SMILES: O/C(=C\C1=Nc2cc(N3CCOCC3)ccc2C2=NCCN12)c1cncc(O)c1 ; Cl] |
| 1-100 | 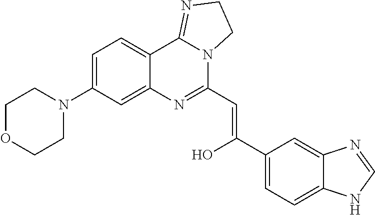 [SMILES: O/C(=C\C1=Nc2cc(N3CCOCC3)ccc2C2=NCCN12)c1ccc2[nH]cnc2c1] |
| 1-101 | 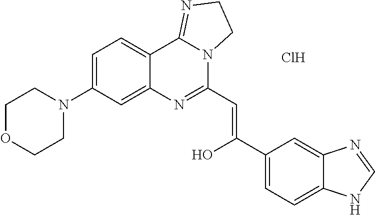 [SMILES: O/C(=C\C1=Nc2cc(N3CCOCC3)ccc2C2=NCCN12)c1ccc2[nH]cnc2c1 ; Cl] |
| 1-102 | 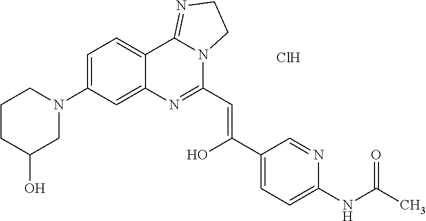 [SMILES: CC(=O)Nc1ccc(/C(O)=C/C2=Nc3cc(N4CCCC(O)C4)ccc3C3=NCCN23)cn1 ; Cl] |
| 1-103 | 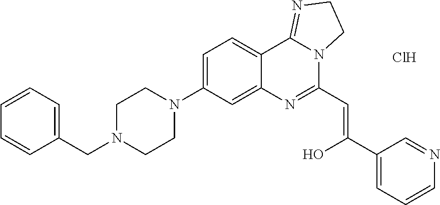 [SMILES: O/C(=C\C1=Nc2cc(N3CCN(Cc4ccccc4)CC3)ccc2C2=NCCN12)c1cccnc1 ; Cl] |
| 1-104 | 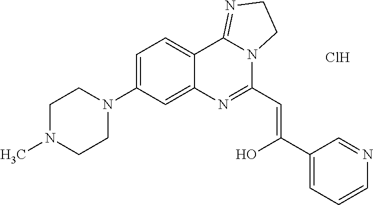 [SMILES: CN1CCN(c2ccc3c(c2)N=C(/C=C(\O)c2cccnc2)N2CCN=C32)CC1 ; Cl] |
| 1-105 | 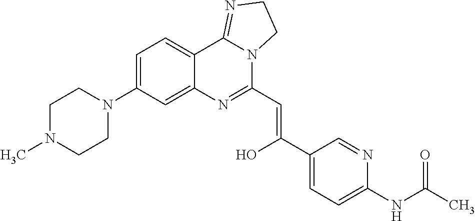 [SMILES: CC(=O)Nc1ccc(/C(O)=C/C2=Nc3cc(N4CCN(C)CC4)ccc3C3=NCCN23)cn1] |
| 1-106 | 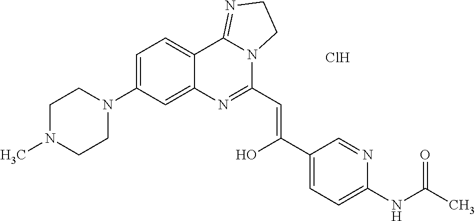 [SMILES: CC(=O)Nc1ccc(/C(O)=C/C2=Nc3cc(N4CCN(C)CC4)ccc3C3=NCCN23)cn1 ; Cl] |
| 1-107 | 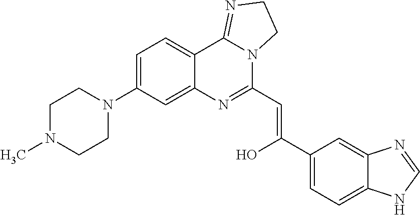 [SMILES: CN1CCN(c2ccc3c(c2)N=C(/C=C(\O)c2ccc4[nH]cnc4c2)N2CCN=C32)CC1] |
| 1-108 | 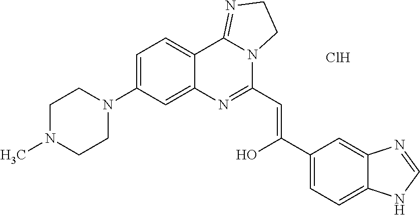 [SMILES: CN1CCN(c2ccc3c(c2)N=C(/C=C(\O)c2ccc4[nH]cnc4c2)N2CCN=C32)CC1 ; Cl] |
| 1-109 | 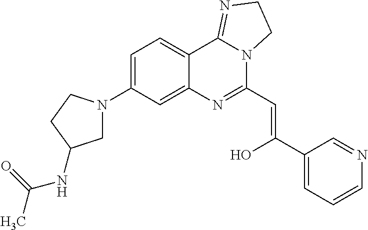 [SMILES: CC(=O)NC1CCN(c2ccc3c(c2)N=C(/C=C(\O)c2cccnc2)N2CCN=C32)C1] |
| 1-110 | 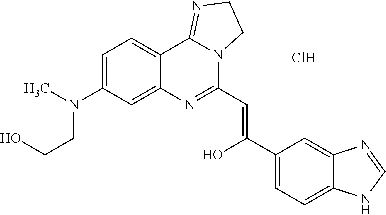 [SMILES: CN(CCO)c1ccc2c(c1)N=C(/C=C(\O)c1ccc3[nH]cnc3c1)N1CCN=C21 ; Cl] |
| 1-111 | 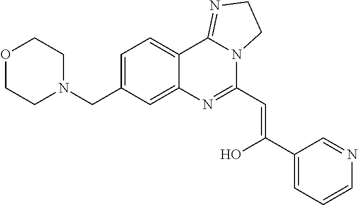 [SMILES: O/C(=C\C1=Nc2cc(CN3CCOCC3)ccc2C2=NCCN12)c1cccnc1] |
| 1-112 | 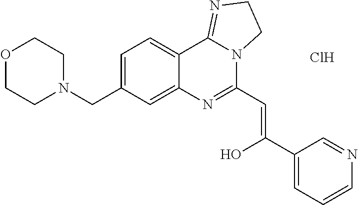 [SMILES: O/C(=C\C1=Nc2cc(CN3CCOCC3)ccc2C2=NCCN12)c1cccnc1 ; Cl] |
| 1-113 | 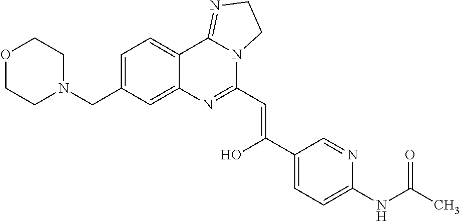 [SMILES: CC(=O)Nc1ccc(/C(O)=C/C2=Nc3cc(CN4CCOCC4)ccc3C3=NCCN23)cn1] |
| 1-114 | 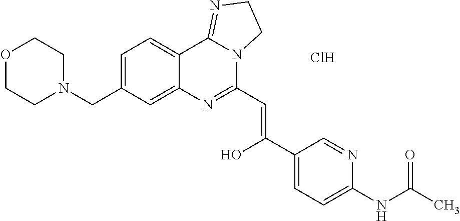 [SMILES: CC(=O)Nc1ccc(/C(O)=C/C2=Nc3cc(CN4CCOCC4)ccc3C3=NCCN23)cn1 ; Cl] |
| 1-115 | 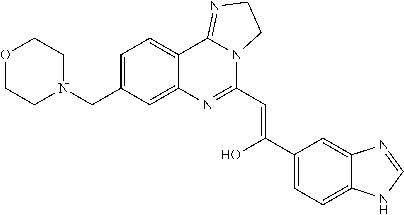 [SMILES: O/C(=C\C1=Nc2cc(CN3CCOCC3)ccc2C2=NCCN12)c1ccc2[nH]cnc2c1] |
| 1-116 | 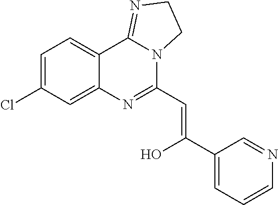 [SMILES: O/C(=C\C1=Nc2cc(Cl)ccc2C2=NCCN12)c1cccnc1] |
| 1-117 | 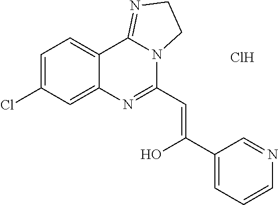 [SMILES: O/C(=C\C1=Nc2cc(Cl)ccc2C2=NCCN12)c1cccnc1 ; Cl] |
| 1-118 | 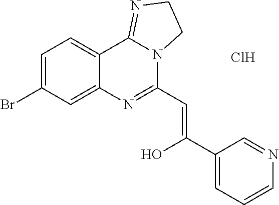 [SMILES: O/C(=C\C1=Nc2cc(Br)ccc2C2=NCCN12)c1cccnc1 ; Cl] |
| 1-119 | 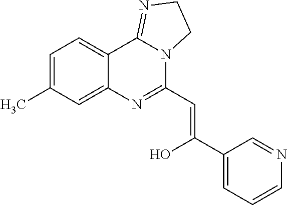 [SMILES: Cc1ccc2c(c1)N=C(/C=C(\O)c1cccnc1)N1CCN=C21] |
| 1-120 | 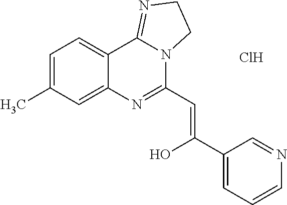 [SMILES: Cc1ccc2c(c1)N=C(/C=C(\O)c1cccnc1)N1CCN=C21 ; Cl] |
| 1-121 | 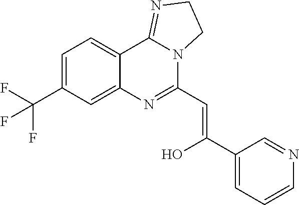 [SMILES: O/C(=C\C1=Nc2cc(C(F)(F)F)ccc2C2=NCCN12)c1cccnc1] |
| 1-122 | 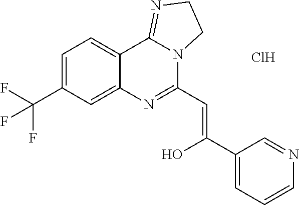 [SMILES: O/C(=C\C1=Nc2cc(C(F)(F)F)ccc2C2=NCCN12)c1cccnc1 ; Cl] |
| 1-123 | 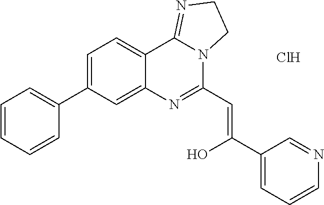 [SMILES: O/C(=C\C1=Nc2cc(-c3ccccc3)ccc2C2=NCCN12)c1cccnc1 ; Cl] |
| 1-124 | 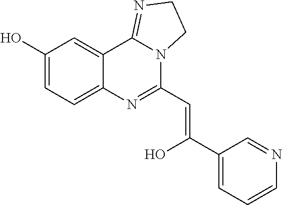 [SMILES: O/C(=C\C1=Nc2ccc(O)cc2C2=NCCN12)c1cccnc1] |
| 1-125 | 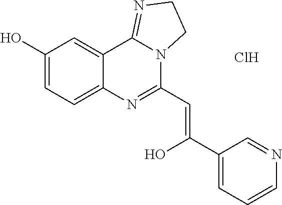 [SMILES: O/C(=C\C1=Nc2ccc(O)cc2C2=NCCN12)c1cccnc1 ; Cl] |
| 1-126 | 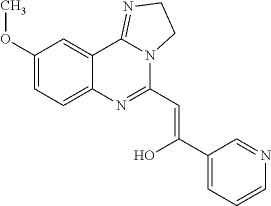 [SMILES: COc1ccc2c(c1)C1=NCCN1C(/C=C(\O)c1cccnc1)=N2] |
| 1-127 | 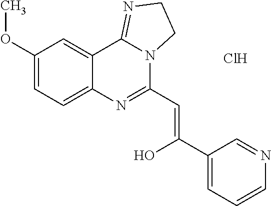 [SMILES: COc1ccc2c(c1)C1=NCCN1C(/C=C(\O)c1cccnc1)=N2 ; Cl] |
| 1-128 | 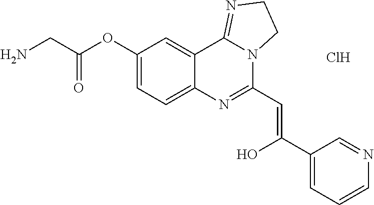 [SMILES: NCC(=O)Oc1ccc2c(c1)C1=NCCN1C(/C=C(\O)c1cccnc1)=N2 ; Cl] |
| 1-129 | 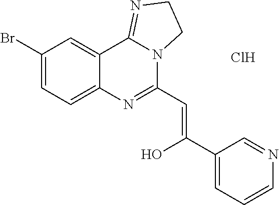 [SMILES: O/C(=C\C1=Nc2ccc(Br)cc2C2=NCCN12)c1cccnc1 ; Cl] |
| 1-130 | 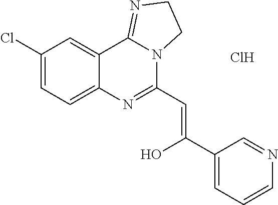 [SMILES: O/C(=C\C1=Nc2ccc(Cl)cc2C2=NCCN12)c1cccnc1 ; Cl] |
| 1-131 | 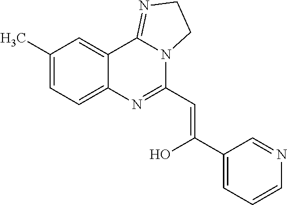 [SMILES: Cc1ccc2c(c1)C1=NCCN1C(/C=C(\O)c1cccnc1)=N2] |
| 1-132 | 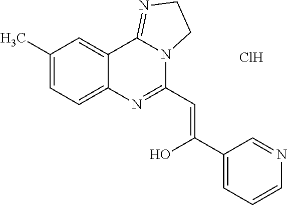 [SMILES: Cc1ccc2c(c1)C1=NCCN1C(/C=C(\O)c1cccnc1)=N2 ; Cl] |
| 1-133 | 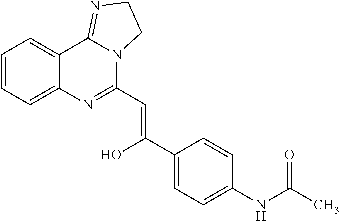 [SMILES: CC(=O)Nc1ccc(/C(O)=C/C2=Nc3ccccc3C3=NCCN23)cc1] |
| 1-134 | 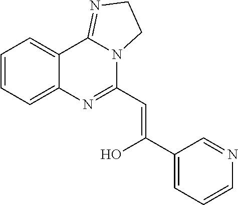 [SMILES: O/C(=C\C1=Nc2ccccc2C2=NCCN12)c1cccnc1] |
| 1-135 | 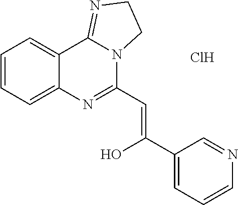 [SMILES: O/C(=C\C1=Nc2ccccc2C2=NCCN12)c1cccnc1 ; Cl] |
| 1-136 | 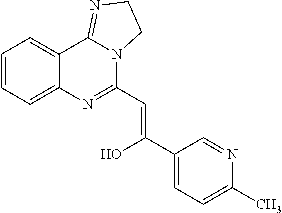 [SMILES: Cc1ccc(/C(O)=C/C2=Nc3ccccc3C3=NCCN23)cn1] |
| 1-137 | 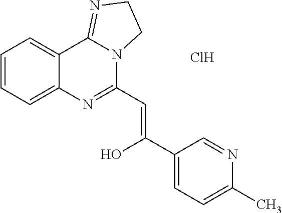 [SMILES: Cc1ccc(/C(O)=C/C2=Nc3ccccc3C3=NCCN23)cn1 ; Cl] |
| 1-138 | 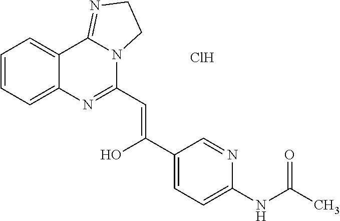 [SMILES: CC(=O)Nc1ccc(/C(O)=C/C2=Nc3ccccc3C3=NCCN23)cn1 ; Cl] |
| 1-139 | 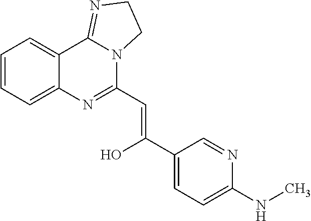 [SMILES: CNc1ccc(/C(O)=C/C2=Nc3ccccc3C3=NCCN23)cn1] |
| 1-140 | 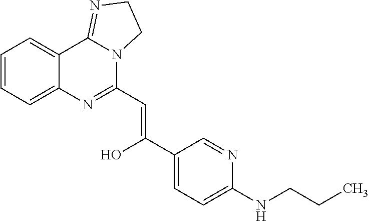 [SMILES: CCCNc1ccc(/C(O)=C/C2=Nc3ccccc3C3=NCCN23)cn1] |
| 1-141 | 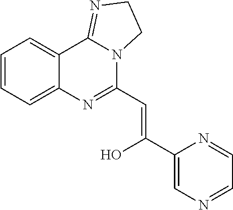 [SMILES: O/C(=C\C1=Nc2ccccc2C2=NCCN12)c1cnccn1] |
| 1-142 | 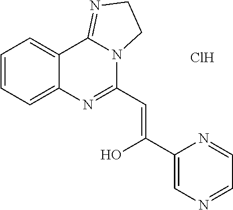 [SMILES: O/C(=C\C1=Nc2ccccc2C2=NCCN12)c1cnccn1 ; Cl] |
| 1-143 | 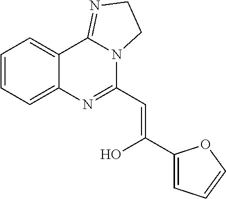 [SMILES: O/C(=C\C1=Nc2ccccc2C2=NCCN12)c1ccco1] |
| 1-144 | 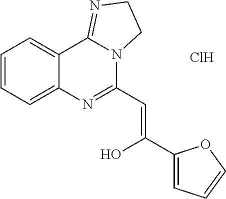 [SMILES: O/C(=C\C1=Nc2ccccc2C2=NCCN12)c1ccco1 ; Cl] |
| 1-145 | 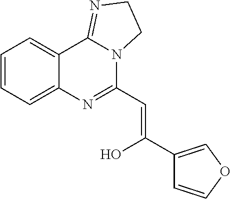 [SMILES: O/C(=C\C1=Nc2ccccc2C2=NCCN12)c1ccoc1] |
| 1-146 | 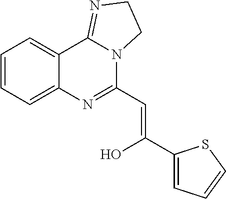 [SMILES: O/C(=C\C1=Nc2ccccc2C2=NCCN12)c1cccs1] |
| 1-147 | 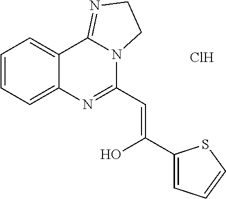 [SMILES: O/C(=C\C1=Nc2ccccc2C2=NCCN12)c1cccs1 ; Cl] |
| 1-148 | 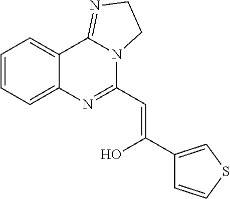 [SMILES: O/C(=C\C1=Nc2ccccc2C2=NCCN12)c1ccsc1] |
| 1-149 | 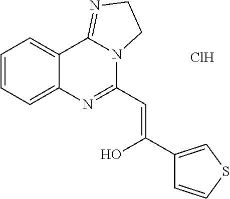 [SMILES: O/C(=C\C1=Nc2ccccc2C2=NCCN12)c1ccsc1 ; Cl] |
| 1-150 | 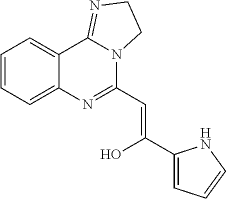 [SMILES: O/C(=C\C1=Nc2ccccc2C2=NCCN12)c1ccc[nH]1] |
| 1-151 | 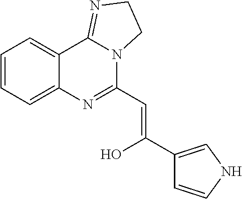 [SMILES: O/C(=C\C1=Nc2ccccc2C2=NCCN12)c1cc[nH]c1] |
| 1-152 | 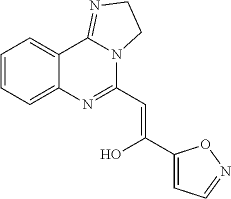 [SMILES: O/C(=C\C1=Nc2ccccc2C2=NCCN12)c1ccno1] |
| 1-153 | 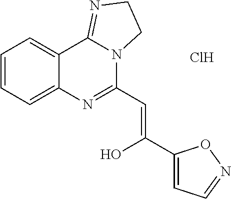 [SMILES: O/C(=C\C1=Nc2ccccc2C2=NCCN12)c1ccno1 ; Cl] |
| 1-154 | 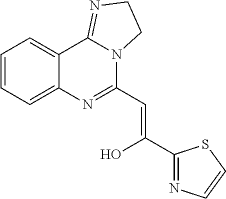 [SMILES: O/C(=C\C1=Nc2ccccc2C2=NCCN12)c1nccs1] |
| 1-155 | 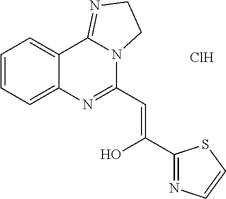 [SMILES: O/C(=C\C1=Nc2ccccc2C2=NCCN12)c1nccs1 ; Cl] |
| 1-156 | 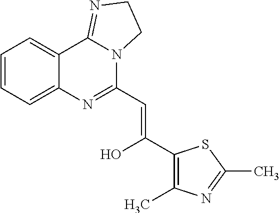 [SMILES: Cc1nc(C)c(/C(O)=C/C2=Nc3ccccc3C3=NCCN23)s1] |
| 1-157 | 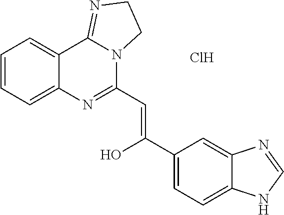 [SMILES: O/C(=C\C1=Nc2ccccc2C2=NCCN12)c1ccc2[nH]cnc2c1 ; Cl] |
| 1-158 | 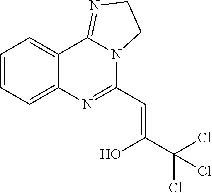 [SMILES: O/C(=C\C1=Nc2ccccc2C2=NCCN12)C(Cl)(Cl)Cl] |
| 1-159 | 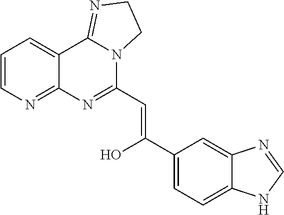 [SMILES: O/C(=C\C1=Nc2ncccc2C2=NCCN12)c1ccc2[nH]cnc2c1] |
| 1-160 | 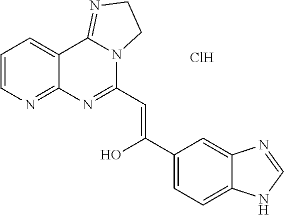 [SMILES: O/C(=C\C1=Nc2ncccc2C2=NCCN12)c1ccc2[nH]cnc2c1 ; Cl] |
| 1-161 | 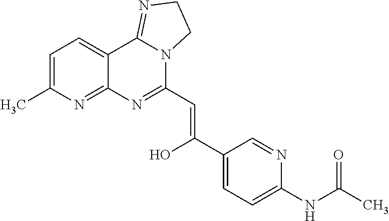 [SMILES: CC(=O)Nc1ccc(/C(O)=C/C2=Nc3nc(C)ccc3C3=NCCN23)cn1] |
| 1-162 | 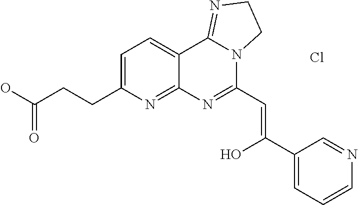 [SMILES: O=C(O)CCc1ccc2c(n1)N=C(/C=C(\O)c1cccnc1)N1CCN=C21 ; Cl] |
| 1-163 | 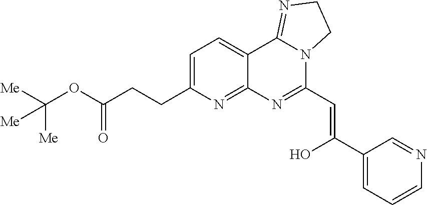 [SMILES: CC(C)(C)OC(=O)CCc1ccc2c(n1)N=C(/C=C(\O)c1cccnc1)N1CCN=C21] |
| 1-164 | 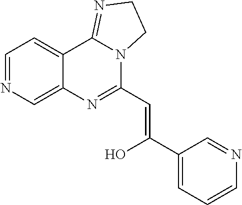 [SMILES: O/C(=C\C1=Nc2cnccc2C2=NCCN12)c1cccnc1] |
| 1-165 | 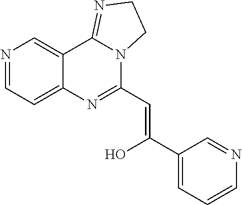 [SMILES: O/C(=C\C1=Nc2ccncc2C2=NCCN12)c1cccnc1] |
| 1-166 | 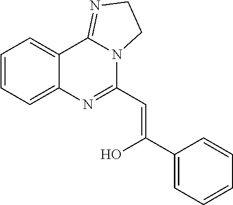 [SMILES: O/C(=C\C1=Nc2ccccc2C2=NCCN12)c1ccccc1] |
| 1-167 | 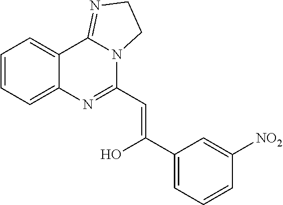 [SMILES: O=[N+]([O-])c1cccc(/C(O)=C/C2=Nc3ccccc3C3=NCCN23)c1] |
| 1-168 | 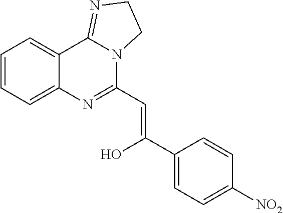 [SMILES: O=[N+]([O-])c1ccc(/C(O)=C/C2=Nc3ccccc3C3=NCCN23)cc1] |
| 1-169 | 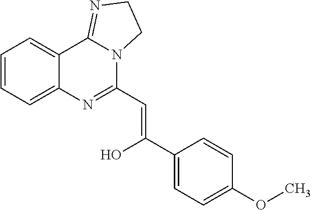 [SMILES: COc1ccc(/C(O)=C/C2=Nc3ccccc3C3=NCCN23)cc1] |
| 1-170 | 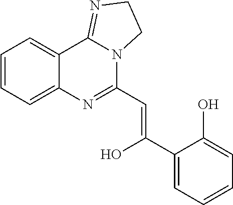 [SMILES: O/C(=C\C1=Nc2ccccc2C2=NCCN12)c1ccccc1O] |
| 1-171 | 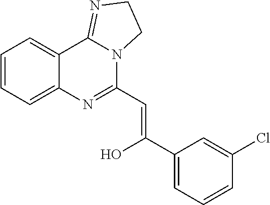 [SMILES: O/C(=C\C1=Nc2ccccc2C2=NCCN12)c1cccc(Cl)c1] |
| 1-172 | 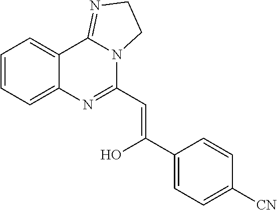 [SMILES: N#Cc1ccc(/C(O)=C/C2=Nc3ccccc3C3=NCCN23)cc1] |
| 1-173 | 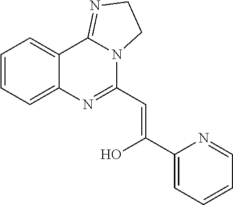 [SMILES: O/C(=C\C1=Nc2ccccc2C2=NCCN12)c1ccccn1] |
| 1-174 | 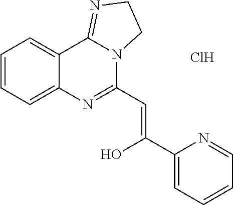 [SMILES: O/C(=C\C1=Nc2ccccc2C2=NCCN12)c1ccccn1 ; Cl] |
| 1-175 | 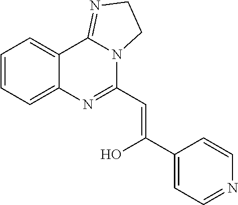 [SMILES: O/C(=C\C1=Nc2ccccc2C2=NCCN12)c1ccncc1] |
| 1-176 | 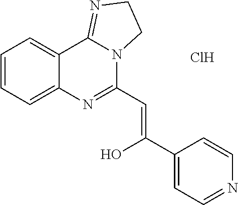 [SMILES: O/C(=C\C1=Nc2ccccc2C2=NCCN12)c1ccncc1 ; Cl] |
| 1-177 | 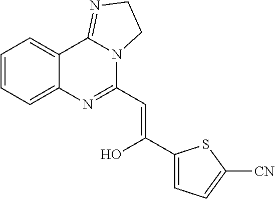 [SMILES: N#Cc1ccc(/C(O)=C/C2=Nc3ccccc3C3=NCCN23)s1] |
| 1-178 | 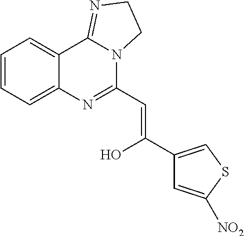 [SMILES: O=[N+]([O-])c1cc(/C(O)=C/C2=Nc3ccccc3C3=NCCN23)cs1] |
| 1-179 | 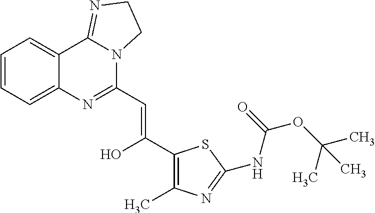 [SMILES: Cc1nc(NC(=O)OC(C)(C)C)sc1/C(O)=C/C1=Nc2ccccc2C2=NCCN12] |
| 1-180 | 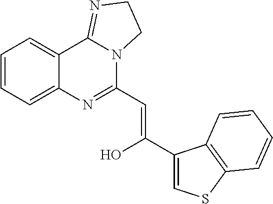 [SMILES: O/C(=C\C1=Nc2ccccc2C2=NCCN12)c1csc2ccccc12] |
| 1-181 | 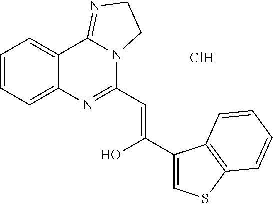 [SMILES: O/C(=C\C1=Nc2ccccc2C2=NCCN12)c1csc2ccccc12 ; Cl] |
| 1-182 | 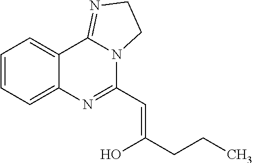 [SMILES: CCC/C(O)=C/C1=Nc2ccccc2C2=NCCN12] |
| 1-183 | 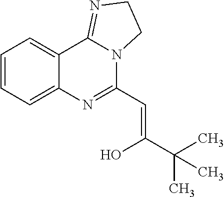 [SMILES: CC(C)(C)/C(O)=C/C1=Nc2ccccc2C2=NCCN12] |
| 1-184 | 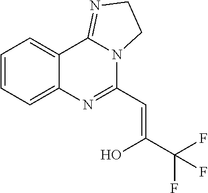 [SMILES: O/C(=C\C1=Nc2ccccc2C2=NCCN12)C(F)(F)F] |
| 1-185 | 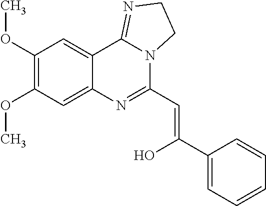 [SMILES: COc1cc2c(cc1OC)C1=NCCN1C(/C=C(\O)c1ccccc1)=N2] |
| 1-186 | 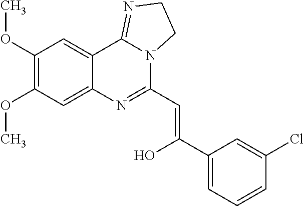 [SMILES: COc1cc2c(cc1OC)C1=NCCN1C(/C=C(\O)c1cccc(Cl)c1)=N2] |
| 1-187 | 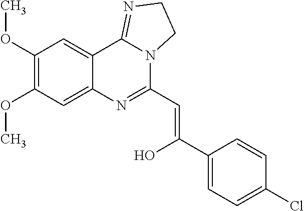 [SMILES: COc1cc2c(cc1OC)C1=NCCN1C(/C=C(\O)c1ccc(Cl)cc1)=N2] |
| 1-188 | 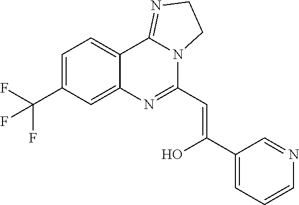 [SMILES: O/C(=C\C1=Nc2cc(C(F)(F)F)ccc2C2=NCCN12)c1cccnc1] |
| 1-189 | 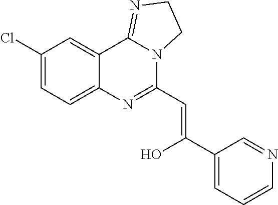 [SMILES: O/C(=C\C1=Nc2ccc(Cl)cc2C2=NCCN12)c1cccnc1] |
| 1-190 | 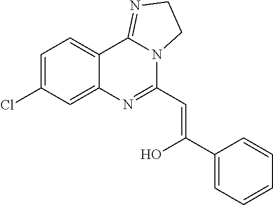 [SMILES: O/C(=C\C1=Nc2cc(Cl)ccc2C2=NCCN12)c1ccccc1] |
| 1-191 | 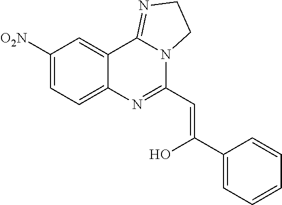 [SMILES: O=[N+]([O-])c1ccc2c(c1)C1=NCCN1C(/C=C(\O)c1ccccc1)=N2] |
| 1-192 | 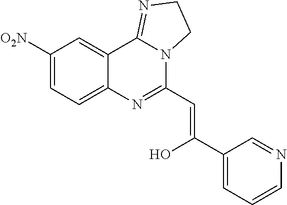 [SMILES: O=[N+]([O-])c1ccc2c(c1)C1=NCCN1C(/C=C(\O)c1cccnc1)=N2] |
| 1-193 | 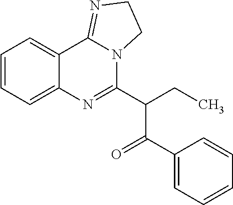 [SMILES: CCC(C(=O)c1ccccc1)C1=Nc2ccccc2C2=NCCN21] |
| 1-194 | 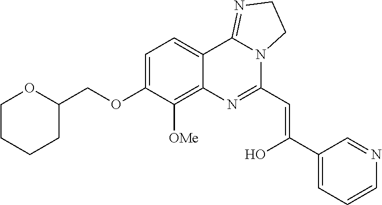 [SMILES: COc1c(OCC2CCCCO2)ccc2c1N=C(/C=C(\O)c1cccnc1)N1CCN=C21] |
| 1-195 | 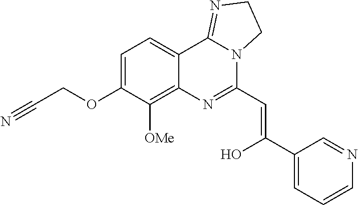 [SMILES: COc1c(OCC#N)ccc2c1N=C(/C=C(\O)c1cccnc1)N1CCN=C21] |
| 1-196 | 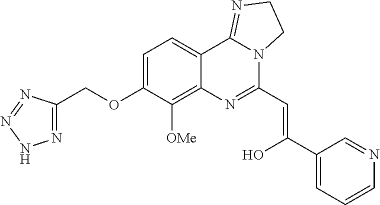 [SMILES: COc1c(OCc2nn[nH]n2)ccc2c1N=C(/C=C(\O)c1cccnc1)N1CCN=C21] |
| 1-197 | 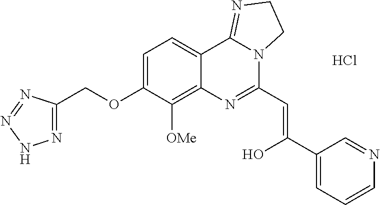 [SMILES: COc1c(OCc2nn[nH]n2)ccc2c1N=C(/C=C(\O)c1cccnc1)N1CCN=C21 ; Cl] |
| 1-198 | 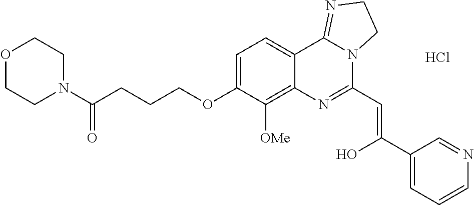 [SMILES: COc1c(OCCCC(=O)N2CCOCC2)ccc2c1N=C(/C=C(\O)c1cccnc1)N1CCN=C21 ; Cl] |
| 1-199 | 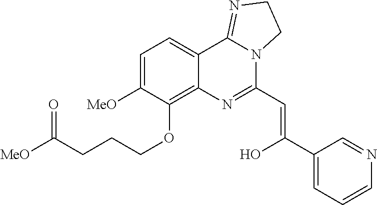 [SMILES: COC(=O)CCCOc1c(OC)ccc2c1N=C(/C=C(\O)c1cccnc1)N1CCN=C21] |
| 1-200 | 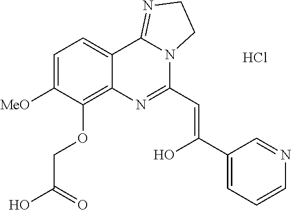 [SMILES: COc1ccc2c(c1OCC(=O)O)N=C(/C=C(\O)c1cccnc1)N1CCN=C21 ; Cl] |
| 1-201 | 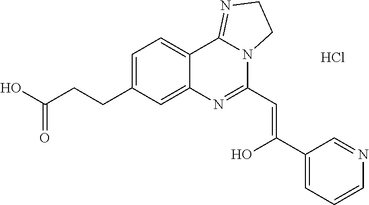 [SMILES: O=C(O)CCc1ccc2c(c1)N=C(/C=C(\O)c1cccnc1)N1CCN=C21 ; Cl] |
| 1-202 | 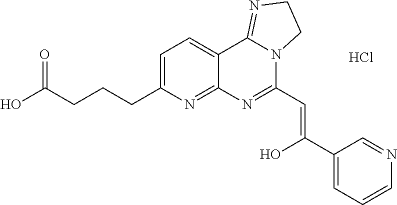 [SMILES: O=C(O)CCCc1ccc2c(n1)N=C(/C=C(\O)c1cccnc1)N1CCN=C21 ; Cl] |
| 1-203 | 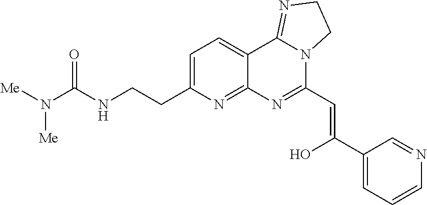 [SMILES: CN(C)C(=O)NCCc1ccc2c(n1)N=C(/C=C(\O)c1cccnc1)N1CCN=C21] |
| 1-204 | 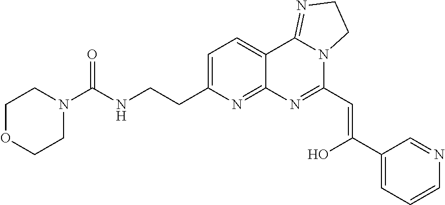 [SMILES: O=C(NCCc1ccc2c(n1)N=C(/C=C(\O)c1cccnc1)N1CCN=C21)N1CCOCC1] |
| 1-205 | 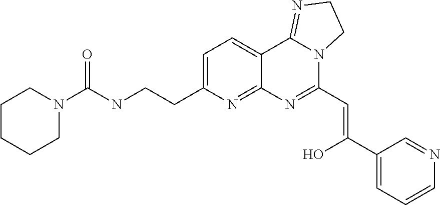 [SMILES: O=C(NCCc1ccc2c(n1)N=C(/C=C(\O)c1cccnc1)N1CCN=C21)N1CCCCC1] |
| 1-206 | 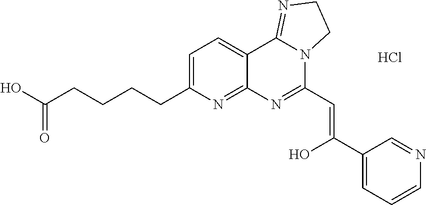 [SMILES: O=C(O)CCCCc1ccc2c(n1)N=C(/C=C(\O)c1cccnc1)N1CCN=C21 ; Cl] |
| 1-207 | 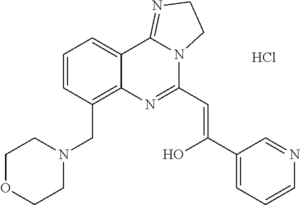 [SMILES: O/C(=C\C1=Nc2c(CN3CCOCC3)cccc2C2=NCCN12)c1cccnc1 ; Cl] |
| 1-208 | 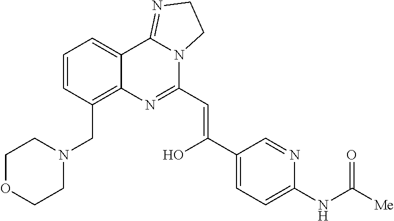 [SMILES: CC(=O)Nc1ccc(/C(O)=C/C2=Nc3c(CN4CCOCC4)cccc3C3=NCCN23)cn1] |
| 1-209 | 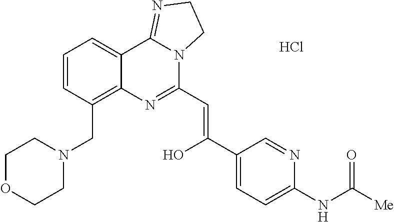 [SMILES: CC(=O)Nc1ccc(/C(O)=C/C2=Nc3c(CN4CCOCC4)cccc3C3=NCCN23)cn1 ; Cl] |
| 1-210 | 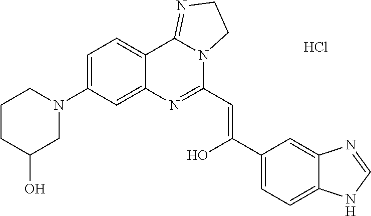 [SMILES: O/C(=C\C1=Nc2cc(N3CCCC(O)C3)ccc2C2=NCCN12)c1ccc2[nH]cnc2c1 ; Cl] |
| 2-1 | 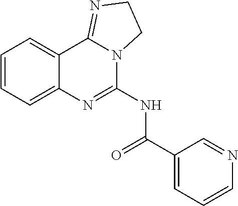 [SMILES: O=C(NC1=Nc2ccccc2C2=NCCN12)c1cccnc1] |
| 2-2 | 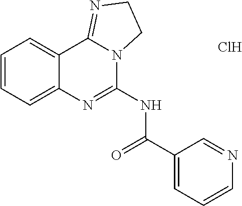 [SMILES: O=C(NC1=Nc2ccccc2C2=NCCN12)c1cccnc1 ; Cl] |
| 2-3 | 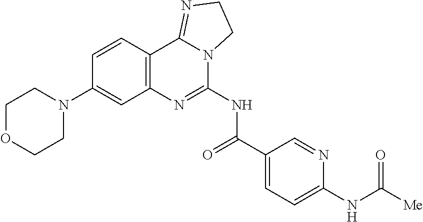 [SMILES: CC(=O)Nc1ccc(C(=O)NC2=Nc3cc(N4CCOCC4)ccc3C3=NCCN23)cn1] |
| 2-4 | 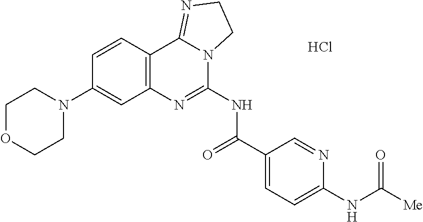 [SMILES: CC(=O)Nc1ccc(C(=O)NC2=Nc3cc(N4CCOCC4)ccc3C3=NCCN23)cn1 ; Cl] |
| 2-5 | 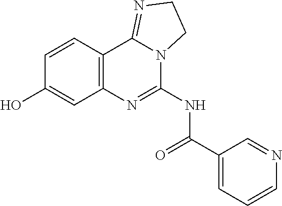 [SMILES: O=C(NC1=Nc2cc(O)ccc2C2=NCCN12)c1cccnc1] |
| 2-6 | 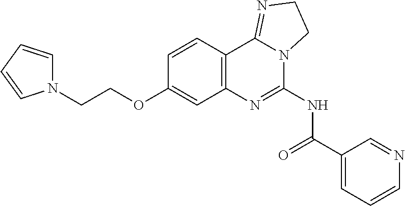 [SMILES: O=C(NC1=Nc2cc(OCCn3cccc3)ccc2C2=NCCN12)c1cccnc1] |
| 2-7 | 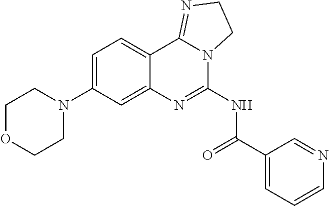 [SMILES: O=C(NC1=Nc2cc(N3CCOCC3)ccc2C2=NCCN12)c1cccnc1] |
| 2-8 | 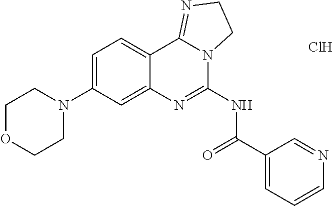 [SMILES: O=C(NC1=Nc2cc(N3CCOCC3)ccc2C2=NCCN12)c1cccnc1 ; Cl] |
| 2-9 | 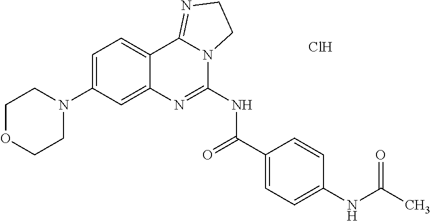 [SMILES: CC(=O)Nc1ccc(C(=O)NC2=Nc3cc(N4CCOCC4)ccc3C3=NCCN23)cc1 ; Cl] |
| 2-10 | 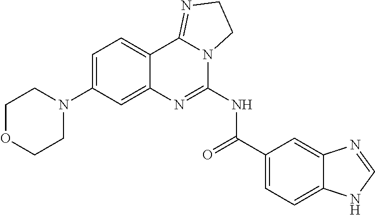 [SMILES: O=C(NC1=Nc2cc(N3CCOCC3)ccc2C2=NCCN12)c1ccc2[nH]cnc2c1] |
| 2-11 | 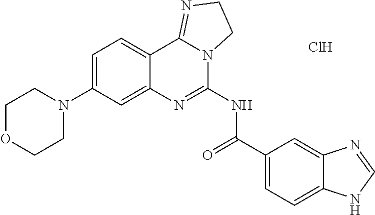 [SMILES: O=C(NC1=Nc2cc(N3CCOCC3)ccc2C2=NCCN12)c1ccc2[nH]cnc2c1 ; Cl] |
| 2-12 | 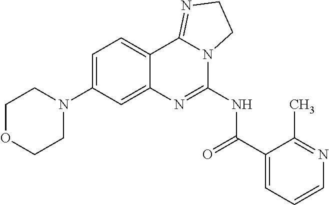 [SMILES: Cc1ncccc1C(=O)NC1=Nc2cc(N3CCOCC3)ccc2C2=NCCN12] |
| 2-13 | 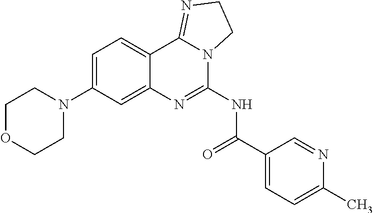 [SMILES: Cc1ccc(C(=O)NC2=Nc3cc(N4CCOCC4)ccc3C3=NCCN23)cn1] |
| 2-14 | 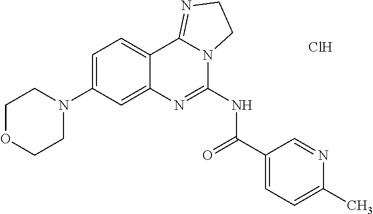 [SMILES: Cc1ccc(C(=O)NC2=Nc3cc(N4CCOCC4)ccc3C3=NCCN23)cn1 ; Cl] |
| 2-15 | 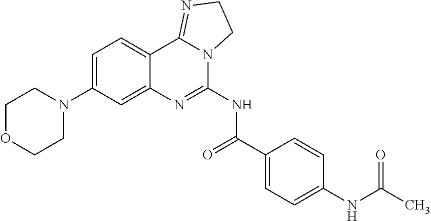 [SMILES: CC(=O)Nc1ccc(C(=O)NC2=Nc3cc(N4CCOCC4)ccc3C3=NCCN23)cc1] |
| 2-16 | 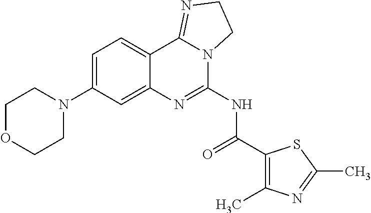 [SMILES: Cc1nc(C)c(C(=O)NC2=Nc3cc(N4CCOCC4)ccc3C3=NCCN23)s1] |
| 2-17 | 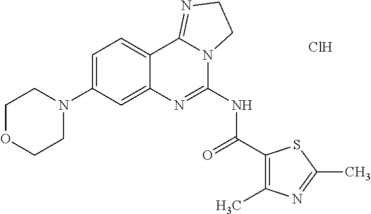 [SMILES: Cc1nc(C)c(C(=O)NC2=Nc3cc(N4CCOCC4)ccc3C3=NCCN23)s1 ; Cl] |
| 2-18 | 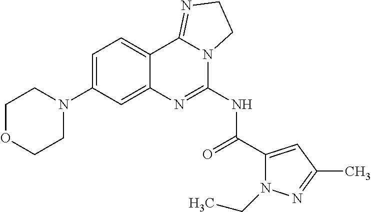 [SMILES: CCn1nc(C)cc1C(=O)NC1=Nc2cc(N3CCOCC3)ccc2C2=NCCN12] |
| 2-19 | 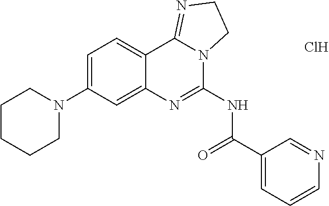 [SMILES: O=C(NC1=Nc2cc(N3CCCCC3)ccc2C2=NCCN12)c1cccnc1 ; Cl] |
| 2-20 | 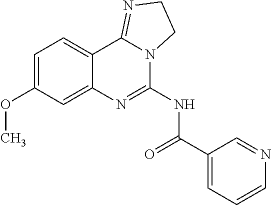 [SMILES: COc1ccc2c(c1)N=C(NC(=O)c1cccnc1)N1CCN=C21] |
| 2-21 | 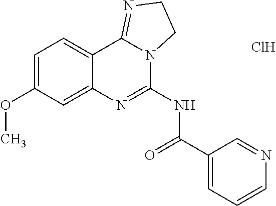 [SMILES: COc1ccc2c(c1)N=C(NC(=O)c1cccnc1)N1CCN=C21 ; Cl] |
| 2-22 | 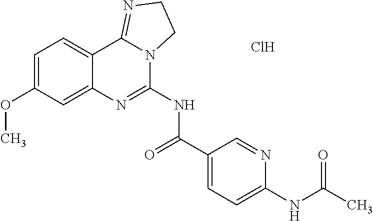 [SMILES: COc1ccc2c(c1)N=C(NC(=O)c1ccc(NC(C)=O)nc1)N1CCN=C21 ; Cl] |
| 2-23 | 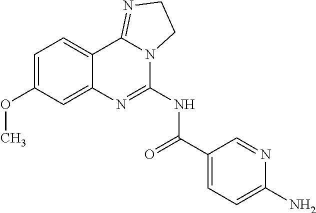 [SMILES: COc1ccc2c(c1)N=C(NC(=O)c1ccc(N)nc1)N1CCN=C21] |
| 2-24 | 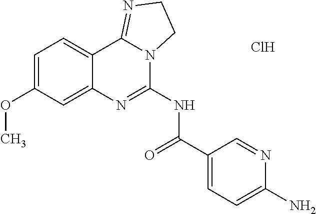 [SMILES: COc1ccc2c(c1)N=C(NC(=O)c1ccc(N)nc1)N1CCN=C21 ; Cl] |
| 2-25 | 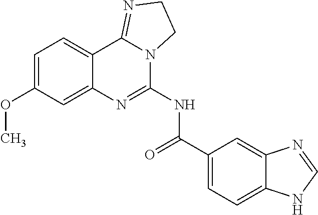 [SMILES: COc1ccc2c(c1)N=C(NC(=O)c1ccc3[nH]cnc3c1)N1CCN=C21] |
| 2-26 | 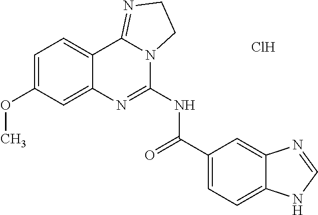 [SMILES: COc1ccc2c(c1)N=C(NC(=O)c1ccc3[nH]cnc3c1)N1CCN=C21 ; Cl] |
| 2-27 | 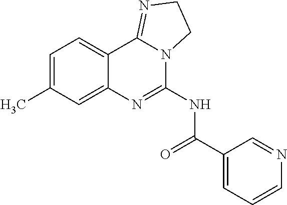 [SMILES: Cc1ccc2c(c1)N=C(NC(=O)c1cccnc1)N1CCN=C21] |
| 2-28 | 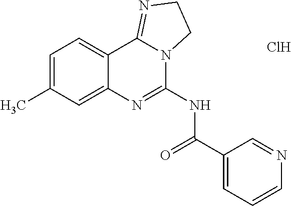 [SMILES: Cc1ccc2c(c1)N=C(NC(=O)c1cccnc1)N1CCN=C21 ; Cl] |
| 2-29 | 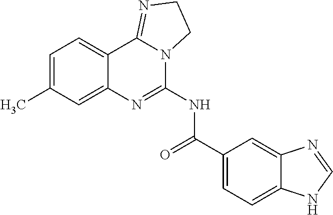 [SMILES: Cc1ccc2c(c1)N=C(NC(=O)c1ccc3[nH]cnc3c1)N1CCN=C21] |
| 2-30 | 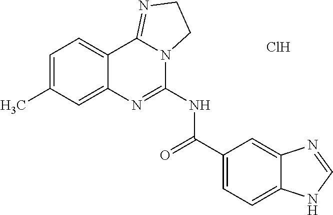 [SMILES: Cc1ccc2c(c1)N=C(NC(=O)c1ccc3[nH]cnc3c1)N1CCN=C21 ; Cl] |
| 2-31 | 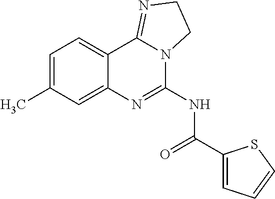 [SMILES: Cc1ccc2c(c1)N=C(NC(=O)c1cccs1)N1CCN=C21] |
| 2-32 | 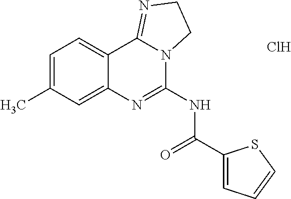 [SMILES: Cc1ccc2c(c1)N=C(NC(=O)c1cccs1)N1CCN=C21 ; Cl] |
| 2-33 | 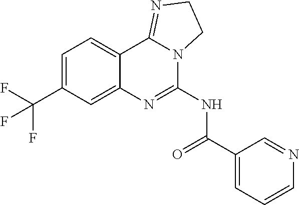 [SMILES: O=C(NC1=Nc2cc(C(F)(F)F)ccc2C2=NCCN12)c1cccnc1] |
| 2-34 | 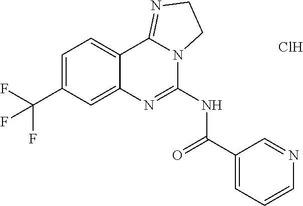 [SMILES: O=C(NC1=Nc2cc(C(F)(F)F)ccc2C2=NCCN12)c1cccnc1 ; Cl] |
| 2-35 | 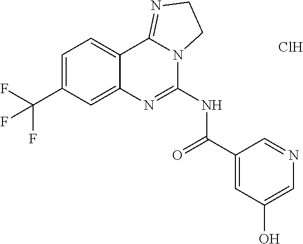 [SMILES: O=C(NC1=Nc2cc(C(F)(F)F)ccc2C2=NCCN12)c1cncc(O)c1 ; Cl] |
| 2-36 | 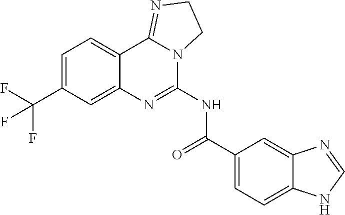 [SMILES: O=C(NC1=Nc2cc(C(F)(F)F)ccc2C2=NCCN12)c1ccc2[nH]cnc2c1] |
| 2-37 | 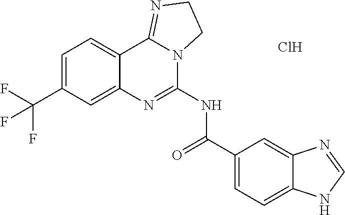 [SMILES: O=C(NC1=Nc2cc(C(F)(F)F)ccc2C2=NCCN12)c1ccc2[nH]cnc2c1 ; Cl] |
| 2-38 | 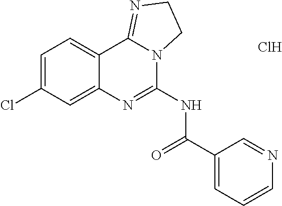 [SMILES: O=C(NC1=Nc2cc(Cl)ccc2C2=NCCN12)c1cccnc1 ; Cl] |
| 2-39 | 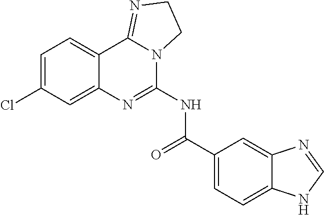 [SMILES: O=C(NC1=Nc2cc(Cl)ccc2C2=NCCN12)c1ccc2[nH]cnc2c1] |
| 2-40 | 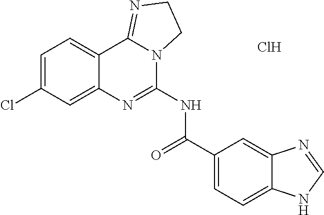 [SMILES: O=C(NC1=Nc2cc(Cl)ccc2C2=NCCN12)c1ccc2[nH]cnc2c1 ; Cl] |
| 2-41 | 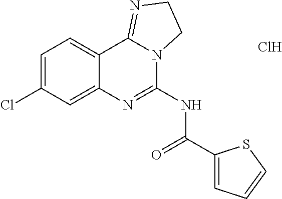 [SMILES: O=C(NC1=Nc2cc(Cl)ccc2C2=NCCN12)c1cccs1 ; Cl] |
| 2-42 | 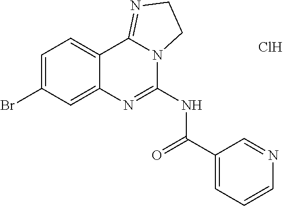 [SMILES: O=C(NC1=Nc2cc(Br)ccc2C2=NCCN12)c1cccnc1 ; Cl] |
| 2-43 | 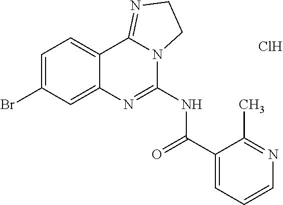 [SMILES: Cc1ncccc1C(=O)NC1=Nc2cc(Br)ccc2C2=NCCN12 ; Cl] |
| 2-44 | 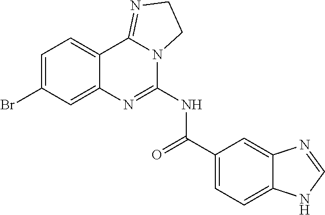 [SMILES: O=C(NC1=Nc2cc(Br)ccc2C2=NCCN12)c1ccc2[nH]cnc2c1] |
| 2-45 | 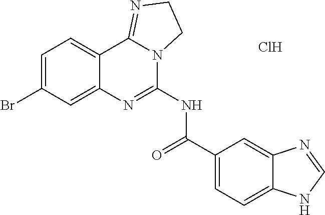 [SMILES: O=C(NC1=Nc2cc(Br)ccc2C2=NCCN12)c1ccc2[nH]cnc2c1 ; Cl] |
| 2-46 | 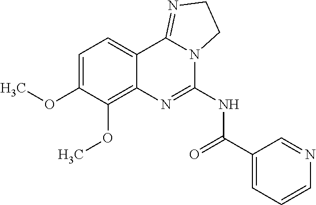 [SMILES: COc1ccc2c(c1OC)N=C(NC(=O)c1cccnc1)N1CCN=C21] |
| 2-47 | 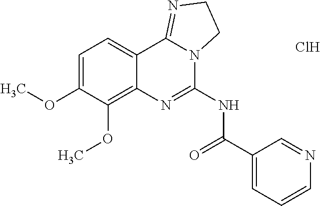 [SMILES: COc1ccc2c(c1OC)N=C(NC(=O)c1cccnc1)N1CCN=C21 ; Cl] |
| 2-48 | 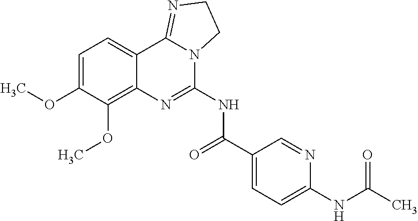 [SMILES: COc1ccc2c(c1OC)N=C(NC(=O)c1ccc(NC(C)=O)nc1)N1CCN=C21] |
| 2-49 | 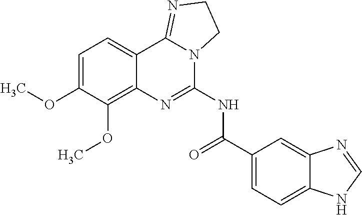 [SMILES: COc1ccc2c(c1OC)N=C(NC(=O)c1ccc3[nH]cnc3c1)N1CCN=C21] |
| 2-50 | 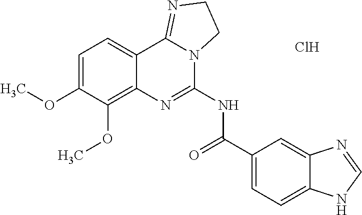 [SMILES: COc1ccc2c(c1OC)N=C(NC(=O)c1ccc3[nH]cnc3c1)N1CCN=C21 ; Cl] |
| 2-51 | 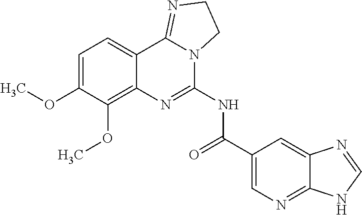 [SMILES: COc1ccc2c(c1OC)N=C(NC(=O)c1cnc3[nH]cnc3c1)N1CCN=C21] |
| 2-52 | 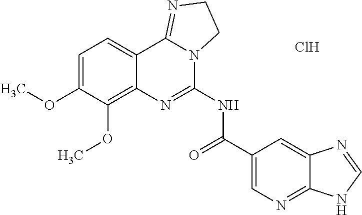 [SMILES: COc1ccc2c(c1OC)N=C(NC(=O)c1cnc3[nH]cnc3c1)N1CCN=C21 ; Cl] |
| 2-53 | 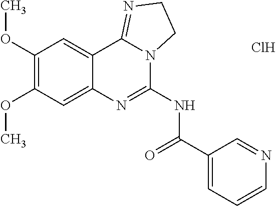 [SMILES: COc1cc2c(cc1OC)C1=NCCN1C(NC(=O)c1cccnc1)=N2 ; Cl] |
| 2-54 | 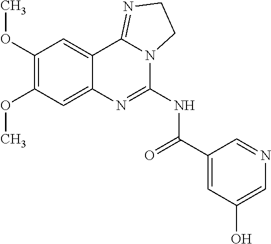 [SMILES: COc1cc2c(cc1OC)C1=NCCN1C(NC(=O)c1cncc(O)c1)=N2] |
| 2-55 | 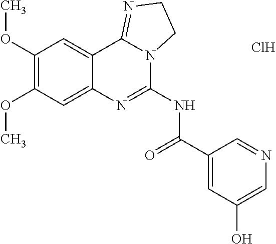 [SMILES: COc1cc2c(cc1OC)C1=NCCN1C(NC(=O)c1cncc(O)c1)=N2 ; Cl] |
| 2-56 | 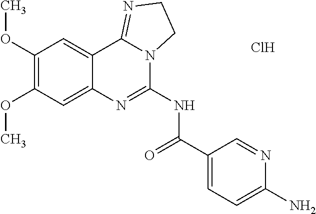 [SMILES: COc1cc2c(cc1OC)C1=NCCN1C(NC(=O)c1ccc(N)nc1)=N2 ; Cl] |
| 2-57 | 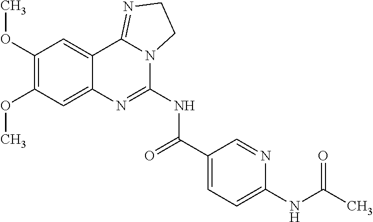 [SMILES: COc1cc2c(cc1OC)C1=NCCN1C(NC(=O)c1ccc(NC(C)=O)nc1)=N2] |
| 2-58 | 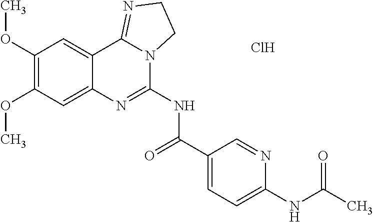 [SMILES: COc1cc2c(cc1OC)C1=NCCN1C(NC(=O)c1ccc(NC(C)=O)nc1)=N2 ; Cl] |
| 2-59 | 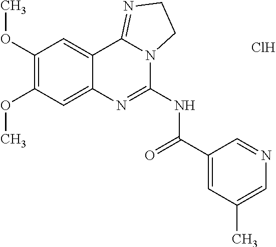 [SMILES: COc1cc2c(cc1OC)C1=NCCN1C(NC(=O)c1cncc(C)c1)=N2 ; Cl] |
| 2-60 | 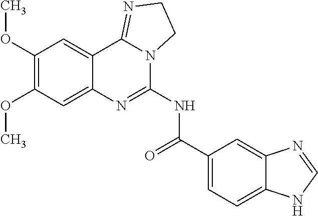 [SMILES: COc1cc2c(cc1OC)C1=NCCN1C(NC(=O)c1ccc3[nH]cnc3c1)=N2] |
| 2-61 | 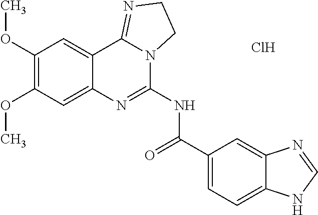 [SMILES: COc1cc2c(cc1OC)C1=NCCN1C(NC(=O)c1ccc3[nH]cnc3c1)=N2 ; Cl] |
| 2-62 | 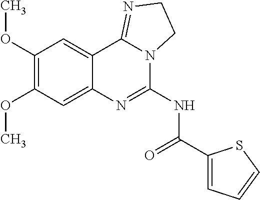 [SMILES: COc1cc2c(cc1OC)C1=NCCN1C(NC(=O)c1cccs1)=N2] |
| 2-63 | 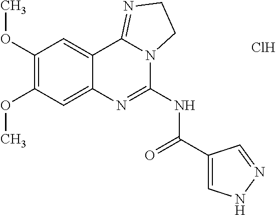 [SMILES: COc1cc2c(cc1OC)C1=NCCN1C(NC(=O)c1cn[nH]c1)=N2 ; Cl] |
| 2-64 | 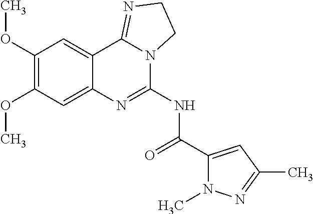 [SMILES: COc1cc2c(cc1OC)C1=NCCN1C(NC(=O)c1cc(C)nn1C)=N2] |
| 2-65 | 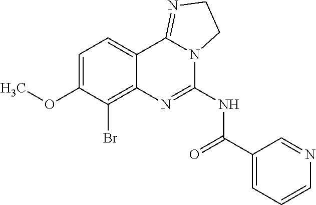 [SMILES: COc1ccc2c(c1Br)N=C(NC(=O)c1cccnc1)N1CCN=C21] |
| 2-66 | 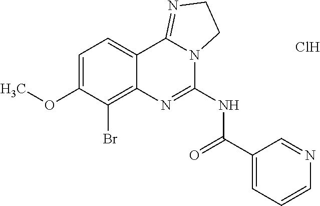 [SMILES: COc1ccc2c(c1Br)N=C(NC(=O)c1cccnc1)N1CCN=C21 ; Cl] |
| 2-67 | 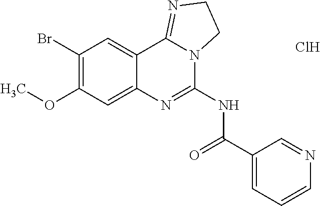 [SMILES: COc1cc2c(cc1Br)C1=NCCN1C(NC(=O)c1cccnc1)=N2 ; Cl] |
| 2-68 | 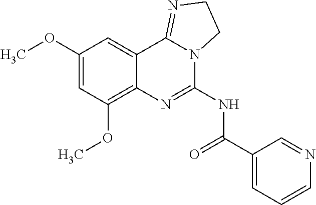 [SMILES: COc1cc(OC)c2c(c1)C1=NCCN1C(NC(=O)c1cccnc1)=N2] |
| 2-69 | 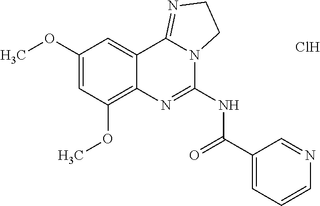 [SMILES: COc1cc(OC)c2c(c1)C1=NCCN1C(NC(=O)c1cccnc1)=N2 ; Cl] |
| 2-70 | 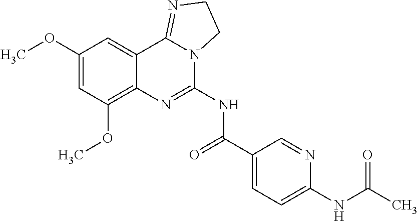 [SMILES: COc1cc(OC)c2c(c1)C1=NCCN1C(NC(=O)c1ccc(NC(C)=O)nc1)=N2] |
| 2-71 | 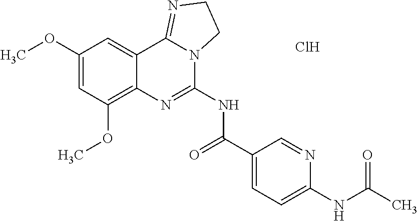 [SMILES: COc1cc(OC)c2c(c1)C1=NCCN1C(NC(=O)c1ccc(NC(C)=O)nc1)=N2 ; Cl] |
| 2-72 | 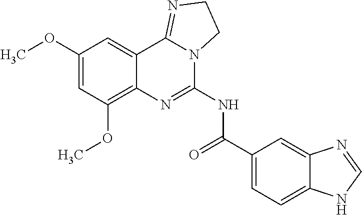 [SMILES: COc1cc(OC)c2c(c1)C1=NCCN1C(NC(=O)c1ccc3[nH]cnc3c1)=N2] |
| 2-73 | 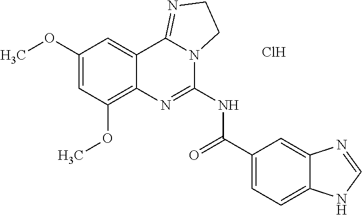 [SMILES: COc1cc(OC)c2c(c1)C1=NCCN1C(NC(=O)c1ccc3[nH]cnc3c1)=N2 ; Cl] |
| 2-74 | 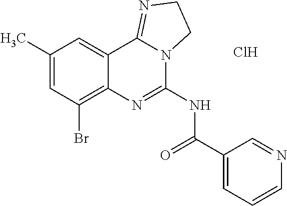 [SMILES: Cc1cc(Br)c2c(c1)C1=NCCN1C(NC(=O)c1cccnc1)=N2 ; Cl] |
| 2-75 | 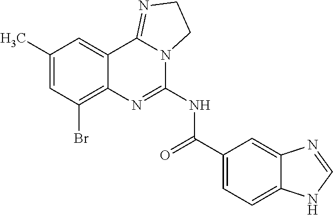 [SMILES: Cc1cc(Br)c2c(c1)C1=NCCN1C(NC(=O)c1ccc3[nH]cnc3c1)=N2] |
| 2-76 | 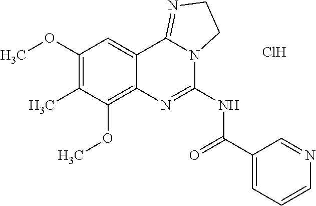 [SMILES: COc1cc2c(c(OC)c1C)N=C(NC(=O)c1cccnc1)N1CCN=C21 ; Cl] |
| 2-77 | 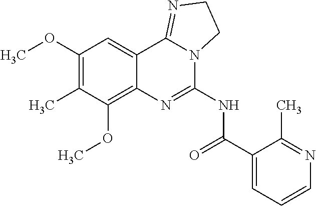 [SMILES: COc1cc2c(c(OC)c1C)N=C(NC(=O)c1cccnc1C)N1CCN=C21] |
| 2-78 | 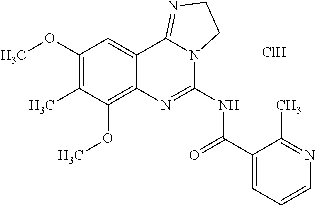 [SMILES: COc1cc2c(c(OC)c1C)N=C(NC(=O)c1cccnc1C)N1CCN=C21 ; Cl] |
| 2-79 | 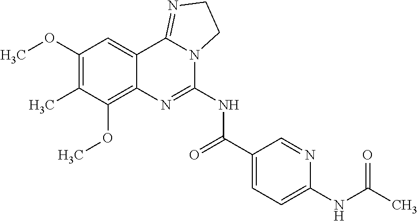 [SMILES: COc1cc2c(c(OC)c1C)N=C(NC(=O)c1ccc(NC(C)=O)nc1)N1CCN=C21] |
| 2-80 | 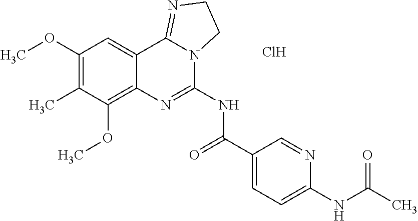 [SMILES: COc1cc2c(c(OC)c1C)N=C(NC(=O)c1ccc(NC(C)=O)nc1)N1CCN=C21 ; Cl] |
| 2-81 | 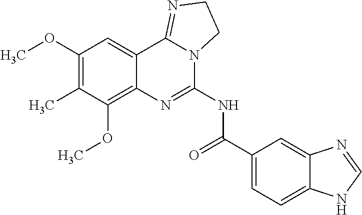 [SMILES: COc1cc2c(c(OC)c1C)N=C(NC(=O)c1ccc3[nH]cnc3c1)N1CCN=C21] |
| 2-82 | 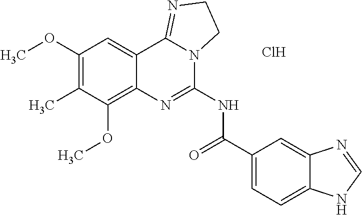 [SMILES: COc1cc2c(c(OC)c1C)N=C(NC(=O)c1ccc3[nH]cnc3c1)N1CCN=C21 ; Cl] |
| 2-83 | 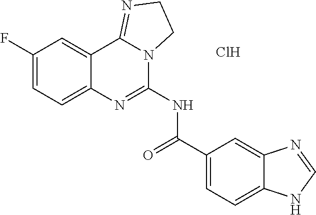 [SMILES: O=C(NC1=Nc2ccc(F)cc2C2=NCCN12)c1ccc2[nH]cnc2c1 ; Cl] |
| 2-84 | 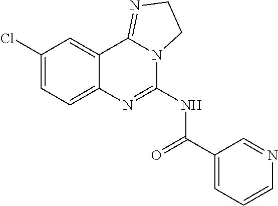 [SMILES: O=C(NC1=Nc2ccc(Cl)cc2C2=NCCN12)c1cccnc1] |
| 2-85 | 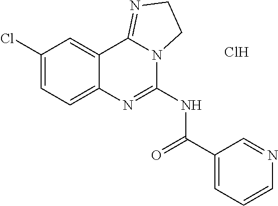 [SMILES: O=C(NC1=Nc2ccc(Cl)cc2C2=NCCN12)c1cccnc1 ; Cl] |
| 2-86 | 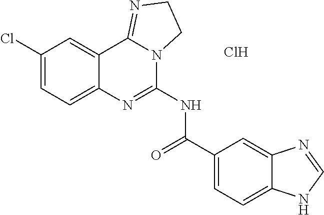 [SMILES: O=C(NC1=Nc2ccc(Cl)cc2C2=NCCN12)c1ccc2[nH]cnc2c1 ; Cl] |
| 2-87 | 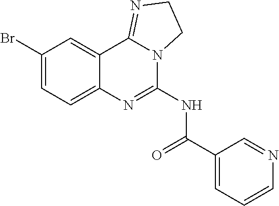 [SMILES: O=C(NC1=Nc2ccc(Br)cc2C2=NCCN12)c1cccnc1] |
| 2-88 | 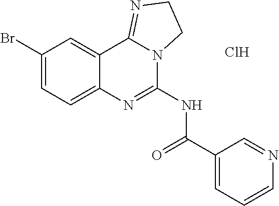 [SMILES: O=C(NC1=Nc2ccc(Br)cc2C2=NCCN12)c1cccnc1 ; Cl] |
| 2-89 | 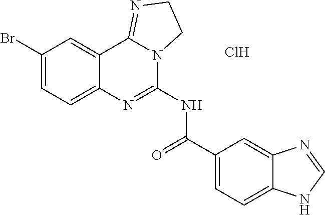 [SMILES: O=C(NC1=Nc2ccc(Br)cc2C2=NCCN12)c1ccc2[nH]cnc2c1 ; Cl] |
| 2-90 | 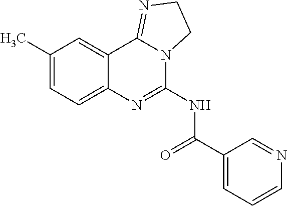 [SMILES: Cc1ccc2c(c1)C1=NCCN1C(NC(=O)c1cccnc1)=N2] |
| 2-91 | 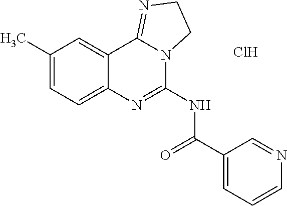 [SMILES: Cc1ccc2c(c1)C1=NCCN1C(NC(=O)c1cccnc1)=N2 ; Cl] |
| 2-92 | 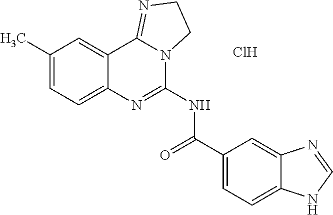 [SMILES: Cc1ccc2c(c1)C1=NCCN1C(NC(=O)c1ccc3[nH]cnc3c1)=N2 ; Cl] |
| 2-93 | 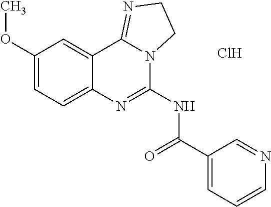 [SMILES: COc1ccc2c(c1)C1=NCCN1C(NC(=O)c1cccnc1)=N2 ; Cl] |
| 2-94 | 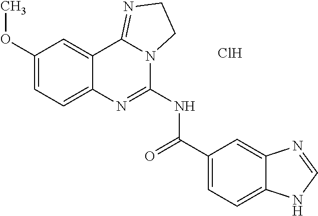 [SMILES: COc1ccc2c(c1)C1=NCCN1C(NC(=O)c1ccc3[nH]cnc3c1)=N2 ; Cl] |
| 2-95 | 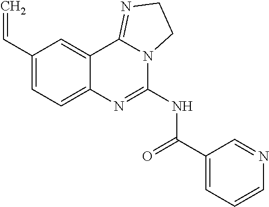 [SMILES: C=Cc1ccc2c(c1)C1=NCCN1C(NC(=O)c1cccnc1)=N2] |
| 2-96 | 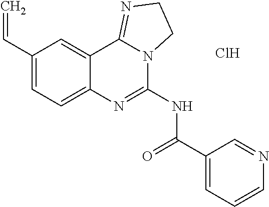 [SMILES: C=Cc1ccc2c(c1)C1=NCCN1C(NC(=O)c1cccnc1)=N2 ; Cl] |
| 2-97 | 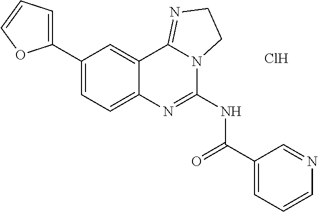 [SMILES: O=C(NC1=Nc2ccc(-c3ccco3)cc2C2=NCCN12)c1cccnc1 ; Cl] |
| 2-98 | 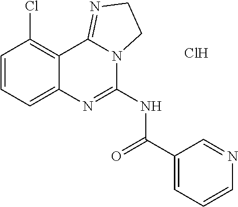 [SMILES: O=C(NC1=Nc2cccc(Cl)c2C2=NCCN12)c1cccnc1 ; Cl] |
| 2-99 | 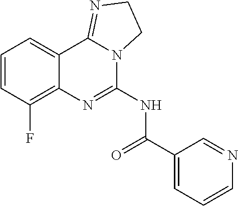 [SMILES: O=C(NC1=Nc2c(F)cccc2C2=NCCN12)c1cccnc1] |
| 2-100 | 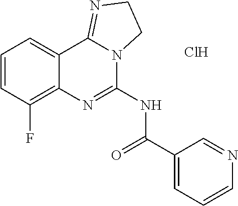 [SMILES: O=C(NC1=Nc2c(F)cccc2C2=NCCN12)c1cccnc1 ; Cl] |
| 2-101 | 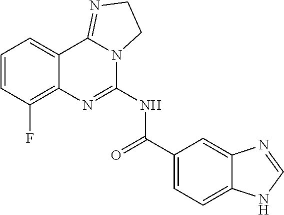 [SMILES: O=C(NC1=Nc2c(F)cccc2C2=NCCN12)c1ccc2[nH]cnc2c1] |
| 2-102 | 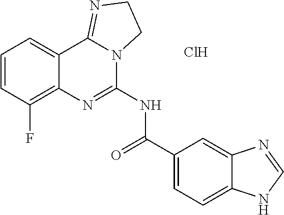 [SMILES: O=C(NC1=Nc2c(F)cccc2C2=NCCN12)c1ccc2[nH]cnc2c1 ; Cl] |
| 2-103 | 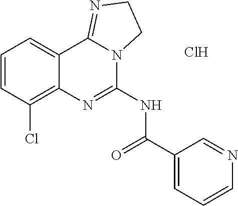 [SMILES: O=C(NC1=Nc2c(Cl)cccc2C2=NCCN12)c1cccnc1 ; Cl] |
| 2-104 | 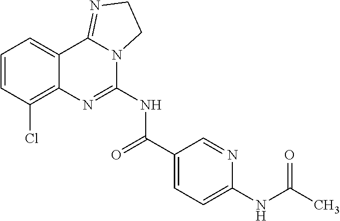 [SMILES: CC(=O)Nc1ccc(C(=O)NC2=Nc3c(Cl)cccc3C3=NCCN23)cn1] |
| 2-105 | 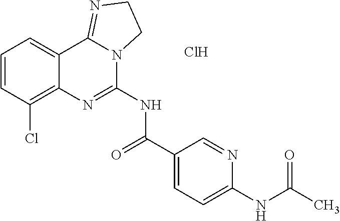 [SMILES: CC(=O)Nc1ccc(C(=O)NC2=Nc3c(Cl)cccc3C3=NCCN23)cn1 ; Cl] |
| 2-106 | 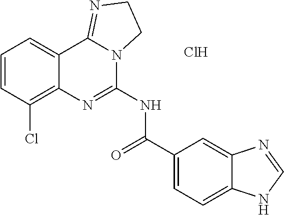 [SMILES: O=C(NC1=Nc2c(Cl)cccc2C2=NCCN12)c1ccc2[nH]cnc2c1 ; Cl] |
| 2-107 | 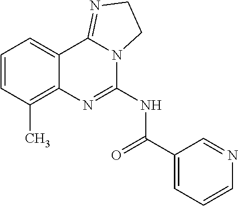 [SMILES: Cc1cccc2c1N=C(NC(=O)c1cccnc1)N1CCN=C21] |
| 2-108 | 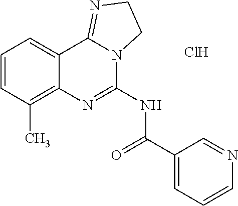 [SMILES: Cc1cccc2c1N=C(NC(=O)c1cccnc1)N1CCN=C21 ; Cl] |
| 2-109 | 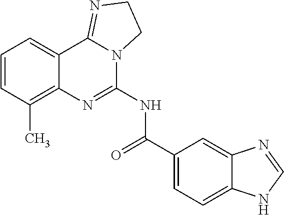 [SMILES: Cc1cccc2c1N=C(NC(=O)c1ccc3[nH]cnc3c1)N1CCN=C21] |
| 2-110 | 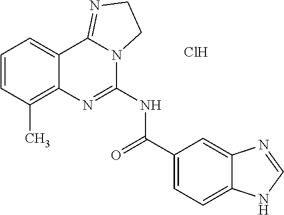 [SMILES: Cc1cccc2c1N=C(NC(=O)c1ccc3[nH]cnc3c1)N1CCN=C21 ; Cl] |
| 2-111 | 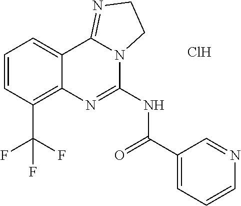 [SMILES: O=C(NC1=Nc2c(cccc2C(F)(F)F)C2=NCCN12)c1cccnc1 ; Cl] |
| 2-112 | 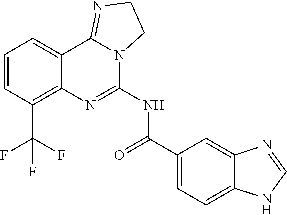 [SMILES: O=C(NC1=Nc2c(cccc2C(F)(F)F)C2=NCCN12)c1ccc2[nH]cnc2c1] |
| 2-113 | 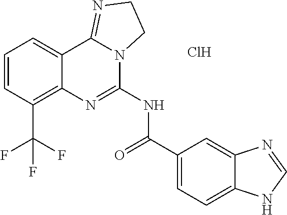 [SMILES: O=C(NC1=Nc2c(cccc2C(F)(F)F)C2=NCCN12)c1ccc2[nH]cnc2c1 ; Cl] |
| 2-114 | 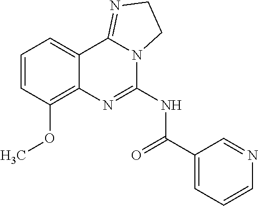 [SMILES: COc1cccc2c1N=C(NC(=O)c1cccnc1)N1CCN=C21] |
| 2-115 | 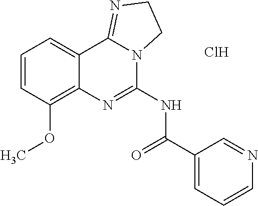 [SMILES: COc1cccc2c1N=C(NC(=O)c1cccnc1)N1CCN=C21 ; Cl] |
| 2-116 | 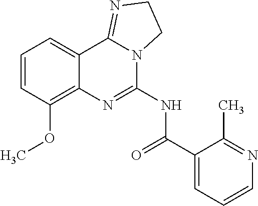 [SMILES: COc1cccc2c1N=C(NC(=O)c1cccnc1C)N1CCN=C21] |
| 2-117 | 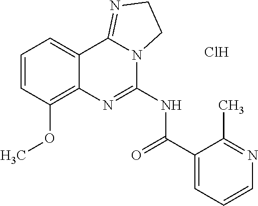 [SMILES: COc1cccc2c1N=C(NC(=O)c1cccnc1C)N1CCN=C21 ; Cl] |
| 2-118 | 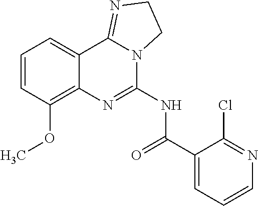 [SMILES: COc1cccc2c1N=C(NC(=O)c1cccnc1Cl)N1CCN=C21] |
| 2-119 | 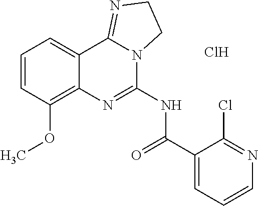 [SMILES: COc1cccc2c1N=C(NC(=O)c1cccnc1Cl)N1CCN=C21 ; Cl] |
| 2-120 | 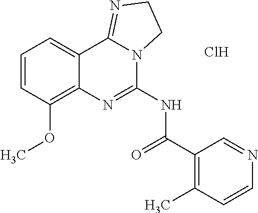 [SMILES: COc1cccc2c1N=C(NC(=O)c1cnccc1C)N1CCN=C21 ; Cl] |
| 2-121 | 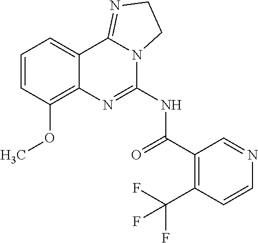 [SMILES: COc1cccc2c1N=C(NC(=O)c1cnccc1C(F)(F)F)N1CCN=C21] |
| 2-122 | 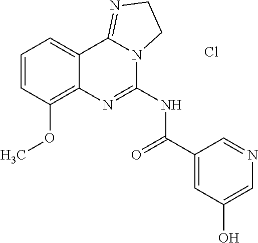 [SMILES: COc1cccc2c1N=C(NC(=O)c1cncc(O)c1)N1CCN=C21 ; Cl] |
| 2-123 | 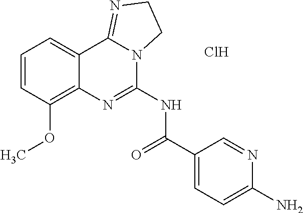 [SMILES: COc1cccc2c1N=C(NC(=O)c1ccc(N)nc1)N1CCN=C21 ; Cl] |
| 2-124 | 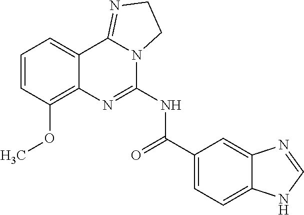 [SMILES: COc1cccc2c1N=C(NC(=O)c1ccc3[nH]cnc3c1)N1CCN=C21] |
| 2-125 | 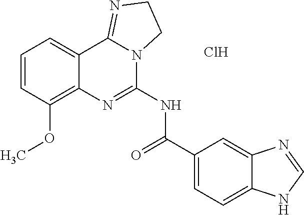 [SMILES: COc1cccc2c1N=C(NC(=O)c1ccc3[nH]cnc3c1)N1CCN=C21 ; Cl] |
| 2-126 | 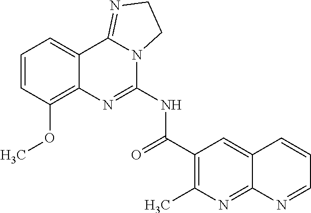 [SMILES: COc1cccc2c1N=C(NC(=O)c1cc3cccnc3nc1C)N1CCN=C21] |
| 2-127 | 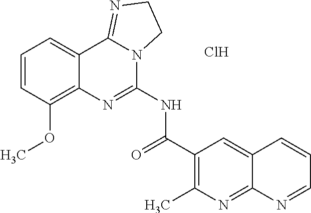 [SMILES: COc1cccc2c1N=C(NC(=O)c1cc3cccnc3nc1C)N1CCN=C21 ; Cl] |
| 2-128 | 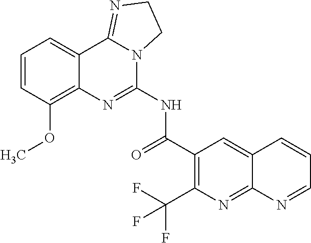 [SMILES: COc1cccc2c1N=C(NC(=O)c1cc3cccnc3nc1C(F)(F)F)N1CCN=C21] |
| 2-129 | 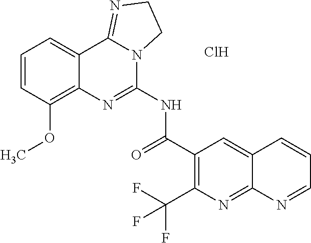 [SMILES: COc1cccc2c1N=C(NC(=O)c1cc3cccnc3nc1C(F)(F)F)N1CCN=C21 ; Cl] |
| 2-130 | 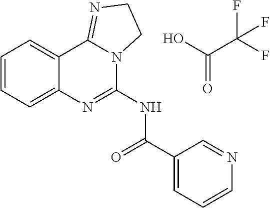 |
| 2-131 | 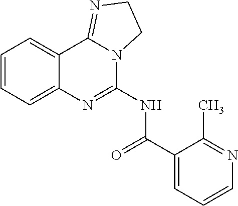 [SMILES: Cc1ncccc1C(=O)NC1=Nc2ccccc2C2=NCCN12] |
| 2-132 | 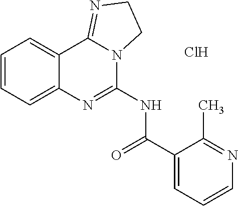 [SMILES: Cc1ncccc1C(=O)NC1=Nc2ccccc2C2=NCCN12 ; Cl] |
| 2-133 | 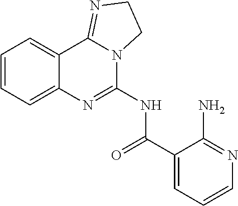 [SMILES: Nc1ncccc1C(=O)NC1=Nc2ccccc2C2=NCCN12] |
| 2-134 | 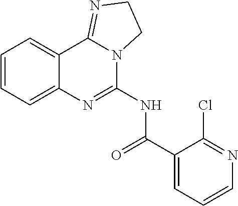 [SMILES: O=C(NC1=Nc2ccccc2C2=NCCN12)c1cccnc1Cl] |
| 2-135 | 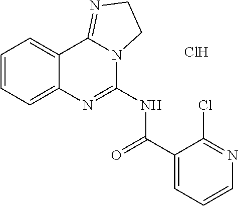 [SMILES: O=C(NC1=Nc2ccccc2C2=NCCN12)c1cccnc1Cl ; Cl] |
| 2-136 | 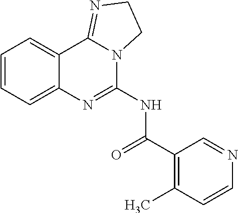 [SMILES: Cc1ccncc1C(=O)NC1=Nc2ccccc2C2=NCCN12] |
| 2-137 | 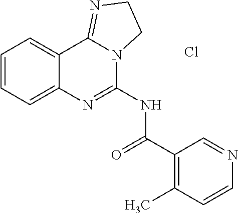 [SMILES: Cc1ccncc1C(=O)NC1=Nc2ccccc2C2=NCCN12 ; Cl] |
| 2-138 | 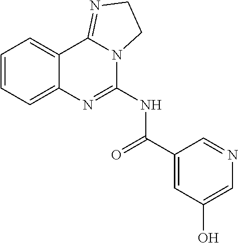 [SMILES: O=C(NC1=Nc2ccccc2C2=NCCN12)c1cncc(O)c1] |
| 2-139 | 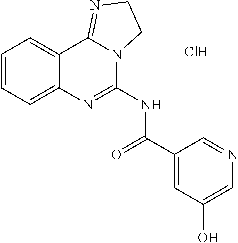 [SMILES: O=C(NC1=Nc2ccccc2C2=NCCN12)c1cncc(O)c1 ; Cl] |
| 2-140 | 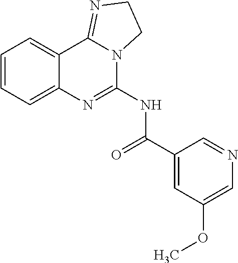 [SMILES: COc1cncc(C(=O)NC2=Nc3ccccc3C3=NCCN23)c1] |
| 2-141 | 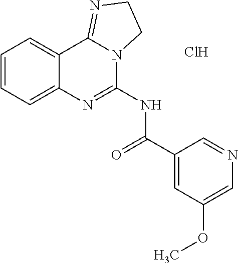 [SMILES: COc1cncc(C(=O)NC2=Nc3ccccc3C3=NCCN23)c1 ; Cl] |
| 2-142 | 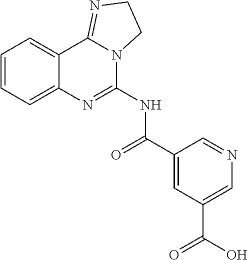 [SMILES: O=C(O)c1cncc(C(=O)NC2=Nc3ccccc3C3=NCCN23)c1] |
| 2-143 | 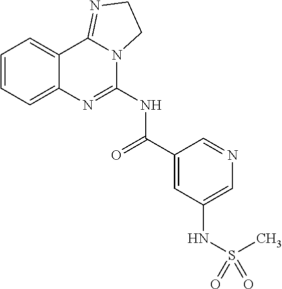 [SMILES: CS(=O)(=O)Nc1cncc(C(=O)NC2=Nc3ccccc3C3=NCCN23)c1] |
| 2-144 | 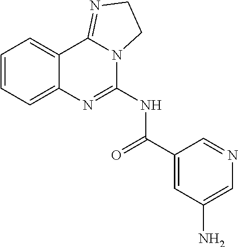 [SMILES: Nc1cncc(C(=O)NC2=Nc3ccccc3C3=NCCN23)c1] |
| 2-145 | 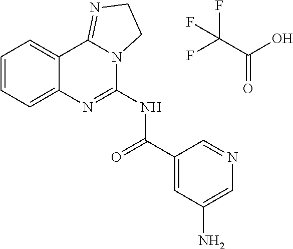 [SMILES: Nc1cncc(C(=O)NC2=Nc3ccccc3C3=NCCN23)c1 ; O=C(O)C(F)(F)F] |
| 2-146 | 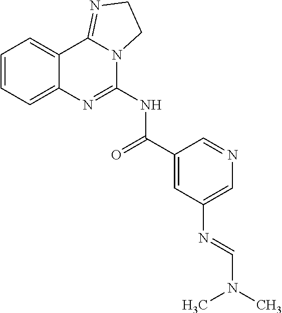 [SMILES: CN(C)/C=N/c1cncc(C(=O)NC2=Nc3ccccc3C3=NCCN23)c1] |
| 2-147 | 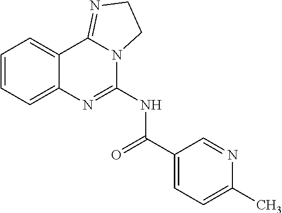 [SMILES: Cc1ccc(C(=O)NC2=Nc3ccccc3C3=NCCN23)cn1] |
| 2-148 | 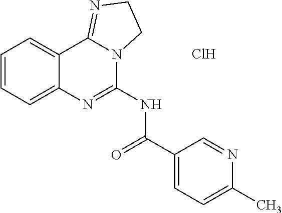 [SMILES: Cc1ccc(C(=O)NC2=Nc3ccccc3C3=NCCN23)cn1 ; Cl] |
| 2-149 | 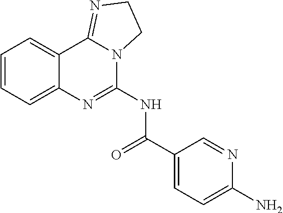 [SMILES: Nc1ccc(C(=O)NC2=Nc3ccccc3C3=NCCN23)cn1] |
| 2-150 | 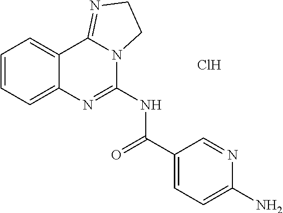 [SMILES: Nc1ccc(C(=O)NC2=Nc3ccccc3C3=NCCN23)cn1 ; Cl] |
| 2-151 | 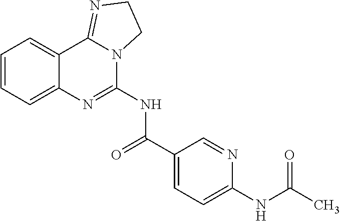 [SMILES: CC(=O)Nc1ccc(C(=O)NC2=Nc3ccccc3C3=NCCN23)cn1] |
| 2-152 | 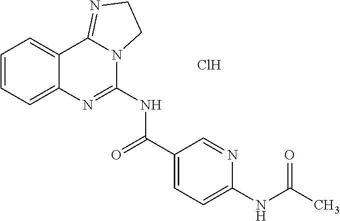 [SMILES: CC(=O)Nc1ccc(C(=O)NC2=Nc3ccccc3C3=NCCN23)cn1 ; Cl] |
| 2-153 | 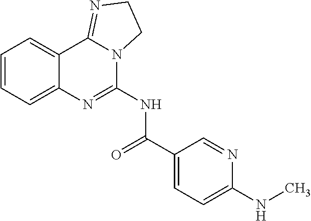 [SMILES: CNc1ccc(C(=O)NC2=Nc3ccccc3C3=NCCN23)cn1] |
| 2-154 | 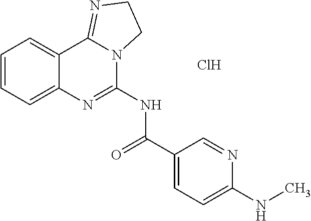 [SMILES: CNc1ccc(C(=O)NC2=Nc3ccccc3C3=NCCN23)cn1 ; Cl] |
| 2-155 | 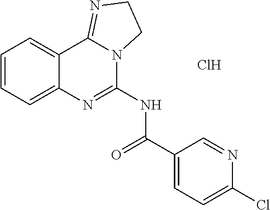 [SMILES: O=C(NC1=Nc2ccccc2C2=NCCN12)c1ccc(Cl)nc1 ; Cl] |
| 2-156 | 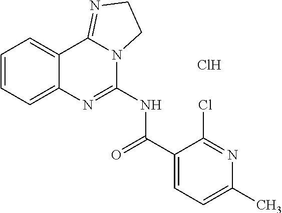 [SMILES: Cc1ccc(C(=O)NC2=Nc3ccccc3C3=NCCN23)c(Cl)n1 ; Cl] |
| 2-157 | 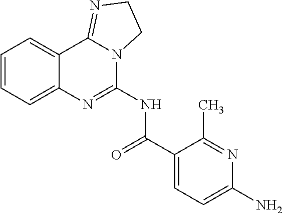 [SMILES: Cc1nc(N)ccc1C(=O)NC1=Nc2ccccc2C2=NCCN12] |
| 2-158 | 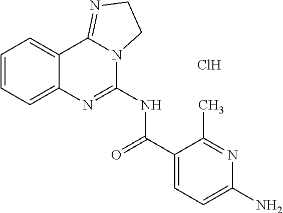 [SMILES: Cc1nc(N)ccc1C(=O)NC1=Nc2ccccc2C2=NCCN12 ; Cl] |
| 2-159 | 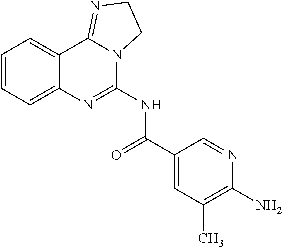 [SMILES: Cc1cc(C(=O)NC2=Nc3ccccc3C3=NCCN23)cnc1N] |
| 2-160 | 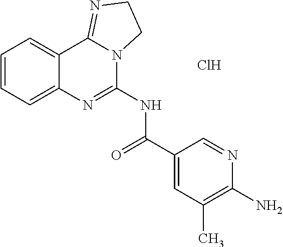 [SMILES: Cc1cc(C(=O)NC2=Nc3ccccc3C3=NCCN23)cnc1N ; Cl] |
| 2-161 | 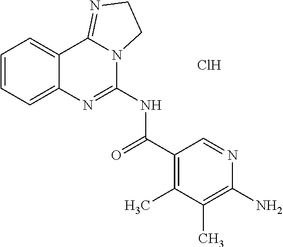 [SMILES: Cc1c(C(=O)NC2=Nc3ccccc3C3=NCCN23)cnc(N)c1C ; Cl] |
| 2-162 | 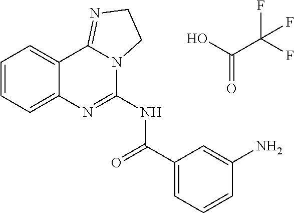 [SMILES: Nc1cccc(C(=O)NC2=Nc3ccccc3C3=NCCN23)c1 ; O=C(O)C(F)(F)F] |
| 2-163 | 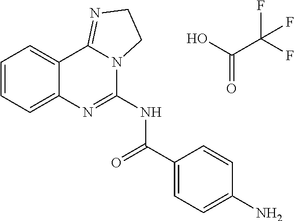 [SMILES: Nc1ccc(C(=O)NC2=Nc3ccccc3C3=NCCN23)cc1 ; O=C(O)C(F)(F)F] |
| 2-164 | 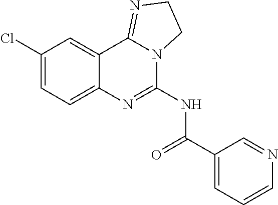 [SMILES: O=C(NC1=Nc2ccc(Cl)cc2C2=NCCN12)c1cccnc1] |
| 2-165 | 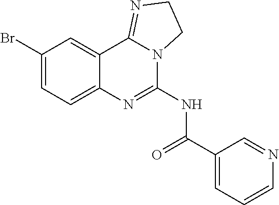 [SMILES: O=C(NC1=Nc2ccc(Br)cc2C2=NCCN12)c1cccnc1] |
| 2-166 | 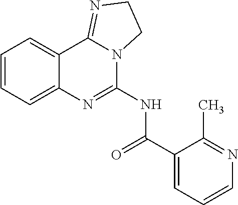 [SMILES: Cc1ncccc1C(=O)NC1=Nc2ccccc2C2=NCCN12] |
| 2-167 | 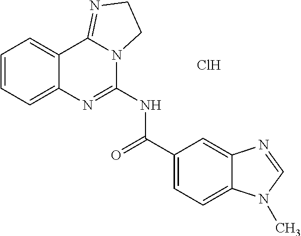 [SMILES: Cn1cnc2cc(C(=O)NC3=Nc4ccccc4C4=NCCN34)ccc21 ; Cl] |
| 2-168 | 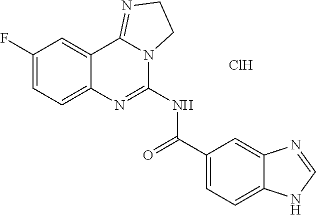 [SMILES: O=C(NC1=Nc2ccc(F)cc2C2=NCCN12)c1ccc2[nH]cnc2c1 ; Cl] |
| 2-169 | 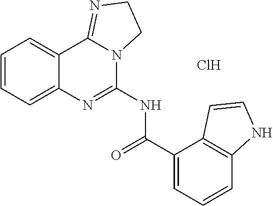 [SMILES: O=C(NC1=Nc2ccccc2C2=NCCN12)c1cccc2[nH]ccc12 ; Cl] |
| 2-170 | 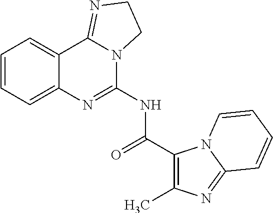 [SMILES: Cc1nc2ccccn2c1C(=O)NC1=Nc2ccccc2C2=NCCN12] |
| 2-171 | 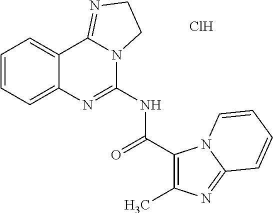 [SMILES: Cc1nc2ccccn2c1C(=O)NC1=Nc2ccccc2C2=NCCN12 ; Cl] |
| 2-172 | 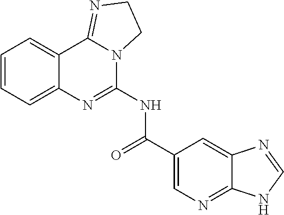 [SMILES: O=C(NC1=Nc2ccccc2C2=NCCN12)c1cnc2[nH]cnc2c1] |
| 2-173 | 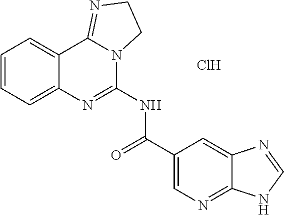 [SMILES: O=C(NC1=Nc2ccccc2C2=NCCN12)c1cnc2[nH]cnc2c1 ; Cl] |
| 2-174 | 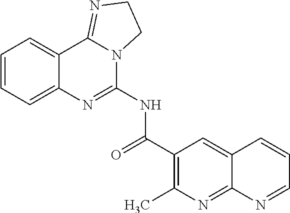 [SMILES: Cc1nc2ncccc2cc1C(=O)NC1=Nc2ccccc2C2=NCCN12] |
| 2-175 | 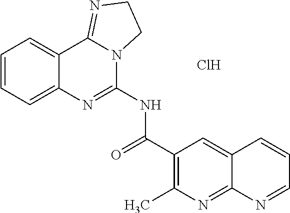 [SMILES: Cc1nc2ncccc2cc1C(=O)NC1=Nc2ccccc2C2=NCCN12 ; Cl] |
| 2-176 | 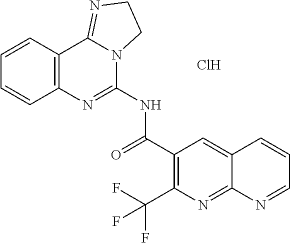 [SMILES: O=C(NC1=Nc2ccccc2C2=NCCN12)c1cc2cccnc2nc1C(F)(F)F ; Cl] |
| 2-177 | 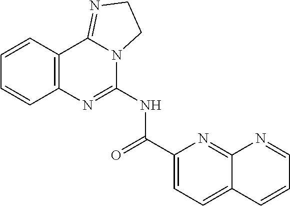 [SMILES: O=C(NC1=Nc2ccccc2C2=NCCN12)c1ccc2cccnc2n1] |
| 2-178 | 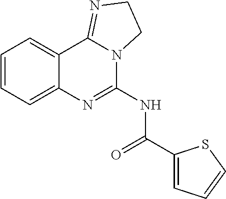 [SMILES: O=C(NC1=Nc2ccccc2C2=NCCN12)c1cccs1] |
| 2-179 | 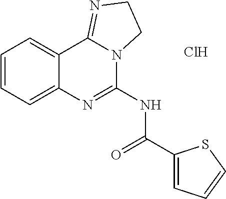 [SMILES: O=C(NC1=Nc2ccccc2C2=NCCN12)c1cccs1 ; Cl] |
| 2-180 | 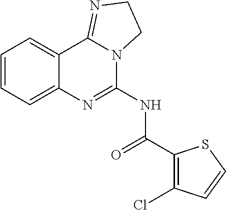 [SMILES: O=C(NC1=Nc2ccccc2C2=NCCN12)c1sccc1Cl] |
| 2-181 | 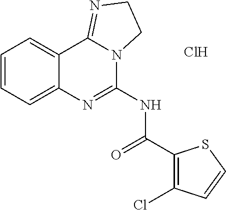 [SMILES: O=C(NC1=Nc2ccccc2C2=NCCN12)c1sccc1Cl ; Cl] |
| 2-182 | 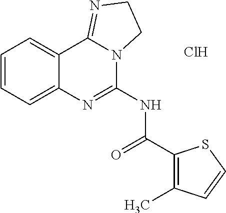 [SMILES: Cc1ccsc1C(=O)NC1=Nc2ccccc2C2=NCCN12 ; Cl] |
| 2-183 | 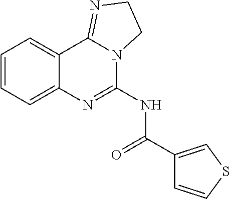 [SMILES: O=C(NC1=Nc2ccccc2C2=NCCN12)c1ccsc1] |
| 2-184 | 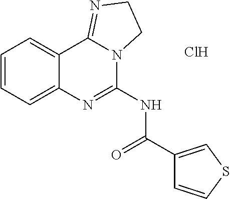 [SMILES: O=C(NC1=Nc2ccccc2C2=NCCN12)c1ccsc1 ; Cl] |
| 2-185 | 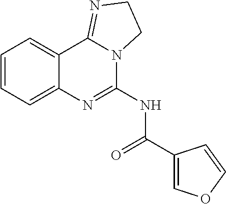 [SMILES: O=C(NC1=Nc2ccccc2C2=NCCN12)c1ccoc1] |
| 2-186 | 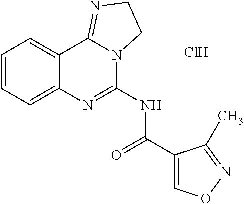 [SMILES: Cc1nocc1C(=O)NC1=Nc2ccccc2C2=NCCN12 ; Cl] |
| 2-187 | 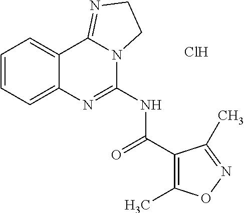 [SMILES: Cc1noc(C)c1C(=O)NC1=Nc2ccccc2C2=NCCN12 ; Cl] |
| 2-188 | 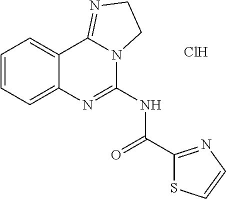 [SMILES: O=C(NC1=Nc2ccccc2C2=NCCN12)c1nccs1 ; Cl] |
| 2-189 | 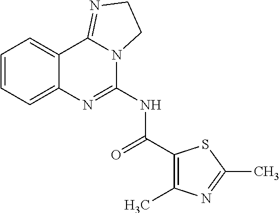 [SMILES: Cc1nc(C)c(C(=O)NC2=Nc3ccccc3C3=NCCN23)s1] |
| 2-190 | 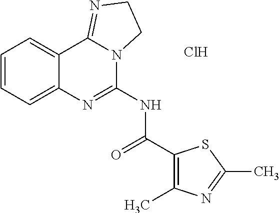 [SMILES: Cc1nc(C)c(C(=O)NC2=Nc3ccccc3C3=NCCN23)s1 ; Cl] |
| 2-191 | 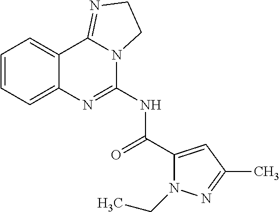 [SMILES: CCn1nc(C)cc1C(=O)NC1=Nc2ccccc2C2=NCCN12] |
| 2-192 | 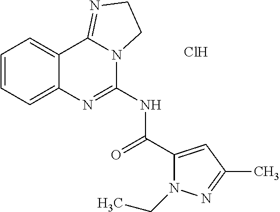 [SMILES: CCn1nc(C)cc1C(=O)NC1=Nc2ccccc2C2=NCCN12 ; Cl] |
| 2-193 | 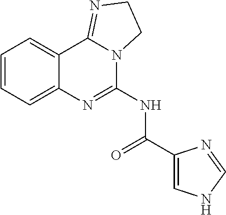 [SMILES: O=C(NC1=Nc2ccccc2C2=NCCN12)c1c[nH]cn1] |
| 2-194 | 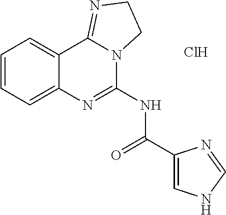 [SMILES: O=C(NC1=Nc2ccccc2C2=NCCN12)c1c[nH]cn1 ; Cl] |
| 2-195 | 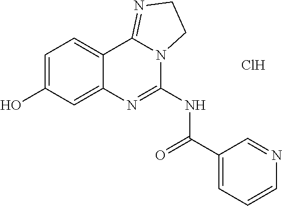 [SMILES: O=C(NC1=Nc2cc(O)ccc2C2=NCCN12)c1cccnc1 ; Cl] |
| 2-196 | 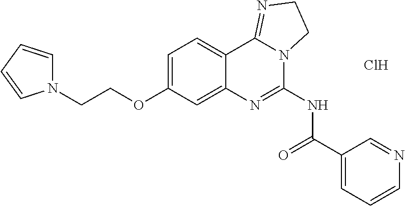 [SMILES: O=C(NC1=Nc2cc(OCCn3cccc3)ccc2C2=NCCN12)c1cccnc1 ; Cl] |
| 2-197 | 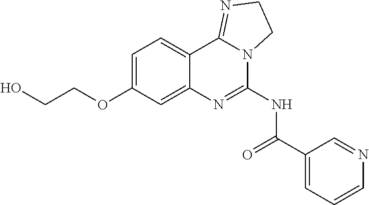 [SMILES: O=C(NC1=Nc2cc(OCCO)ccc2C2=NCCN12)c1cccnc1] |
| 2-198 | 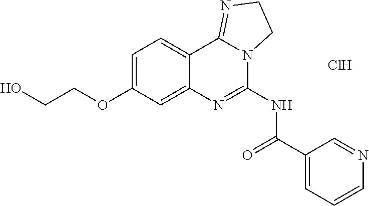 [SMILES: O=C(NC1=Nc2cc(OCCO)ccc2C2=NCCN12)c1cccnc1 ; Cl] |
| 2-199 | 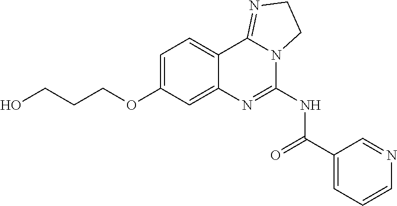 [SMILES: O=C(NC1=Nc2cc(OCCCO)ccc2C2=NCCN12)c1cccnc1] |
| 2-200 | 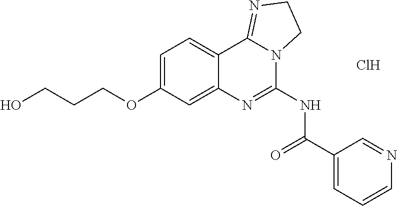 [SMILES: O=C(NC1=Nc2cc(OCCCO)ccc2C2=NCCN12)c1cccnc1 ; Cl] |
| 2-201 | 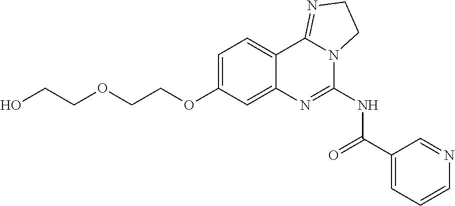 [SMILES: O=C(NC1=Nc2cc(OCCOCCO)ccc2C2=NCCN12)c1cccnc1] |
| 2-202 | 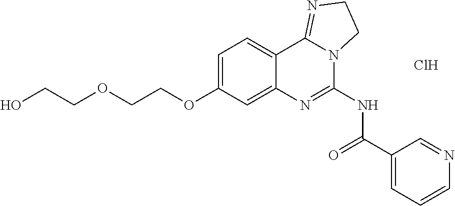 [SMILES: O=C(NC1=Nc2cc(OCCOCCO)ccc2C2=NCCN12)c1cccnc1 ; Cl] |
| 2-203 | 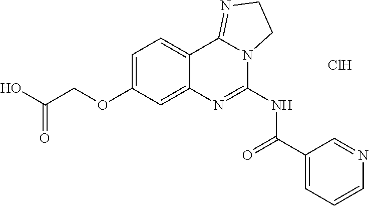 [SMILES: O=C(O)COc1ccc2c(c1)N=C(NC(=O)c1cccnc1)N1CCN=C21 ; Cl] |
| 2-204 | 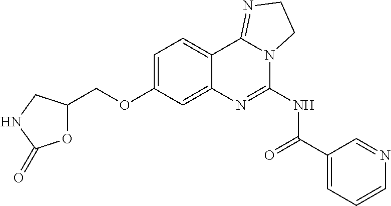 [SMILES: O=C1NCC(COc2ccc3c(c2)N=C(NC(=O)c2cccnc2)N2CCN=C32)O1] |
| 2-205 | 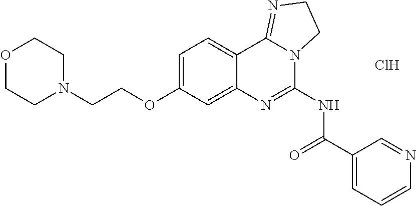 [SMILES: O=C(NC1=Nc2cc(OCCN3CCOCC3)ccc2C2=NCCN12)c1cccnc1 ; Cl] |
| 2-206 | 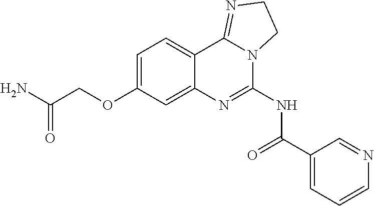 [SMILES: NC(=O)COc1ccc2c(c1)N=C(NC(=O)c1cccnc1)N1CCN=C21] |
| 2-207 | 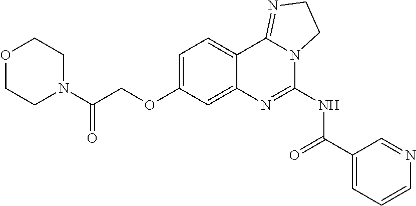 [SMILES: O=C(NC1=Nc2cc(OCC(=O)N3CCOCC3)ccc2C2=NCCN12)c1cccnc1] |
| 2-208 | 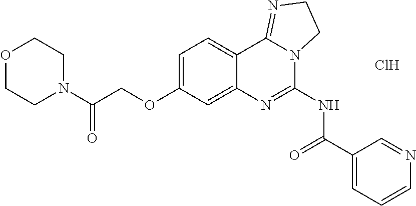 [SMILES: O=C(NC1=Nc2cc(OCC(=O)N3CCOCC3)ccc2C2=NCCN12)c1cccnc1 ; Cl] |
| 2-209 | 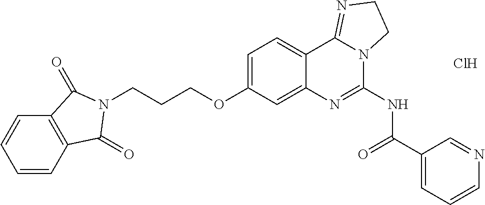 [SMILES: O=C(NC1=Nc2cc(OCCCN3C(=O)c4ccccc4C3=O)ccc2C2=NCCN12)c1cccnc1 ; Cl] |
| 2-210 | 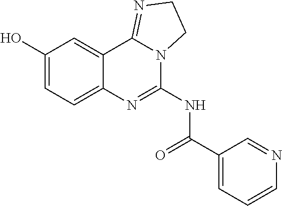 [SMILES: O=C(NC1=Nc2ccc(O)cc2C2=NCCN12)c1cccnc1] |
| 2-211 | 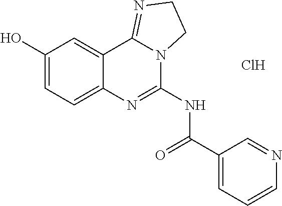 [SMILES: O=C(NC1=Nc2ccc(O)cc2C2=NCCN12)c1cccnc1 ; Cl] |
| 2-212 | 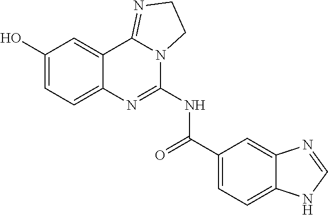 [SMILES: O=C(NC1=Nc2ccc(O)cc2C2=NCCN12)c1ccc2[nH]cnc2c1] |
| 2-213 | 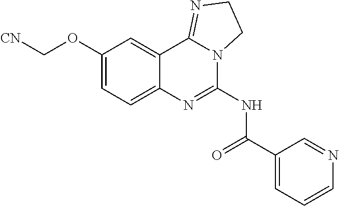 |
| 2-214 | 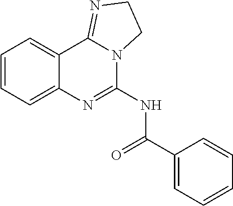 [SMILES: O=C(NC1=Nc2ccccc2C2=NCCN12)c1ccccc1] |
| 2-215 | 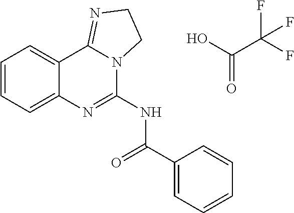 [SMILES: O=C(NC1=Nc2ccccc2C2=NCCN12)c1ccccc1 ; O=C(O)C(F)(F)F] |
| 2-216 | 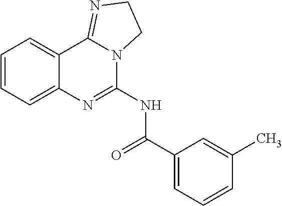 [SMILES: Cc1cccc(C(=O)NC2=Nc3ccccc3C3=NCCN23)c1] |
| 2-217 | 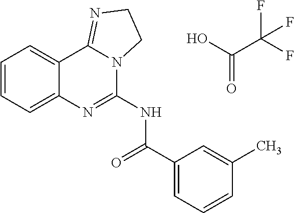 [SMILES: Cc1cccc(C(=O)NC2=Nc3ccccc3C3=NCCN23)c1 ; O=C(O)C(F)(F)F] |
| 2-218 | 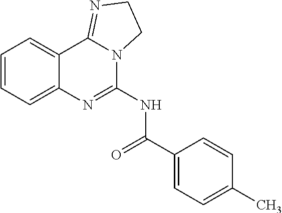 [SMILES: Cc1ccc(C(=O)NC2=Nc3ccccc3C3=NCCN23)cc1] |
| 2-219 | 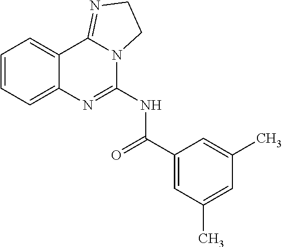 [SMILES: Cc1cc(C)cc(C(=O)NC2=Nc3ccccc3C3=NCCN23)c1] |
| 2-220 | 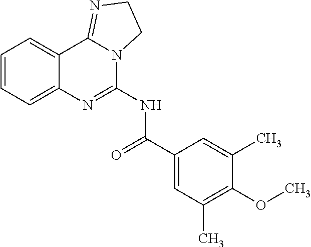 [SMILES: COc1c(C)cc(C(=O)NC2=Nc3ccccc3C3=NCCN23)cc1C] |
| 2-221 | 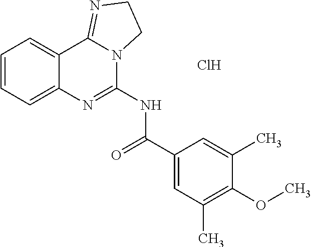 [SMILES: COc1c(C)cc(C(=O)NC2=Nc3ccccc3C3=NCCN23)cc1C ; Cl] |
| 2-222 | 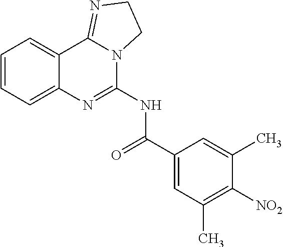 [SMILES: Cc1cc(C(=O)NC2=Nc3ccccc3C3=NCCN23)cc(C)c1[N+](=O)[O-]] |
| 2-223 | 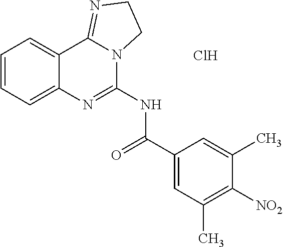 [SMILES: Cc1cc(C(=O)NC2=Nc3ccccc3C3=NCCN23)cc(C)c1[N+](=O)[O-] ; Cl] |
| 2-224 | 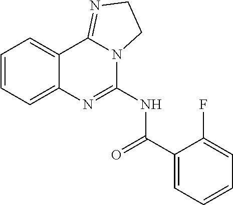 [SMILES: O=C(NC1=Nc2ccccc2C2=NCCN12)c1ccccc1F] |
| 2-225 | 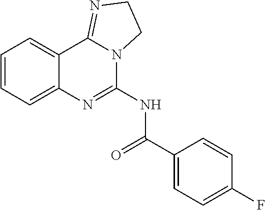 [SMILES: O=C(NC1=Nc2ccccc2C2=NCCN12)c1ccc(F)cc1] |
| 2-226 | 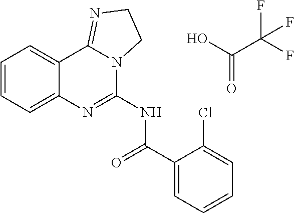 |
| 2-227 | 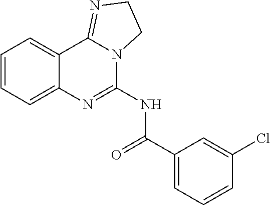 [SMILES: O=C(NC1=Nc2ccccc2C2=NCCN12)c1cccc(Cl)c1] |
| 2-228 | 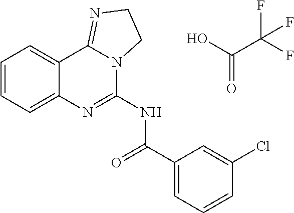 [SMILES: O=C(NC1=Nc2ccccc2C2=NCCN12)c1cccc(Cl)c1 ; O=C(O)C(F)(F)F] |
| 2-229 | 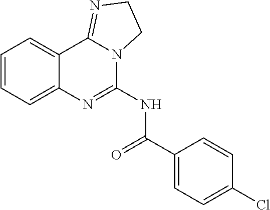 [SMILES: O=C(NC1=Nc2ccccc2C2=NCCN12)c1ccc(Cl)cc1] |
| 2-230 | 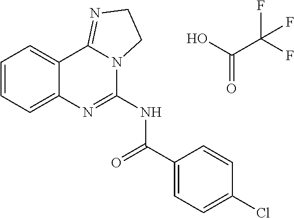 [SMILES: O=C(NC1=Nc2ccccc2C2=NCCN12)c1ccc(Cl)cc1 ; O=C(O)C(F)(F)F] |
| 2-231 | 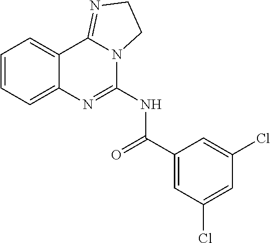 [SMILES: O=C(NC1=Nc2ccccc2C2=NCCN12)c1cc(Cl)cc(Cl)c1] |
| 2-232 | 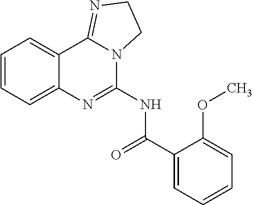 [SMILES: COc1ccccc1C(=O)NC1=Nc2ccccc2C2=NCCN12] |
| 2-233 | 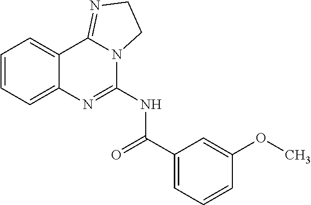 [SMILES: COc1cccc(C(=O)NC2=Nc3ccccc3C3=NCCN23)c1] |
| 2-234 | 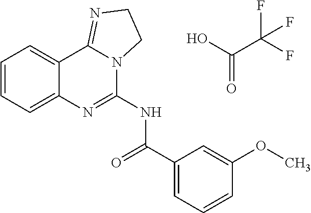 [SMILES: COc1cccc(C(=O)NC2=Nc3ccccc3C3=NCCN23)c1 ; O=C(O)C(F)(F)F] |
| 2-235 | 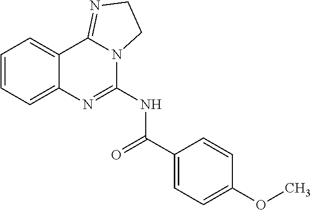 [SMILES: COc1ccc(C(=O)NC2=Nc3ccccc3C3=NCCN23)cc1] |
| 2-236 | 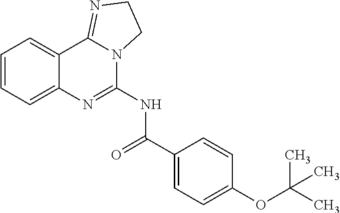 [SMILES: CC(C)(C)Oc1ccc(C(=O)NC2=Nc3ccccc3C3=NCCN23)cc1] |
| 2-237 | 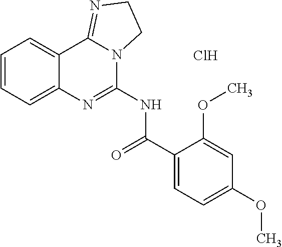 [SMILES: COc1ccc(C(=O)NC2=Nc3ccccc3C3=NCCN23)c(OC)c1 ; Cl] |
| 2-238 | 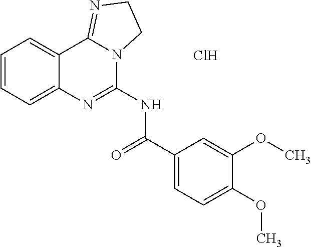 [SMILES: COc1ccc(C(=O)NC2=Nc3ccccc3C3=NCCN23)cc1OC ; Cl] |
| 2-239 | 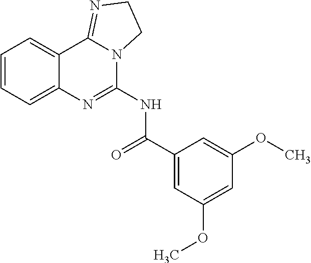 [SMILES: COc1cc(OC)cc(C(=O)NC2=Nc3ccccc3C3=NCCN23)c1] |
| 2-240 | 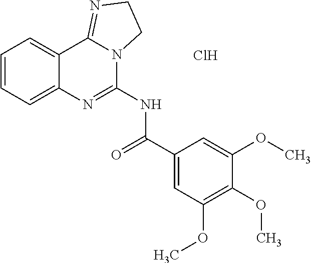 [SMILES: COc1cc(C(=O)NC2=Nc3ccccc3C3=NCCN23)cc(OC)c1OC ; Cl] |
| 2-241 | 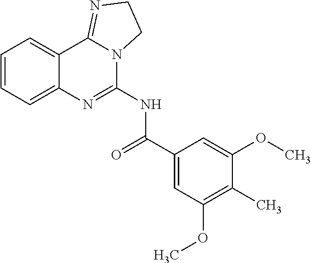 [SMILES: COc1cc(C(=O)NC2=Nc3ccccc3C3=NCCN23)cc(OC)c1C] |
| 2-242 | 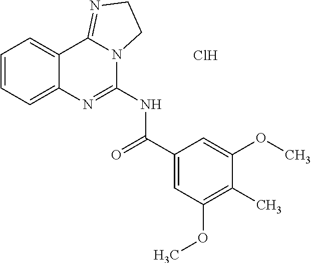 [SMILES: COc1cc(C(=O)NC2=Nc3ccccc3C3=NCCN23)cc(OC)c1C ; Cl] |
| 2-243 | 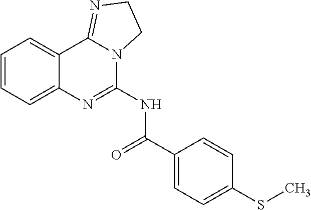 [SMILES: CSc1ccc(C(=O)NC2=Nc3ccccc3C3=NCCN23)cc1] |
| 2-244 | 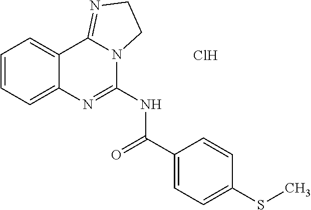 [SMILES: CSc1ccc(C(=O)NC2=Nc3ccccc3C3=NCCN23)cc1 ; Cl] |
| 2-245 | 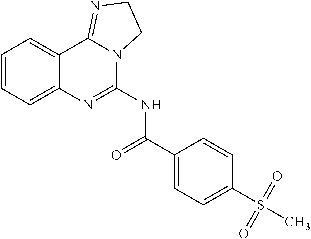 [SMILES: CS(=O)(=O)c1ccc(C(=O)NC2=Nc3ccccc3C3=NCCN23)cc1] |
| 2-246 | 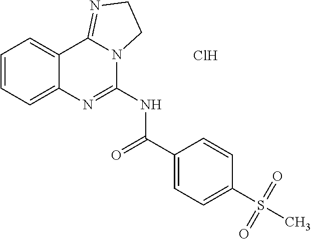 [SMILES: CS(=O)(=O)c1ccc(C(=O)NC2=Nc3ccccc3C3=NCCN23)cc1 ; Cl] |
| 2-247 | 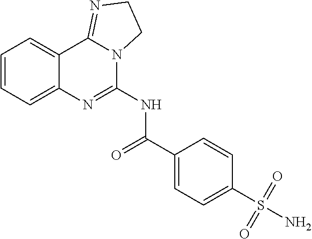 [SMILES: NS(=O)(=O)c1ccc(C(=O)NC2=Nc3ccccc3C3=NCCN23)cc1] |
| 2-248 | 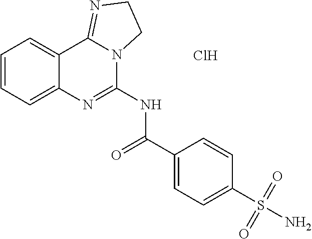 [SMILES: NS(=O)(=O)c1ccc(C(=O)NC2=Nc3ccccc3C3=NCCN23)cc1 ; Cl] |
| 2-249 | 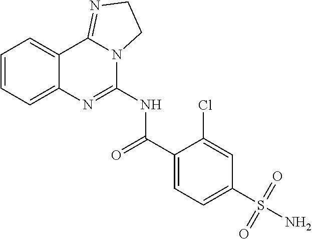 [SMILES: NS(=O)(=O)c1ccc(C(=O)NC2=Nc3ccccc3C3=NCCN23)c(Cl)c1] |
| 2-250 | 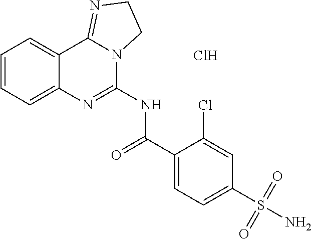 [SMILES: NS(=O)(=O)c1ccc(C(=O)NC2=Nc3ccccc3C3=NCCN23)c(Cl)c1 ; Cl] |
| 2-251 | 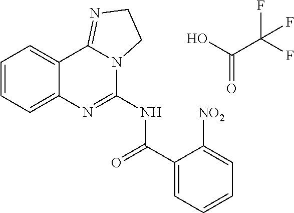 |
| 2-252 | 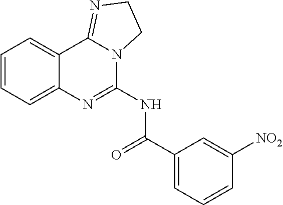 [SMILES: O=C(NC1=Nc2ccccc2C2=NCCN12)c1cccc([N+](=O)[O-])c1] |
| 2-253 | 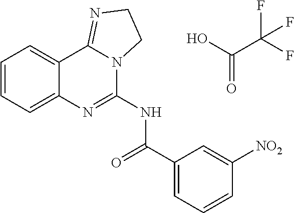 [SMILES: O=C(NC1=Nc2ccccc2C2=NCCN12)c1cccc([N+](=O)[O-])c1 ; O=C(O)C(F)(F)F] |
| 2-254 | 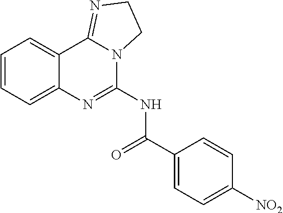 [SMILES: O=C(NC1=Nc2ccccc2C2=NCCN12)c1ccc([N+](=O)[O-])cc1] |
| 2-255 | 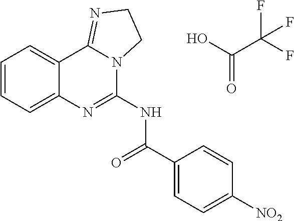 [SMILES: O=C(NC1=Nc2ccccc2C2=NCCN12)c1ccc([N+](=O)[O-])cc1 ; O=C(O)C(F)(F)F] |
| 2-256 | 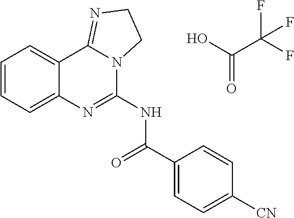 [SMILES: N#Cc1ccc(C(=O)NC2=Nc3ccccc3C3=NCCN23)cc1 ; O=C(O)C(F)(F)F] |
| 2-257 | 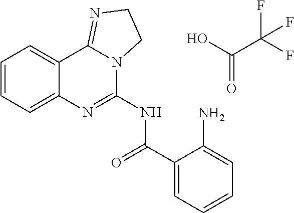 |
| 2-258 | 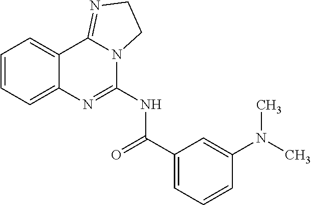 [SMILES: CN(C)c1cccc(C(=O)NC2=Nc3ccccc3C3=NCCN23)c1] |
| 2-259 | 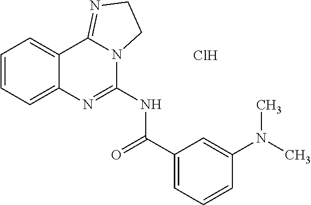 [SMILES: CN(C)c1cccc(C(=O)NC2=Nc3ccccc3C3=NCCN23)c1 ; Cl] |
| 2-260 | 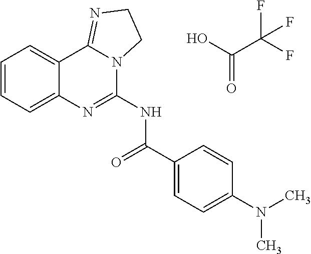 [SMILES: CN(C)c1ccc(C(=O)NC2=Nc3ccccc3C3=NCCN23)cc1 ; O=C(O)C(F)(F)F] |
| 2-261 | 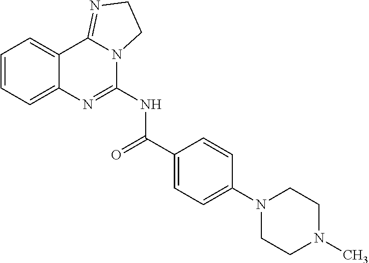 [SMILES: CN1CCN(c2ccc(C(=O)NC3=Nc4ccccc4C4=NCCN34)cc2)CC1] |
| 2-262 | 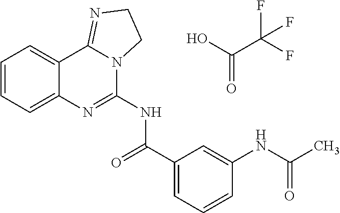 [SMILES: CC(=O)Nc1cccc(C(=O)NC2=Nc3ccccc3C3=NCCN23)c1 ; O=C(O)C(F)(F)F] |
| 2-263 | 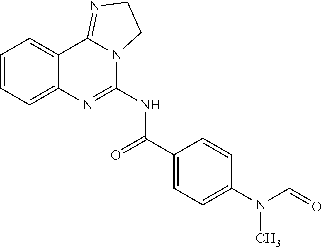 [SMILES: CN(C=O)c1ccc(C(=O)NC2=Nc3ccccc3C3=NCCN23)cc1] |
| 2-264 | 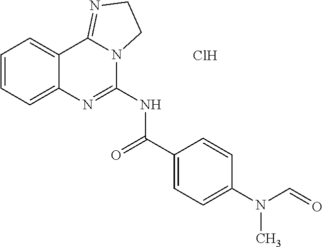 [SMILES: CN(C=O)c1ccc(C(=O)NC2=Nc3ccccc3C3=NCCN23)cc1 ; Cl] |
| 2-265 | 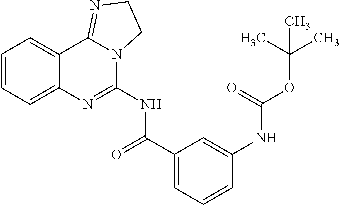 [SMILES: CC(C)(C)OC(=O)Nc1cccc(C(=O)NC2=Nc3ccccc3C3=NCCN23)c1] |
| 2-266 | 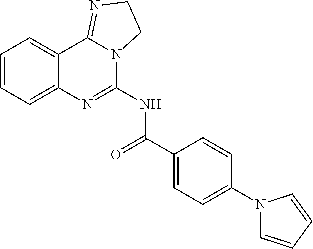 [SMILES: O=C(NC1=Nc2ccccc2C2=NCCN12)c1ccc(-n2cccc2)cc1] |
| 2-267 | 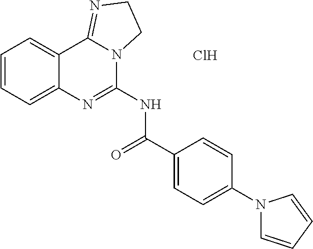 [SMILES: O=C(NC1=Nc2ccccc2C2=NCCN12)c1ccc(-n2cccc2)cc1 ; Cl] |
| 2-268 | 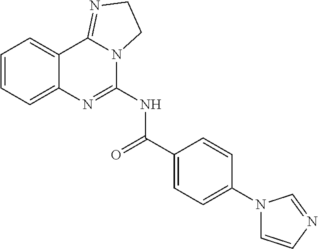 [SMILES: O=C(NC1=Nc2ccccc2C2=NCCN12)c1ccc(-n2ccnc2)cc1] |
| 2-269 | 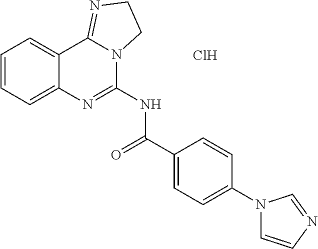 [SMILES: O=C(NC1=Nc2ccccc2C2=NCCN12)c1ccc(-n2ccnc2)cc1 ; Cl] |
| 2-270 | 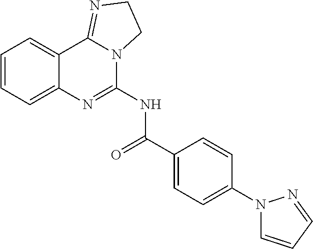 [SMILES: O=C(NC1=Nc2ccccc2C2=NCCN12)c1ccc(-n2cccn2)cc1] |
| 2-271 | 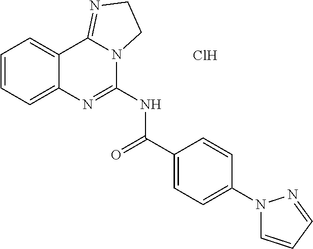 [SMILES: O=C(NC1=Nc2ccccc2C2=NCCN12)c1ccc(-n2cccn2)cc1 ; Cl] |
| 2-272 | 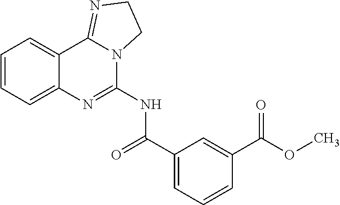 [SMILES: COC(=O)c1cccc(C(=O)NC2=Nc3ccccc3C3=NCCN23)c1] |
| 2-273 | 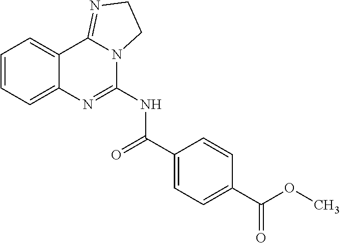 [SMILES: COC(=O)c1ccc(C(=O)NC2=Nc3ccccc3C3=NCCN23)cc1] |
| 2-274 | 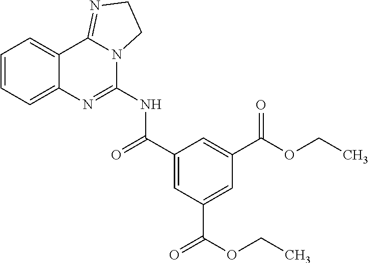 [SMILES: CCOC(=O)c1cc(C(=O)NC2=Nc3ccccc3C3=NCCN23)cc(C(=O)OCC)c1] |
| 2-275 | 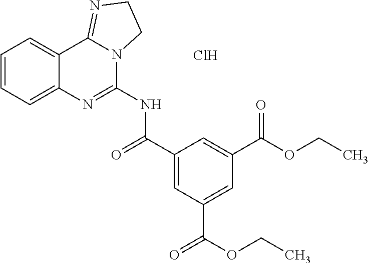 [SMILES: CCOC(=O)c1cc(C(=O)NC2=Nc3ccccc3C3=NCCN23)cc(C(=O)OCC)c1 ; Cl] |
| 2-276 | 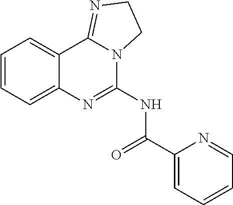 [SMILES: O=C(NC1=Nc2ccccc2C2=NCCN12)c1ccccn1] |
| 2-277 | 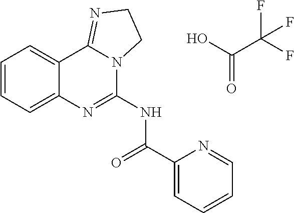 [SMILES: O=C(NC1=Nc2ccccc2C2=NCCN12)c1ccccn1 ; O=C(O)C(F)(F)F] |
| 2-278 | 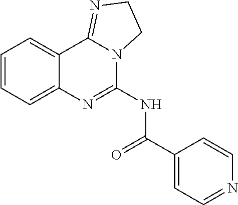 [SMILES: O=C(NC1=Nc2ccccc2C2=NCCN12)c1ccncc1] |
| 2-279 | 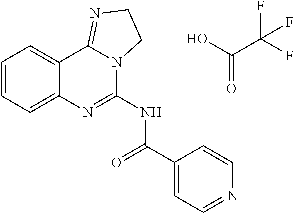 [SMILES: O=C(NC1=Nc2ccccc2C2=NCCN12)c1ccncc1 ; O=C(O)C(F)(F)F] |
| 2-280 | 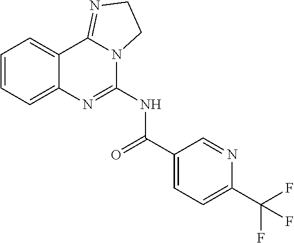 [SMILES: O=C(NC1=Nc2ccccc2C2=NCCN12)c1ccc(C(F)(F)F)nc1] |
| 2-281 | 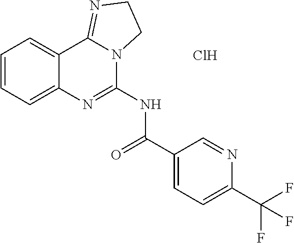 [SMILES: O=C(NC1=Nc2ccccc2C2=NCCN12)c1ccc(C(F)(F)F)nc1 ; Cl] |
| 2-282 | 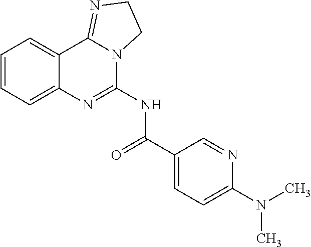 [SMILES: CN(C)c1ccc(C(=O)NC2=Nc3ccccc3C3=NCCN23)cn1] |
| 2-283 | 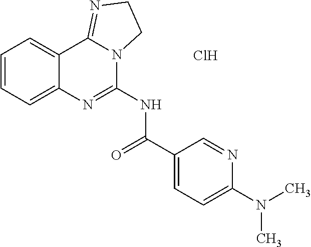 [SMILES: CN(C)c1ccc(C(=O)NC2=Nc3ccccc3C3=NCCN23)cn1 ; Cl] |
| 2-284 | 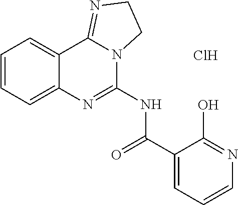 [SMILES: O=C(NC1=Nc2ccccc2C2=NCCN12)c1cccnc1O ; Cl] |
| 2-285 | 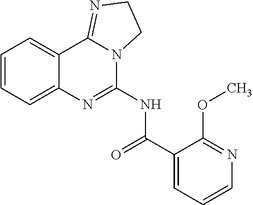 [SMILES: COc1ncccc1C(=O)NC1=Nc2ccccc2C2=NCCN12] |
| 2-286 | 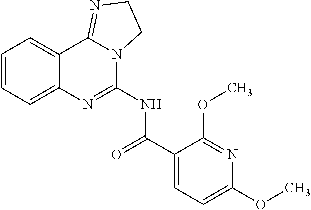 [SMILES: COc1ccc(C(=O)NC2=Nc3ccccc3C3=NCCN23)c(OC)n1] |
| 2-287 | 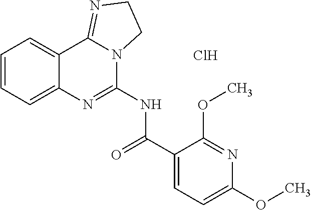 [SMILES: COc1ccc(C(=O)NC2=Nc3ccccc3C3=NCCN23)c(OC)n1 ; Cl] |
| 2-288 | 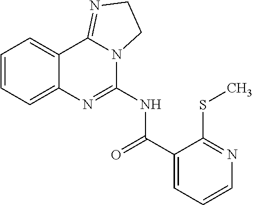 [SMILES: CSc1ncccc1C(=O)NC1=Nc2ccccc2C2=NCCN12] |
| 2-289 | 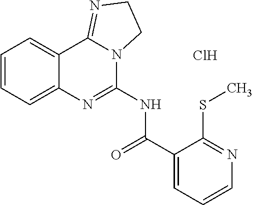 [SMILES: CSc1ncccc1C(=O)NC1=Nc2ccccc2C2=NCCN12 ; Cl] |
| 2-290 | 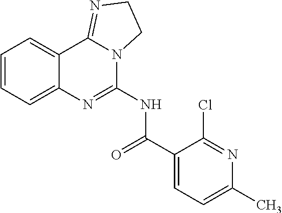 [SMILES: Cc1ccc(C(=O)NC2=Nc3ccccc3C3=NCCN23)c(Cl)n1] |
| 2-291 | 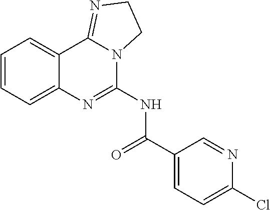 [SMILES: O=C(NC1=Nc2ccccc2C2=NCCN12)c1ccc(Cl)nc1] |
| 2-292 | 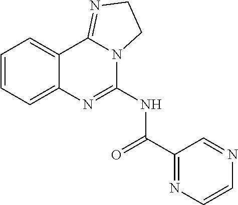 [SMILES: O=C(NC1=Nc2ccccc2C2=NCCN12)c1cnccn1] |
| 2-293 | 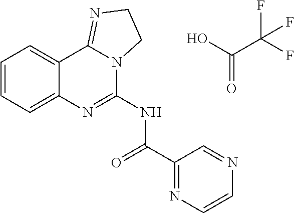 [SMILES: O=C(NC1=Nc2ccccc2C2=NCCN12)c1cnccn1 ; O=C(O)C(F)(F)F] |
| 2-294 | 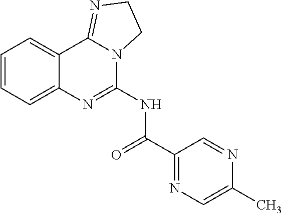 [SMILES: Cc1cnc(C(=O)NC2=Nc3ccccc3C3=NCCN23)cn1] |
| 2-295 | 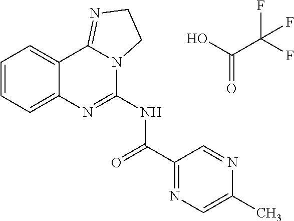 [SMILES: Cc1cnc(C(=O)NC2=Nc3ccccc3C3=NCCN23)cn1 ; O=C(O)C(F)(F)F] |
| 2-296 | 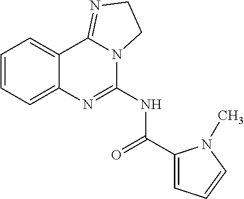 [SMILES: Cn1cccc1C(=O)NC1=Nc2ccccc2C2=NCCN12] |
| 2-297 | 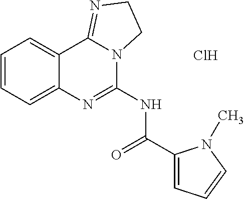 [SMILES: Cn1cccc1C(=O)NC1=Nc2ccccc2C2=NCCN12 ; Cl] |
| 2-298 | 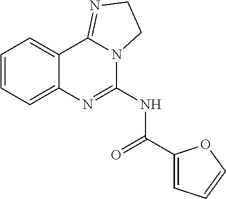 [SMILES: O=C(NC1=Nc2ccccc2C2=NCCN12)c1ccco1] |
| 2-299 | 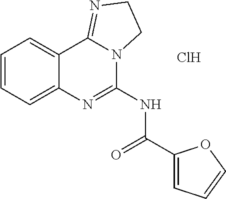 [SMILES: O=C(NC1=Nc2ccccc2C2=NCCN12)c1ccco1 ; Cl] |
| 2-300 | 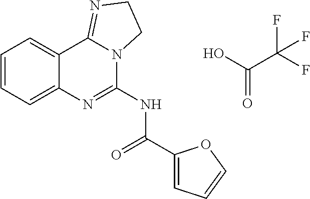 [SMILES: O=C(NC1=Nc2ccccc2C2=NCCN12)c1ccco1 ; O=C(O)C(F)(F)F] |
| 2-301 | 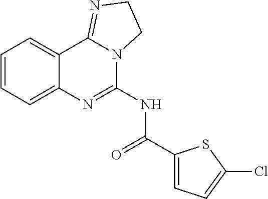 [SMILES: O=C(NC1=Nc2ccccc2C2=NCCN12)c1ccc(Cl)s1] |
| 2-302 | 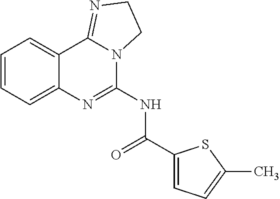 [SMILES: Cc1ccc(C(=O)NC2=Nc3ccccc3C3=NCCN23)s1] |
| 2-303 | 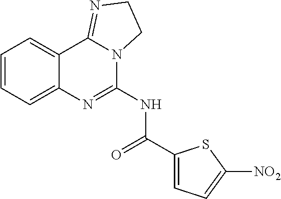 [SMILES: O=C(NC1=Nc2ccccc2C2=NCCN12)c1ccc([N+](=O)[O-])s1] |
| 2-304 | 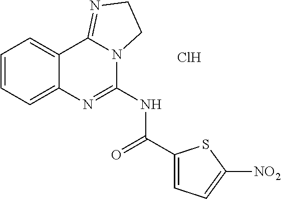 [SMILES: O=C(NC1=Nc2ccccc2C2=NCCN12)c1ccc([N+](=O)[O-])s1 ; Cl] |
| 2-305 | 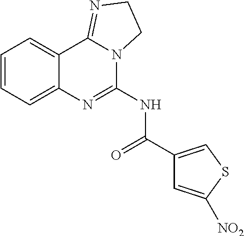 [SMILES: O=C(NC1=Nc2ccccc2C2=NCCN12)c1csc([N+](=O)[O-])c1] |
| 2-306 | 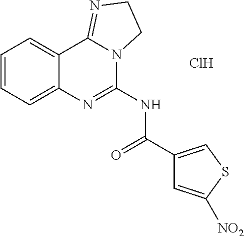 [SMILES: O=C(NC1=Nc2ccccc2C2=NCCN12)c1csc([N+](=O)[O-])c1 ; Cl] |
| 2-307 | 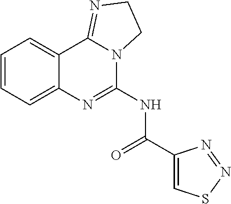 [SMILES: O=C(NC1=Nc2ccccc2C2=NCCN12)c1csnn1] |
| 2-308 | 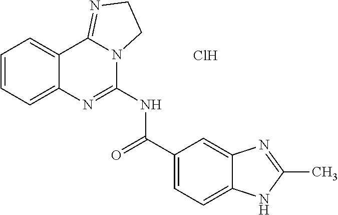 [SMILES: Cc1nc2cc(C(=O)NC3=Nc4ccccc4C4=NCCN34)ccc2[nH]1 ; Cl] |
| 2-309 | 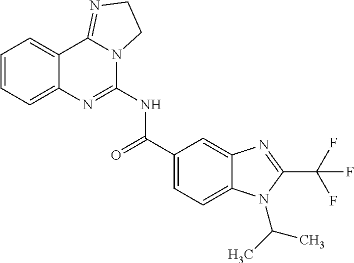 [SMILES: CC(C)n1c(C(F)(F)F)nc2cc(C(=O)NC3=Nc4ccccc4C4=NCCN34)ccc21] |
| 2-310 | 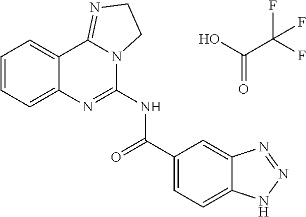 [SMILES: O=C(NC1=Nc2ccccc2C2=NCCN12)c1ccc2[nH]nnc2c1 ; O=C(O)C(F)(F)F] |
| 2-311 | 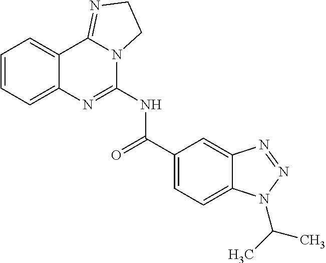 [SMILES: CC(C)n1nnc2cc(C(=O)NC3=Nc4ccccc4C4=NCCN34)ccc21] |
| 2-312 | 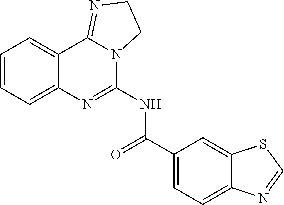 [SMILES: O=C(NC1=Nc2ccccc2C2=NCCN12)c1ccc2ncsc2c1] |
| 2-313 | 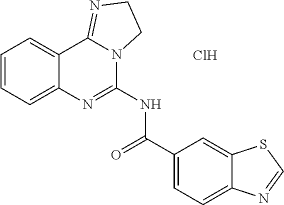 [SMILES: O=C(NC1=Nc2ccccc2C2=NCCN12)c1ccc2ncsc2c1 ; Cl] |
| 2-314 | 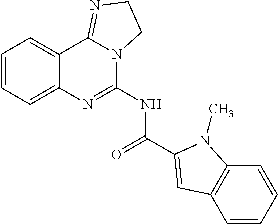 [SMILES: Cn1c(C(=O)NC2=Nc3ccccc3C3=NCCN23)cc2ccccc21] |
| 2-315 | 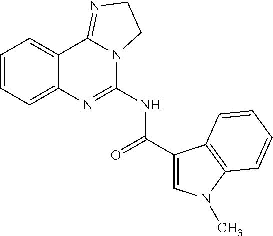 [SMILES: Cn1cc(C(=O)NC2=Nc3ccccc3C3=NCCN23)c2ccccc21] |
| 2-316 | 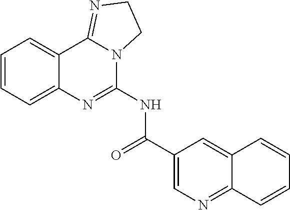 [SMILES: O=C(NC1=Nc2ccccc2C2=NCCN12)c1cnc2ccccc2c1] |
| 2-317 | 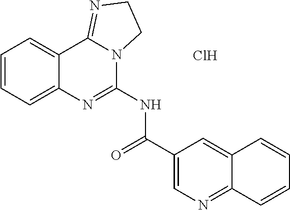 [SMILES: O=C(NC1=Nc2ccccc2C2=NCCN12)c1cnc2ccccc2c1 ; Cl] |
| 2-318 | 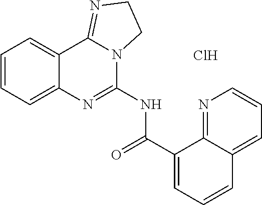 [SMILES: O=C(NC1=Nc2ccccc2C2=NCCN12)c1cccc2cccnc12 ; Cl] |
| 2-319 | 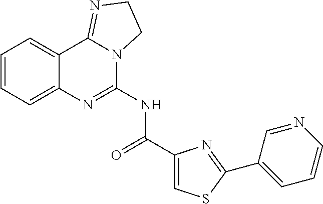 [SMILES: O=C(NC1=Nc2ccccc2C2=NCCN12)c1csc(-c2cccnc2)n1] |
| 2-320 | 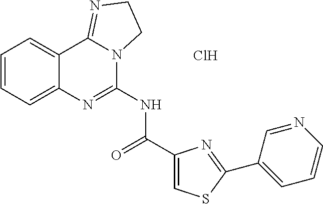 [SMILES: O=C(NC1=Nc2ccccc2C2=NCCN12)c1csc(-c2cccnc2)n1 ; Cl] |
| 2-321 | 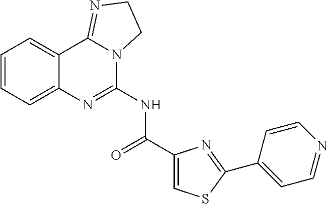 [SMILES: O=C(NC1=Nc2ccccc2C2=NCCN12)c1csc(-c2ccncc2)n1] |
| 2-322 | 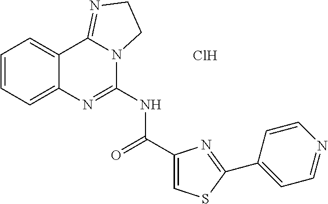 [SMILES: O=C(NC1=Nc2ccccc2C2=NCCN12)c1csc(-c2ccncc2)n1 ; Cl] |
| 2-323 | 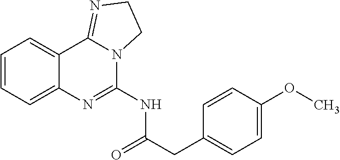 [SMILES: COc1ccc(CC(=O)NC2=Nc3ccccc3C3=NCCN23)cc1] |
| 2-324 | 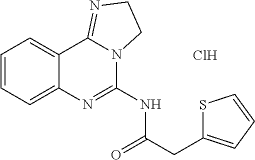 [SMILES: O=C(Cc1cccs1)NC1=Nc2ccccc2C2=NCCN12 ; Cl] |
| 2-325 | 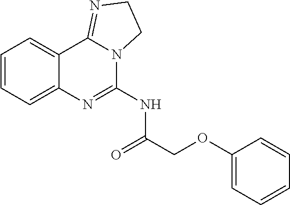 [SMILES: O=C(COc1ccccc1)NC1=Nc2ccccc2C2=NCCN12] |
| 2-326 | 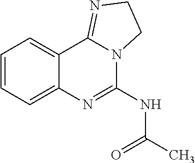 [SMILES: CC(=O)NC1=Nc2ccccc2C2=NCCN12] |
| 2-327 | 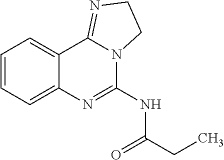 [SMILES: CCC(=O)NC1=Nc2ccccc2C2=NCCN12] |
| 2-328 | 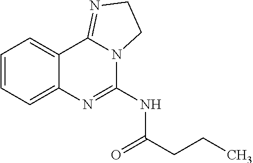 [SMILES: CCCC(=O)NC1=Nc2ccccc2C2=NCCN12] |
| 2-329 | 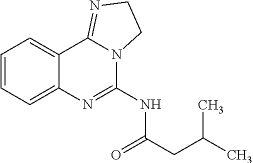 [SMILES: CC(C)CC(=O)NC1=Nc2ccccc2C2=NCCN12] |
| 2-330 | 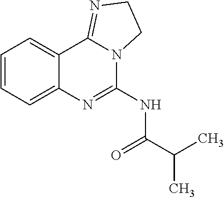 [SMILES: CC(C)C(=O)NC1=Nc2ccccc2C2=NCCN12] |
| 2-331 | 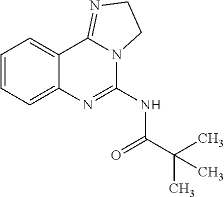 [SMILES: CC(C)(C)C(=O)NC1=Nc2ccccc2C2=NCCN12] |
| 2-332 | 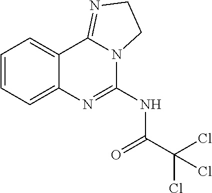 [SMILES: O=C(NC1=Nc2ccccc2C2=NCCN12)C(Cl)(Cl)Cl] |
| 2-333 | 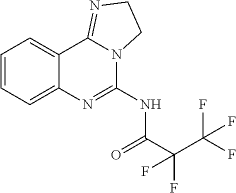 [SMILES: O=C(NC1=Nc2ccccc2C2=NCCN12)C(F)(F)C(F)(F)F] |
| 2-334 | 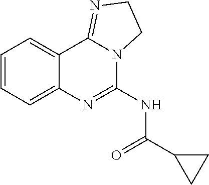 [SMILES: O=C(NC1=Nc2ccccc2C2=NCCN12)C1CC1] |
| 2-335 | 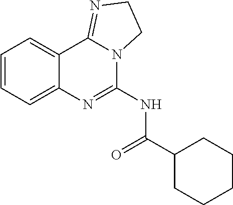 [SMILES: O=C(NC1=Nc2ccccc2C2=NCCN12)C1CCCCC1] |
| 2-336 | 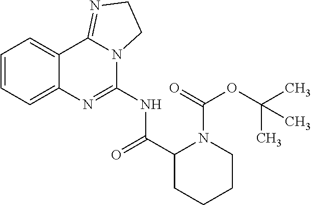 [SMILES: CC(C)(C)OC(=O)N1CCCCC1C(=O)NC1=Nc2ccccc2C2=NCCN12] |
| 2-337 | 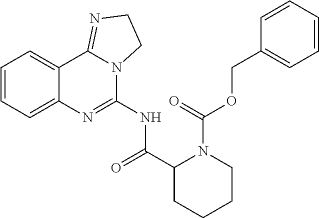 [SMILES: O=C(NC1=Nc2ccccc2C2=NCCN12)C1CCCCN1C(=O)OCc1ccccc1] |
| 2-338 | 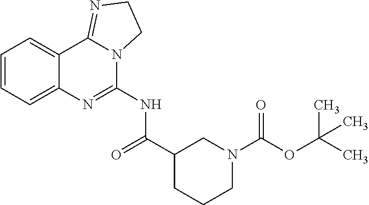 [SMILES: CC(C)(C)OC(=O)N1CCCC(C(=O)NC2=Nc3ccccc3C3=NCCN23)C1] |
| 2-339 | 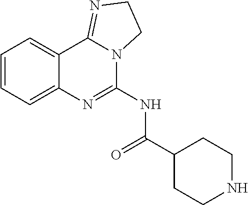 [SMILES: O=C(NC1=Nc2ccccc2C2=NCCN12)C1CCNCC1] |
| 2-340 | 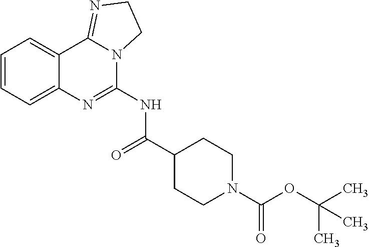 [SMILES: CC(C)(C)OC(=O)N1CCC(C(=O)NC2=Nc3ccccc3C3=NCCN23)CC1] |
| 2-341 | 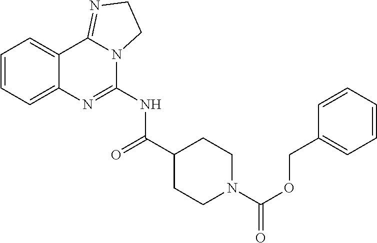 [SMILES: O=C(NC1=Nc2ccccc2C2=NCCN12)C1CCN(C(=O)OCc2ccccc2)CC1] |
| 2-342 | 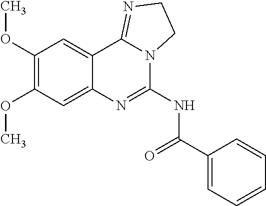 [SMILES: COc1cc2c(cc1OC)C1=NCCN1C(NC(=O)c1ccccc1)=N2] |
| 2-343 | 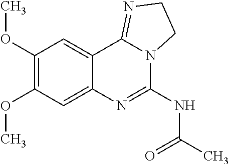 [SMILES: COc1cc2c(cc1OC)C1=NCCN1C(NC(C)=O)=N2] |
| 2-344 | 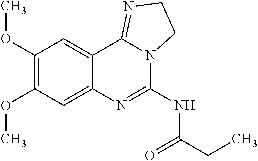 [SMILES: CCC(=O)NC1=Nc2cc(OC)c(OC)cc2C2=NCCN12] |
| 2-345 | 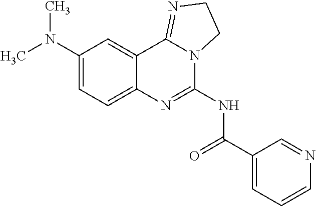 [SMILES: CN(C)c1ccc2c(c1)C1=NCCN1C(NC(=O)c1cccnc1)=N2] |
| 2-346 | 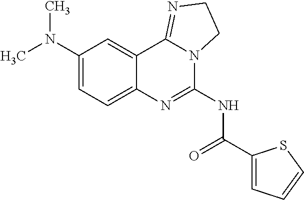 [SMILES: CN(C)c1ccc2c(c1)C1=NCCN1C(NC(=O)c1cccs1)=N2] |
| 2-347 | 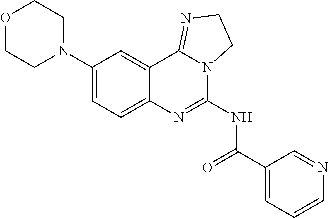 [SMILES: O=C(NC1=Nc2ccc(N3CCOCC3)cc2C2=NCCN12)c1cccnc1] |
| 2-348 | 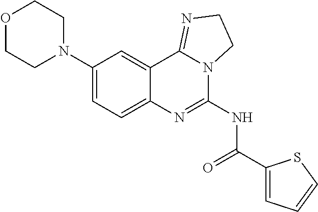 [SMILES: O=C(NC1=Nc2ccc(N3CCOCC3)cc2C2=NCCN12)c1cccs1] |
| 2-349 | 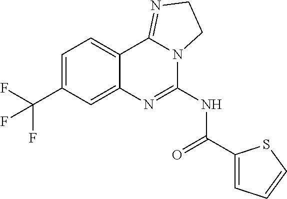 [SMILES: O=C(NC1=Nc2cc(C(F)(F)F)ccc2C2=NCCN12)c1cccs1] |
| 2-350 | 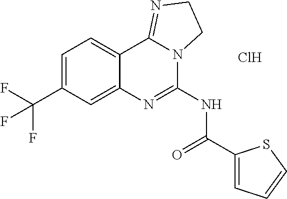 [SMILES: O=C(NC1=Nc2cc(C(F)(F)F)ccc2C2=NCCN12)c1cccs1 ; Cl] |
| 2-351 | 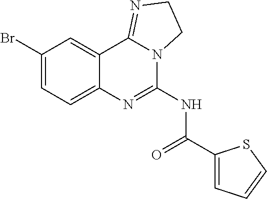 [SMILES: O=C(NC1=Nc2ccc(Br)cc2C2=NCCN12)c1cccs1] |
| 2-352 | 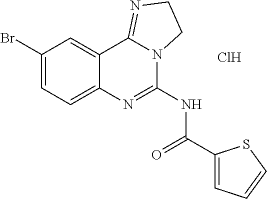 [SMILES: O=C(NC1=Nc2ccc(Br)cc2C2=NCCN12)c1cccs1 ; Cl] |
| 2-353 | 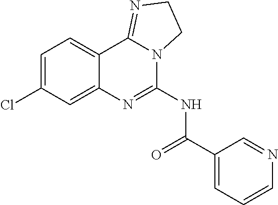 [SMILES: O=C(NC1=Nc2cc(Cl)ccc2C2=NCCN12)c1cccnc1] |
| 2-354 | 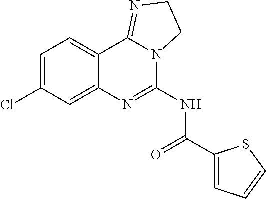 [SMILES: O=C(NC1=Nc2cc(Cl)ccc2C2=NCCN12)c1cccs1] |
| 2-355 | 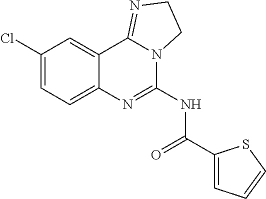 [SMILES: O=C(NC1=Nc2ccc(Cl)cc2C2=NCCN12)c1cccs1] |
| 2-356 | 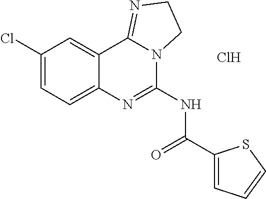 [SMILES: O=C(NC1=Nc2ccc(Cl)cc2C2=NCCN12)c1cccs1 ; Cl] |
| 2-357 | 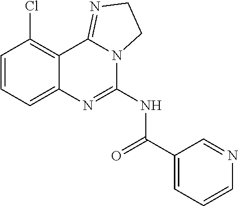 [SMILES: O=C(NC1=Nc2cccc(Cl)c2C2=NCCN12)c1cccnc1] |
| 2-358 | 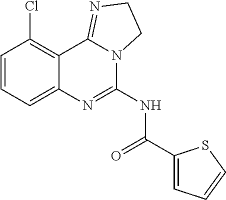 [SMILES: O=C(NC1=Nc2cccc(Cl)c2C2=NCCN12)c1cccs1] |
| 2-359 | 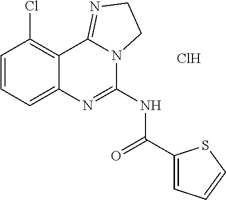 [SMILES: O=C(NC1=Nc2cccc(Cl)c2C2=NCCN12)c1cccs1 ; Cl] |
| 2-360 | 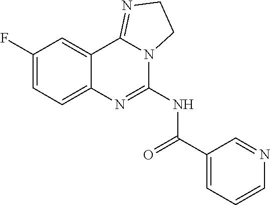 [SMILES: O=C(NC1=Nc2ccc(F)cc2C2=NCCN12)c1cccnc1] |
| 2-361 | 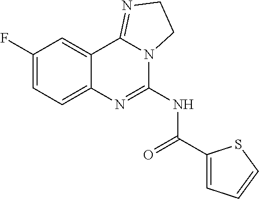 [SMILES: O=C(NC1=Nc2ccc(F)cc2C2=NCCN12)c1cccs1] |
| 2-362 | 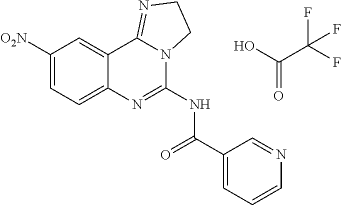 [SMILES: O=C(NC1=Nc2ccc([N+](=O)[O-])cc2C2=NCCN12)c1cccnc1 ; O=C(O)C(F)(F)F] |
| 2-363 | 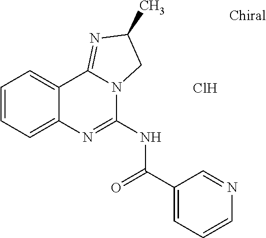 [SMILES: C[C@H]1CN2C(NC(=O)c3cccnc3)=Nc3ccccc3C2=N1 ; Cl ; "Chiral"] |
| 2-364 | 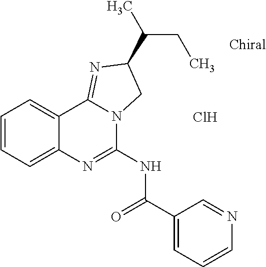 |
| 2-365 | 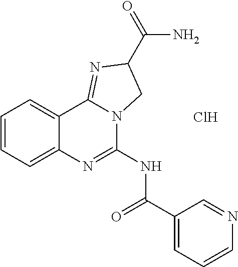 [SMILES: NC(=O)C1CN2C(NC(=O)c3cccnc3)=Nc3ccccc3C2=N1 ; Cl] |
| 2-366 | 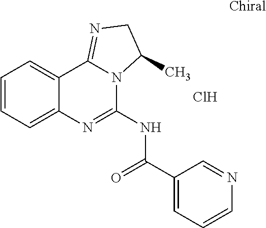 [SMILES: "Chiral" ; C[C@@H]1CN=C2c3ccccc3N=C(NC(=O)c3cccnc3)N21 ; Cl] |
| 2-367 | 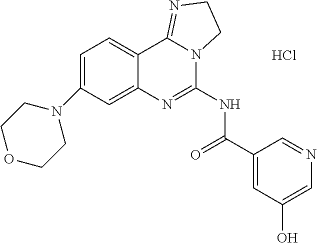 [SMILES: O=C(NC1=Nc2cc(N3CCOCC3)ccc2C2=NCCN12)c1cncc(O)c1 ; Cl] |
| 2-368 | 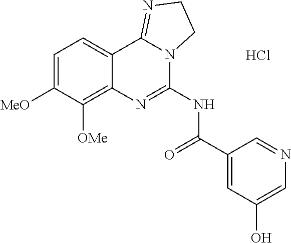 [SMILES: COc1ccc2c(c1OC)N=C(NC(=O)c1cncc(O)c1)N1CCN=C21 ; Cl] |
| 3-1 | 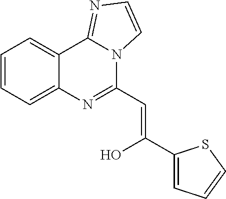 [SMILES: O/C(=C\c1nc2ccccc2c2nccn12)c1cccs1] |
| 3-2 | 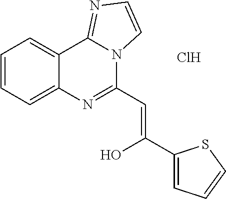 [SMILES: O/C(=C\c1nc2ccccc2c2nccn12)c1cccs1 ; Cl] |
| 4-1 | 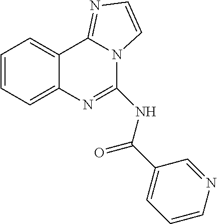 [SMILES: O=C(Nc1nc2ccccc2c2nccn12)c1cccnc1] |
| 4-2 | 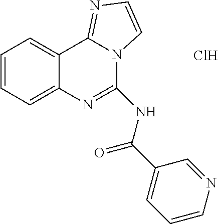 [SMILES: O=C(Nc1nc2ccccc2c2nccn12)c1cccnc1 ; Cl] |

or a physiologically acceptable salt, solvate, hydrate or stereoisomer thereof.

4. The combination according to claim 2, wherein:
said component A is one or more 2,3-dihydroimidazo[1,2-c]quinazoline compounds selected from:
Example 1: N-[7-methoxy-8-(3-morpholin-4-ylpropoxy)-2,3-dihydroimidazo[1,2-c]quinazolin-5-yl]pyrimidine-5-carboxamide
Example 2: N-(8-{3-[(2R,6S)-2,6-dimethylmorpholin-4-yl]propoxy}-7-methoxy-2,3-dihydroimidazo[1,2-c]quinazolin-5-yl)nicotinamide
Example 3: N-(8-{3-[(2R,6S)-2,6-dimethylmorpholin-4-yl]propoxy}-7-methoxy-2,3-dihydroimidazo[1,2-c]quinazolin-5-yl)-2,4-dimethyl-1,3-thiazole-5-carboxamide
Example 4: 2-amino-N-[7-methoxy-8-(3-morpholin-4-ylpropoxy)-2,3-dihydroimidazo[1,2-c]quinazolin-5-yl]-1,3-thiazole-5-carboxamide.
Example 5: 2-amino-N-[7-methoxy-8-(3-morpholin-4-ylpropoxy)-2,3-dihydroimidazo[1,2-c]quinazolin-5-yl]isonicotinamide
Example 6: 2-amino-N-[7-methoxy-8-(3-morpholin-4-ylpropoxy)-2,3-dihydroimidazo[1,2-c]quinazolin-5-yl]-4-methyl-1,3-thiazole-5-carboxamide
Example 7: 2-amino-N-[7-methoxy-8-(3-morpholin-4-ylpropoxy)-2,3-dihydroimidazo[1,2-c]quinazolin-5-yl]-4-propylpyrimidine-5-carboxamide
Example 8: N-{8-[2-(4-ethylmorpholin-2-yl)ethoxy]-7-methoxy-2,3-dihydroimidazo[1,2-c]quinazolin-5-yl}nicotinamide
Example 9: N-{8-[2-(dimethylamino)ethoxy]-7-methoxy-2,3-dihydroimidazo[1,2-c]quinazolin-5-yl}pyrimidine-5-carboxamide
Example 10: N-(8-{3-[2-(hydroxymethyl)morpholin-4-yl]propoxy}-7-methoxy-2,3-dihydroimidazo[1,2-c]quinazolin-5-yl)nicotinamide
Example 11: N-(8-{3-[2-(hydroxymethyl)morpholin-4-yl]propoxy}-7-methoxy-2,3-dihydroimidazo[1,2-c]quinazolin-5-yl)nicotinamide
Example 12: N-{8-[3-(dimethylamino)propoxy]-7-methoxy-2,3-dihydroimidazo[1,2-c]quinazolin-5-yl}nicotinamide 1-oxide
Example 13: 2-amino-N-[7-methoxy-8-(3-morpholin-4-ylpropoxy)-2,3-dihydroimidazo[1,2-c]quinazolin-5-yl]pyrimidine-5-carboxamide.
Example 14: N-[7-methoxy-8-(3-morpholin-4-ylpropoxy)-2,3-dihydroimidazo[1,2-c]quinazolin-5-yl]-6-(2-pyrrolidin-1-ylethyl)nicotinamide.
Example 15: 6-(cyclopentylamino)-N-[7-methoxy-8-(3-morpholin-4-ylpropoxy)-2,3-dihydroimidazo[1,2-c]quinazolin-5-yl]nicotinamide

| Example | Structure |
|---|---|
| 16 | 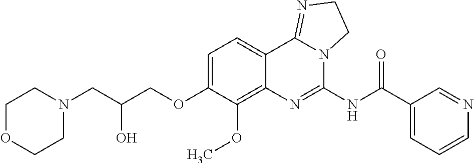 [SMILES: COc1c(OCC(O)CN2CCOCC2)ccc2c1N=C(NC(=O)c1cccnc1)N1CCN=C21] |
| 17 | 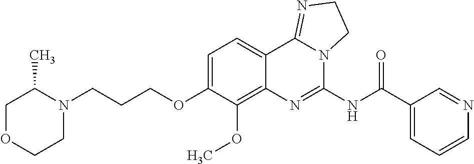 [SMILES: COc1c(OCCCN2CCOC[C@@H]2C)ccc2c1N=C(NC(=O)c1cccnc1)N1CCN=C21] |
| 18 | 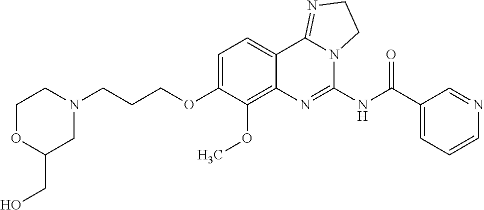 [SMILES: COc1c(OCCCN2CCOC(CO)C2)ccc2c1N=C(NC(=O)c1cccnc1)N1CCN=C21] |
| 19 | 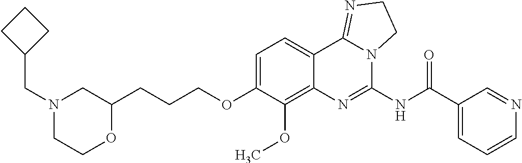 [SMILES: COc1c(OCCCC2CN(CC3CCC3)CCO2)ccc2c1N=C(NC(=O)c1cccnc1)N1CCN=C21] |
| 20 | 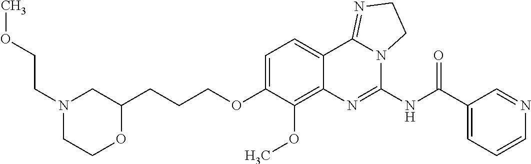 [SMILES: COCCN1CCOC(CCCOc2ccc3c(c2OC)N=C(NC(=O)c2cccnc2)N2CCN=C32)C1] |
| 21 | 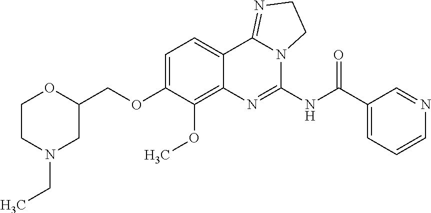 [SMILES: CCN1CCOC(COc2ccc3c(c2OC)N=C(NC(=O)c2cccnc2)N2CCN=C32)C1] |
| 22 | 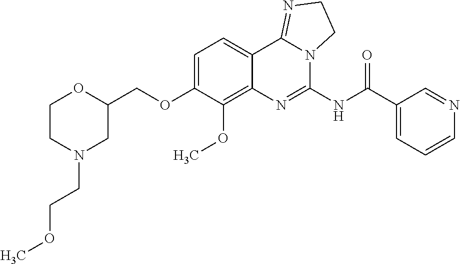 [SMILES: COCCN1CCOC(COc2ccc3c(c2OC)N=C(NC(=O)c2cccnc2)N2CCN=C32)C1] |
| 23 | 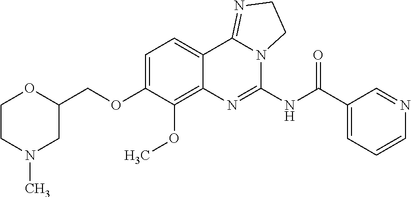 [SMILES: COc1c(OCC2CN(C)CCO2)ccc2c1N=C(NC(=O)c1cccnc1)N1CCN=C21] |
| 24 | 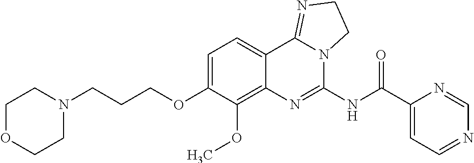 [SMILES: COc1c(OCCCN2CCOCC2)ccc2c1N=C(NC(=O)c1ccncn1)N1CCN=C21] |
| 25 | 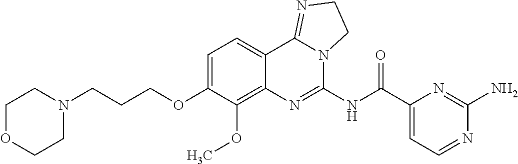 [SMILES: COc1c(OCCCN2CCOCC2)ccc2c1N=C(NC(=O)c1ccnc(N)n1)N1CCN=C21] |
| 26 | 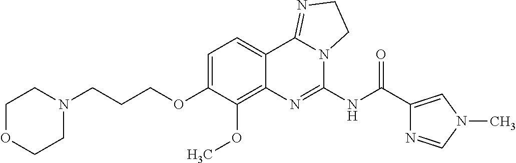 [SMILES: COc1c(OCCCN2CCOCC2)ccc2c1N=C(NC(=O)c1cn(C)cn1)N1CCN=C21] |
| 27 | 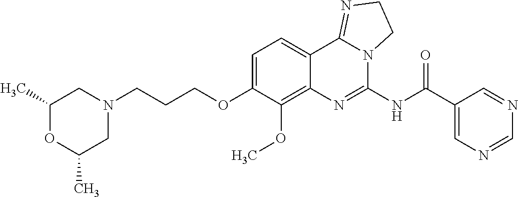 [SMILES: COc1c(OCCCN2C[C@@H](C)O[C@@H](C)C2)ccc2c1N=C(NC(=O)c1cncnc1)N1CCN=C21] |
| 28 | 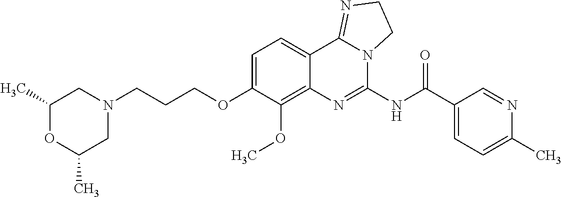 |
| 29 | 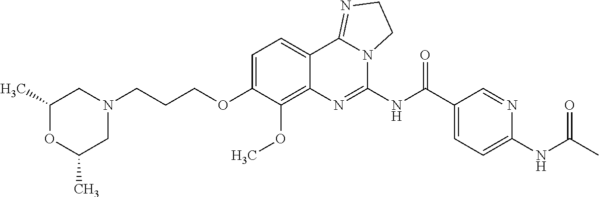 [SMILES: COc1c(OCCCN2C[C@@H](C)O[C@@H](C)C2)ccc2c1N=C(NC(=O)c1ccc(NC(C)=O)nc1)N1CCN=C21] |
| 30 | 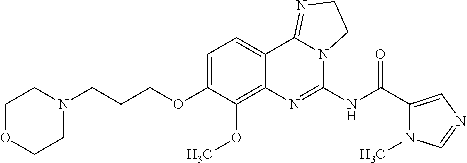 [SMILES: COc1c(OCCCN2CCOCC2)ccc2c1N=C(NC(=O)c1cncn1C)N1CCN=C21] |
| 31 | 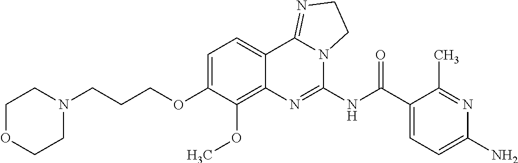 [SMILES: COc1c(OCCCN2CCOCC2)ccc2c1N=C(NC(=O)c1ccc(N)nc1C)N1CCN=C21] |
| 32 | 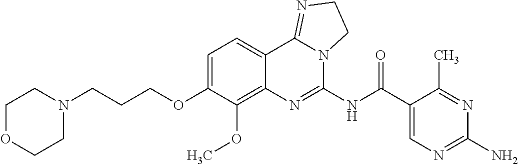 [SMILES: COc1c(OCCCN2CCOCC2)ccc2c1N=C(NC(=O)c1cnc(N)nc1C)N1CCN=C21] |
| 33 | 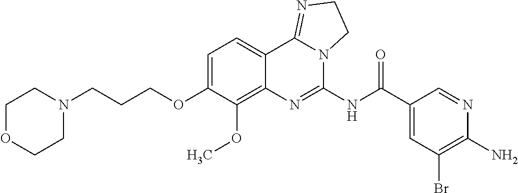 [SMILES: COc1c(OCCCN2CCOCC2)ccc2c1N=C(NC(=O)c1cnc(N)c(Br)c1)N1CCN=C21] |
| 34 | 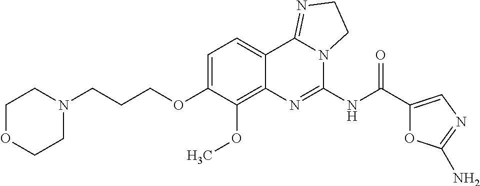 [SMILES: COc1c(OCCCN2CCOCC2)ccc2c1N=C(NC(=O)c1cnc(N)o1)N1CCN=C21] |
| 35 | 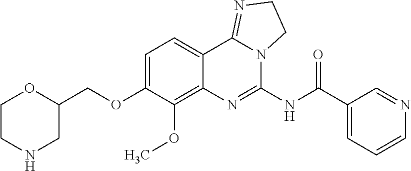 [SMILES: COc1c(OCC2CNCCO2)ccc2c1N=C(NC(=O)c1cccnc1)N1CCN=C21] |
| 36 | 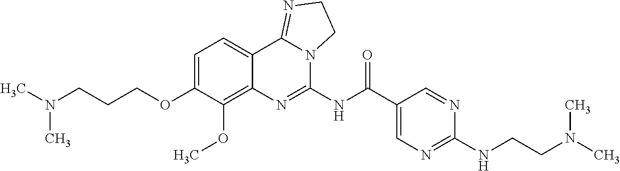 |
| 37 | 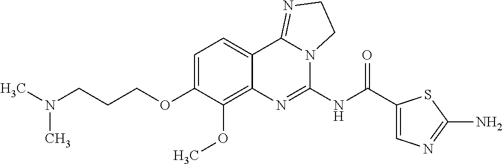 [SMILES: COc1c(OCCCN(C)C)ccc2c1N=C(NC(=O)c1cnc(N)s1)N1CCN=C21] |
| 38 | 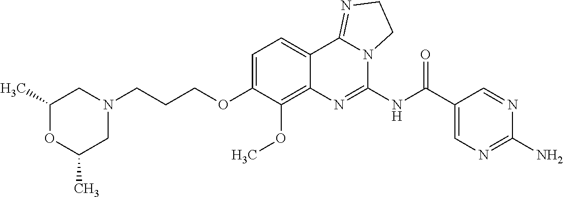 [SMILES: COc1c(OCCCN2C[C@@H](C)O[C@@H](C)C2)ccc2c1N=C(NC(=O)c1cnc(N)nc1)N1CCN=C21] |
| 39 | 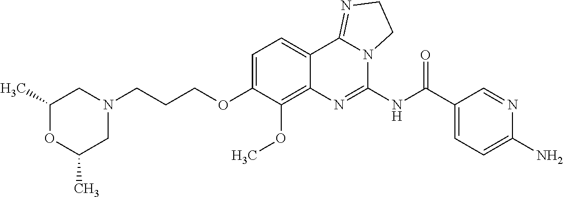 [SMILES: COc1c(OCCCN2C[C@@H](C)O[C@@H](C)C2)ccc2c1N=C(NC(=O)c1ccc(N)nc1)N1CCN=C21] |
| 40 | 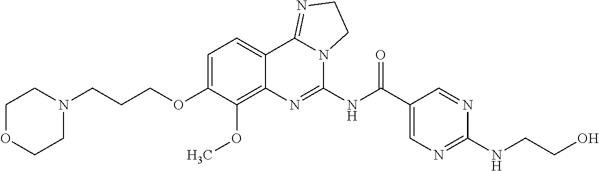 [SMILES: COc1c(OCCCN2CCOCC2)ccc2c1N=C(NC(=O)c1cnc(NCCO)nc1)N1CCN=C21] |
| 41 | 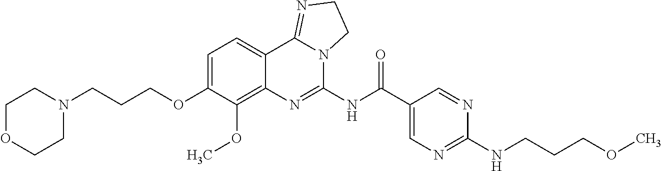 [SMILES: COCCCNc1ncc(C(=O)NC2=Nc3c(ccc(OCCCN4CCOCC4)c3OC)C3=NCCN23)cn1] |
| 42 | 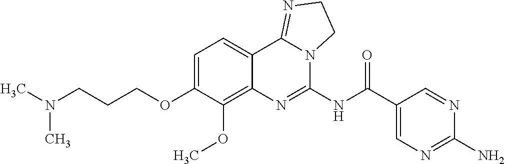 [SMILES: COc1c(OCCCN(C)C)ccc2c1N=C(NC(=O)c1cnc(N)nc1)N1CCN=C21] |
| 43 | 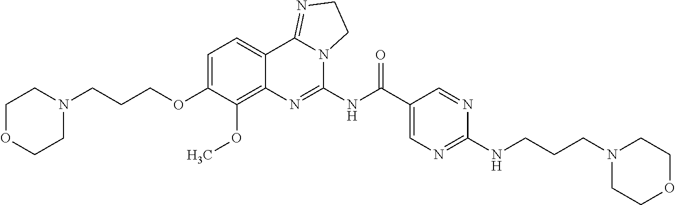 [SMILES: COc1c(OCCCN2CCOCC2)ccc2c1N=C(NC(=O)c1cnc(NCCCN3CCOCC3)nc1)N1CCN=C21] |
| 44 | 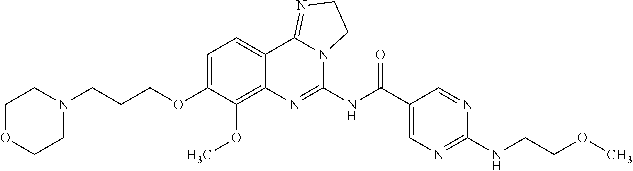 [SMILES: COCCNc1ncc(C(=O)NC2=Nc3c(ccc(OCCCN4CCOCC4)c3OC)C3=NCCN23)cn1] |
| 45 | 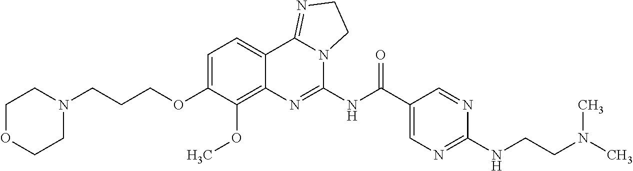 [SMILES: COc1c(OCCCN2CCOCC2)ccc2c1N=C(NC(=O)c1cnc(NCCN(C)C)nc1)N1CCN=C21] |
| 46 | 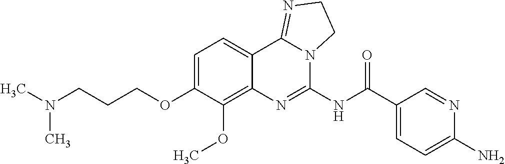 [SMILES: COc1c(OCCCN(C)C)ccc2c1N=C(NC(=O)c1ccc(N)nc1)N1CCN=C21] |
| 47 | 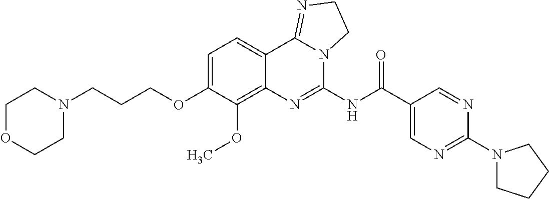 [SMILES: COc1c(OCCCN2CCOCC2)ccc2c1N=C(NC(=O)c1cnc(N3CCCC3)nc1)N1CCN=C21] |
| 48 | 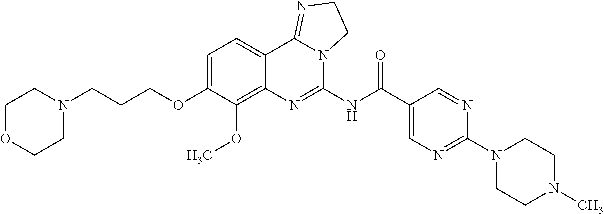 [SMILES: COc1c(OCCCN2CCOCC2)ccc2c1N=C(NC(=O)c1cnc(N3CCN(C)CC3)nc1)N1CCN=C21] |
| 49 | 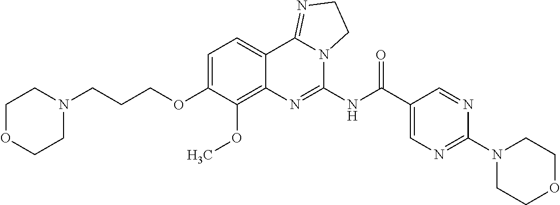 [SMILES: COc1c(OCCCN2CCOCC2)ccc2c1N=C(NC(=O)c1cnc(N3CCOCC3)nc1)N1CCN=C21] |
| 50 | 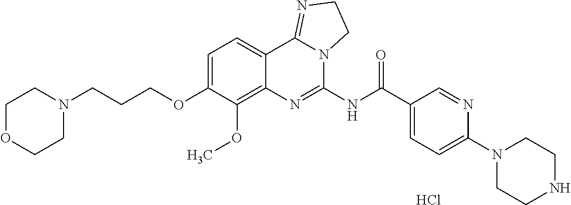 [SMILES: COc1c(OCCCN2CCOCC2)ccc2c1N=C(NC(=O)c1ccc(N3CCNCC3)nc1)N1CCN=C21 ; Cl] |
| 51 | 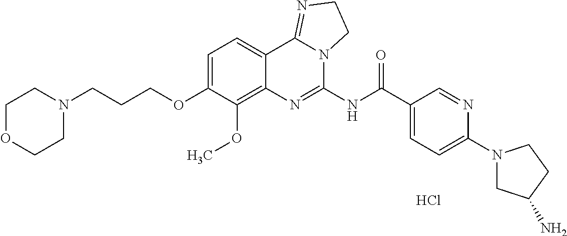 [SMILES: COc1c(OCCCN2CCOCC2)ccc2c1N=C(NC(=O)c1ccc(N3CC[C@H](N)C3)nc1)N1CCN=C21 ; Cl] |
| 52 | 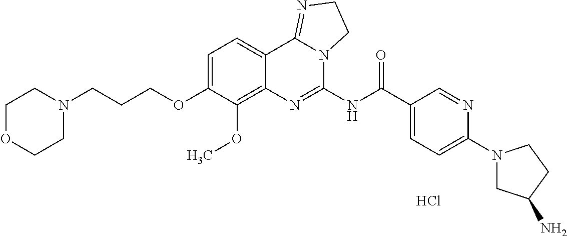 [SMILES: COc1c(OCCCN2CCOCC2)ccc2c1N=C(NC(=O)c1ccc(N3CC[C@@H](N)C3)nc1)N1CCN=C21 ; Cl] |
| 53 | 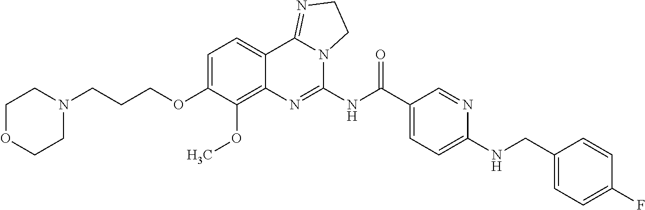 [SMILES: COc1c(OCCCN2CCOCC2)ccc2c1N=C(NC(=O)c1ccc(NCc3ccc(F)cc3)nc1)N1CCN=C21] |
| 54 | 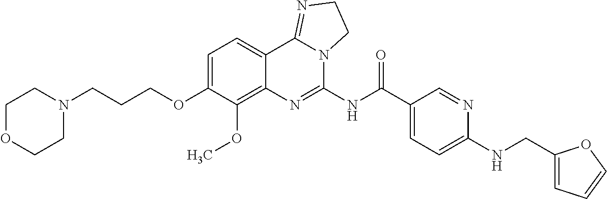 [SMILES: COc1c(OCCCN2CCOCC2)ccc2c1N=C(NC(=O)c1ccc(NCc3ccco3)nc1)N1CCN=C21] |
| 55 | 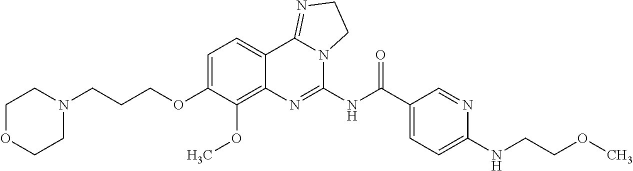 [SMILES: COCCNc1ccc(C(=O)NC2=Nc3c(ccc(OCCCN4CCOCC4)c3OC)C3=NCCN23)cn1] |
| 56 | 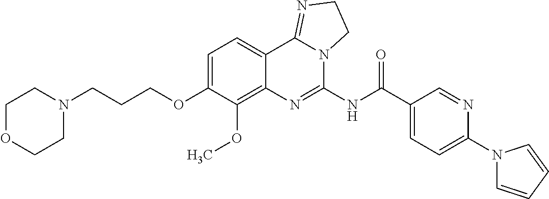 [SMILES: COc1c(OCCCN2CCOCC2)ccc2c1N=C(NC(=O)c1ccc(-n3cccc3)nc1)N1CCN=C21] |
| 57 | 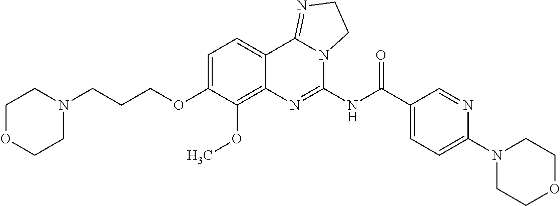 [SMILES: COc1c(OCCCN2CCOCC2)ccc2c1N=C(NC(=O)c1ccc(N3CCOCC3)nc1)N1CCN=C21] |
| 58 | 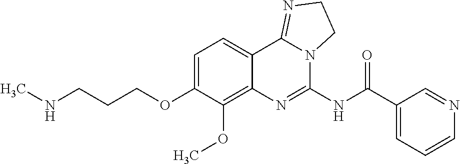 [SMILES: CNCCCOc1ccc2c(c1OC)N=C(NC(=O)c1cccnc1)N1CCN=C21] |
| 59 | 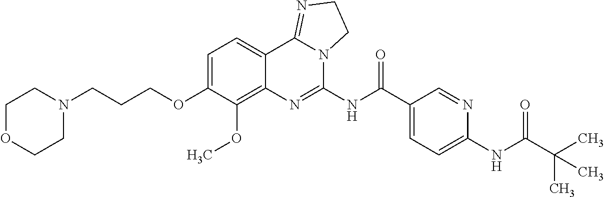 [SMILES: COc1c(OCCCN2CCOCC2)ccc2c1N=C(NC(=O)c1ccc(NC(=O)C(C)(C)C)nc1)N1CCN=C21] |
| 60 | 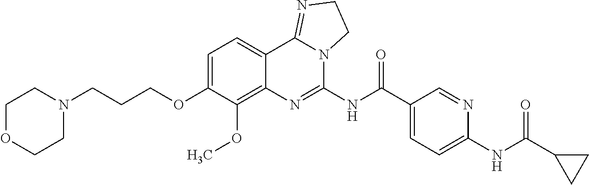 [SMILES: COc1c(OCCCN2CCOCC2)ccc2c1N=C(NC(=O)c1ccc(NC(=O)C3CC3)nc1)N1CCN=C21] |
| 61 | 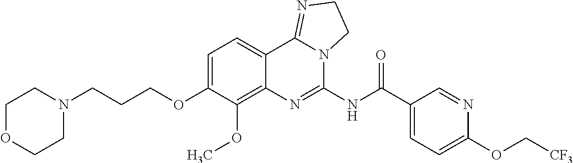 [SMILES: COc1c(OCCCN2CCOCC2)ccc2c1N=C(NC(=O)c1ccc(OCC(F)(F)F)nc1)N1CCN=C21] |
| 62 | 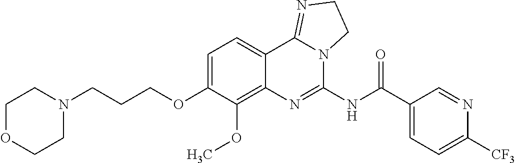 [SMILES: COc1c(OCCCN2CCOCC2)ccc2c1N=C(NC(=O)c1ccc(C(F)(F)F)nc1)N1CCN=C21] |
| 63 | 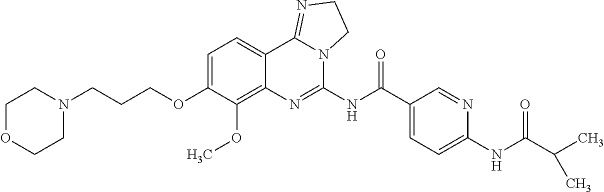 [SMILES: COc1c(OCCCN2CCOCC2)ccc2c1N=C(NC(=O)c1ccc(NC(=O)C(C)C)nc1)N1CCN=C21] |
| 64 | 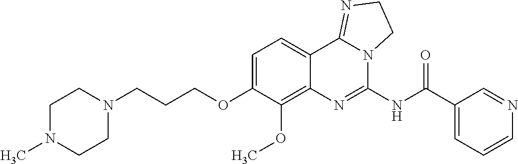 [SMILES: COc1c(OCCCN2CCN(C)CC2)ccc2c1N=C(NC(=O)c1cccnc1)N1CCN=C21] |
| 65 | 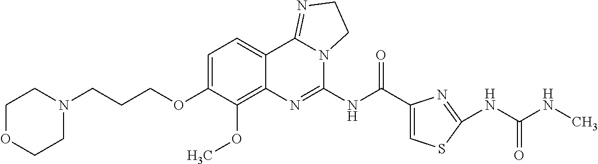 [SMILES: CNC(=O)Nc1nc(C(=O)NC2=Nc3c(ccc(OCCCN4CCOCC4)c3OC)C3=NCCN23)cs1] |
| 66 | 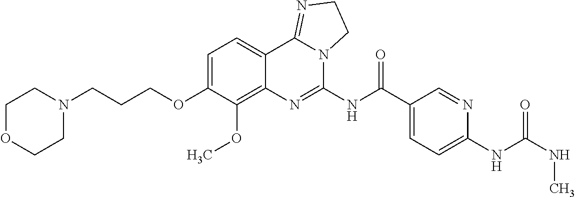 [SMILES: CNC(=O)Nc1ccc(C(=O)NC2=Nc3c(ccc(OCCCN4CCOCC4)c3OC)C3=NCCN23)cn1] |
| 67 | 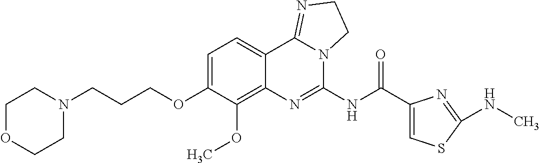 [SMILES: CNc1nc(C(=O)NC2=Nc3c(ccc(OCCCN4CCOCC4)c3OC)C3=NCCN23)cs1] |
| 68 | 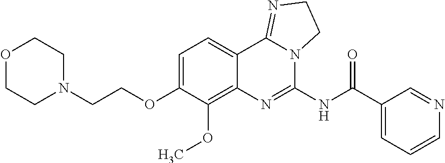 [SMILES: COc1c(OCCN2CCOCC2)ccc2c1N=C(NC(=O)c1cccnc1)N1CCN=C21] |
| 69 | 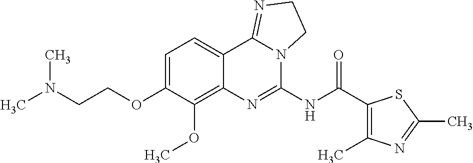 |
| 70 | 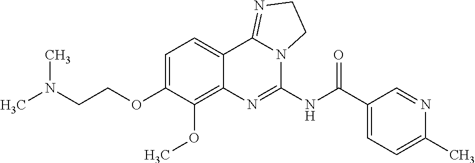 |
| 71 | 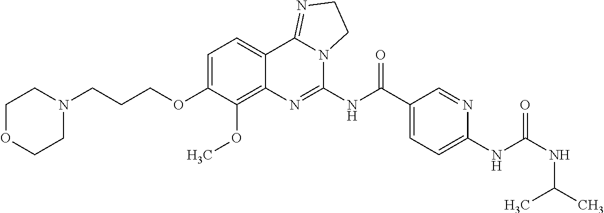 [SMILES: COc1c(OCCCN2CCOCC2)ccc2c1N=C(NC(=O)c1ccc(NC(=O)NC(C)C)nc1)N1CCN=C21] |
| 72 | 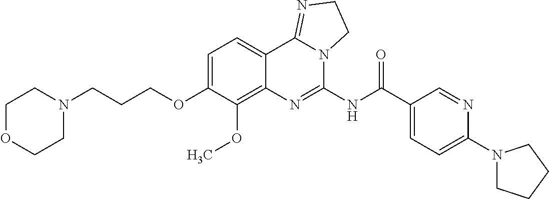 [SMILES: COc1c(OCCCN2CCOCC2)ccc2c1N=C(NC(=O)c1ccc(N3CCCC3)nc1)N1CCN=C21] |
| 73 | 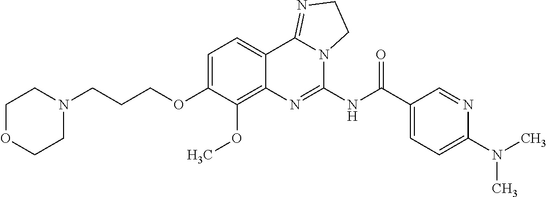 [SMILES: COc1c(OCCCN2CCOCC2)ccc2c1N=C(NC(=O)c1ccc(N(C)C)nc1)N1CCN=C21] |
| 74 | 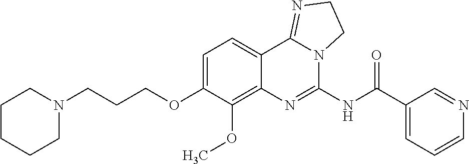 [SMILES: COc1c(OCCCN2CCCCC2)ccc2c1N=C(NC(=O)c1cccnc1)N1CCN=C21] |
| 75 | 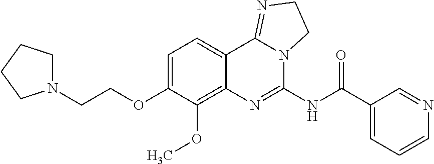 [SMILES: COc1c(OCCN2CCCC2)ccc2c1N=C(NC(=O)c1cccnc1)N1CCN=C21] |
| 76 | 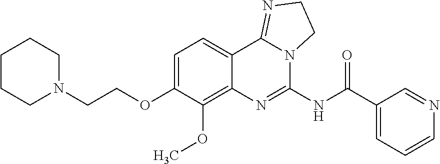 [SMILES: COc1c(OCCN2CCCCC2)ccc2c1N=C(NC(=O)c1cccnc1)N1CCN=C21] |
| 77 | 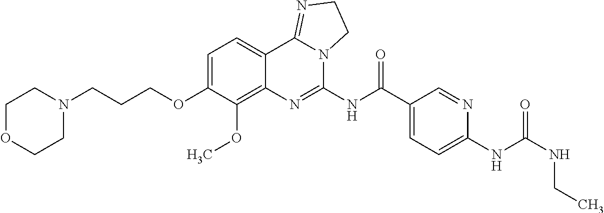 [SMILES: CCNC(=O)Nc1ccc(C(=O)NC2=Nc3c(ccc(OCCCN4CCOCC4)c3OC)C3=NCCN23)cn1] |
| 78 | 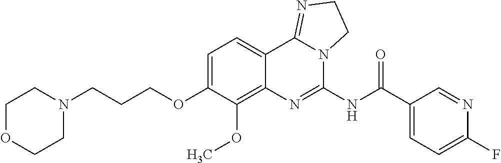 [SMILES: COc1c(OCCCN2CCOCC2)ccc2c1N=C(NC(=O)c1ccc(F)nc1)N1CCN=C21] |
| 79 | 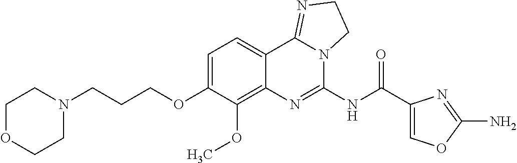 [SMILES: COc1c(OCCCN2CCOCC2)ccc2c1N=C(NC(=O)c1coc(N)n1)N1CCN=C21] |
| 80 | 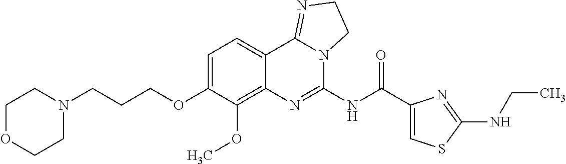 [SMILES: CCNc1nc(C(=O)NC2=Nc3c(ccc(OCCCN4CCOCC4)c3OC)C3=NCCN23)cs1] |
| 81 | 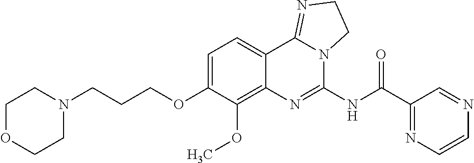 [SMILES: COc1c(OCCCN2CCOCC2)ccc2c1N=C(NC(=O)c1cnccn1)N1CCN=C21] |
| 82 | 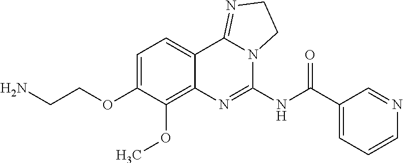 [SMILES: COc1c(OCCN)ccc2c1N=C(NC(=O)c1cccnc1)N1CCN=C21] |
| 83 | 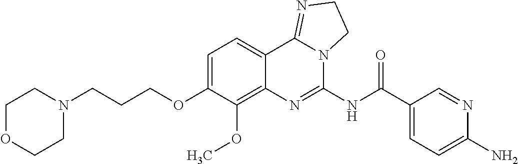 [SMILES: COc1c(OCCCN2CCOCC2)ccc2c1N=C(NC(=O)c1ccc(N)nc1)N1CCN=C21] |
| 84 | 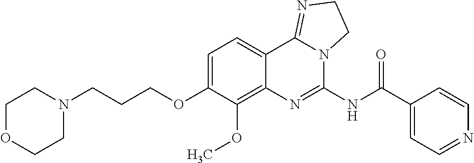 [SMILES: COc1c(OCCCN2CCOCC2)ccc2c1N=C(NC(=O)c1ccncc1)N1CCN=C21] |
| 85 | 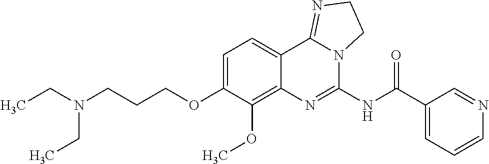 [SMILES: CCN(CC)CCCOc1ccc2c(c1OC)N=C(NC(=O)c1cccnc1)N1CCN=C21] |
| 86 | 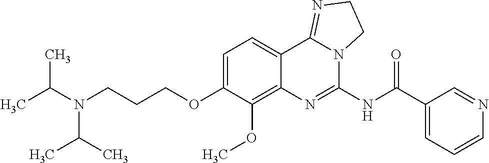 [SMILES: COc1c(OCCCN(C(C)C)C(C)C)ccc2c1N=C(NC(=O)c1cccnc1)N1CCN=C21] |
| 87 | 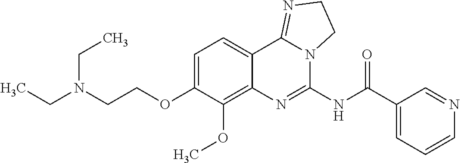 [SMILES: CCN(CC)CCOc1ccc2c(c1OC)N=C(NC(=O)c1cccnc1)N1CCN=C21] |
| 88 | 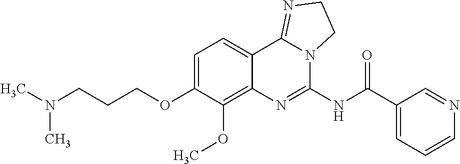 [SMILES: COc1c(OCCCN(C)C)ccc2c1N=C(NC(=O)c1cccnc1)N1CCN=C21] |
| 89 | 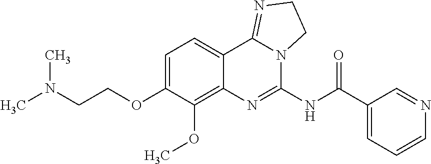 [SMILES: COc1c(OCCN(C)C)ccc2c1N=C(NC(=O)c1cccnc1)N1CCN=C21] |
| 90 | 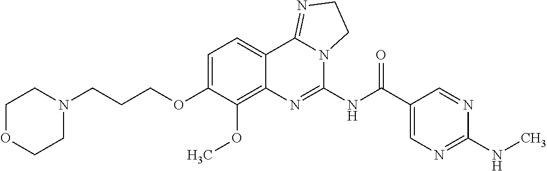 [SMILES: CNc1ncc(C(=O)NC2=Nc3c(ccc(OCCCN4CCOCC4)c3OC)C3=NCCN23)cn1] |
| 91 | 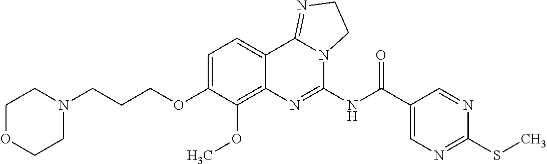 [SMILES: COc1c(OCCCN2CCOCC2)ccc2c1N=C(NC(=O)c1cnc(SC)nc1)N1CCN=C21] |
| 92 | 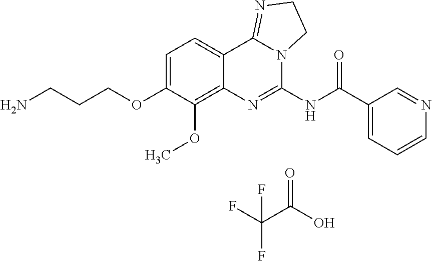 [SMILES: COc1c(OCCCN)ccc2c1N=C(NC(=O)c1cccnc1)N1CCN=C21 ; O=C(O)C(F)(F)F] |
| 93 | 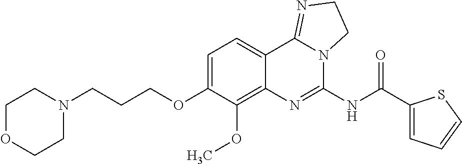 [SMILES: COc1c(OCCCN2CCOCC2)ccc2c1N=C(NC(=O)c1cccs1)N1CCN=C21] |
| 94 | 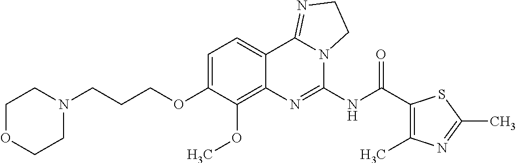 [SMILES: COc1c(OCCCN2CCOCC2)ccc2c1N=C(NC(=O)c1sc(C)nc1C)N1CCN=C21] |
| 95 | 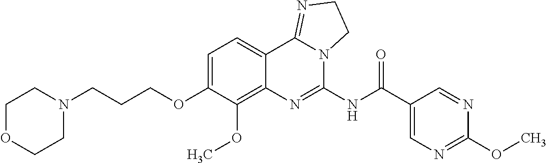 [SMILES: COc1ncc(C(=O)NC2=Nc3c(ccc(OCCCN4CCOCC4)c3OC)C3=NCCN23)cn1] |
| 96 | 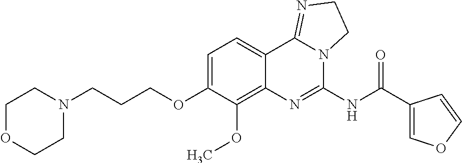 [SMILES: COc1c(OCCCN2CCOCC2)ccc2c1N=C(NC(=O)c1ccoc1)N1CCN=C21] |
| 97 | 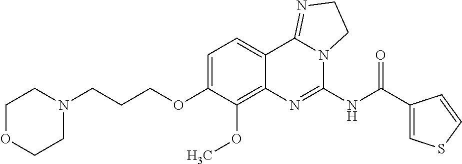 [SMILES: COc1c(OCCCN2CCOCC2)ccc2c1N=C(NC(=O)c1ccsc1)N1CCN=C21] |
| 98 | 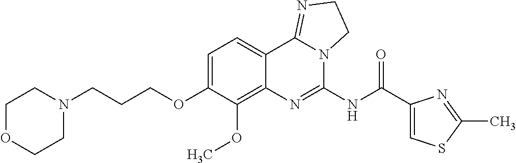 [SMILES: COc1c(OCCCN2CCOCC2)ccc2c1N=C(NC(=O)c1csc(C)n1)N1CCN=C21] |
| 99 | 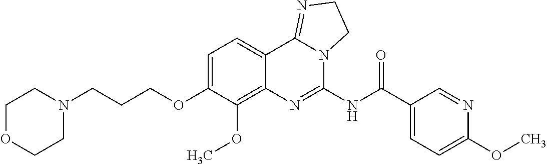 [SMILES: COc1ccc(C(=O)NC2=Nc3c(ccc(OCCCN4CCOCC4)c3OC)C3=NCCN23)cn1] |
| 100 | 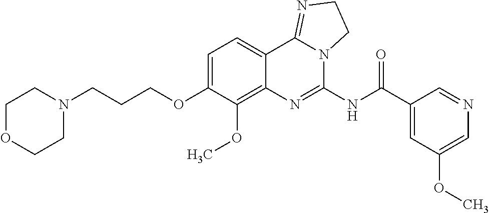 [SMILES: COc1cncc(C(=O)NC2=Nc3c(ccc(OCCCN4CCOCC4)c3OC)C3=NCCN23)c1] |
| 101 | 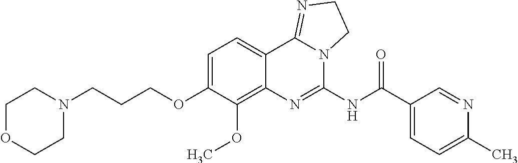 [SMILES: COc1c(OCCCN2CCOCC2)ccc2c1N=C(NC(=O)c1ccc(C)nc1)N1CCN=C21] |
| 102 | 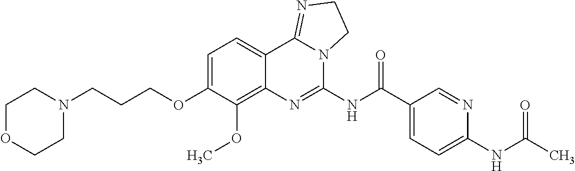 [SMILES: COc1c(OCCCN2CCOCC2)ccc2c1N=C(NC(=O)c1ccc(NC(C)=O)nc1)N1CCN=C21] |
| 103 | 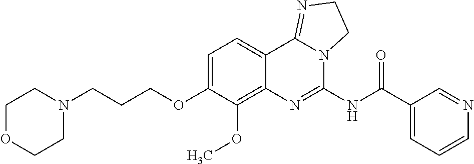 [SMILES: COc1c(OCCCN2CCOCC2)ccc2c1N=C(NC(=O)c1cccnc1)N1CCN=C21] |

or a physiologically acceptable salt, solvate, hydrate or stereoisomer thereof.

4. The combination according to claim 1, wherein:
said component B is one or more 2,3-dihydroimidazo[1,2-c]quinazoline compounds selected from: Example 1: N-(3,4-difluoro-2-(2-fluoro-4-iodophenylamino)phenyl)-methanesulfonamide:
Example 2: N-(3,4-difluoro-2-(2-fluoro-4-iodophenylamino)phenyl)cyclopropanesulfonamide:
Example 3: N-(3,4-difluoro-2-(2-fluoro-4-iodophenylamino)phenyl)propane-2-sulfonamide:
Example 4: N-(3,4-difluoro-2-(2-fluoro-4-iodophenylamino)phenyl)butane-1-sulfonamide:
Example 5: N-(3,4-difluoro-2-(2-fluoro-4-iodophenylamino)phenyl)-2,2,2-trifluoro ethane sulfonamide:
Example 6: N-(3,4-difluoro-2-(2-fluoro-4-iodophenylamino)phenyl)butane-2-sulfonamide:
Example 7: N-(3,4-difluoro-2-(2-fluoro-4-iodophenylamino)phenyl)-N-methyl cyclopropane sulfonamide:
Example 8: 1-Chloro-N-(3,4-difluoro-2-(2-fluoro-4-iodophenylamino)phenyl)methane sulfonamide:
Example 9: N-(3,4-difluoro-2-(2-fluoro-4-iodophenylamino)phenyl)-2-methylpropane-2-sulfonamide:
Example 10: N-(3,4-difluoro-2-(2-fluoro-4-iodophenylamino)phenyl)cyclopentanesulfonamide:
Example 11: N-(3,4-difluoro-2-(2-fluoro-4-iodophenylamino)phenyl)cyclohexanesulfonamide:
Example 12: N-(3,4-difluoro-2-(2-fluoro-4-iodophenylamino)phenyl)-1-methylcyclopropane-1-sulfonamide:
Example 13: N-(3,4-difluoro-2-(2-fluoro-4-iodophenylamino)phenyl)-1-(2,3-dihydroxypropyl)cyclopropane-1-sulfonamide:
Example 14: (S)—N-(3,4-difluoro-2-(2-fluoro-4-iodophenylamino)phenyl)-1-(2,3-dihydroxypropyl)cyclopropane-1-sulfonamide:
Example 15: (R)—N-(3,4-difluoro-2-(2-fluoro-4-iodophenylamino)phenyl)-1-(2,3-dihydroxypropyl)cyclopropane-1-sulfonamide:
Example 16: N-(3,4-difluoro-2-(2-fluoro-4-iodophenylamino)phenyl)-1-(2-hydroxyethyl)cyclopropane-1-sulfonamide:
Example 17: N-(3,4-difluoro-2-(2-fluoro-4-iodophenylamino)phenyl)-3-hydroxypropane-1-sulfonamide:
Example 18: N-(3,4-difluoro-2-(2-fluoro-4-iodophenylamino)phenyl)-2-methyl-5-(trifluoromethyl)furan-3-sulfonamide:
Example 19: N-(5-(N-(3,4-difluoro-2-(2-fluoro-4-iodophenylamino)phenyl)sulfamoyl)-methylthiazol-2-yl)acetamide:
Example 20: 5-(5-Chloro-1,2,4-thiadiazol-3-yl)-N-(3,4-difluoro-2-(2-fluoro-4-iodophenylamino)phenyl)thiophene-2-sulfonamide:
Example 21: N-(3,4-difluoro-2-(2-fluoro-4-iodophenylamino)phenyl)-3,5-dimethylisoxazole-4-sulfonamide:
Example 22: 5-Chloro-N-(3,4-difluoro-2-(2-fluoro-4-iodophenylamino)phenyl)-1,3-dimethyl-1H-pyrazole-4-sulfonamide:
Example 23: N-(3,4-difluoro-2-(2-fluoro-4-iodophenylamino)phenyl)-2,5-dimethylfuran-3-sulfonamide:
Example 24: N-(3,4-difluoro-2-(2-fluoro-4-iodophenylamino)phenyl)-1-methyl-3-(trifluoromethyl)-1H-pyrazole-4-sulfonamide:
Example 25: N-(3,4-difluoro-2-(2-fluoro-4-iodophenylamino)phenyl)-2,4-dimethylthiazole-5-sulfonamide:
Example 26: N-(3,4-difluoro-2-(2-fluoro-4-iodophenylamino)phenyl)-1,2-dimethyl-1H-imidazole-4-sulfonamide:
Example 27: N-(3,4-difluoro-2-(2-fluoro-4-iodophenylamino)phenyl)thiophene-3-sulfonamide:
Example 28: N-(3,4-difluoro-2-(2-fluoro-4-iodophenylamino)phenyl)furan-2-sulfonamide:
Example 29: N-(3,4-difluoro-2-(2-fluoro-4-iodophenylamino)phenyl)-5-methylthiophene-2-sulfonamide:
Example 30: 5-Chloro-N-(3,4-difluoro-2-(2-fluoro-4-iodophenylamino)phenyl)thiophene-2-sulfonamide:
Example 31: 5-Bromo-N-(3,4-difluoro-2-(2-fluoro-4-iodophenylamino)phenyl)thiophene-2-sulfonamide:
Example 32: 4-Bromo-N-(3,4-difluoro-2-(2-fluoro-4-iodophenylamino)phenyl)thiophene-3-sulfonamide:
Example 33: 4-Brom-5-chloro-N-(3,4-difluoro-2-(2-fluoro-4-iodophenylamino)phenyl)thiophene-2-sulfonamide:
Example 34: 3-Brom-5-chloro-N-(3,4-difluoro-2-(2-fluoro-4-iodophenylamino) phenyl)thiophene-2-sulfonamide:
Example 35: N-(3,4-difluoro-2-(2-fluoro-4-iodophenylamino)phenyl)-2,5-dimethylthiophene-3-sulfonamide:
Example 36: 2,5-Dichloro-N-(3,4-difluoro-2-(2-fluoro-4-iodophenylamino)phenyl)thiophene-3-sulfonamide:
Example 37: Methyl 3-(N-(3,4-difluoro-2-(2-fluoro-4-iodophenylamino)phenyl) sulfamoyl)thiophene-2-carboxylate:
Example 38 Methyl 5-(N-(3,4-difluoro-2-(2-fluoro-4-iodophenylamino)phenyl)sulfamoyl)-1-methyl-1H-pyrrole-2-carboxylate:
Example 39: N-(3,4-difluoro-2-(2-fluoro-4-iodophenylamino)phenyl)-5-methylisoxazole-4-sulfonamide:
Example 40: 3-Chloro-N-(3,4-difluoro-2-(2-fluoro-4-iodophenylamino)phenyl)propane-1-sulfonamide:
Example 41: N-(2-(4-chloro-2-fluorophenylamino)-3,4-difluorophenyl)cyclopropanesulfonamide:
Example 42: N-(3,4-difluoro-2-(4-iodo-2-methylphenylamino)phenyl)cyclopropanesulfonamide:
Example 43: N-(2-(4-tert-butyl-2-chlorophenylamino)-3,4-difluorophenyl)cyclopropanesulfonamide:
Example 44: N-(2-(2,4-dichlorophenylamino)-3,4-difluorophenyl)cyclopropanesulfonamide:
Example 45: 3-Chloro-N-(3,4-difluoro-2-(2-fluoro-4-trifluoromethyl) phenylamino)phenyl)propane-1-sulfonamide:
Example 46: N-(3,4-difluoro-2-(2-chloro-4-trifluoromethyl)phenylamino)methanesulfonamide:
Example 47: 3-Chloro-N-(3,4-difluoro-2-(2-chloro-4-trifluoromethyl) phenylamino)phenyl)propane-1-sulfonamide:
Example 48: 3-Chloro-N-(3,4-difluoro-2-(2-bromo-4-trifluoromethyl) phenylamino)phenyl)propane-1-sulfonamide:
Example 49: Cyclopropanesulfonic acid (3,4,6-trifluoro-2-(2-fluoro-4-iodo-phenylamino)-phenyl)-amide:
Example 50: N-(3,4-difluoro-2-(4-fluoro-2-iodophenylamino)-6-ethoxyphenyl)cyclopropane sulfonamide:
Example 51: Methylsulfonic acid (3,4-difluoro-2-(2-fluoro-4-iodo-phenylamino)-6-methoxy-phenyl)-amide:
Example 52: 1-(2,3-Dihydroxy-propyl)-cyclopropanesulfonic acid [3,4,6-trifluoro-2-(4-fluoro-2-iodo-phenylamino)-phenyl]-amide:
Example 53: (S)-1-(2,3-dihydroxypropyl)-N-(3,4,6-trifluoro-2-(2-fluoro-4-iodophenylamino)phenyl)cyclopropane-1-sulfonamide:
Example 54: (R)-1-(2,3-dihydroxypropyl)-N-(3,4,6-trifluoro-2-(2-fluoro-4-iodophenylamino)phenyl)cyclopropane-1-sulfonamide:
Example 55: N-(3,4-difluoro-2-(2-fluoro-4-iodophenylamino)phenyl)-1-(2,3-dihydroxypropyl)cyclopropane-1-sulfonamide:
Example 56: (S)—N-(3,4-difluoro-2-(2-fluoro-4-iodophenylamino)-6-methoxyphenyl)-1-(2,3-dihydroxypropyl)cyclopropane-1-sulfonamide:
Example 57: (R)—N-(3,4-difluoro-2-(2-fluoro-4-iodophenylamino)-6-methoxyphenyl)-1-(2,3-dihydroxypropyl)cyclopropane-1-sulfonamide:
Example 58: 1-(2-hydroxyethyl)-N-(3,4,6-trifluoro-2-(2-fluoro-4-iodophenylamino)phenyl)cyclopropane-1-sulfonamide:
Example 59: N-(3,4-difluoro-2-(2-fluoro-4-iodophenylamino)-6-methoxyphenyl)-1-(2-hydroxyethyl)cyclopropane-1-sulfonamide:
Example 60: N-(3,4-difluoro-2-(2-fluoro-4-iodophenylamino)-6-methoxyphenyl)-1-(3-hydroxy-2-(hydroxymethyl)propyl)cyclopropane-1-sulfonamide:
Example 61: N-(3,4-difluoro-2-(2-fluoro-4-iodophenylamino)-6-methoxyphenyl)cyclobutane sulfonamide:
Example 62: N-(3,4-difluoro-2-(2-fluoro-4-iodophenylamino)-6-methylphenyl)-1-(2,3-dihydroxypropyl)cyclopropane-1-sulfonamide:
Example 63: 1-(2,3-Dihydroxypropyl)-N-(6-ethyl-3,4-difluoro-2-(2-fluoro-4-iodophenylamino)phenyl)cyclopropane-1-sulfonamide:
Example 64: N-(3,4-difluoro-2-(2-fluoro-4-iodophenylamino)-6-(2-methoxyethoxy)phenyl)-1-(2,3-dihydroxypropyl)cyclopropane-1-sulfonamide:
Example 65: 2,4-dichloro-N-(3,4-difluoro-2-(2-fluoro-4-iodophenylamino)phenyl)benzene sulfonamide:
Example 66: 2-chloro-N-(3,4-difluoro-2-(2-fluoro-4-iodophenylamino)phenyl)-4-(trifluoromethyl)benzenesulfonamide:
Example 67: N-(3,4-difluoro-2-(2-fluoro-4-iodophenylamino)phenyl)-2-(trifluoromethoxy)benzene sulfonamide:
Example 68: 4-(N-(3,4-difluoro-2-(2-fluoro-4-iodophenylamino)phenyl)sulfamoyl)benzoic acid:
Example 69: N-(3,4-difluoro-2-(2-fluoro-4-iodophenylamino)phenyl)benzenesulfonamide:
Example 70: N-(3,4-difluoro-2-(2-fluoro-4-iodophenylamino)phenyl)-2-fluorobenzene sulfonamide:
Example 71: N-(3,4-difluoro-2-(2-fluoro-4-methylphenylamino)phenyl)cyclopropanesulfonamide;
or a physiologically acceptable salt, solvate, hydrate or stereoisomer thereof.

5. The combination according to claim 1, wherein said component A is (S)—N-(3,4-difluoro-2-(2-fluoro-4-iodophenylamino)-6-methoxyphenyl)-1-(2,3-dihydroxypropyl)cyclopropane-1-sulfonamide.

6. The combination according to claim 1, wherein said component B is 2-amino-N-[7-methoxy-8-(3-morpholin-4-ylpropoxy)-2,3-dihydroimidazo[1,2-c]quinazolin-5-yl]pyrimidine-5-carboxamide.

7. The combination according to claim 1, wherein said component A is (S)—N-(3,4-difluoro-2-(2-fluoro-4-iodophenylamino)-6-methoxyphenyl)-1-(2,3-dihydroxypropyl)cyclopropane-1-sulfonamide and said component B is 2-amino-N-[7-methoxy-8-(3-morpholin-4-ylpropoxy)-2,3-dihydroimidazo[1,2-c]quinazolin-5-yl]pyrimidine-5-carboxamide.

8. (canceled)

9. A method for the treatment or prophylaxis of a cancer, comprising administering to a human or animal in need thereof a therapeutically effective amount of a combination according to claim 1.

10. A kit comprising a combination according to claim 1:
wherein both or either of said components A) and B) are formulated for simultaneous, concurrent, separate, or sequential administration.

11. The kit according to claim 10, wherein said component A is (S)—N-(3,4-difluoro-2-(2-fluoro-4-iodophenylamino)-6-methoxyphenyl)-1-(2,3-dihydroxypropyl)cyclopropane-1-sulfonamide and said component B is 2-amino-N-[7-methoxy-8-(3-morpholin-4-ylpropoxy)-2,3-dihydroimidazo[1,2-c]quinazolin-5-yl]pyrimidine-5-carboxamide.

12. The method according to claim 9, wherein the cancer is selected from lung cancer, colorectal cancer, melanoma, pancreatic cancer, hepatocyte carcinoma and breast cancer.

13. The method according to claim 9, wherein the cancer is non-small cell lung carcinoma.
